(19) United States
(12) Patent Application Publication
(10) Pub. No.: US 2009/0317850 A1
(43) Pub. Date: Dec. 24, 2009

(54) Crystal Structure of Human 70KD Ribosomal Protein S6 Kinase 1 Kinase Domain

(76) Inventors: Tomoko Sunami, Naka-Gun (JP); Mari Machida, Moriya-Shi (JP); Sanjeev K. Munshi, Ambler, PA (US); Sujata Sharma, Eagleville, PA (US)

Correspondence Address: MERCK AND CO., INC, P O BOX 2000, RAHWAY, NJ, 07065-0907, US

(21) Appl. No.: 12/481,103

(22) Filed: Jun. 9, 2009

Related U.S. Application Data
(60) Provisional application No. 61/132,761, filed on Jun. 20, 2008.

(51) Int. Cl. C12Q 1/48; C12N 9/12

(52) U.S. Cl. 435/15; 435/194

(57) ABSTRACT

Crystallization of the 70 kDa ribosomal protein S6 kinase polypeptide 1 (p70S6K1) kinase domain for X-ray crystallography analysis to generate the three-dimensional structure of the p70S6K1 kinase domain is described. Further described is the use of the three 5 dimensional structure of p70S6K1 kinase domain for identifying and designing ligands or low molecular weight molecules that specifically bind to and modulate (inhibit) the kinase activity of p70S6K1. These ligands or molecules can be used for the treatment of metabolic disorders such as diabetes and for the treatment of various cancers.

### BACKGROUND OF THE INVENTION

(1) Field of the Invention

The present invention relates to crystallization of the 70kDa ribosomal protein S6 kinase polypeptide 1 (p70S6K1) kinase domain for X-ray crystallography analysis to generate a three-dimensional structure of the p70S6K1kinase domain. The present invention further relates to the use of the three dimensional structure of p70S6K1 kinase domain for identifying and designing ligands or low molecular weight molecules that specifically bind to and modulate (inhibit) the kinase activity of the p70S6K1. These ligands or molecules can be used for the treatment of metabolic disorders such as diabetes and for the treatment of various cancers.

(2) Description of Related Art

The 70kDa ribosomal protein S6 kinase polypeptide 1 belongs to the AGC family of serine-threonine protein kinases. In humans, two forms of 70kDa ribosomal S6 kinases (p70S6K1 and p70S6K2) have been reported and are encoded by two different genes (RPS6KB1 and RPS6KB2), respectively. The two p70S6K1 isoforms differ only in their amino termini by 23 amino acid residues resulting in a 70 kD protein and a 85 kD protein. The isoforms are referred to in the literature as p70^{S6k}/p85^{S6k} or p70αS6 kinase or p70S6K1. Both isoforms share similar activity towards ribosomal protein S6 in vitro but are expressed in different cells and tissues. The two isoforms are produced by two mRNA products and are not a result of post-translational modifications. They are serine/threonine kinases and are known to act on the substrate KKRNRTLSVA (SEQ ID No. 6) (Pai et al., Eur. J. Immunol. 24: 2364-8 (1994); and Leighton et al., FEBS Letters 375: 289-93 (1995)). The gene encoding the human p70αS6K1 was reported by Grove et al., in Mol. Cell. Biol. 11: 5541-5550 (1991) and GenBank under accession number NP_{—}003152. Other p70S6K1 kinase sequences have been described for Mus musculus (GenBank Accession No. SEG_AB015196S, AB015197, and AB015196), Xenopus laevis (GenBank Accession No. X66179), and rat (GenBank Accession No. M57428). A beta form of the S6 kinase was reported in U.S. Pat. No. 6,830,909.

The p70S6K1 is involved in the regulation of cellular growth and metabolism. It plays an important role in the progression of cells from G1 to S phase of the cell cycle and in the initiation of protein synthesis. Over-activation of p70S6K has been implicated in cancer and diabetes and there is currently much interest in identifying inhibitors of p70S6K1 that can be used in anticancer or diabetic therapies (Dan et al., mTOR Complex1-S6K1 signaling: at the crossroads of obesity, diabetes and cancer, TRENDS in Molec. Med. 13: 252-259 (2007); Sinclair et al., The 17q23 Amplicon and Breast Cancer, Breast Cancer Res. Treat. 78: 313-322 (2003); Bärlund et al., Detecting Activation of Ribosomal Protein S6 Kinase by Complementary DNA and Tissue Microarray Analysis, J. Natl. Cancer Inst. 92: 1252-1259 (2000); Klos et al., ErbB2 Increases Vascular Endothelial Growth Factor Protein Synthesis via Activation of Mammalian Target of Rapamycin/p70S6K Leading to Increased Angiogenesis and Spontaneous Metastasis of Human Breast Cancer Cells, Cancer Res. 66(4):2028-2037 (2006); Rojo et al., 4E-Binding Protein 1, A Cell Signaling Hallmark in Breast Cancer that Correlates with Pathologic Grade and Prognosis, Clin. Cancer Res. 13: 81-89 (2007); Noh et al., Breast Cancer Res Treat Sep 6: Epub ahead of print: (2006); Sung Hee Um et al., Absence of S6K1 protects against age- and diet-induced obesity while enhancing insulin sensitivity, Nature 431: 200 - 205 (2004); Manning, Balancing Akt with S6K: implications for both metabolic diseases and tumorigenesis, J. Cell. Biol. 167: 399-403 (2004)).

Activation of p70S6K requires a cascade of reactions that phosphorylate specific amino acid residues of the p70S6K (Pullen et al., Phosphorylation and activation of p70S6K by PDK1, Science 279: 707-710 (1998); Balendran et al., Evidence that 3-phosphoinositide-dependent protein kinase-1 mediates phosphorylation of p70 S6 kinase in vivo at Thr-412 as well as Thr-252, J. Biol. Chem. 274: 37400-27406 (1999); Isotani et al., Immunopurified mammalian target of rapamycin phosphorylates and activates p70 S6 kinase alpha in vitro, J. Biol. Chem. 274: 34493-34498 (1999); Moser et al., Dual requirement for a newly identified phosphorylation site in p70s6k, Mol. Cell. Biol. 17: 5648-55 (1997); Ferrari et al., Activation of p70S6K is associated with phosphorylation of four clustered sites displaying Ser/Thr-Pro motifs, Proc. Natl. Acad. Sci. USA 89: 7282-7285 (1992); Mukhopadhayay et al., An array of insulin-activated, proline-directed serine/threonine protein kinases phosphorylate the p70 S6 kinase, J. Biol. Chem. 267: 3325-3335 (1992).). A schematic arrangement of the various domains of the p70S6K is shown in FIG. 1. First, the C-terminal autoinhibitory domain, which blocks T-loop and hydrophobic motif (HM) phosphorylation, is phosphorylated by ERK (extracellular signal-regulated kinase). This is followed by phosphorylation of the threonine residue at position 412 (T412) in the HM region by a kinase such as mTOR (mammalian target of rapamycin) and the threonine residue at position 252 (T252) in the T-loop by PDK1 (3′-phosphoinositide-dependent kinase-1), which leads to activation of the p70S6K1. PDK1 has been identified as the kinase, which phosphorylates amino acid T252, and several possible kinases, which phosphorylates amino acid T412, have been identified including mTOR. In addition, phosphorylation of the serine residue at position 394 (S394) in the turn motif is also necessary for activation.

In traditional drug discovery, modulators of a biomolecule such as p70S6K1 are identified by screening large collections of ligands or small molecule libraries using an enzymatic assay designed to identify those compounds that modulate the activity of the biomolecule. Direct binding of the ligands or small molecules is evaluated by biophysical methods but these methods do not enable visualization of the binding interaction of the ligand or small molecule to the biomolecule. Thus, traditional drug discovery methods for identifying modulators of a biomolecule are time and personnel intensive.

To overcome the limitations inherent in traditional drug discovery methods, rational or structure-based drug design methods have been developed that involve the use of three-dimensional information about a biomolecule obtained from such techniques as x-ray crystallography and NMR spectroscopy and molecular modeling to identify molecules that modulate activity of the biomolecule. X-ray crystallography depends on the availability of the crystals of the biomolecule. Once crystals are produced, crystallographic data can be generated to provide useful structural information that assists in the design of small molecules that bind to the biomolecule and inhibit its activity in vivo. If the biomolecule is crystallized as a complex with a ligand, one can determine both the shape of the binding pocket, as well as the amino acid residues that interact with the ligand. By knowing the shape and amino acid residues that define the binding pocket, one can design new ligands that will interact favorably with the protein. With such structural information, available computational methods may be used to predict how strong the ligand binding interaction will be. Such methods aid in the design of inhibitors that bind strongly, as well as selectively to the biomolecule. Thus, in order to design efficacious inhibitors of p70S6K1, it is necessary to provide crystal structures of the kinase domain of p70S6K1 bound to ligands.

### BRIEF SUMMARY OF THE INVENTION

The present invention provides a means for a rational approach to discovery and design of ligands and small molecules that can inhibit the activity of human 70kDa ribosomal protein S6 kinase polypeptide 1 (p70S6K1). The present invention provides crystals of the p70S6K1 kinase domain comprising the catalytic domain with or without an inhibitor or other ligand or low molecular weight compound, a method of crystallizing the p70S6K1 kinase domain; and methods of using the three-dimensional coordinates derived from the p70S6K1 kinase domain crystal to identify and or design ligands and small molecules capable of inhibiting p70S6K1 kinase activity.

Therefore, in one aspect, provided is a composition comprising a 70kDa ribosomal protein S6 kinase polypeptide 1 (p70S6K1) kinase domain-ligand complex in crystalline form wherein the crystal has a crystal lattice in a P2₁2₁2₁ space group and has unit cell dimensions of ±5% of a=56.6 Å, b=63.0 Å, and c=98.3 Å or P2₁ space group and has unit cell dimensions of ±5% of a=78.6 Å, b=62.9 Å, c=87.0 Å, and β=94.3 Å.

In particular embodiments of the above aspect, the ligand is an inhibitor of the p70S6K1 kinase activity. In particular aspects, the inhibitor is a ligand or small molecule that inhibits binding of ATP to the p70S6K1 kinase domain. Examples of such ligands include but are not limited to staurosporine.

Further provided is a method for identifying a compound that associates with at least a portion of 70kDa ribosomal protein S6 kinase polypeptide 1 (p70S6K1) kinase domain and inhibits kinase activity of the p70S6K1, the method comprising the steps of (a) obtaining a crystallized complex of p70S6K1 kinase domain complexed with a known inhibitor of the p70S6K1 kinase activity, the crystal belonging to a space group P2₁2₁2₁ and has unit cell dimensions of ±5% of a=56.6 Å, b=63.0 Å, and c=98.3 Å or a P2₁ space group and has unit cell dimensions of ±5% of a=78.6 Å, b=62.9 Å, c=87.0 Å, and β=94.3 Å; (b) obtaining the structural coordinates of the crystallized complex of step (a); (c) generating a three dimensional model of the p70S6K1 kinase domain using the structural coordinates of the amino acids generated in step (b), ±. a root mean square deviation from the backbone atoms of said amino acids of not more than 1.5 angstrom; (d) determining an active site of the p70S6K1 kinase domain from the three dimensional model; (e) performing computer modeling analysis to identify said compound that associates with the p70S6K1 kinase domain; and (f) synthesizing the compound and contacting the compound with the p70S6K1 kinase to determine the ability of the compound to inhibit the kinase activity of the p70S6K1. In particular embodiments, the known inhibitor is staurosporine.

Further provided is a method for identifying a compound that associates with at least a portion of 70kDa ribosomal protein S6 kinase polypeptide 1 (p70S6K1) kinase domain and inhibits kinase activity of the p70S6K1, the method comprising the steps of: (a) obtaining a crystallized p70S6K1 kinase domain, the crystal belonging to a space group P2₁2₁2₁ and has unit cell dimensions of ±5% of a=56.6 Å, b=63.0 Å, and c=98.3 Å or a P2₁ space group and has unit cell dimensions of ±5% of a=78.6 Å, b=62.9 Å, c=87.0 Å, and β=94.3 Å; (b) obtaining the structural coordinates of the crystallized kinase domain of step (a); (c) generating a three dimensional model of the p70S6K1 kinase domain using the structural coordinates of the amino acids generated in step (b), ±. a root mean square deviation from the backbone atoms of said amino acids of not more than 1.5 angstrom; (d) determining an active site of the p70S6K1 kinase domain from the three dimensional model; (e) performing computer modeling analysis to identify the compound that associates with the p70S6K1 kinase domain; and (f) synthesizing the compound and contacting the compound with the p70S6K1 kinase to determine the ability of the compound to inhibit the kinase activity of the p70S6K1.

In particular embodiments of the above methods, the step of performing computer modeling analysis to identify said compound that associates with p70S6K1 kinase domain comprises identifying the compound from a library of compounds. In further embodiments, the step of performing computer modeling analysis to identify the compound that associates with the p70S6K1 kinase domain comprises identifying the compound in a database. In further embodiments, the step of performing computer modeling analysis to identify the compound that associates with the p70S6K1 kinase domain comprises designing the compound from a known p70S6K1 antagonist or partial antagonist.

Further provided is method for making a crystal of the 70kDa ribosomal protein S6 kinase polypeptide 1 (p70S6K1) kinase domain, comprising: (a) providing a solution of the p70S6K1 kinase domain polypeptide and a known inhibitor of the kinase activity of the p70S6K1 kinase domain; (b) mixing the solution with a crystallization solution comprising polyethylene glycol, a buffer, and Li₂SO₄; and (c) incubating the mixture under conditions that facilitate vapor diffusion for a time sufficient to produce the crystal of the p70S6K1 kinase domain wherein the crystal belongs to a space group P2₁2₁2₁ and has unit cell dimensions of ±5% of a=56.6 Å, b=63.0 Å, and c=98.3 Å or a P2₁ space group and has unit cell dimensions of ±5% of a=78.6 Å, b=62.9 Å, c=87.0 Å, and β=94.3 Å.

In particular embodiments of the above method, the pH of the crystallization solution can be between 4 and 7 pH units; the crystallization solution can comprise about 20-35% (w/v) PEG3350, about 100-200 mM Bis-Tris or Na citrate pH 5-6.5, about 200-500 mM Li₂SO₄. In a further still embodiment, the crystallization solution consists essentially of 20-35% (w/v) PEG3350, 100-200 mM Bis-Tris, pH 5-6, 200-500 mM Li₂SO₄.

In further embodiments, the p70SK1 kinase domain comprises an amino acid sequence from between amino acid 90 to amino acid 360 of SEQ ID NO:7. The amino acid sequence can include amino acids 90 through 360 or an amino acid sequence between amino acids 90 through 360 that encodes a polypeptide fragment of the p70S6K1 kinase domain. In further aspects, the p70S6K1 kinase domain comprises the amino acid sequence set forth in SEQ ID NO:3. In particular aspects, the p70S6K1 kinase domain comprises amino acids 3-311 of SEQ ID NO:3. In further aspects, the p70S6K1 kinase domain consists of amino acids 3-311 of SEQ ID NO:3. In further still embodiments, the p70SK1 kinase domain comprises one or more amino acid substitutions, insertions, or deletions. The amino acid substitutions can be conservative or non-conservative. The amino acid substitutions, insertions, or deletions can either have an effect the activity of the kinase or can have no or little effect on the activity of the kinase.

### BRIEF DESCRIPTION OF THE DRAWINGS

FIG. 1 shows the structure of the p70S6K1 protein. Shown are the catalytic domain, the hydrophobic motif (HM), and the auto-inhibitory domain with the phosphorylation sites therein. PDK1 phosphorylates the threonine residue at position 252 (T252) in the catalytic domain; mTOR phosphorylates the serine residue at position 394 (S394) and the threonine residue at position 412 (T412) in the HM, and ERK phosphorylates several amino acids in the auto-inhibitory domain.

FIG. 2 shows the nucleotide sequence of nucleic acid encoding the p70S6K1 kinase domain polypeptide Variant 9 (v9) comprising amino acid residues 75-399 (SEQ ID NO:1).

FIG. 3 shows the amino acid sequence of the p70S6K1 kinase domain polypeptide v9 His6-tagged (SEQ ID NO:2).

FIG. 4 shows the amino acid sequence of the p70S6K1 kinase domain polypeptide v9 (SEQ ID NO:3).

FIG. 5 shows a ribbon diagram of the three-dimensional structure of the p70S6K1 kinase domain v9 drawn according to the coordinates in the P2₁ space group. The diagram shows the inhibitor staurosporine bound to the hydrophobic cleft between the N- and C-lobes.

FIG. 6 shows a ribbon diagram of the three-dimensional structure of the p70S6K1 kinase domain v9 drawn according to the coordinates in the P2₁2₁2₁ space group. The diagram shows the inhibitor staurosporine bound to the hydrophobic cleft between the N- and C-lobes.

FIG. 7A to FIG. 7JJJJJJ shows the P2₁ space group atomic coordinates for the p70S6K1 kinase domain crystal.

FIG. 8A to FIG. 8RRR shows the P2₁2₁2₁ space group atomic coordinates for the p70S6K1 kinase domain crystal.

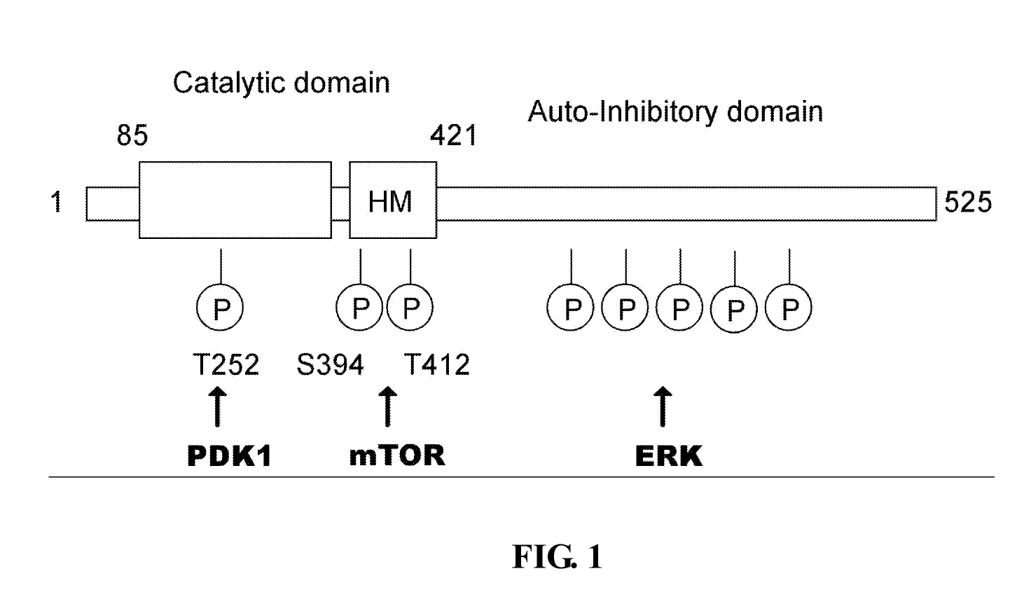

### DETAILED DESCRIPTION OF THE INVENTION

The present invention relates to crystalline forms of human 70kDa ribosomal protein S6 kinase polypeptide 1 (p70S6K1) kinase domain, where the crystals are of sufficient quality and size to allow for the determination of the three-dimensional X-ray diffraction structure of the kinase domain to a resolution of about 2.8 angstrom to about 3.4 angstroms in P2₁ and P2₁2₁2₁ space groups, respectively. The invention also relates to methods for preparing and crystallizing the p70S6K1 kinase domain polypeptide. The crystalline forms of the p70S6K1 kinase domain, as well as information derived from their crystal structures, can be used to analyze and modify p70S6K1 kinase activity as well as to identify compounds that interact with the kinase domain polypeptide.

The crystals include both apo crystals and co-crystals. The apo crystals generally comprise substantially pure p70S6K1 kinase domain polypeptide. The co-crystals generally comprise substantially pure p70S6K1 kinase domain polypeptide with a ligand bound to the kinase domain polypeptide.

It should be understood that the crystalline p70S6K1 kinase domain polypeptide is not limited to that obtainable from naturally occurring or native p70S6K1 kinases. The crystals include mutants that have one or more amino acid insertions, deletions, or substitutions in the native p70S6K1 kinase domain. Therefore, mutants of native p70S6K1 kinase domain polypeptides are obtained by replacing at least one amino acid residue in a native p70S6K1 kinase domain with a different amino acid residue, or by adding or deleting amino acid residues within the native domain or at the N- or C-terminus of the native domain, and have substantially the same three-dimensional structure as the native p70S6K1 kinase domain polypeptide from which the mutant is derived.

By having substantially the same three-dimensional structure is meant as having a set of atomic structure coordinates from an apo- or co-crystal that have a root mean square deviation of less than or equal to about 2 angstroms when superimposed with the atomic structure coordinates of the native p70S6K1 kinase domain from which the mutant is derived when at least about 50% to about 100% of the alpha carbon atoms of the native p70S6K1 kinase domain are included in the superposition.

In some instances, it may be particularly advantageous or convenient to substitute, delete, and/or add amino acid residues to a native p70S6K1 kinase domain in order to provide convenient cloning sites in the cDNA encoding the domain, to aid in purification of the domain, and the like. Such substitutions, deletions, and/or additions, which do not substantially alter the three dimensional structure of the native p70S6K1 kinase domain will be apparent to those skilled in the art.

It should be noted that the mutant polypeptides contemplated herein need not exhibit p70S6K1 kinase activity. Indeed, amino acid substitutions, additions, or deletions that interfere with the kinase activity of the p70S6K1 kinase but which do not significantly alter the three-dimensional structure of the kinase domain are also included. Such crystalline polypeptides, or the atomic structure coordinates obtained therefrom, can be used to identify compounds that bind to the native kinase domain and which may affect the activity or the native kinase domain.

The derivative crystals of the invention generally comprise a crystalline p70S6K1 kinase domain polypeptide in non-covelent/covalent association with one or more heavy metal atoms. The polypeptide may correspond to a native or a mutated p70S6K1 kinase domain polypeptide. Heavy metal atoms useful for providing derivative crystals include, by way of example and not limitation, gold and mercury. Alternatively, derivative crystals can be formed from proteins which have heavy atoms incorporated into one or more amino acids, such as seleno-methionine substitutions for methionine.

The co-crystals of the p70S6K1 kinase domain generally comprise a crystalline p70S6K1 kinase domain polypeptide in association with one or more compounds bound to the kinase domain polypeptide. The association may be covalent or non-covalent.

### Production of Polypeptides

The native and mutated p70S6K1 kinase domain polypeptides described herein may be isolated from natural sources or produced by methods well known to those skilled in the art of molecular biology. Expression vectors to be used may contain a native or mutated p70S6K1 kinase domain polypeptide coding sequence and appropriate transcriptional and/or translational control signals. These methods include in vitro recombinant DNA techniques, synthetic techniques and in vivo recombination/genetic recombination. See, for example, the techniques described in Maniatis et al., 1989, Molecular Cloning: A Laboratory Manual, Cold Spring Harbor Laboratory, NY; and Ausubel et al., 1989, Current Protocols in Molecular Biology, Greene Publishing Associates and Wiley Interscience, NY.

A variety of host-expression vector systems may be utilized to express the p70S6K1 kinase domain polypeptide coding sequence. These include but are not limited to microorganisms such as bacteria transformed with recombinant bacteriophage DNA, plasmid DNA or cosmid DNA expression vectors containing the p70S6K1 kinase domain polypeptide coding sequence; yeast or filamentous fungi transformed with recombinant yeast expression vectors containing the p70S6K1 kinase domain coding sequence; insect cell systems infected with recombinant virus expression vectors (e.g. baculovirus) containing the p70S6K1 kinase domain polypeptide coding sequence; plant cell systems infected with recombinant virus expression vectors (e.g., cauliflower mosaic virus, CaMV; tobacco mosaic virus, TMV) or transformed with recombinant plasmid expression vectors (for example, Ti plasmid) containing the p70S6K1 kinase domain polypeptide coding sequence; or animal cell systems. The expression elements of these systems vary in their strength and specificities. Depending on the host/vector system utilized, any of a number of suitable transcription and translation elements, including constitutive and inducible promoters such as pL of bacteriophage μ, plac, ptrp, ptac (ptrp-lac hybrid promoter) and the like may be used; when cloning in insect cell systems, promoters such as the baculovirus polyhedrin promoter may be used; when cloning in plant cell systems, promoters derived from the genome of plant cells (for example, heat shock promoters; the promoter for the small subunit of RUBISCO; the promoter for the chlorophyll a/b binding protein) or from plant viruses (e.g., the 35S RNA promoter of CaMV; the coat protein promoter of TMV) may be used; when cloning in mammalian cell systems, promoters derived from the genome of mammalian cells (e.g., metallothionein promoter) or from mammalian viruses (e.g., the adenovirus late promoter; the vaccinia virus 7.5K promoter) may be used; when generating cell lines that contain multiple copies of the p70S6K1 kinase domain coding sequence, SV40-, BPV- and EBV-based vectors may be used with an appropriate selectable marker.

### Crystallization of Polypeptides and Characterization of Crystal Structure

The apo, derivative and co-crystals of the p70S6K1 kinase domain polypeptide can be obtained by techniques well-known in the art of protein crystallography, including batch, liquid bridge, dialysis, vapor diffusion, and the like (See for example, McPherson, 1982, Preparation and Analysis of Protein Crystals, John Wiley, NY; McPherson, 1990, Eur. J. Biochem. 189:1-23; Webber, 1991, Adv. Protein Chem. 41:1-36; Crystallization of Nucleic Acids and Proteins, Edited by Ducruix and Giege, Oxford University Press; Protein Crystallization Techniques, Strategies, and Tips, Edited by Bergfors, International University Line, 1999). Generally, the apo- or co-crystals of the p70S6K1 kinase domain polypeptide are grown by placing a substantially pure p70S6K1 kinase domain polypeptide in an aqueous buffer containing a precipitant at a concentration just below that necessary to precipitate the protein. Water is then removed from the solution by controlled evaporation to produce crystallizing conditions, which are maintained until crystal growth ceases.

In a further embodiment of the p70S6K1 kinase domain polypeptide crystals, apo or co-crystals are grown by vapor diffusion. In this method, the polypeptide/precipitant solution is allowed to equilibrate in a closed container with a larger aqueous reservoir having a precipitant concentration optimal for producing crystals. Generally, less than about 10 μL of substantially pure p70S6K1 kinase domain polypeptide solution is mixed with an equal volume of reservoir solution, giving a precipitant concentration about half that required for crystallization. This solution is suspended as a droplet underneath a coverslip, which is sealed onto the top of a reservoir. The sealed container is allowed to stand, from one day to one year, usually for about 1-6 weeks, until crystals grow.

Crystallization of the p70S6K1 kinase domain polypeptide to produce co-crystals can be carried out as described below and in the Examples. As described, the purified p70S6K1 kinase domain polypeptide samples are concentrated to 15 mg/mL and the ligand staurosporine is added to a final concentration of 1.0 mM. Orthorhombic and monoclinic crystal forms are grown by vapor diffusion at 20° C. by mixing equal volume of protein solution with a crystallization solution consisting of 20-35% (w/v) PEG3350, 100-200 mM Bis-Tris, pH 5-6, 200-300 mM Li₂SO₄. To produce apo-crystals, the ligand is omitted in the above protocol.

Crystals may be frozen prior to data collection. The crystals can be cryo-protected with for example, either (a) 20-30% saturated glucose present in the crystallization setup, (b) ethanol added to 15-20%, (c) ethylene glycol added to 10-20% and PEG10,000 brought up to 25%, or (d) glycerol added to 15%. The crystals can be either briefly immersed in the cryo-protectant or soaked in the cryo-protectant for periods as long as a day. Freezing can be accomplished by immersing the crystal in a bath of liquid nitrogen or by placing the crystal in a stream of nitrogen gas at 100 K. As shown in the examples, the crystals can be transferred to a cryoprotection buffer comprising 15%-20% (w/v) glycerol with 20-35% (w/v) PEG3350 (corresponding to the crystallization condition), 200-300 mM Li₂SO₄, 100-200 mM Bis-Tris pH 5-6. Crystals can then be frozen by plunging into liquid nitrogen at 100° K.

The mosaic spread of the frozen crystals can sometimes be reduced by annealing, wherein the stream of cold nitrogen gas is briefly blocked, allowing the frozen crystal to thaw momentarily before re-freezing in the nitrogen gas stream. Another technique which is sometimes helpful in data collection was to center one of the ends of the hexagonal bipyramid in the x-ray beam, rather than the mid portion of the crystal. The mosaic spread could sometimes be reduced by this technique.

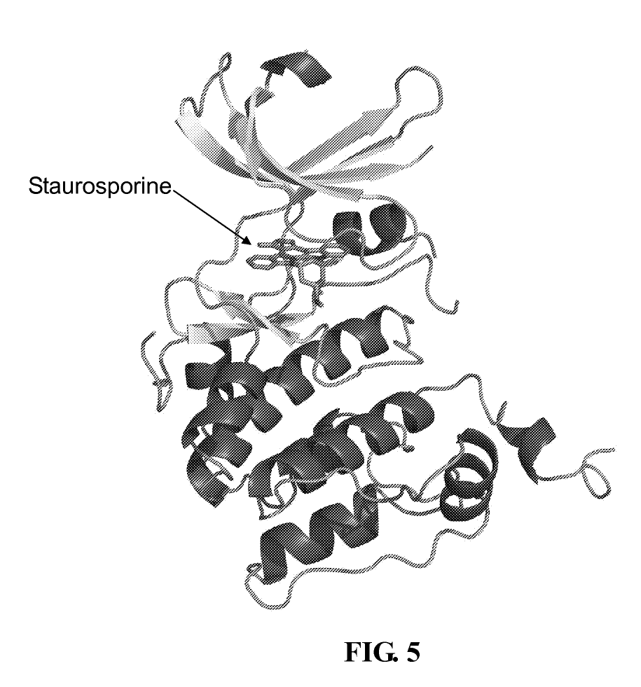

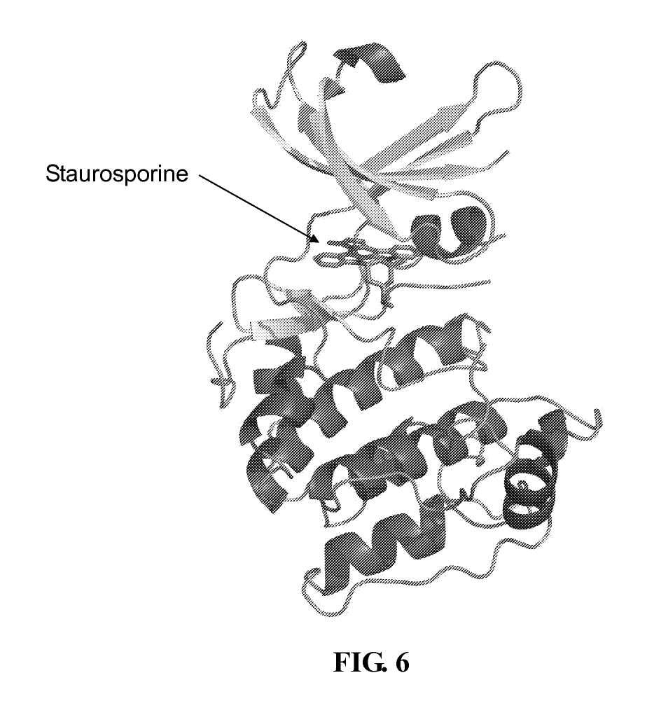

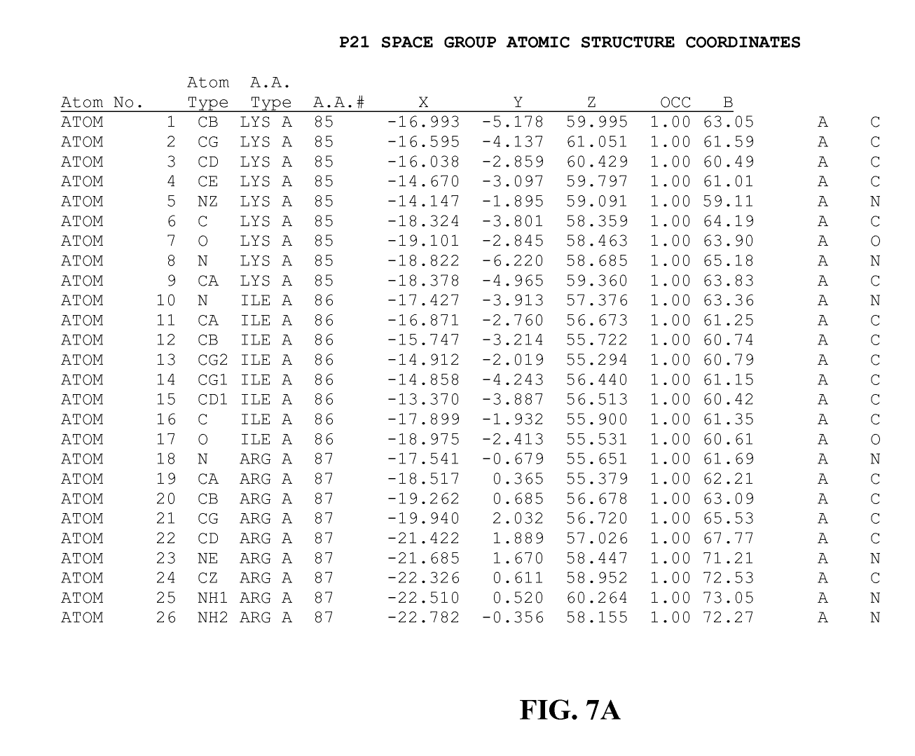

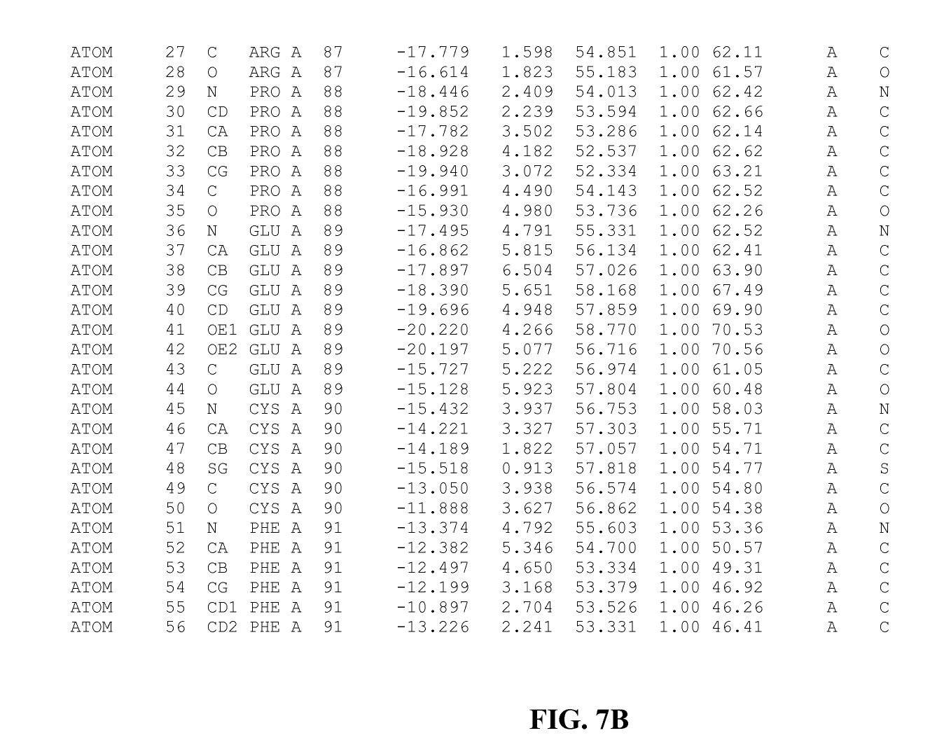

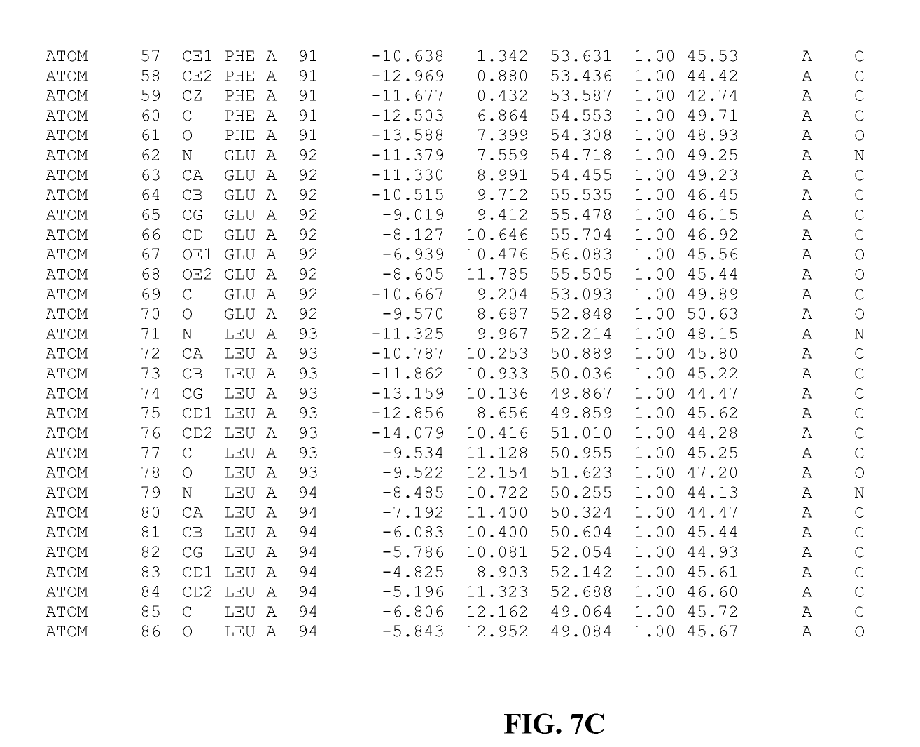

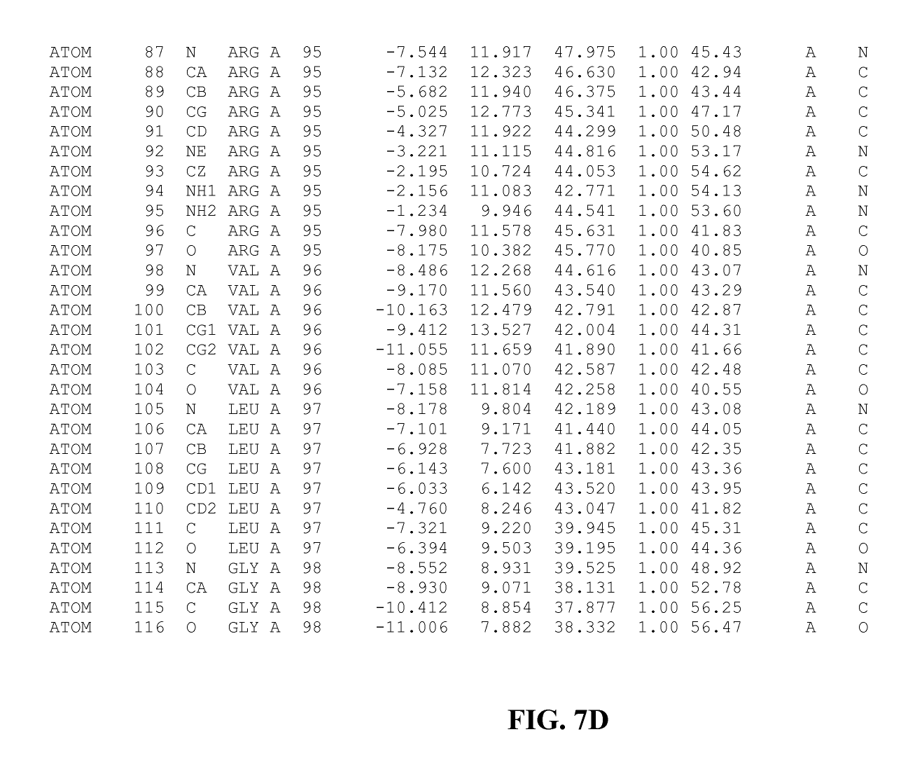

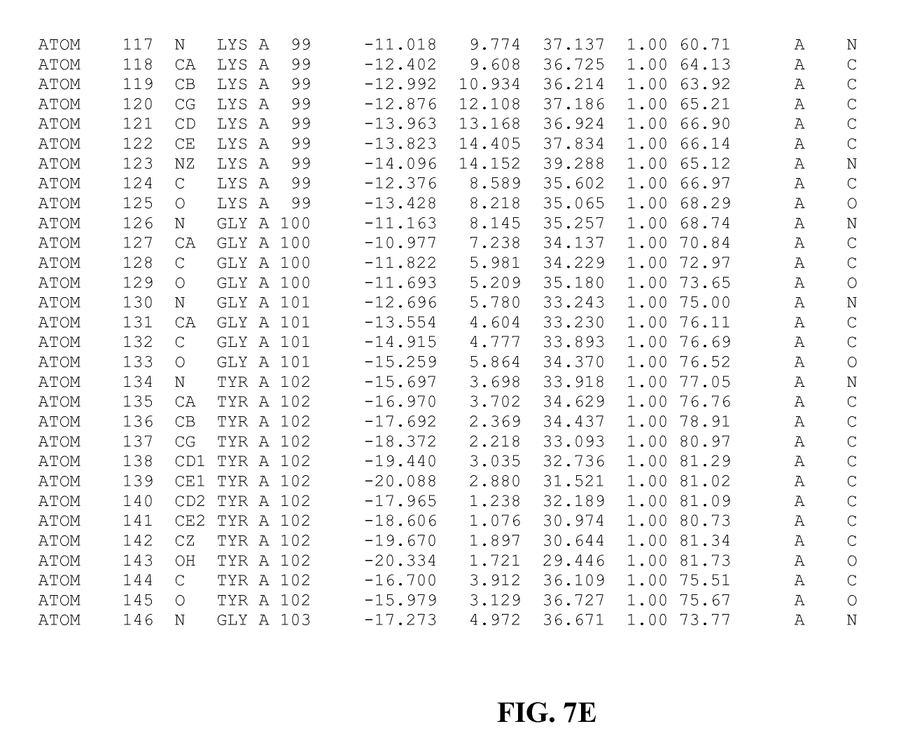

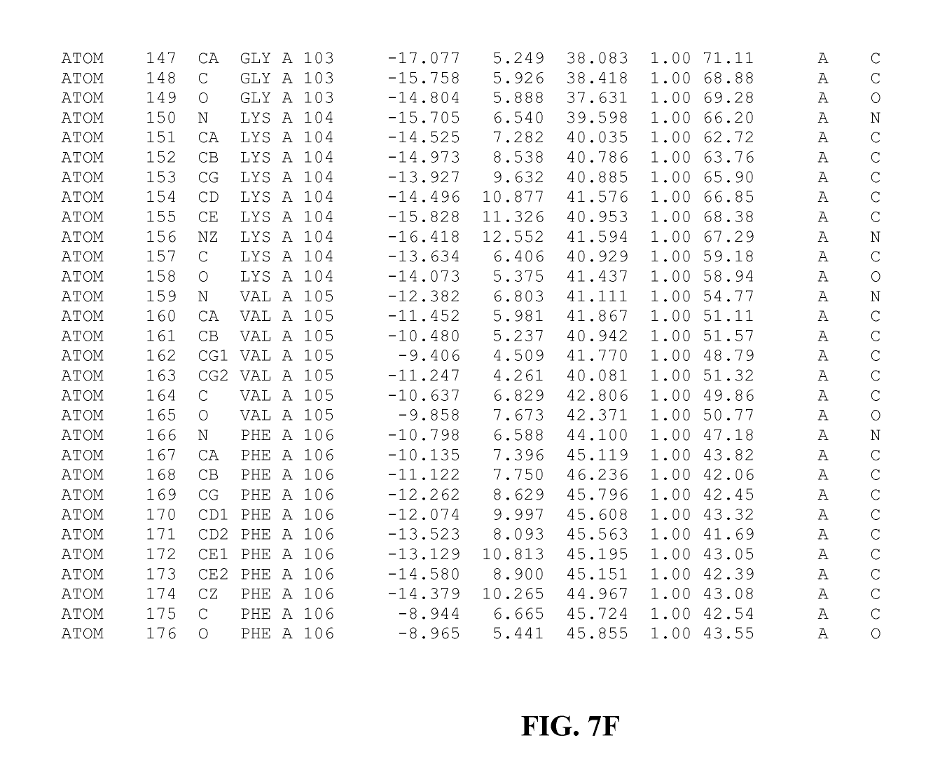

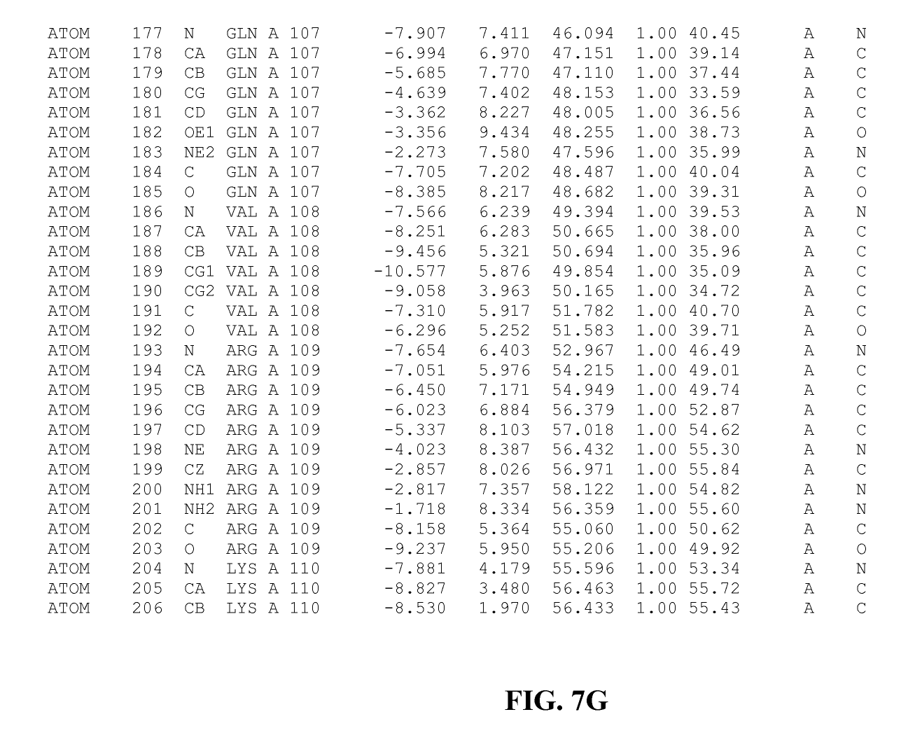

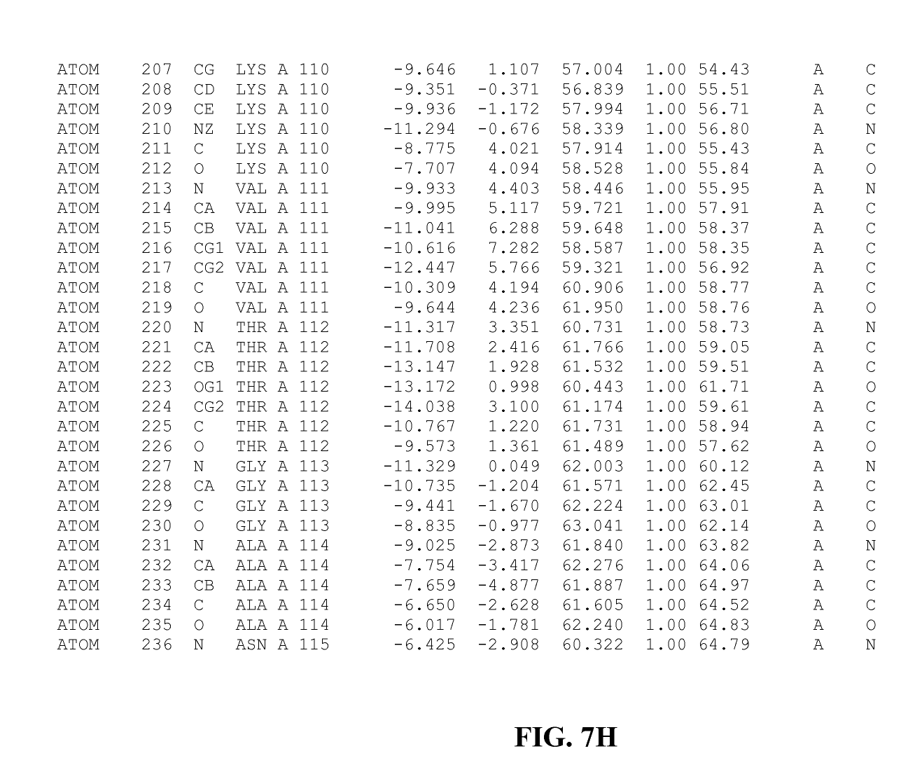

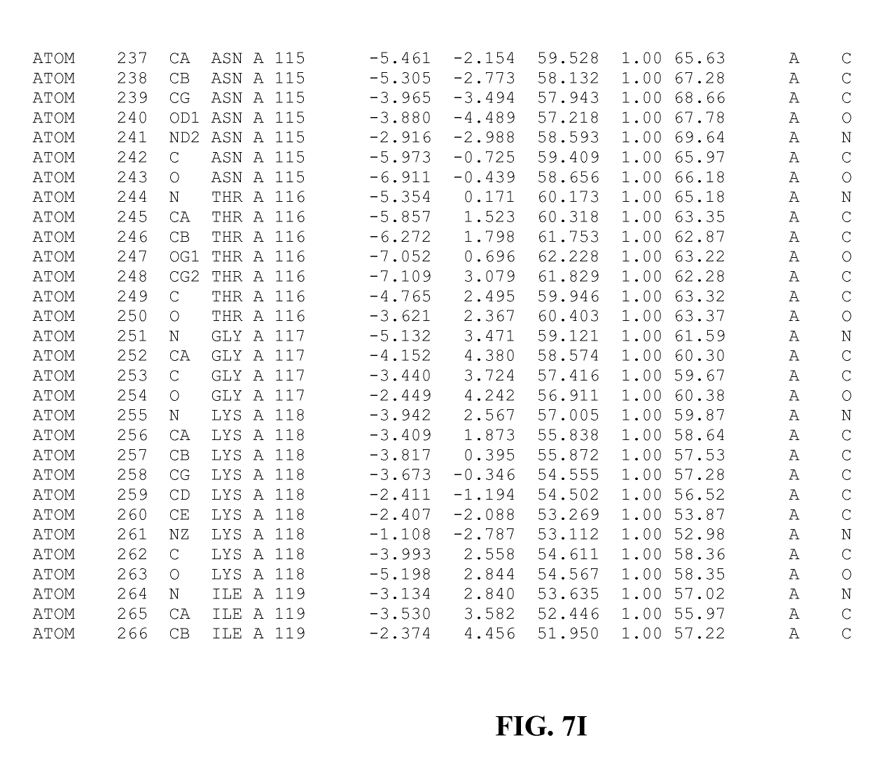

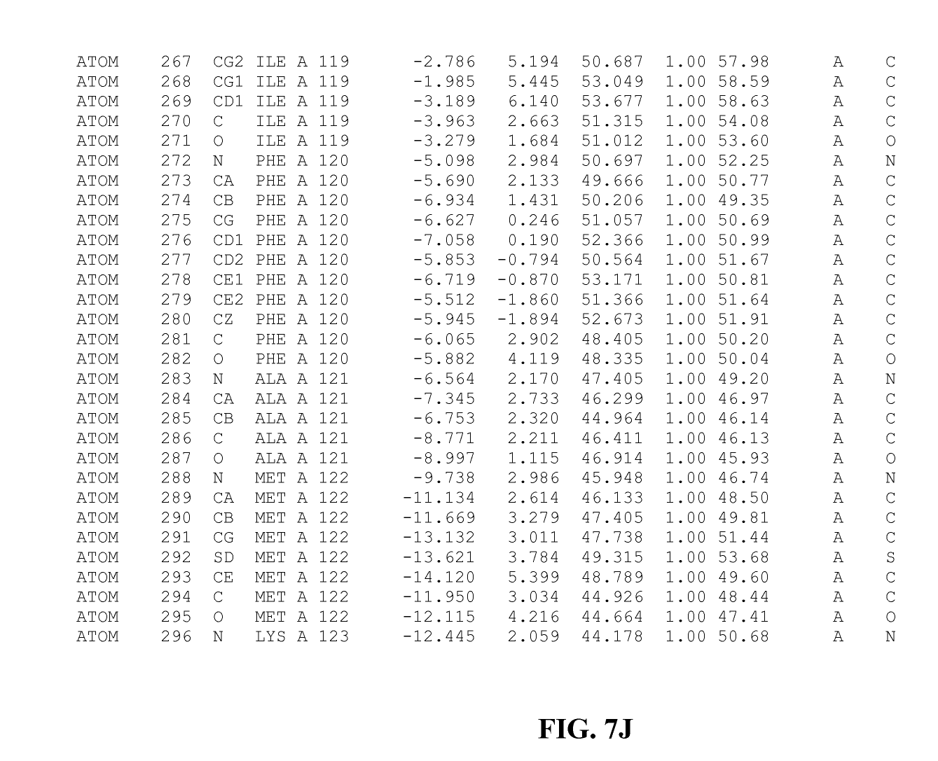

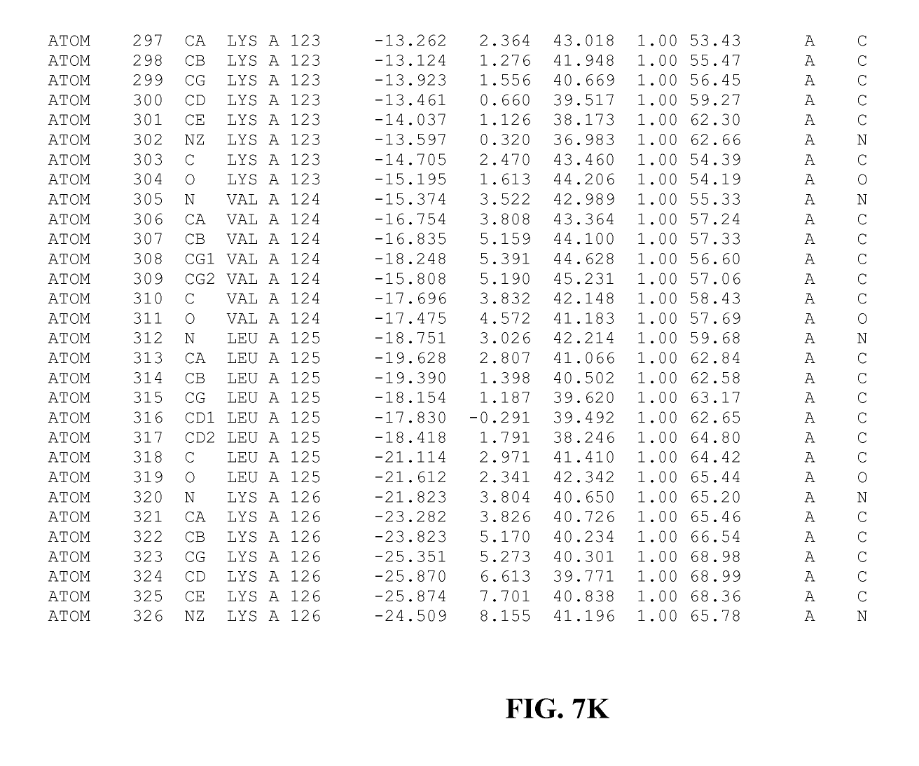

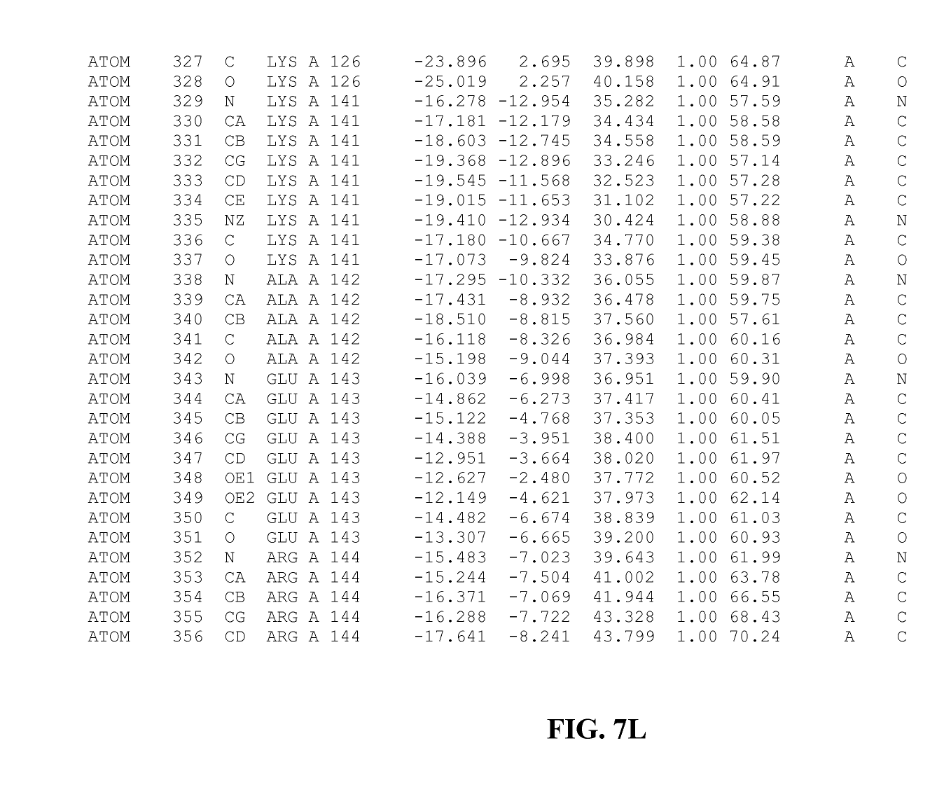

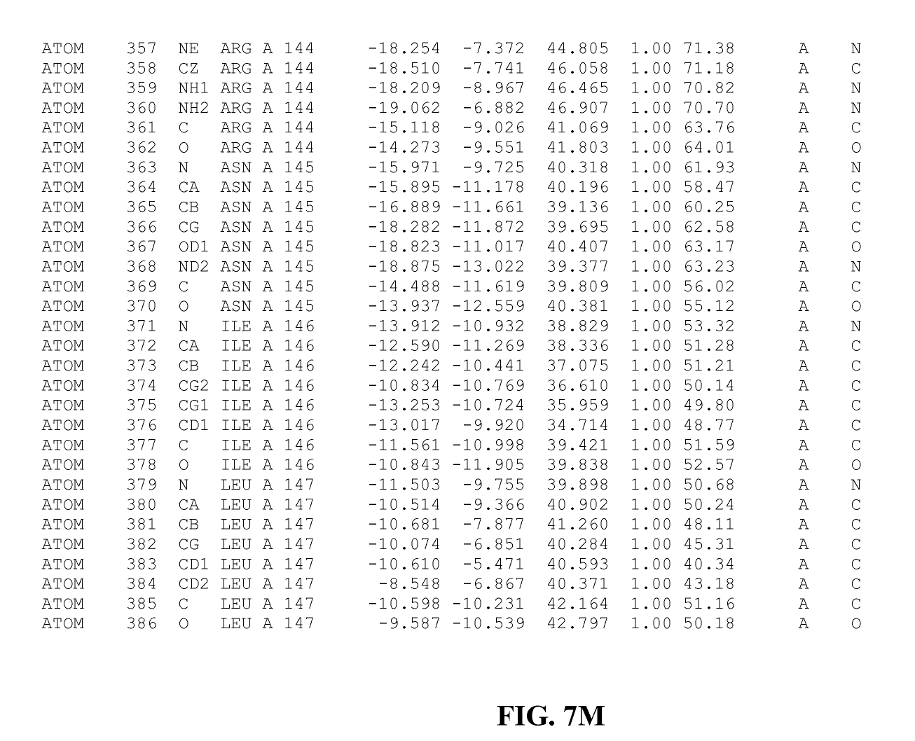

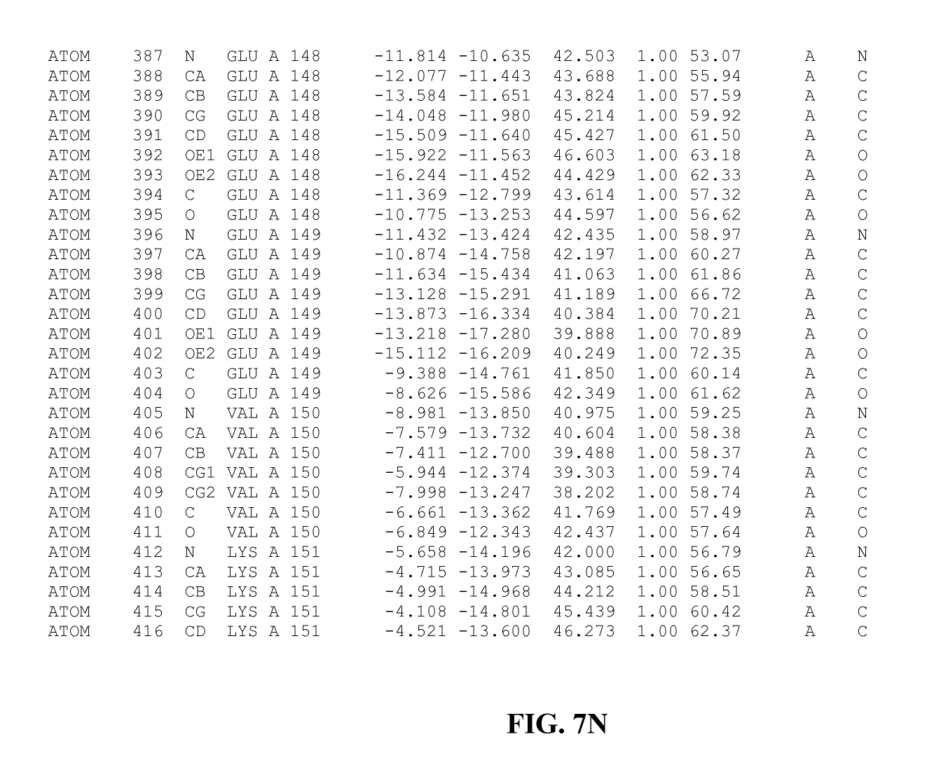

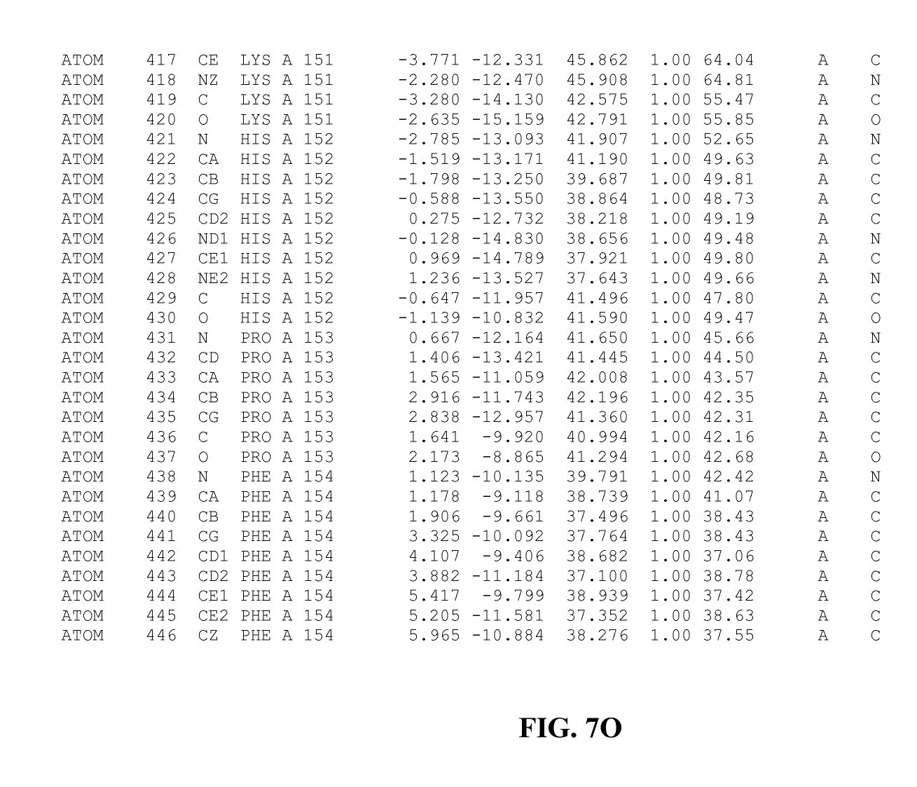

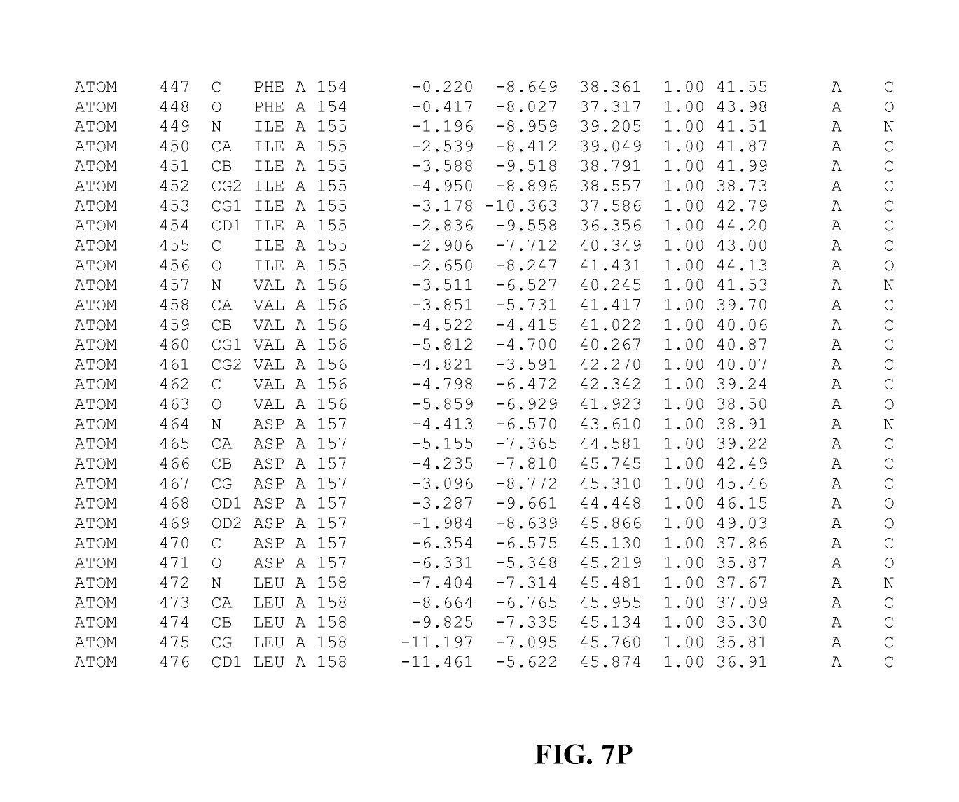

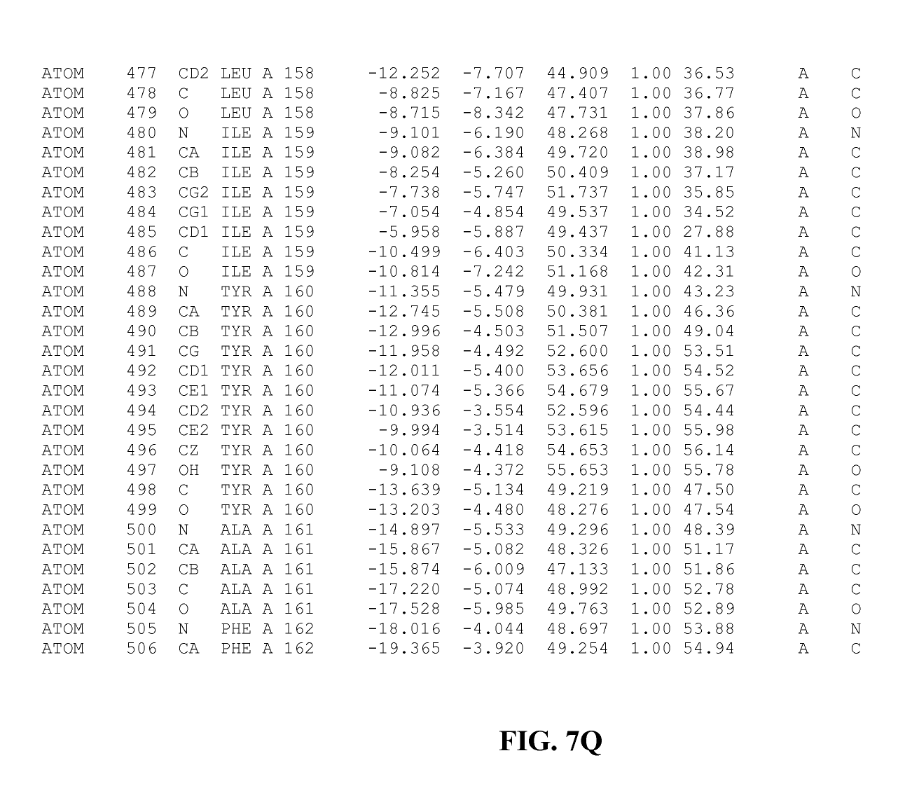

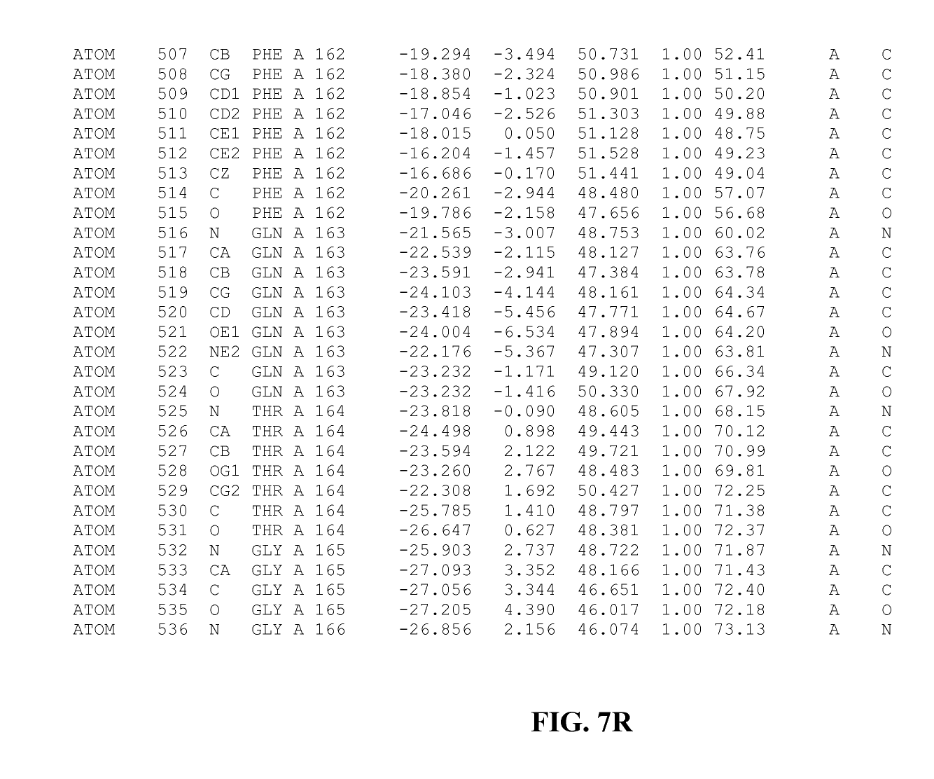

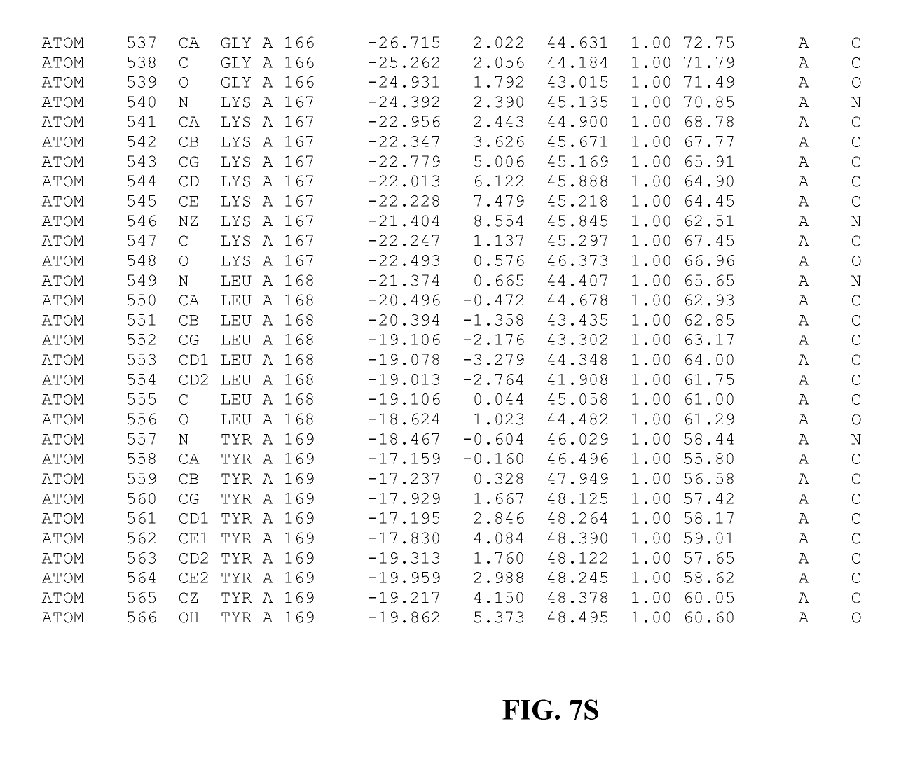

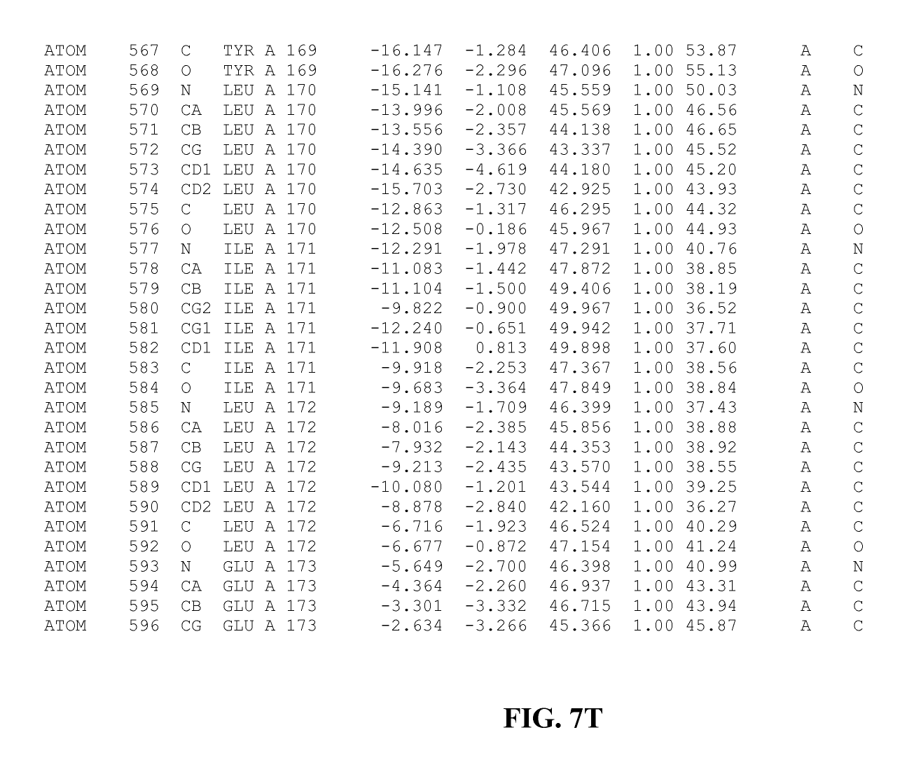

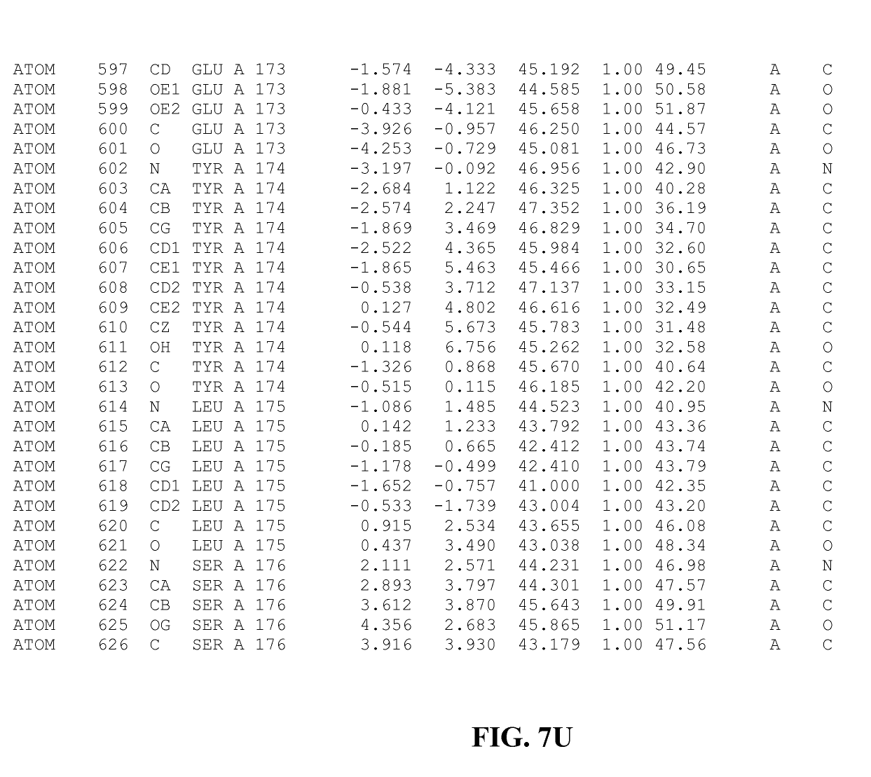

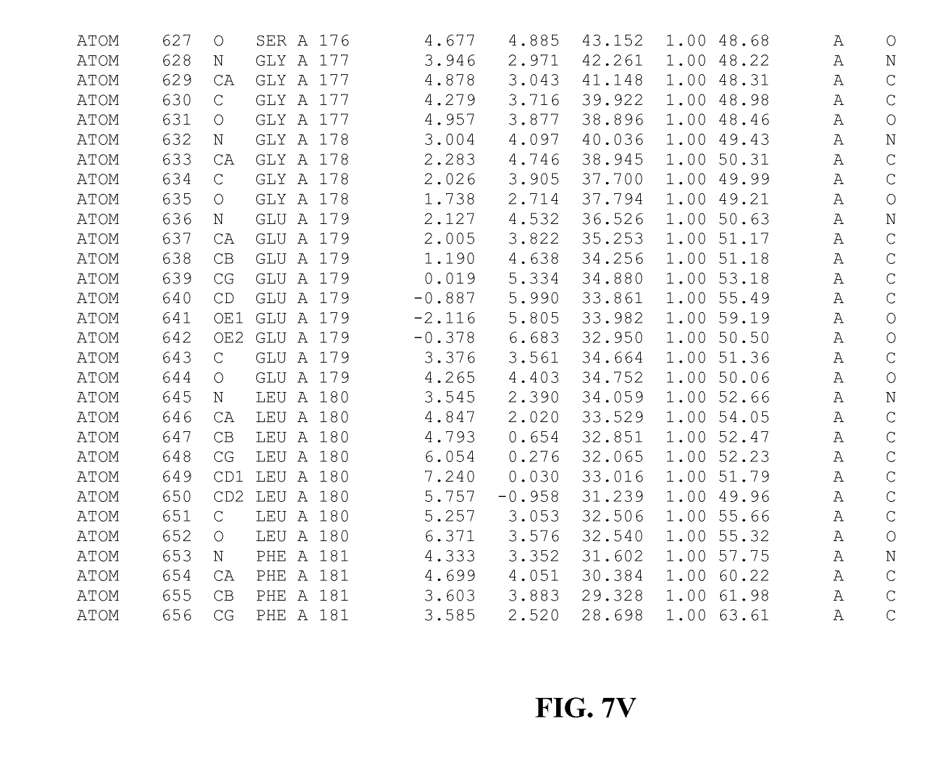

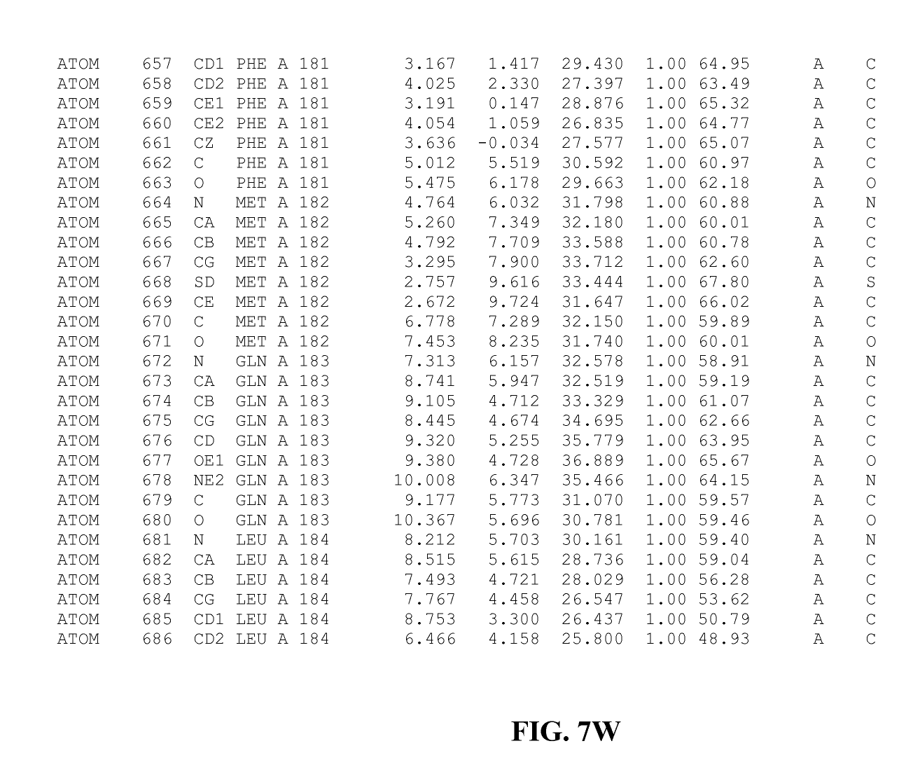

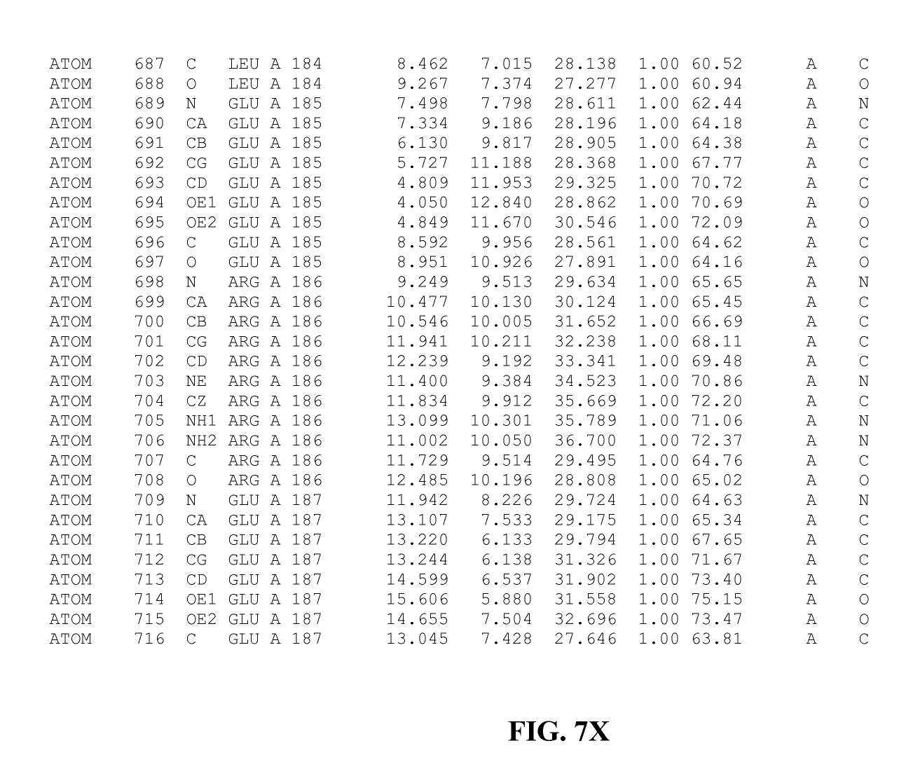

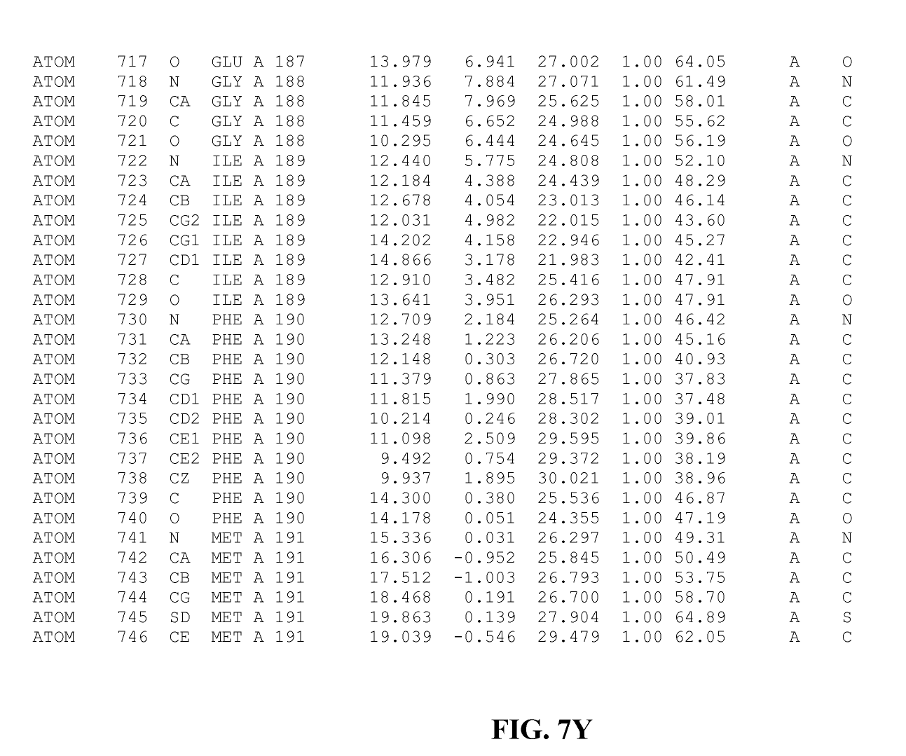

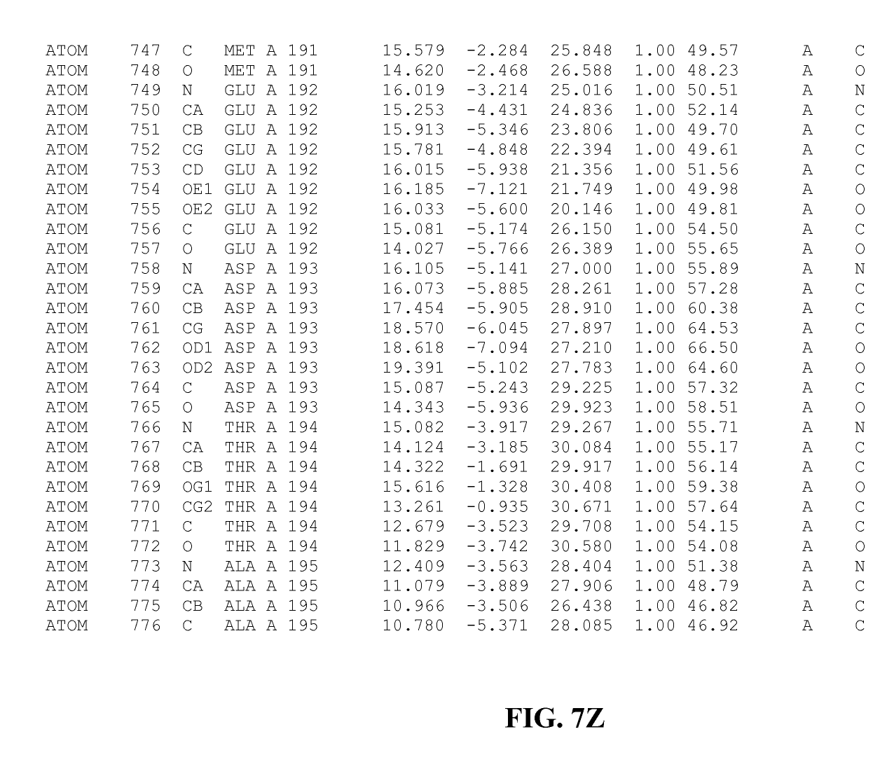

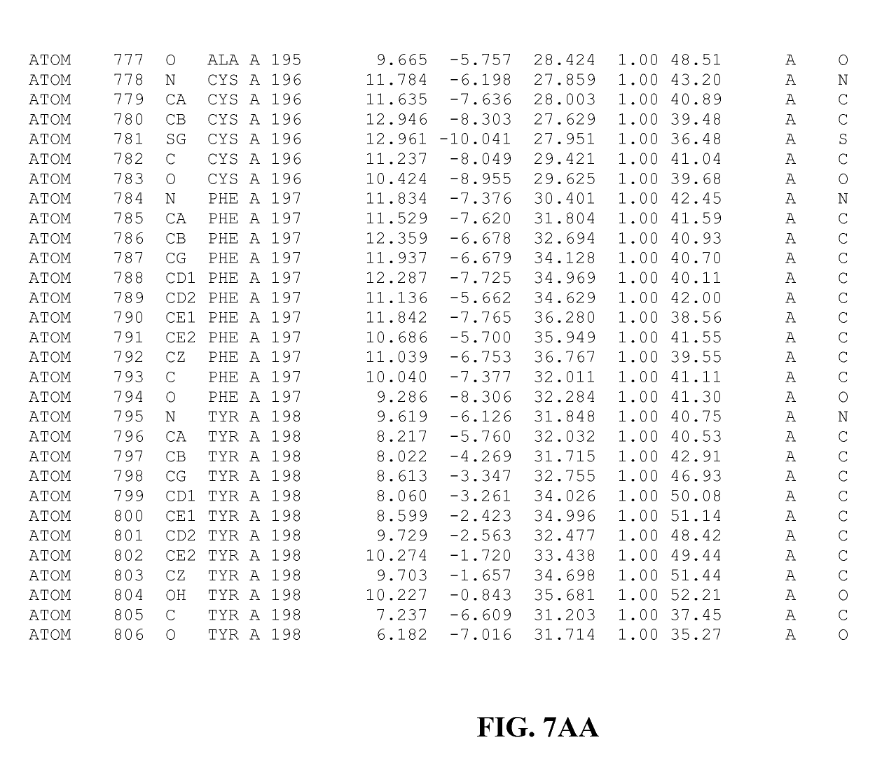

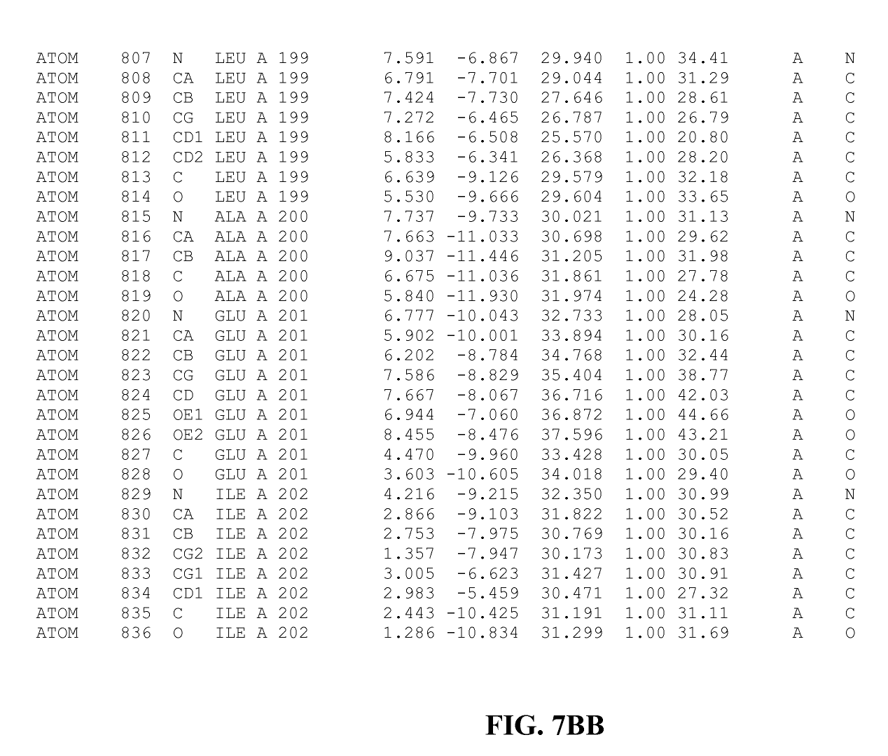

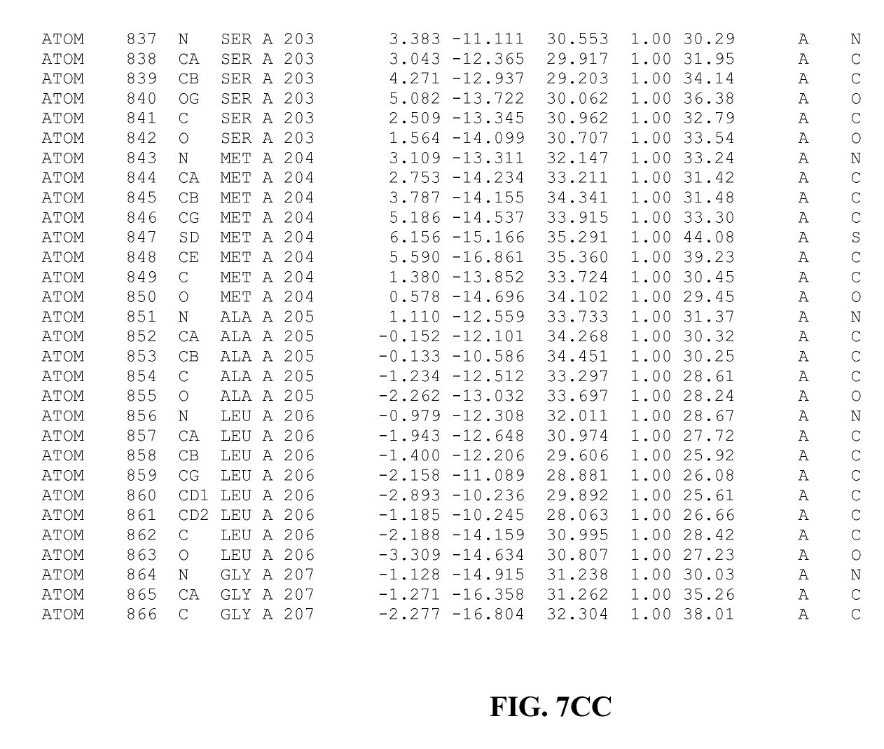

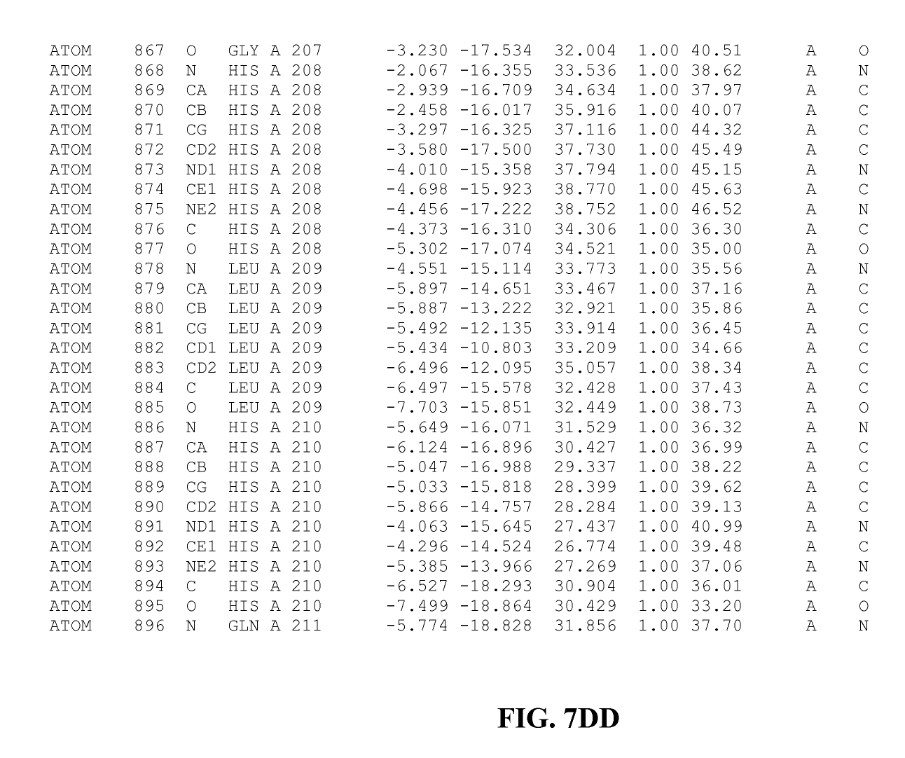

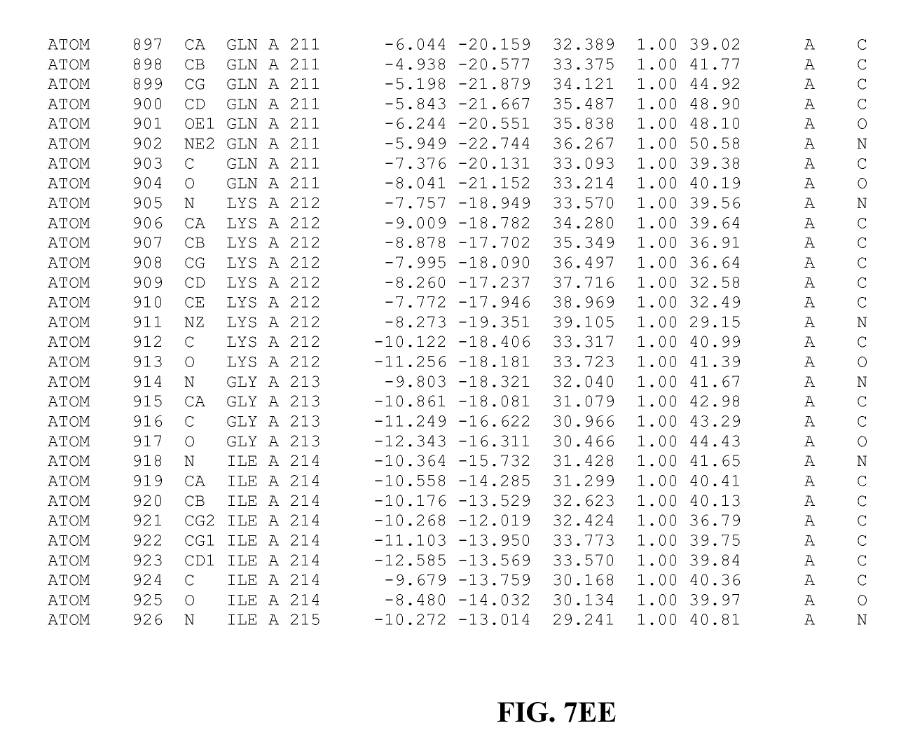

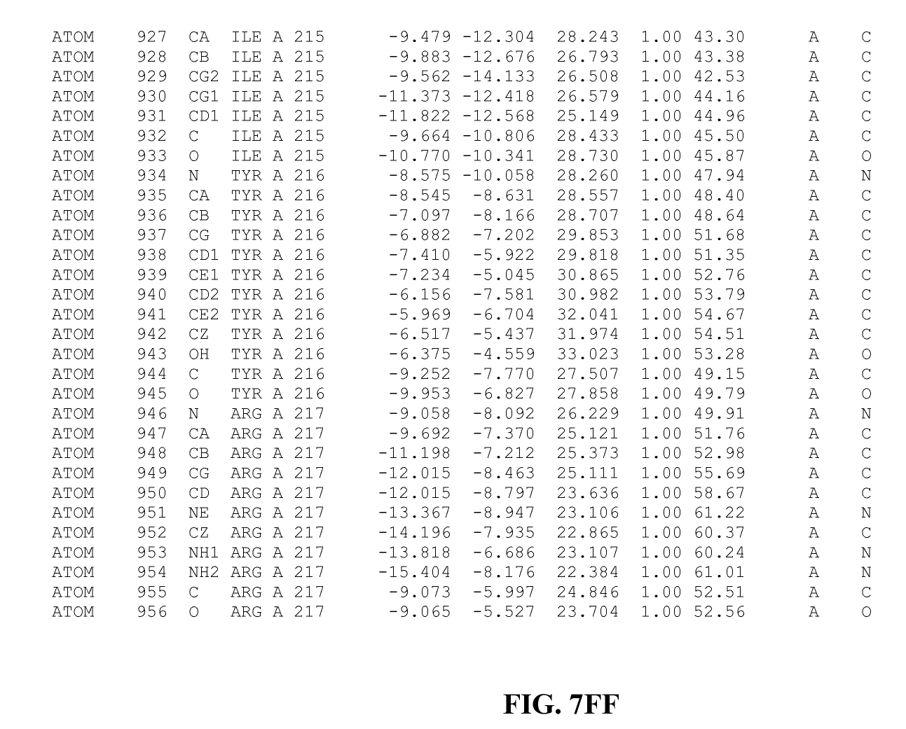

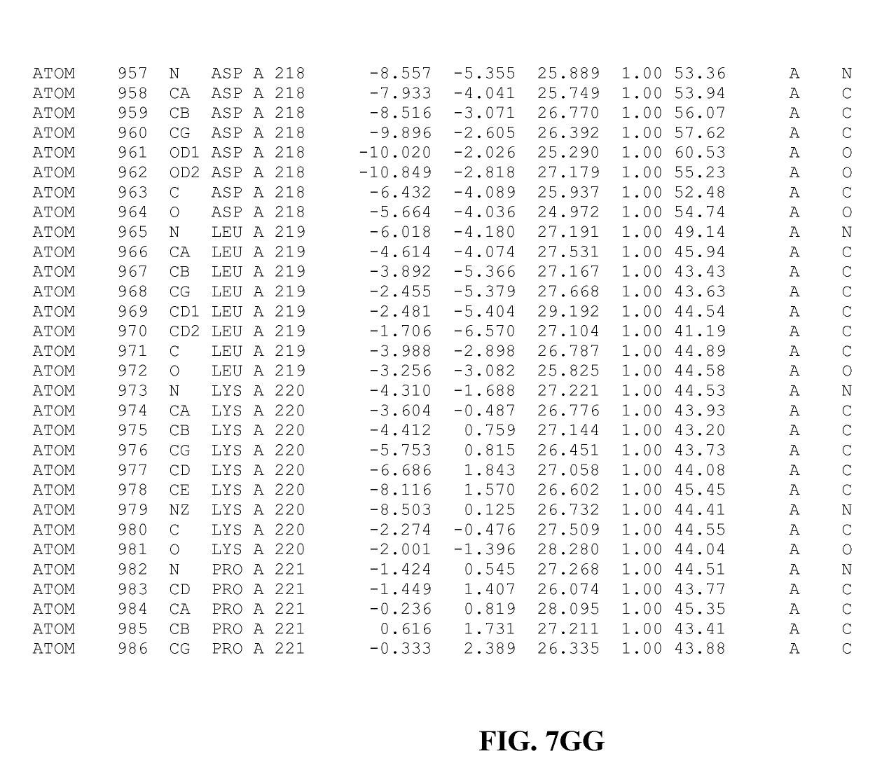

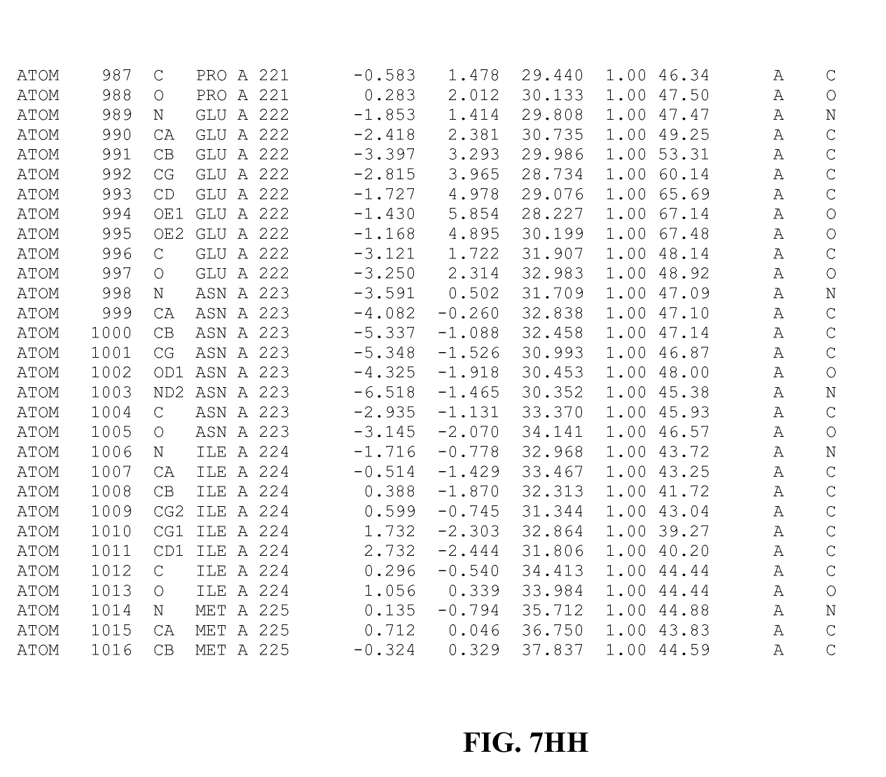

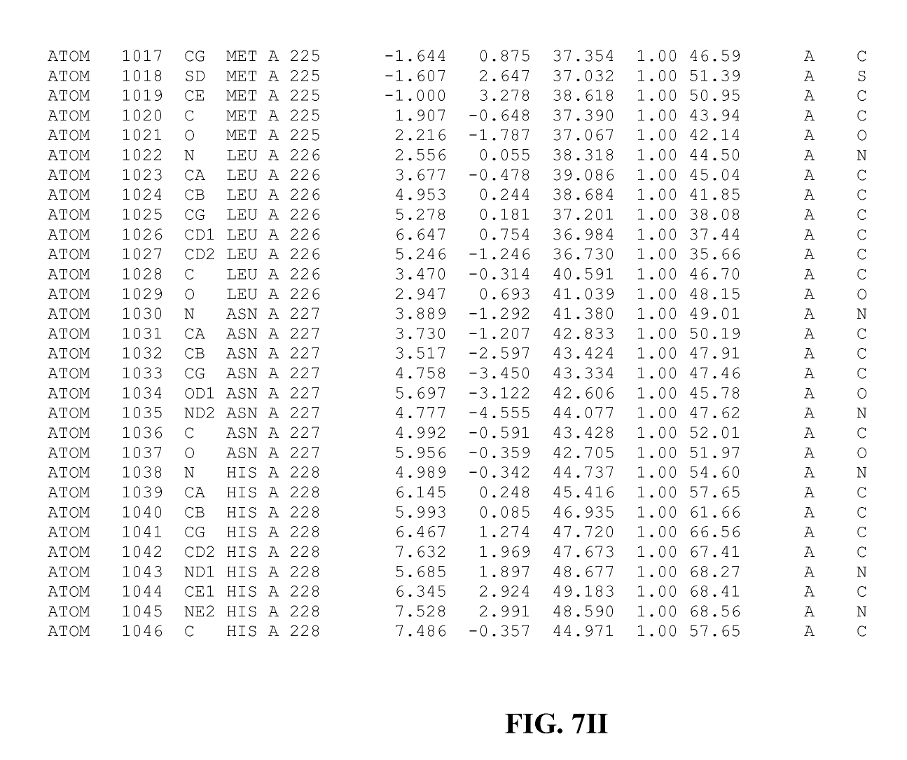

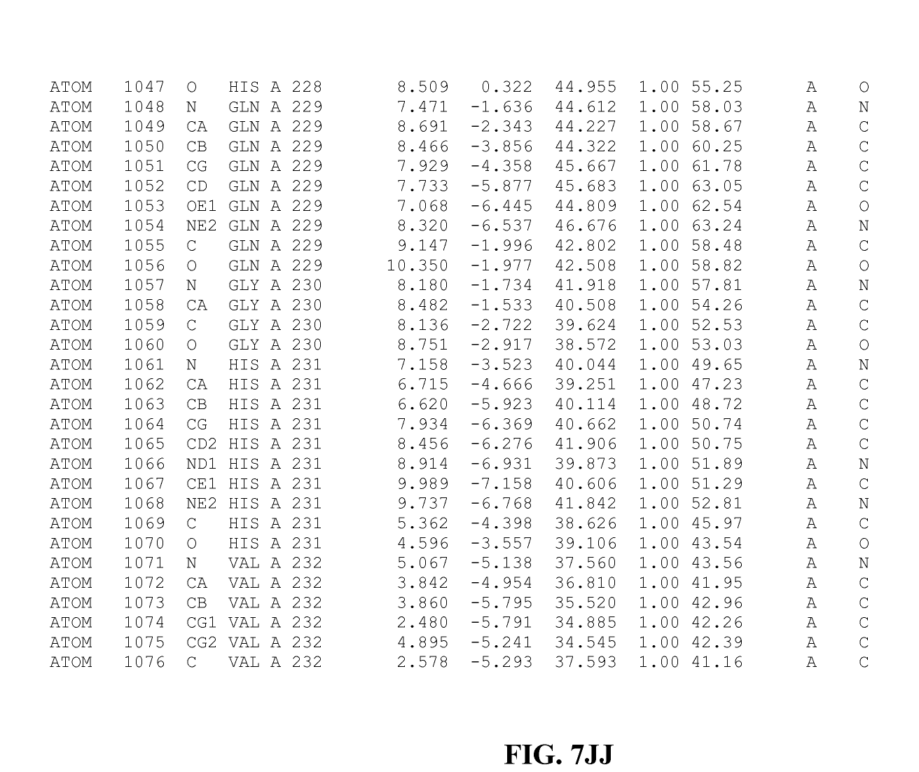

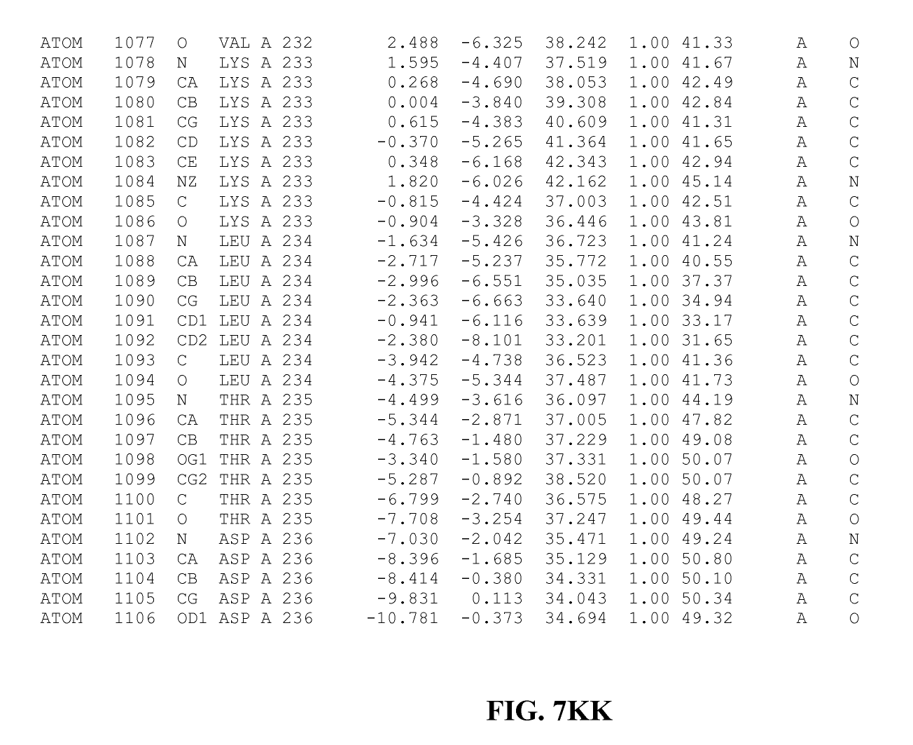

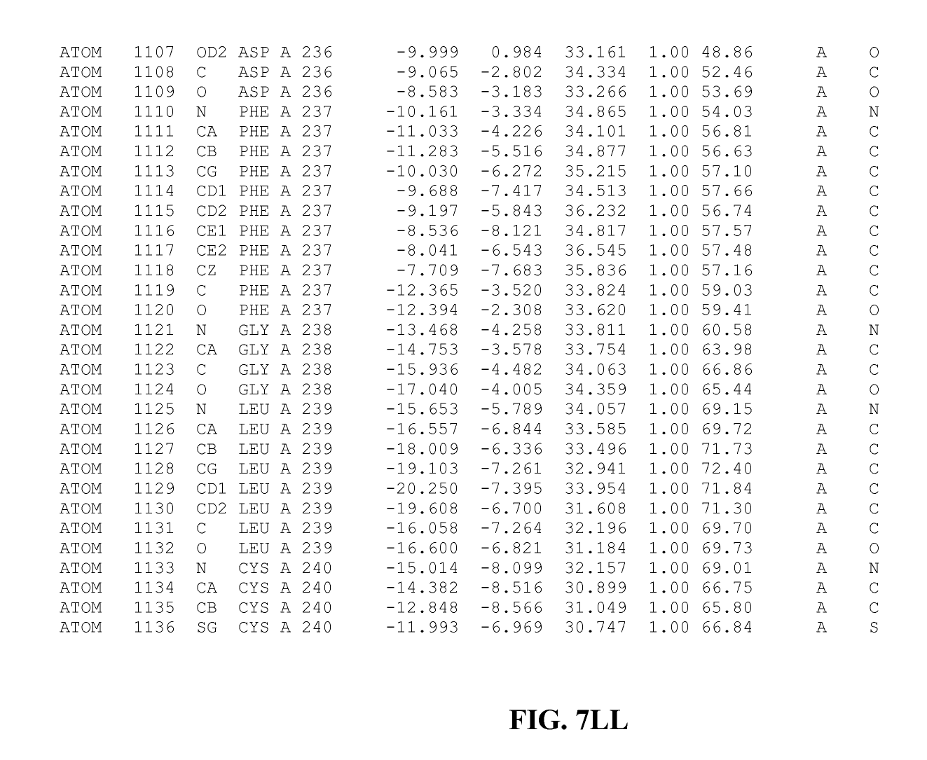

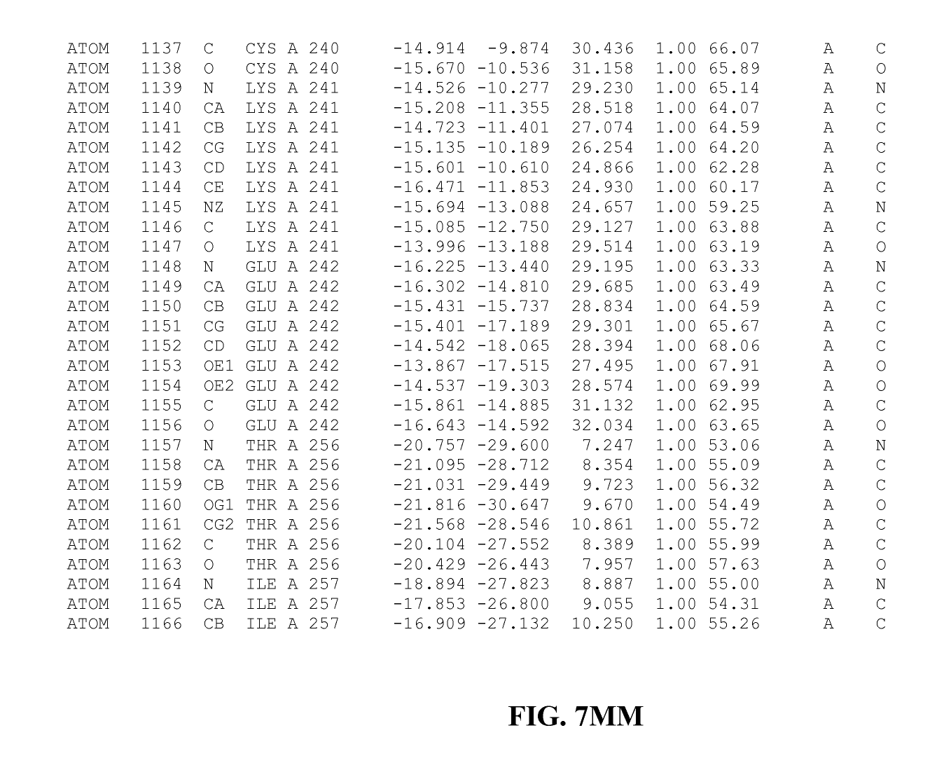

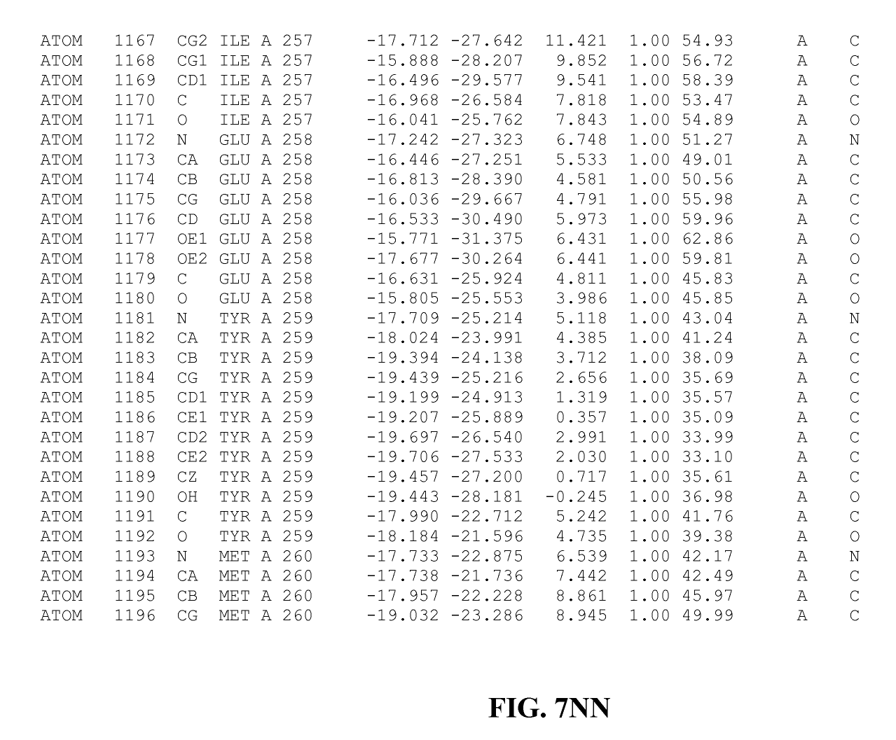

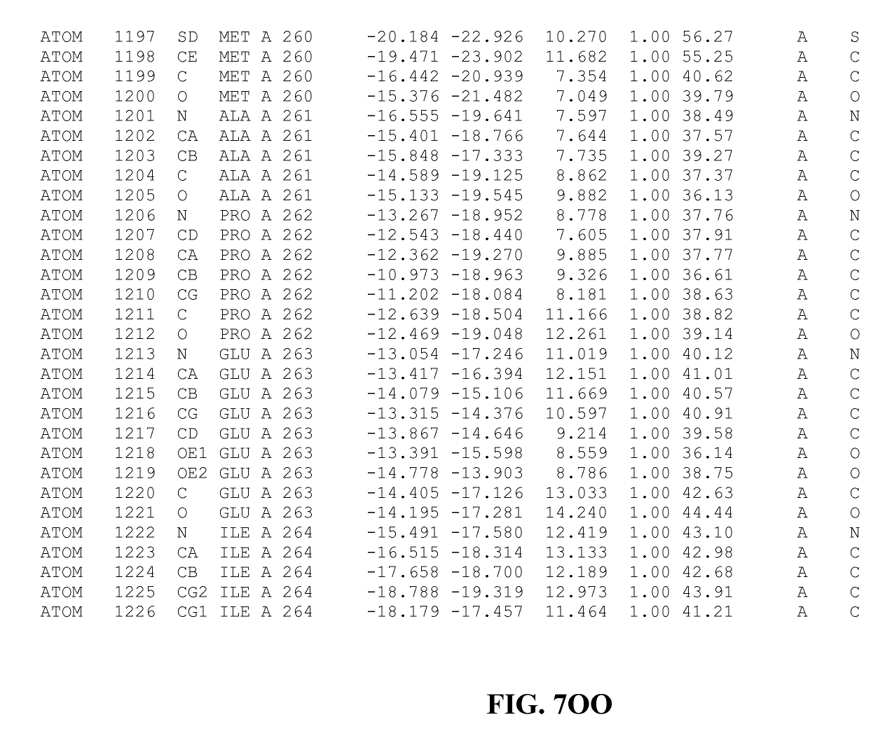

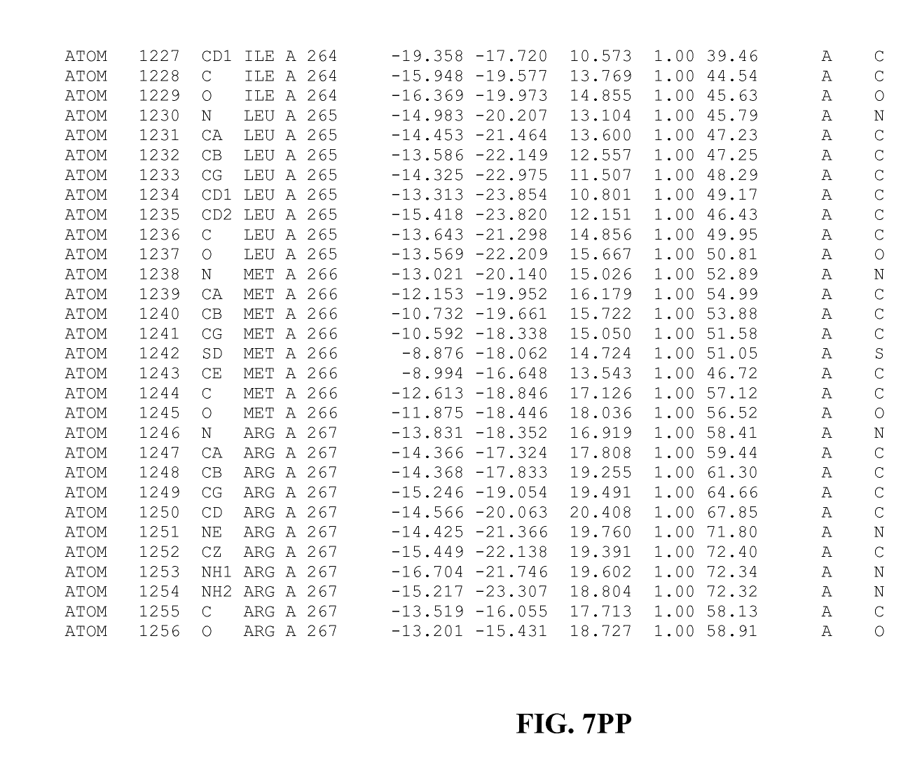

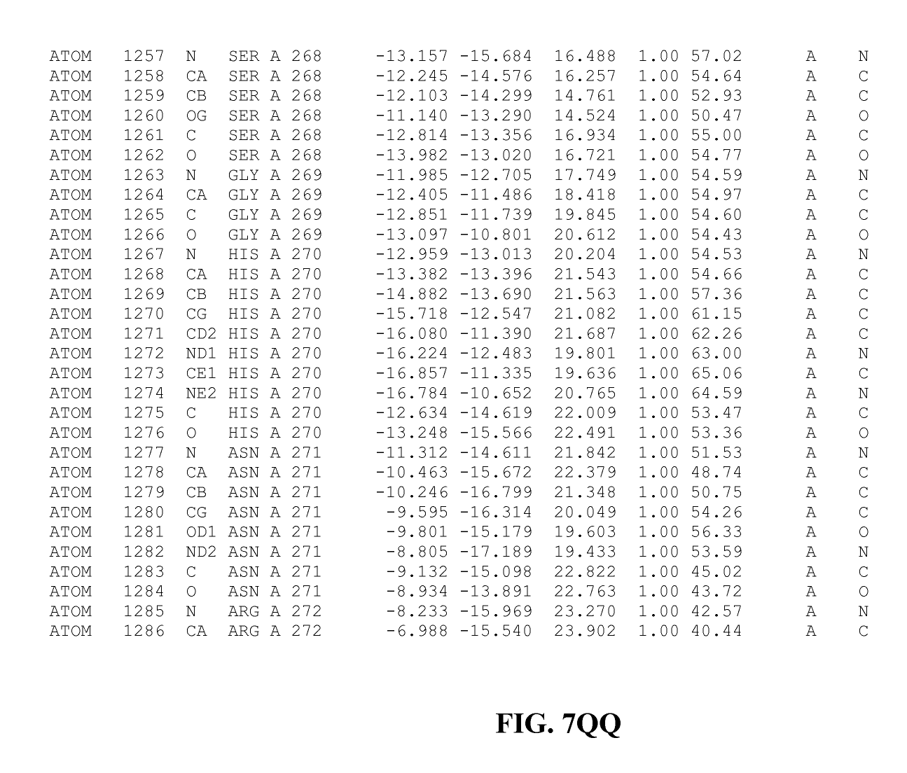

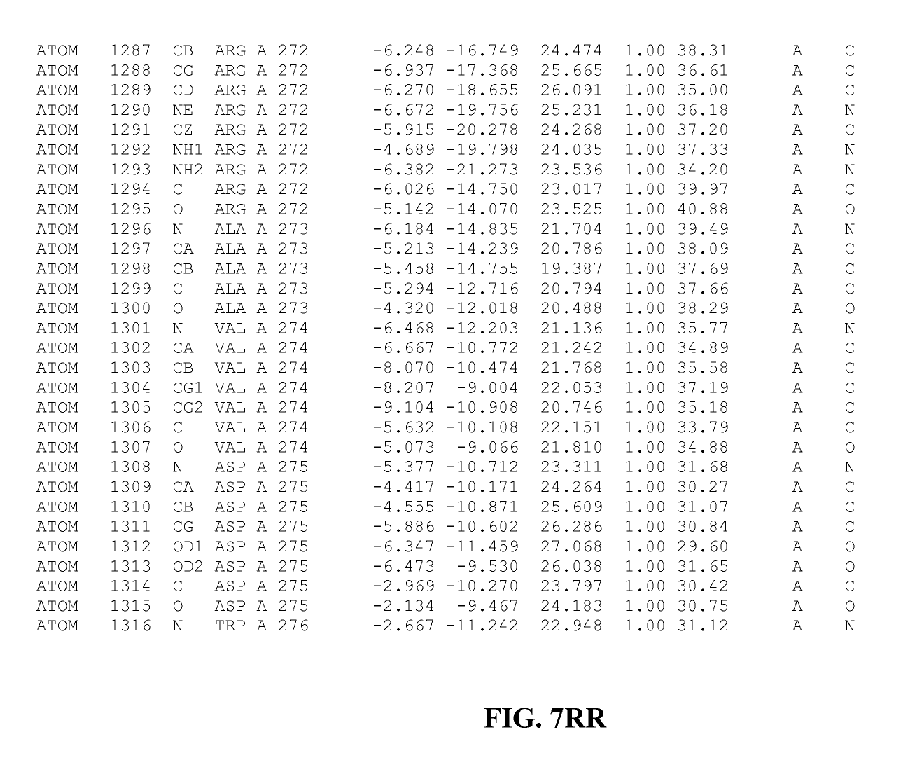

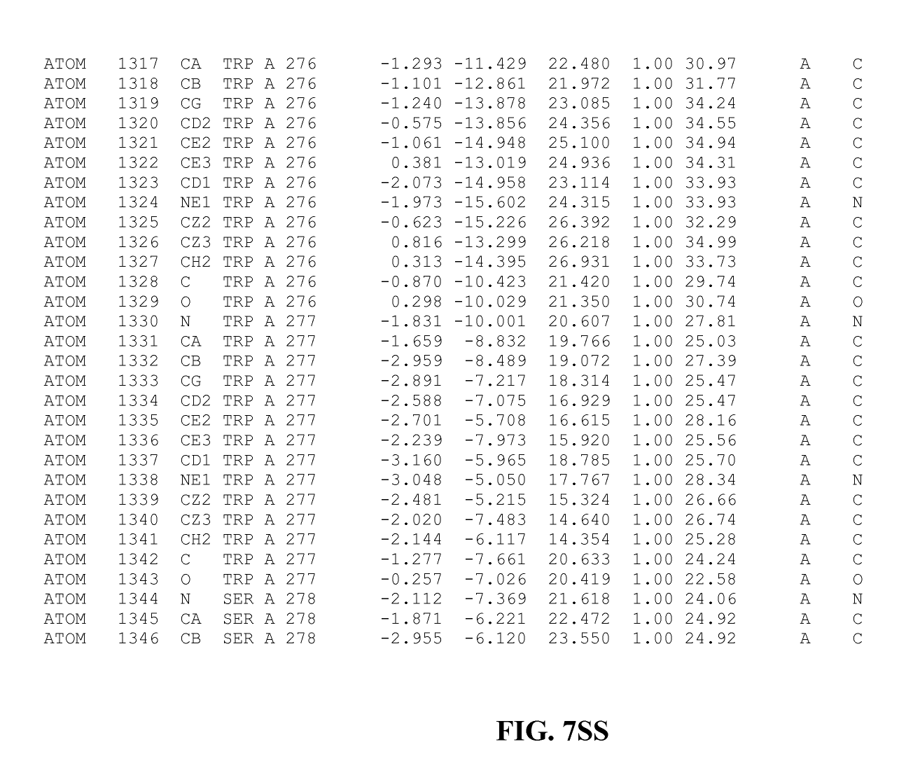

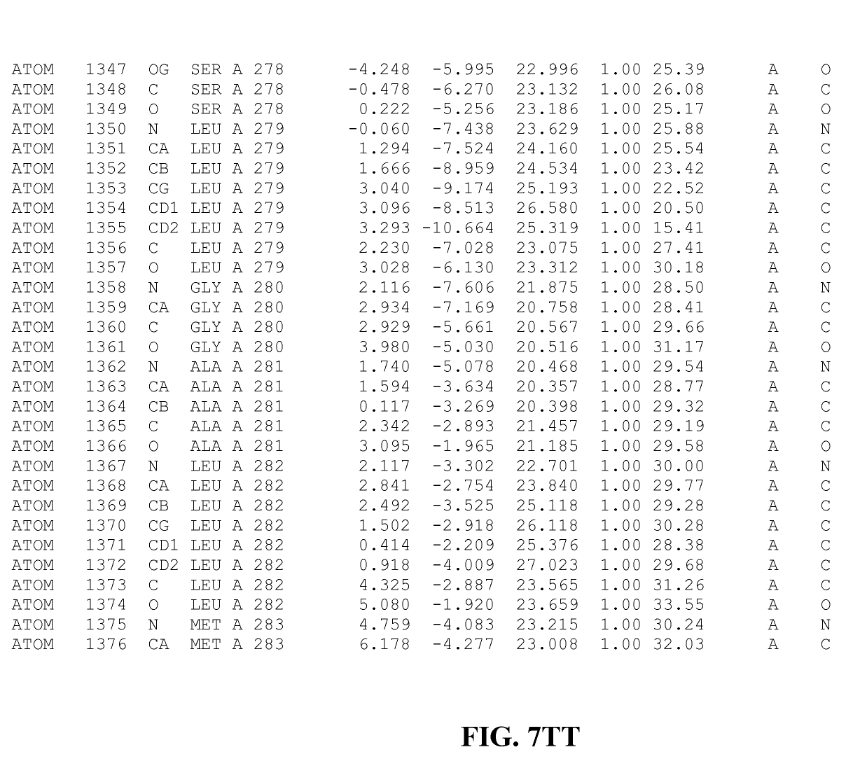

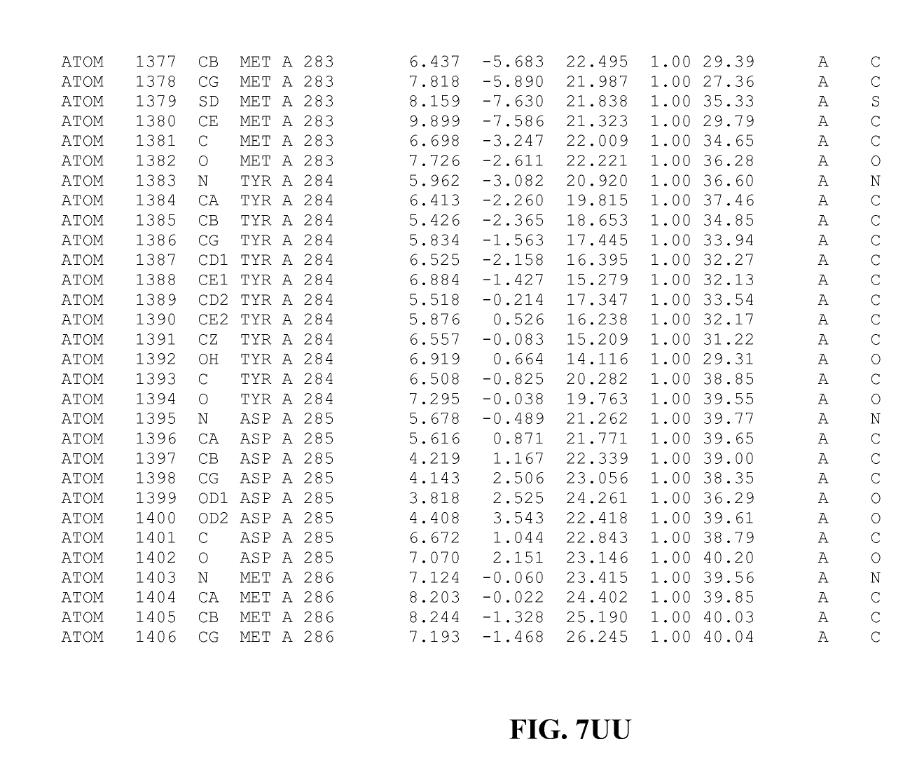

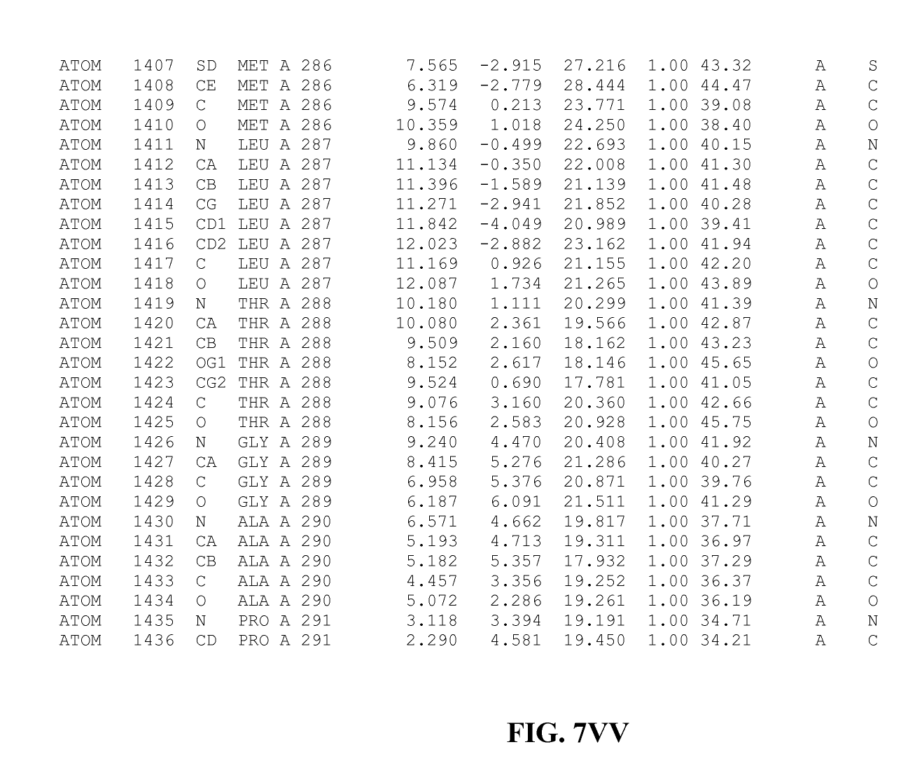

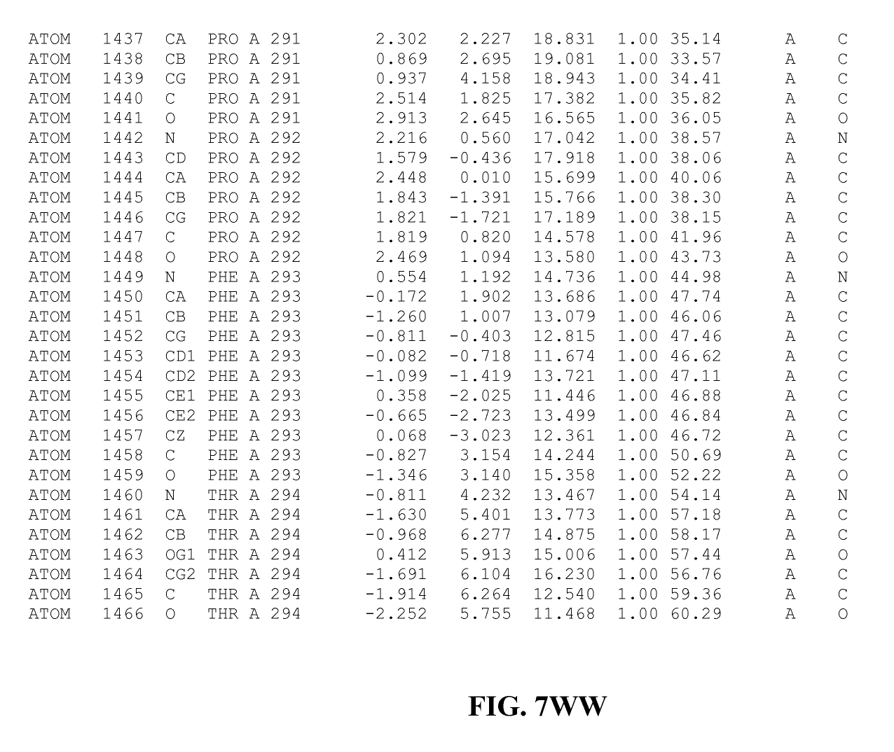

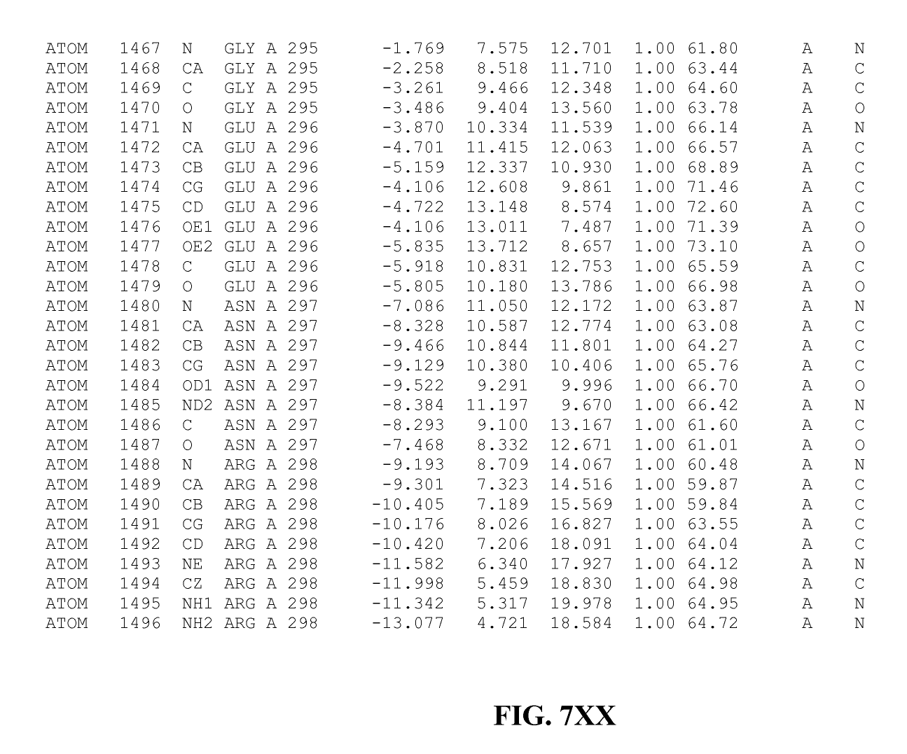

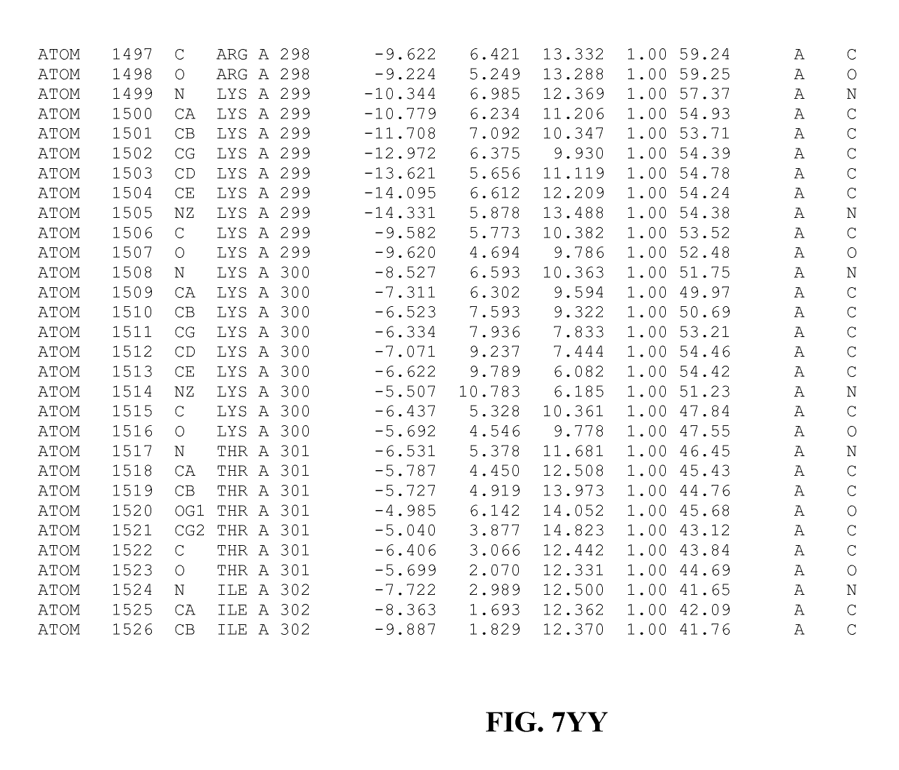

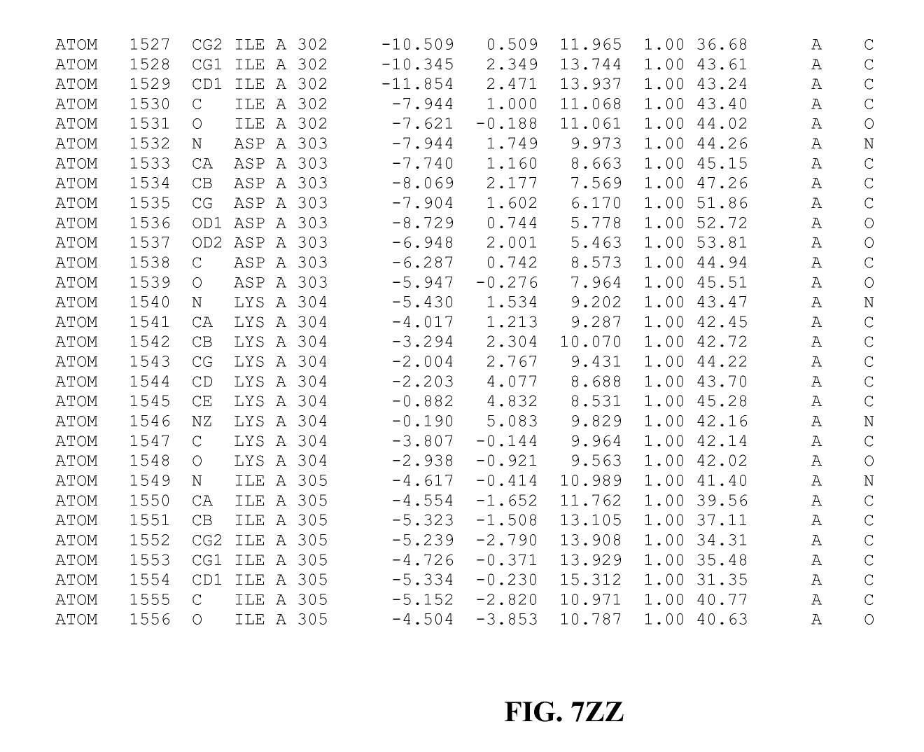

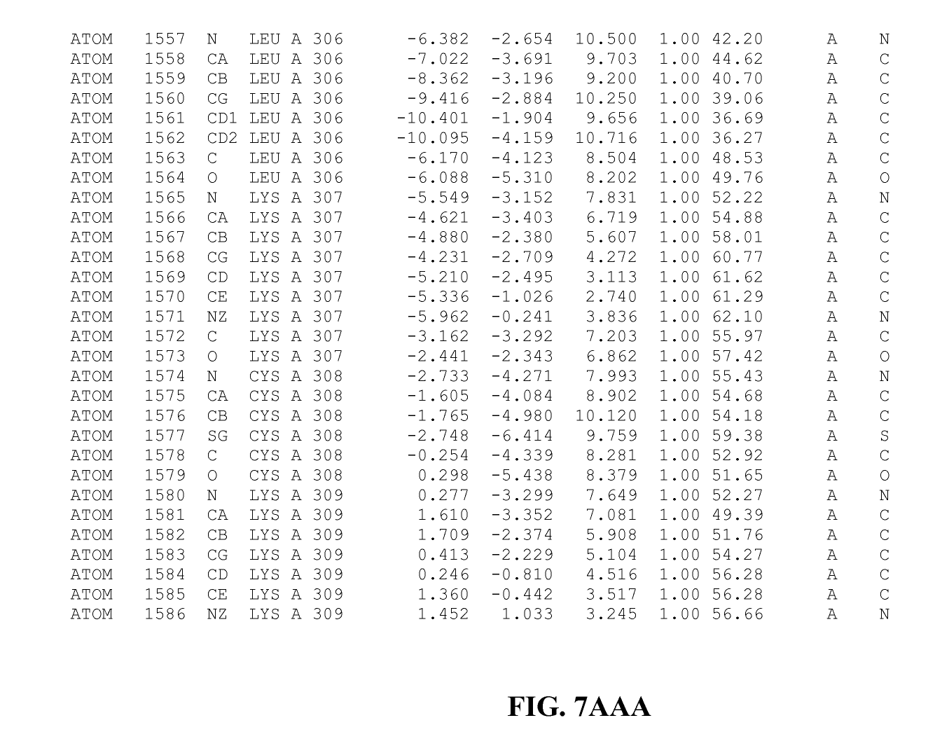

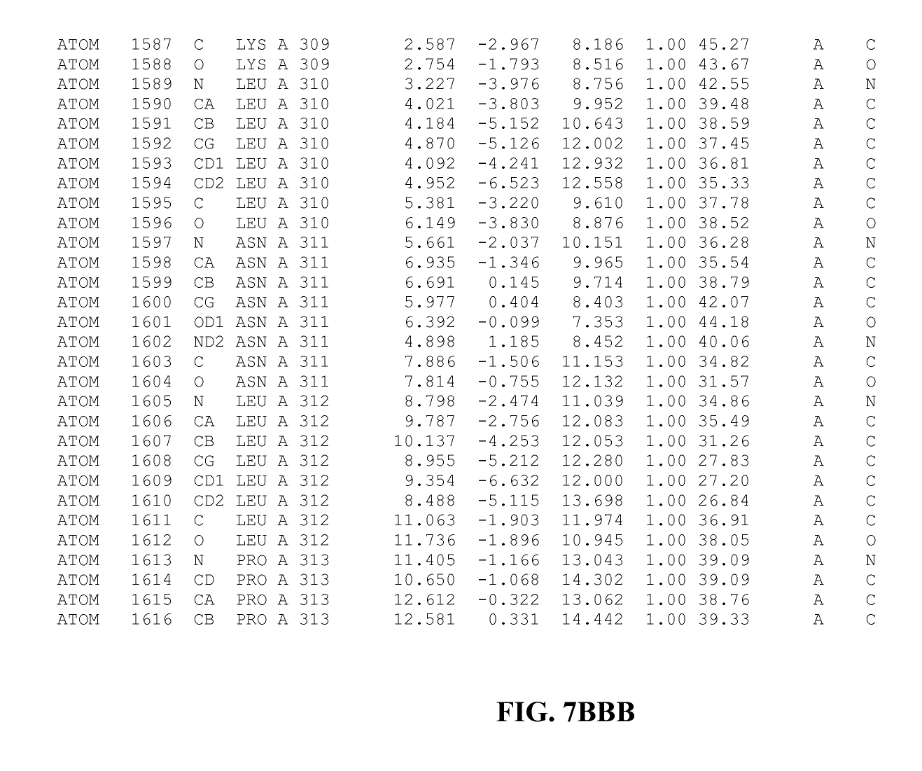

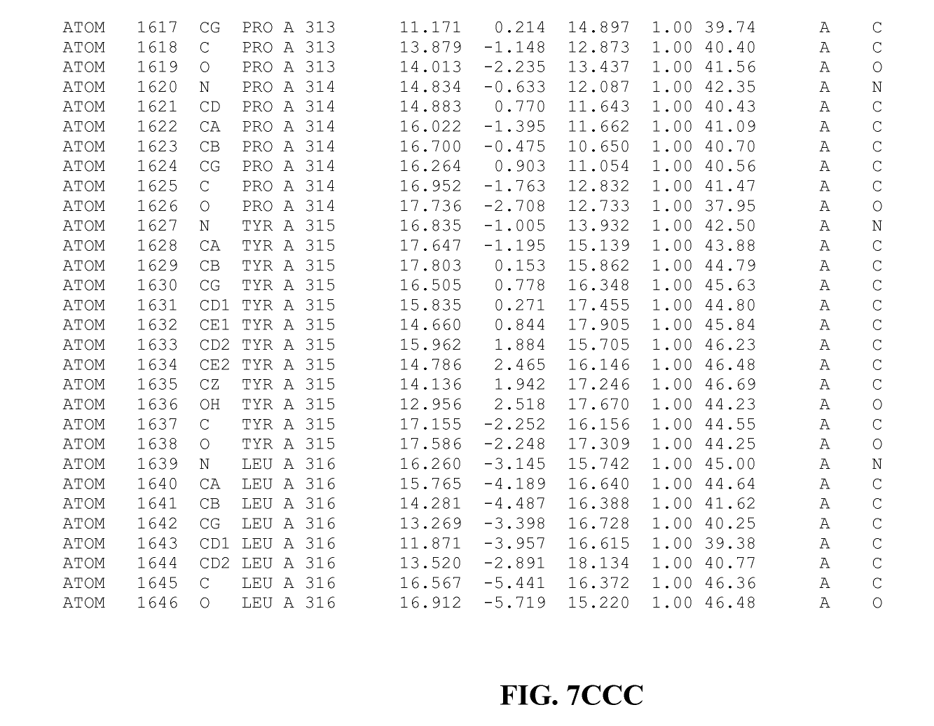

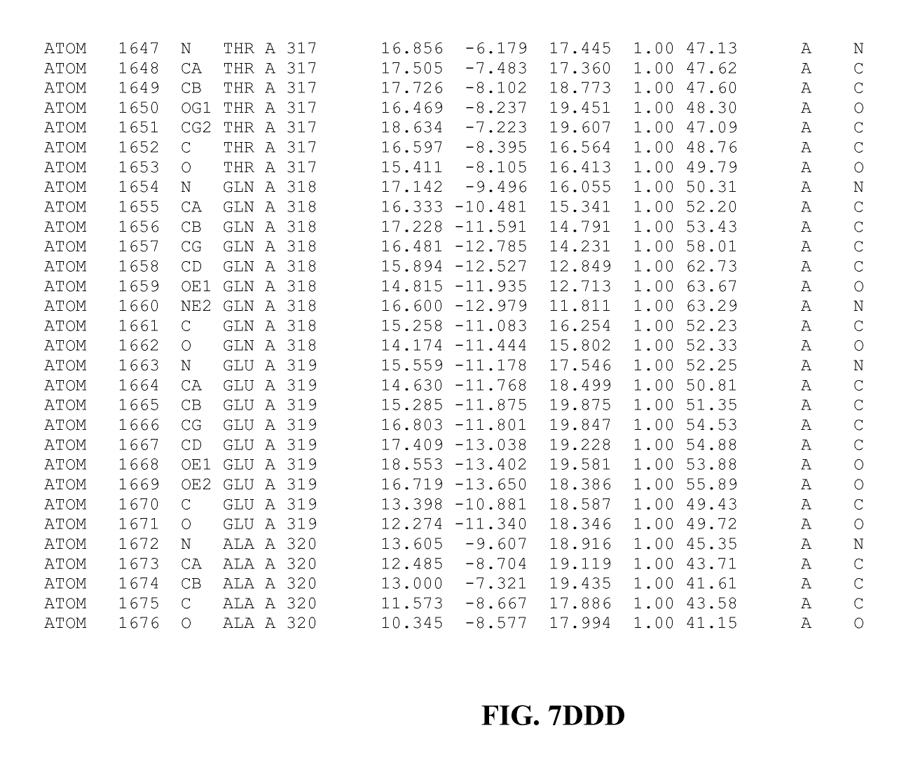

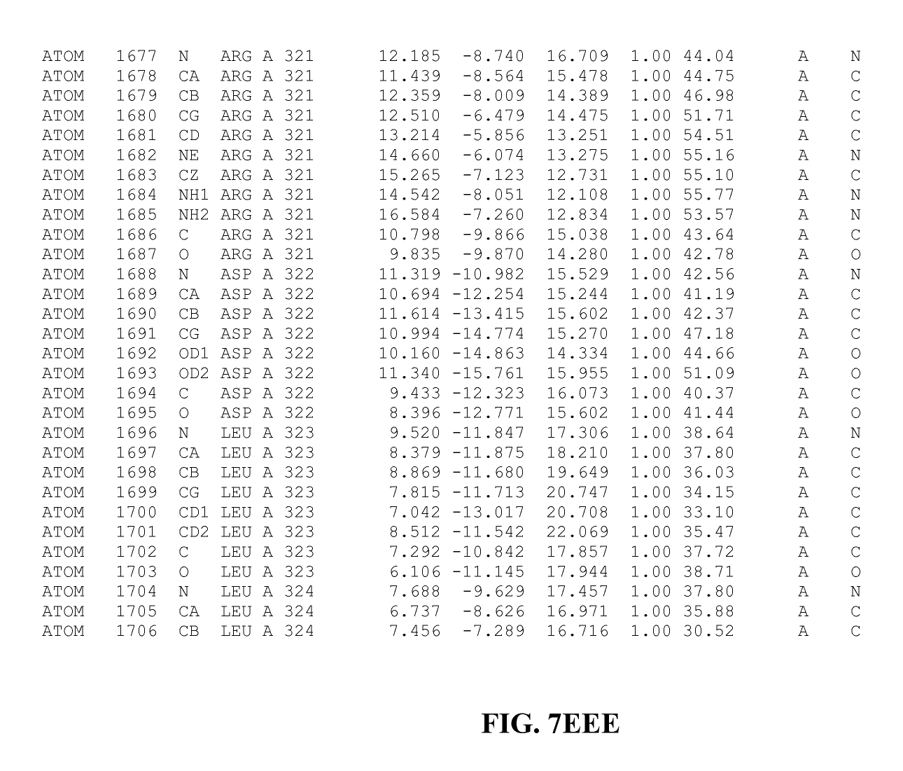

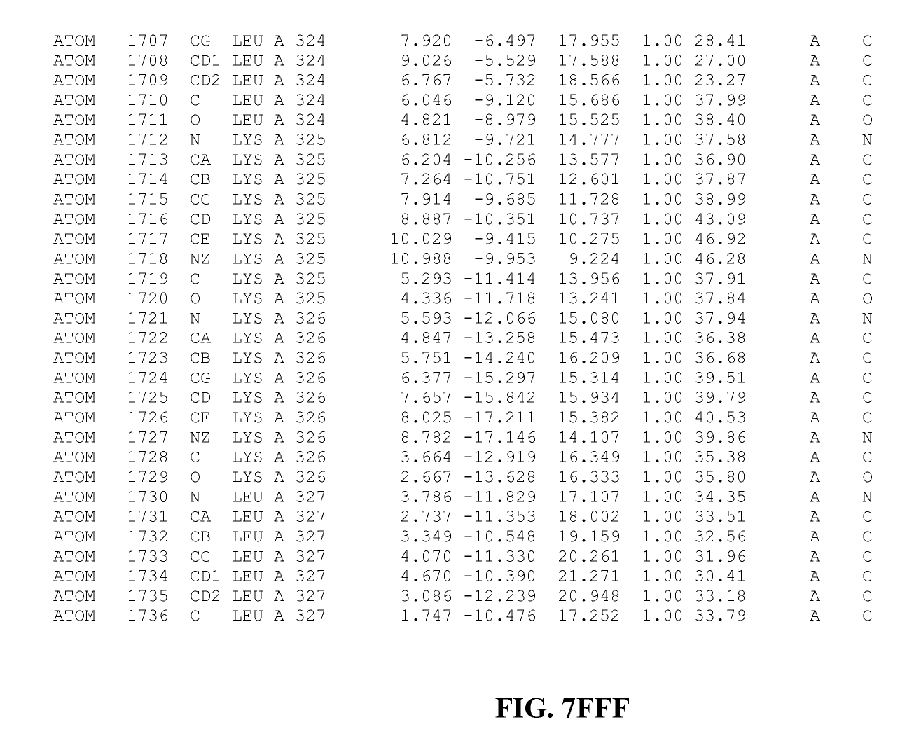

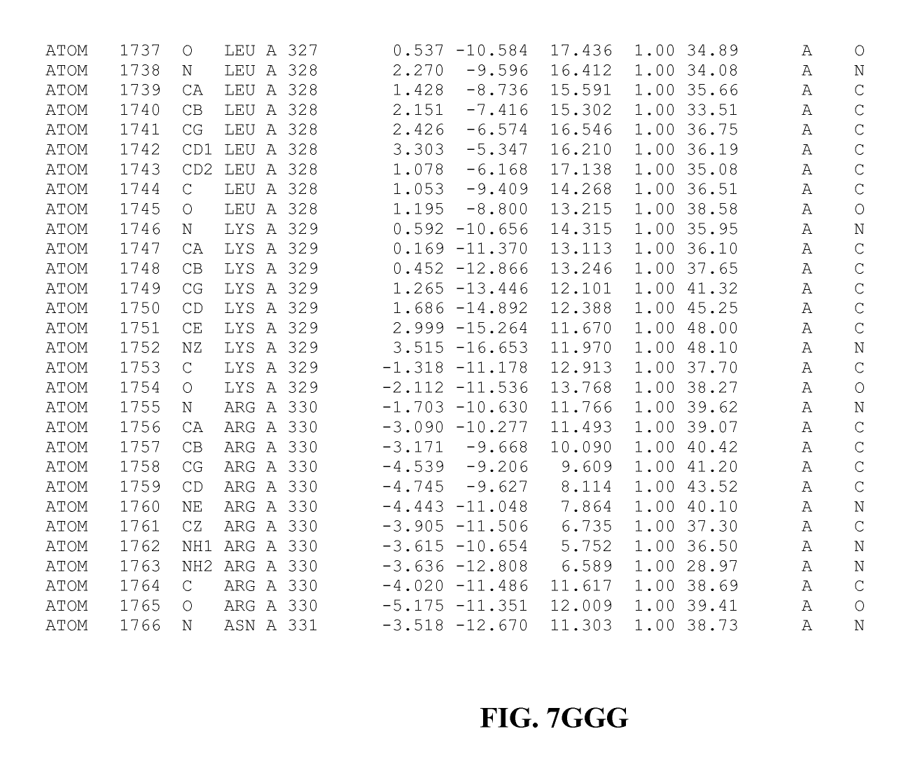

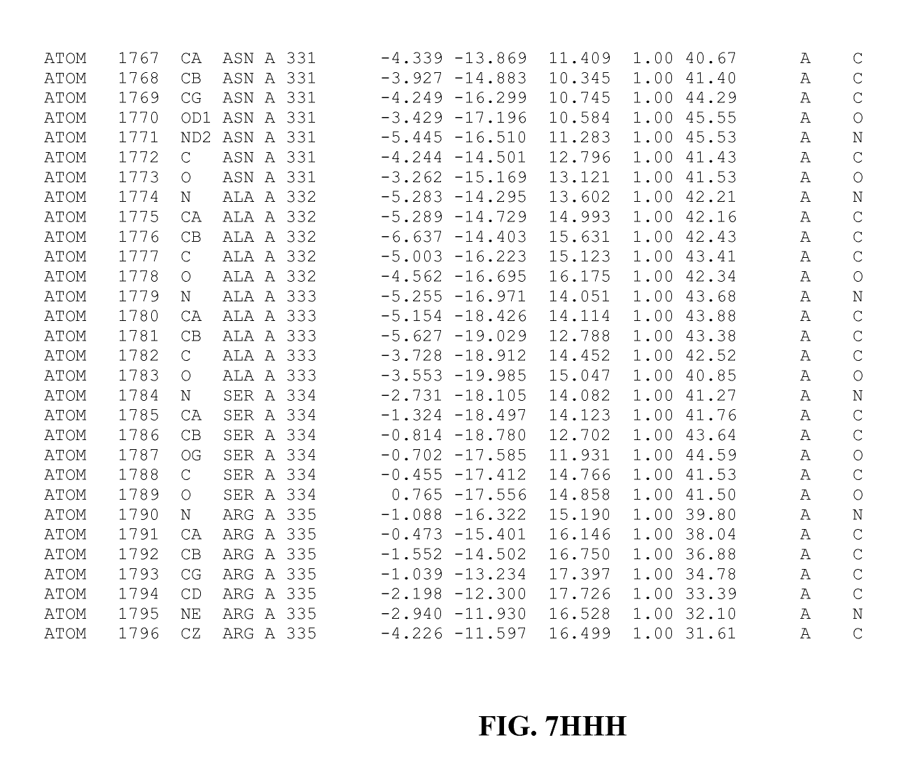

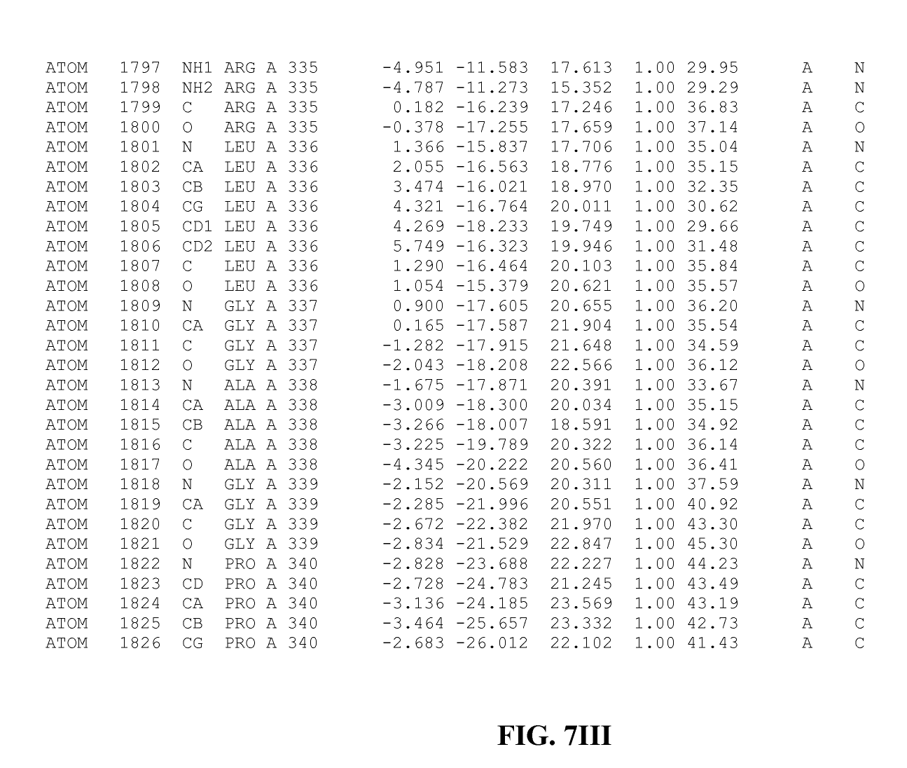

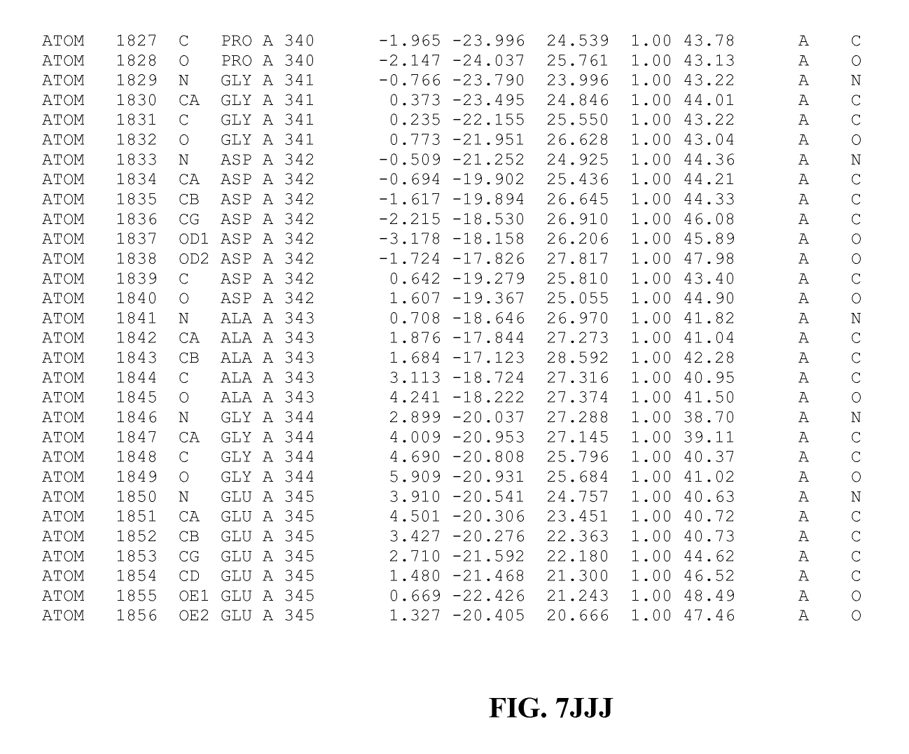

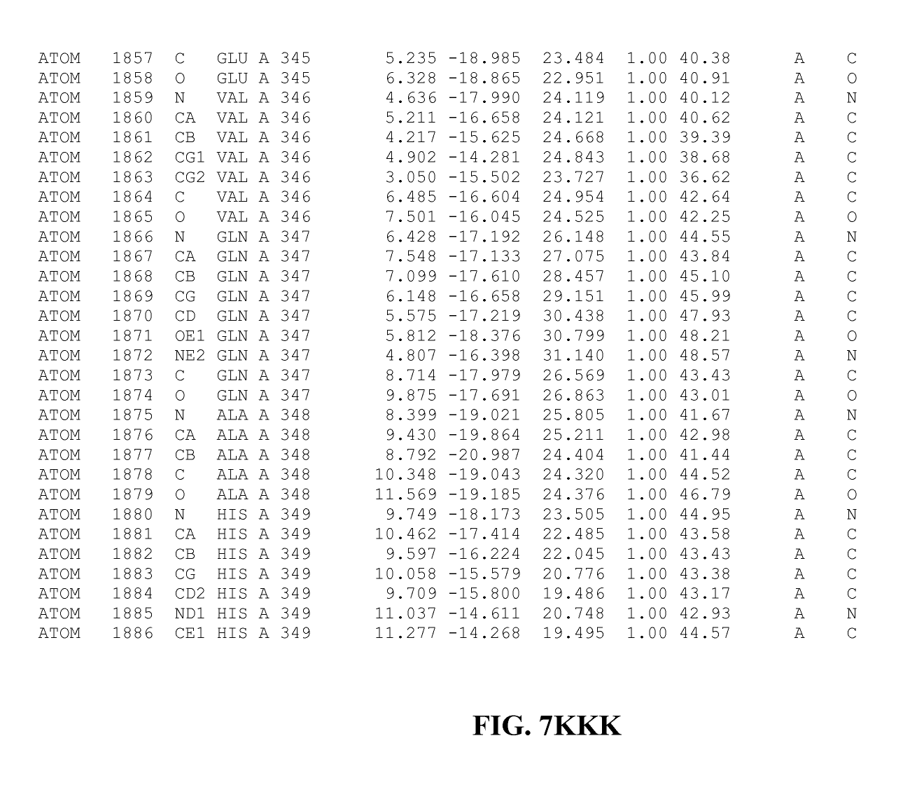

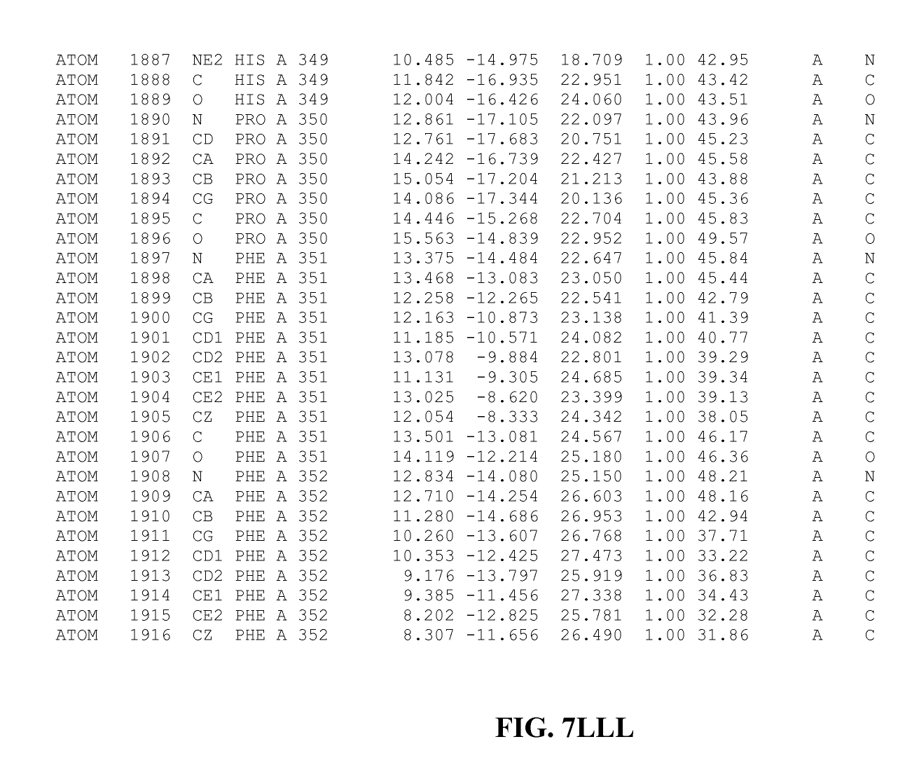

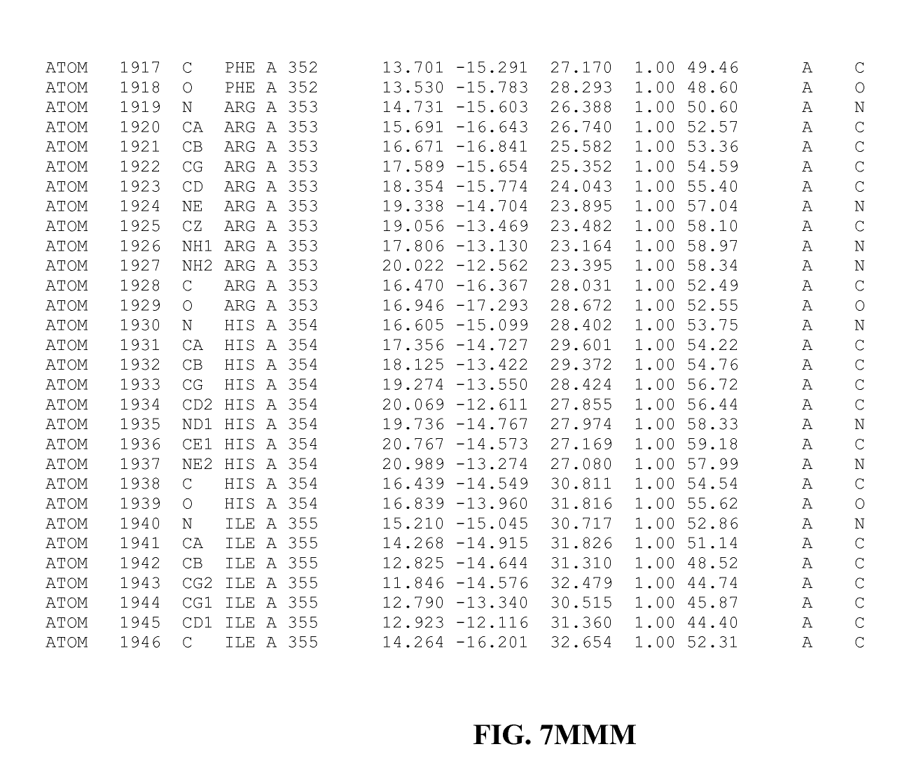

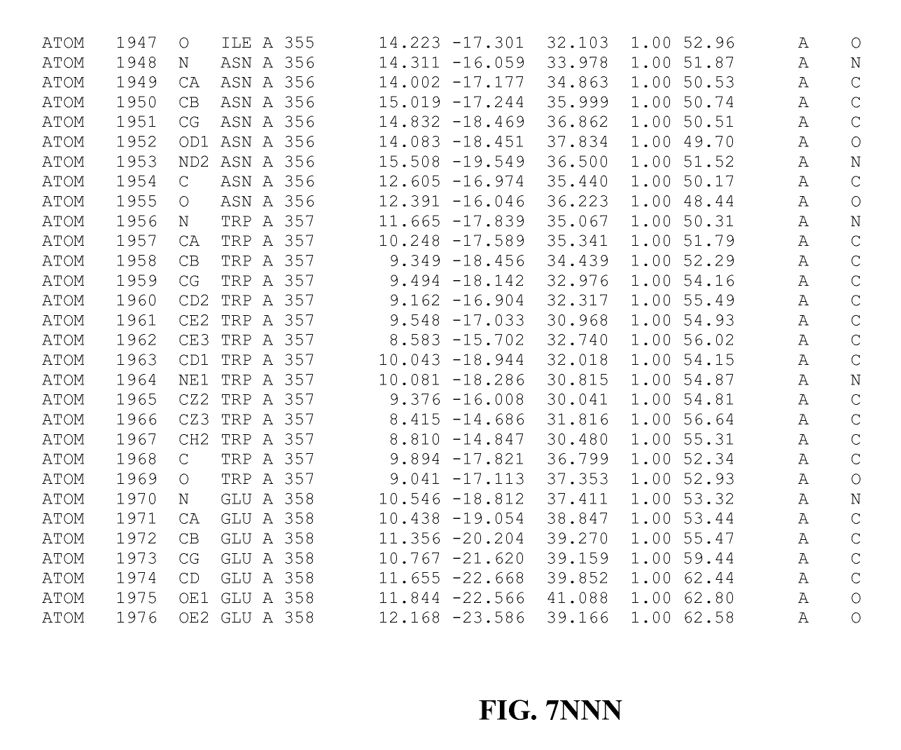

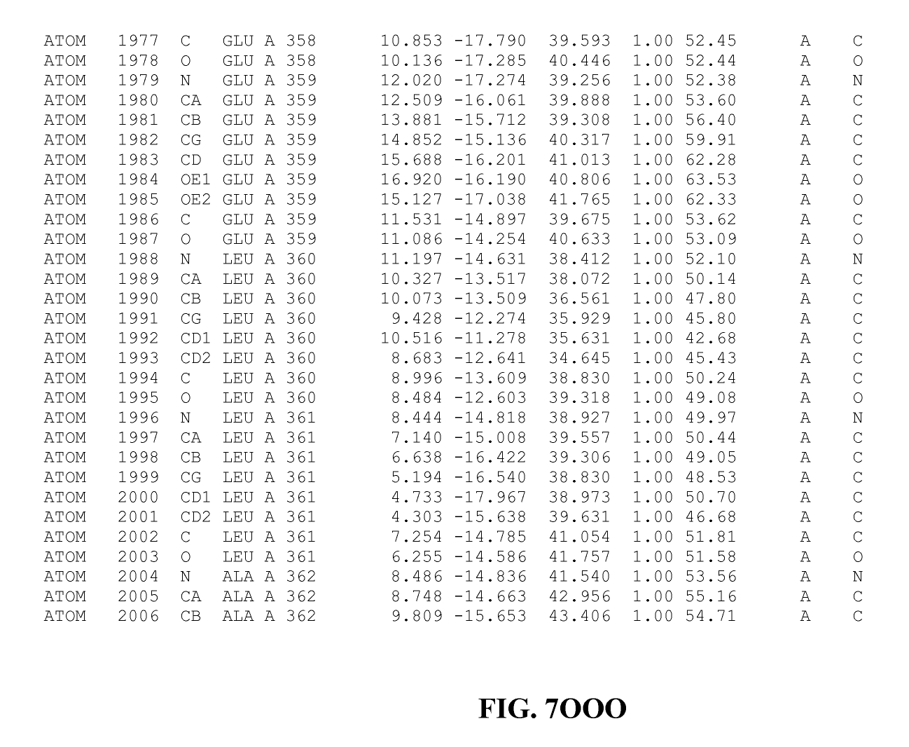

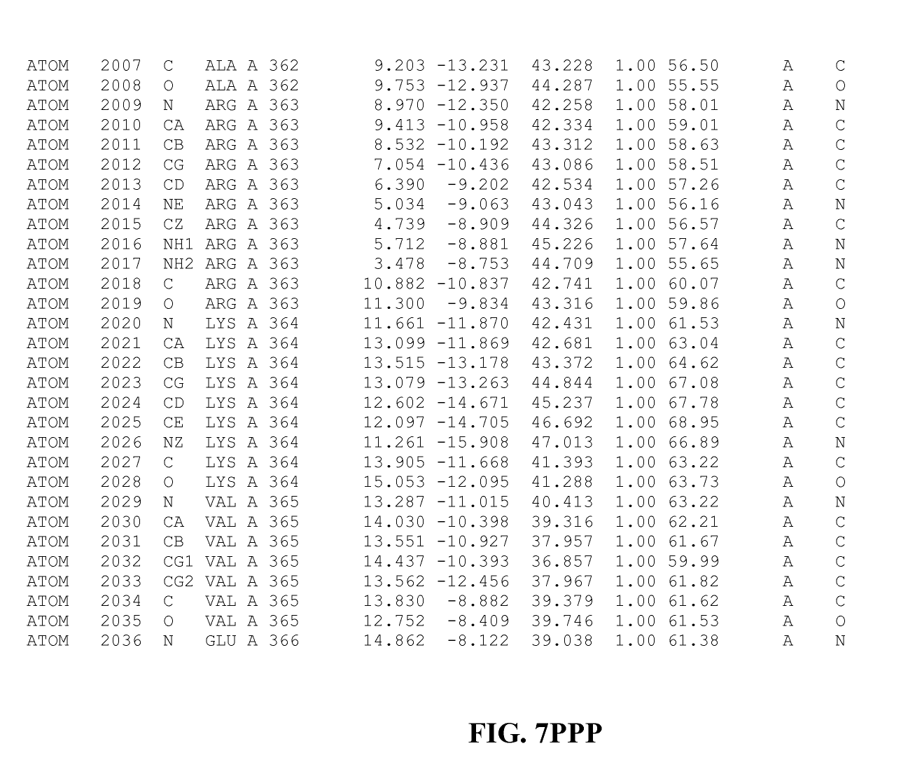

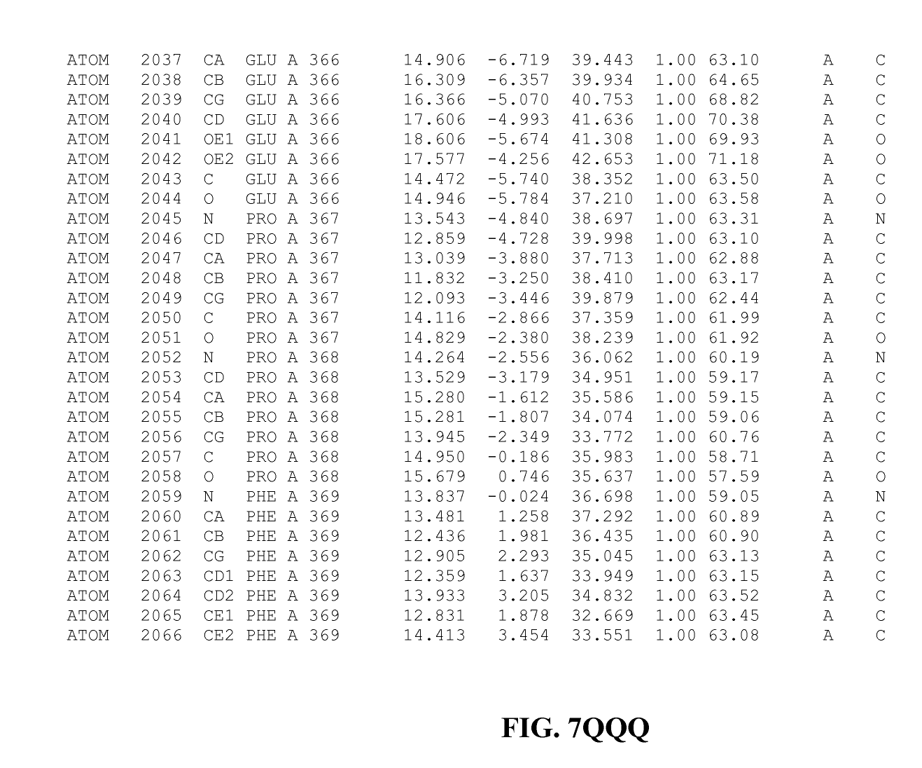

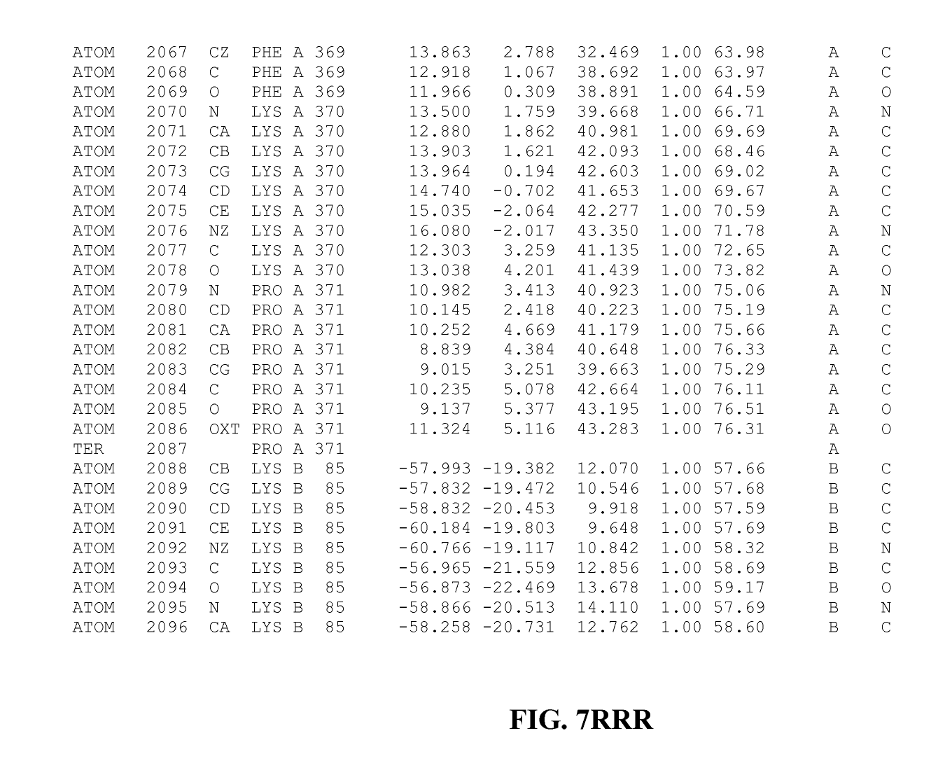

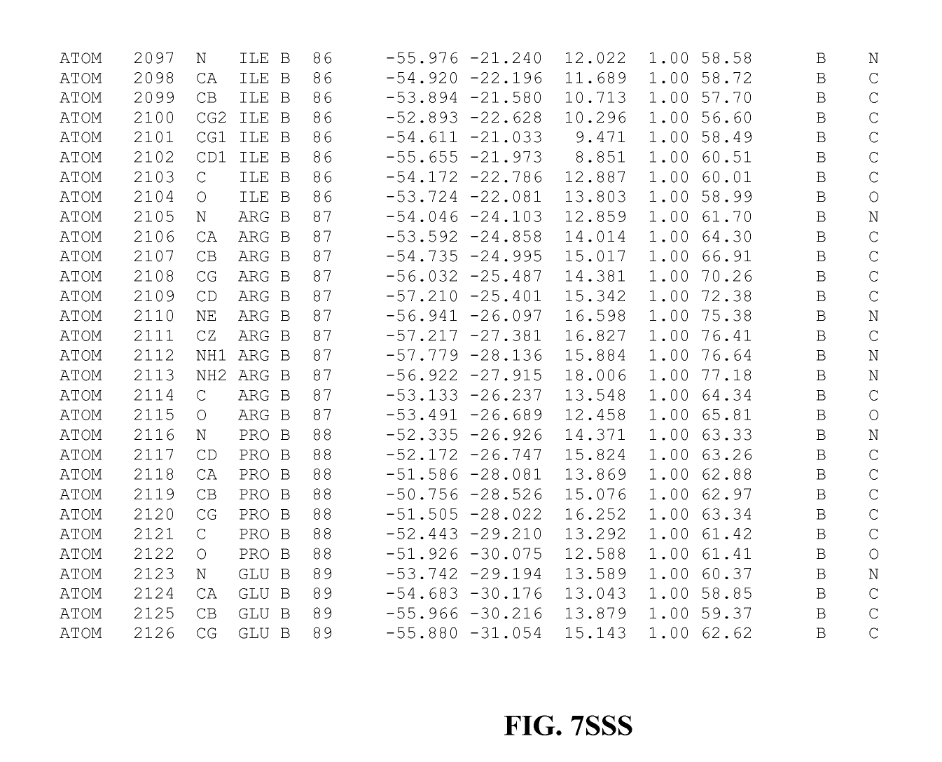

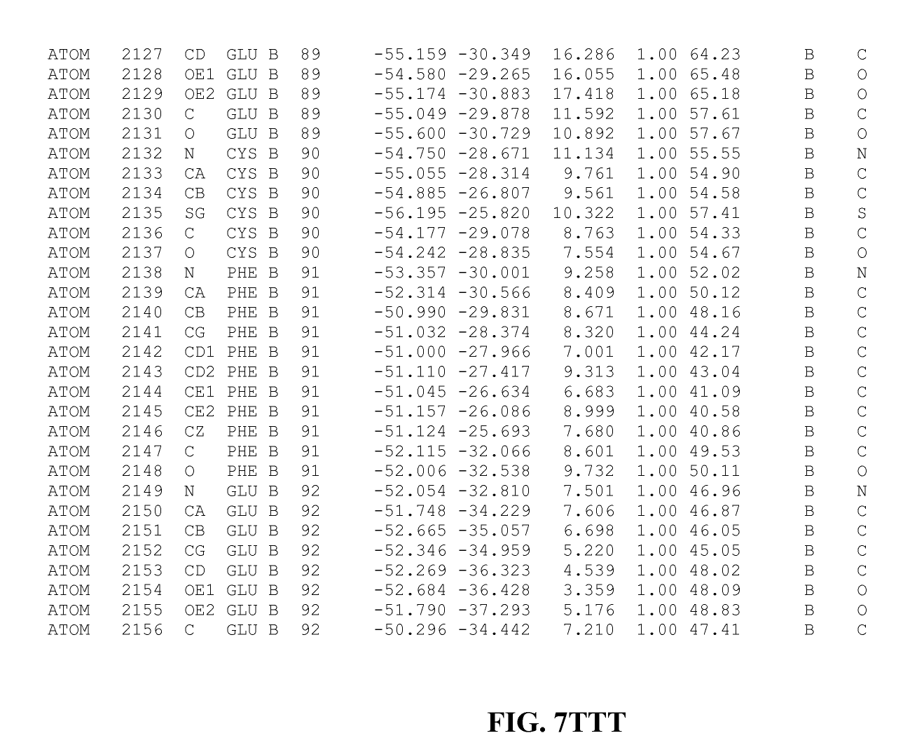

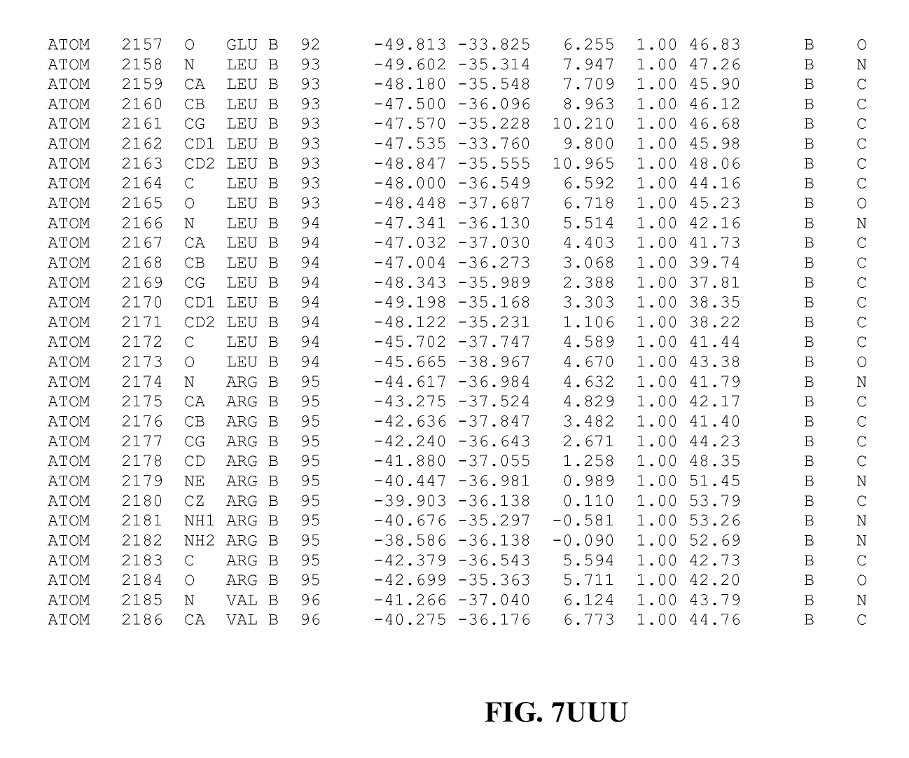

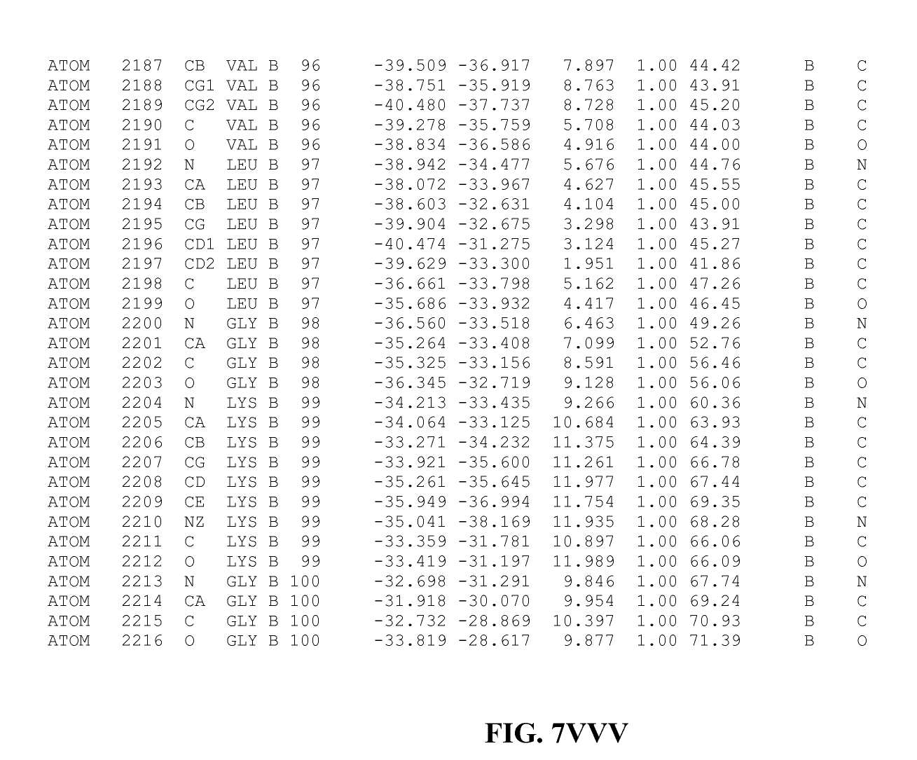

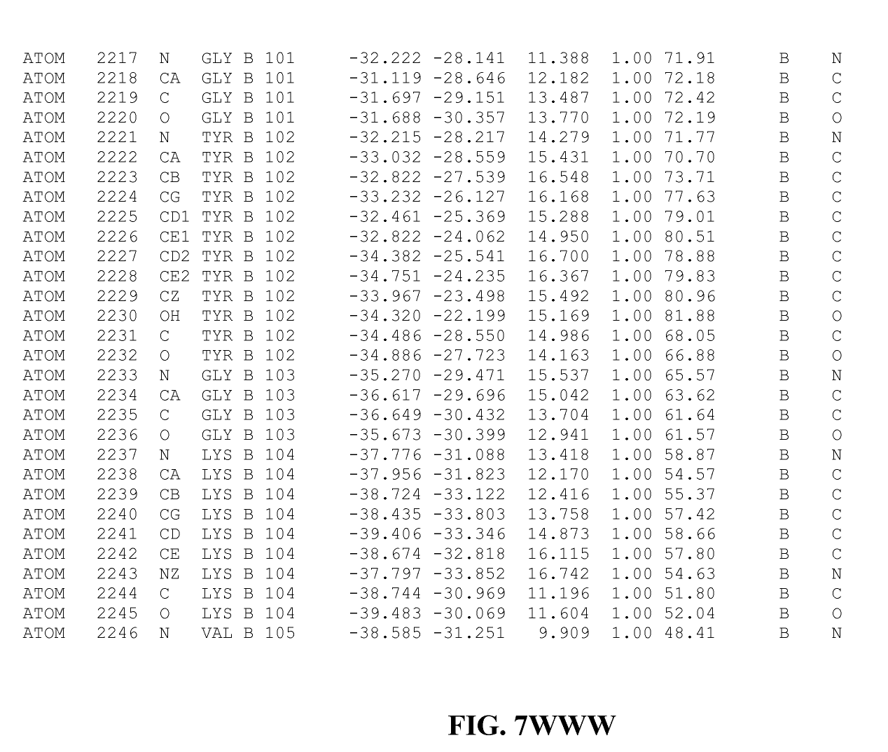

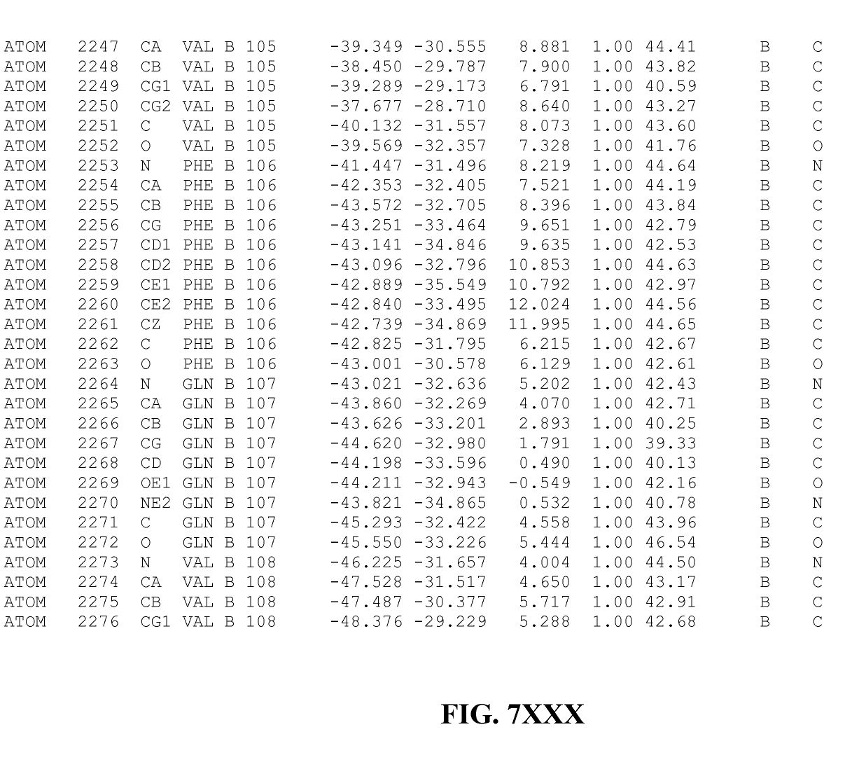

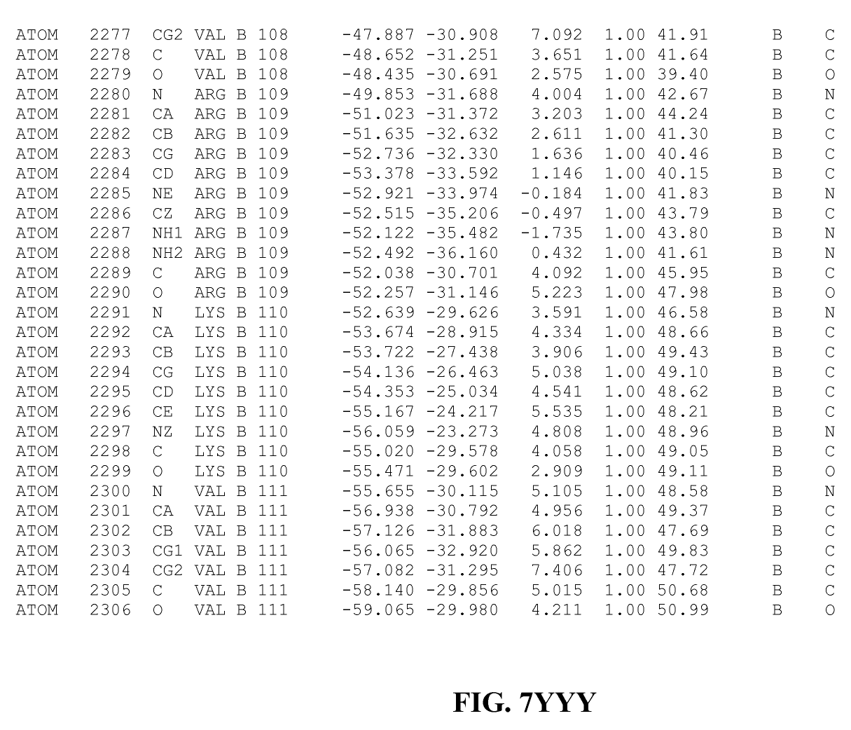

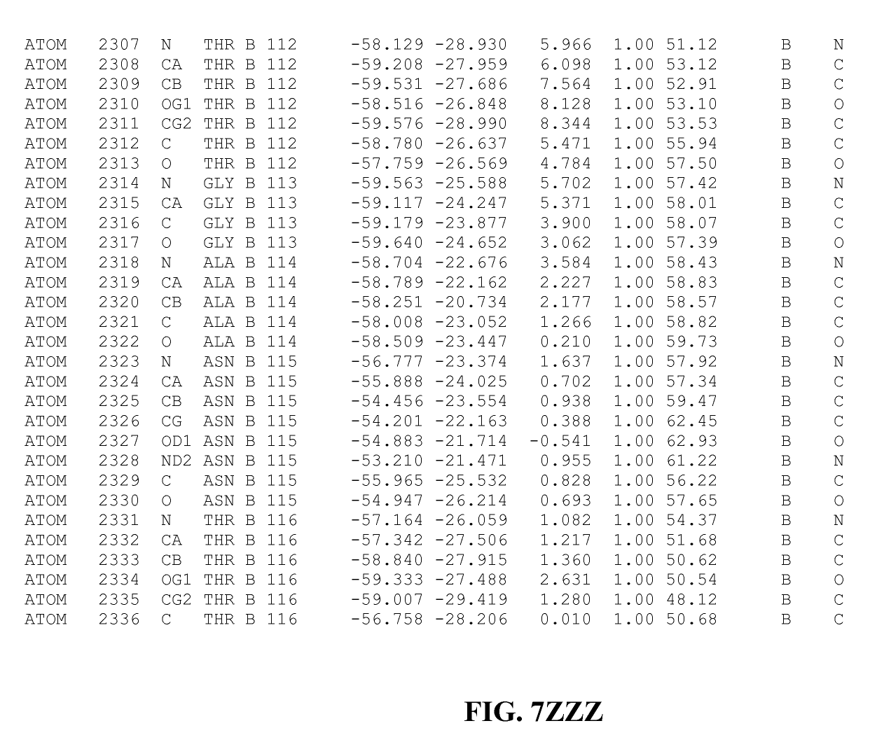

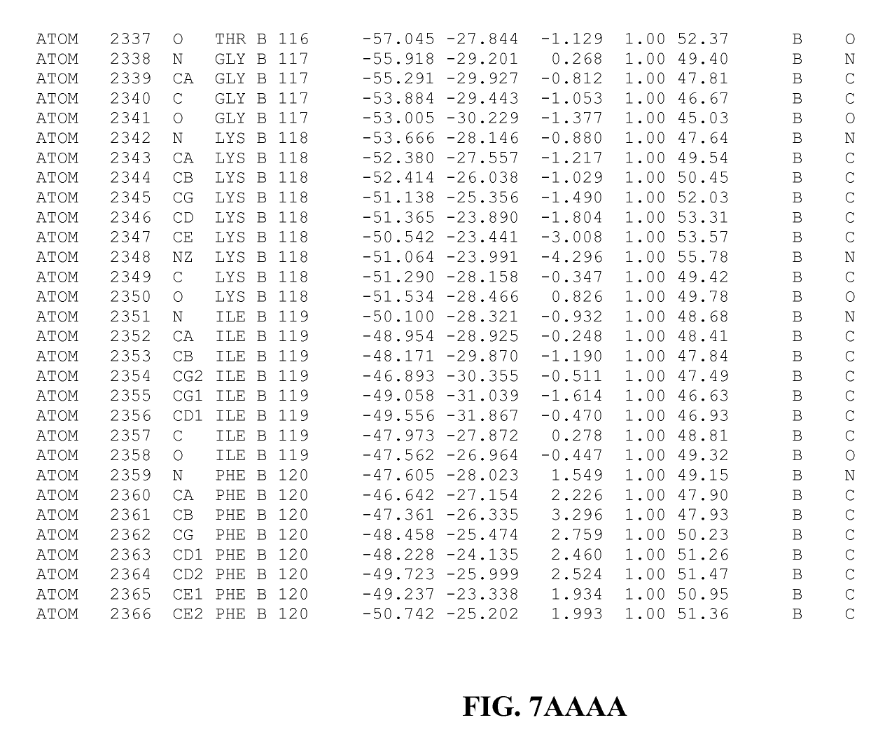

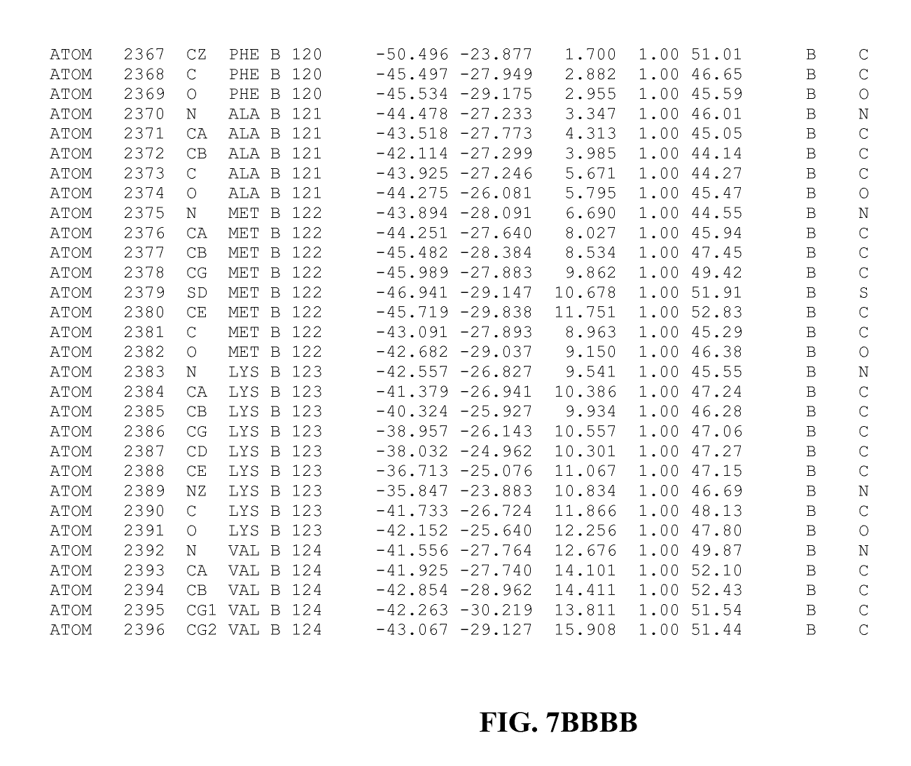

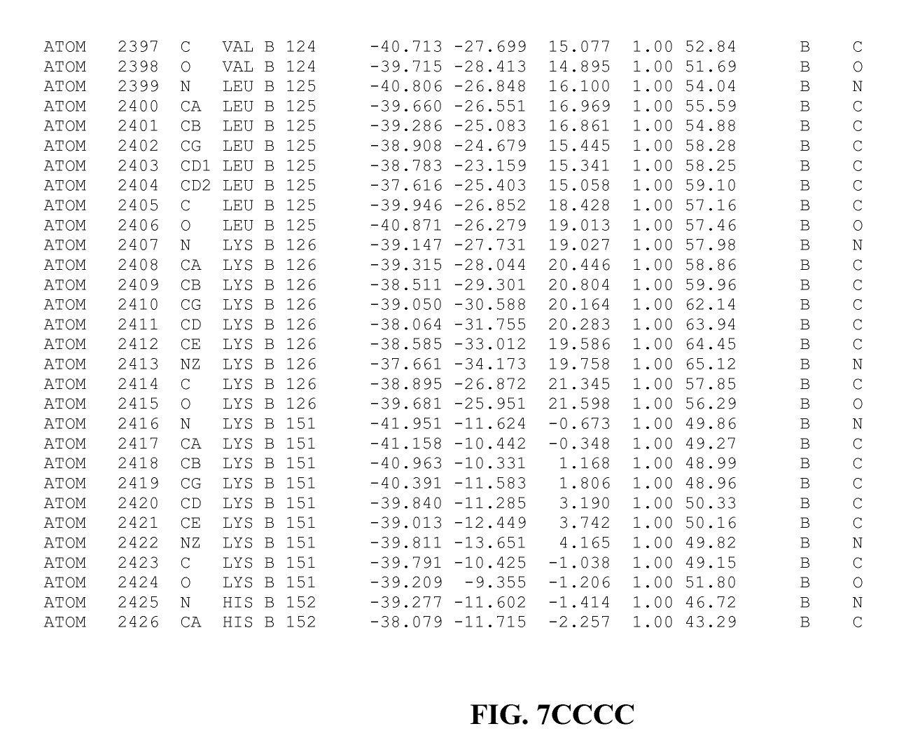

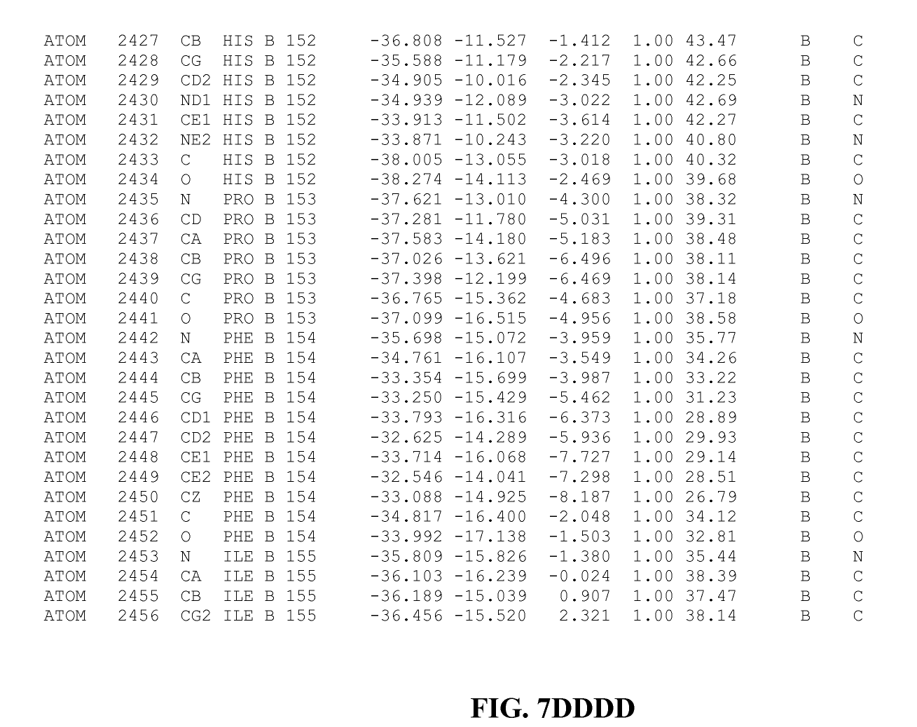

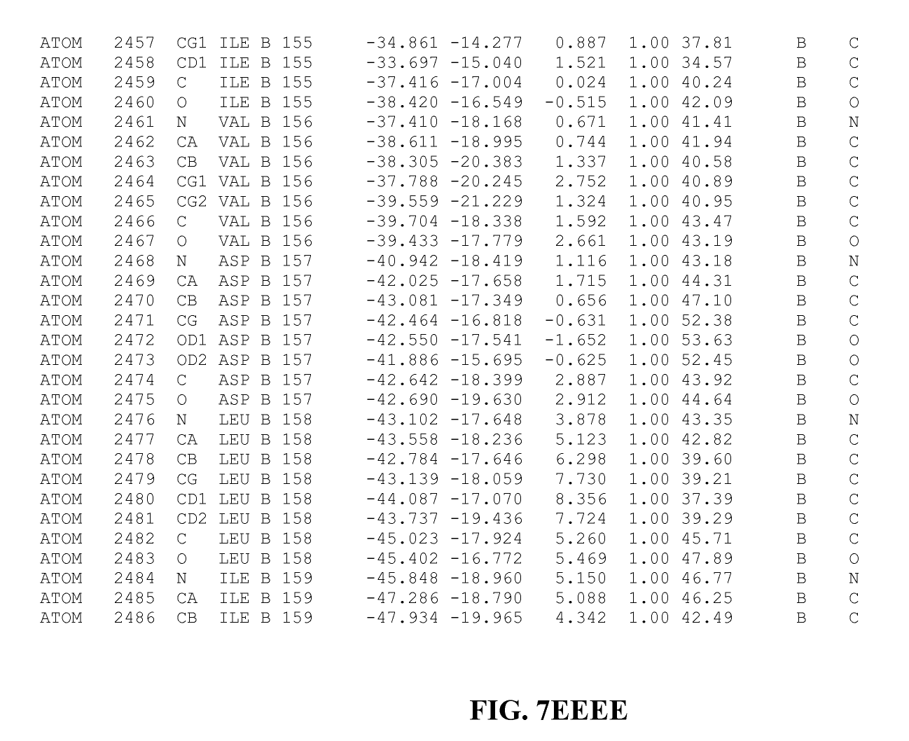

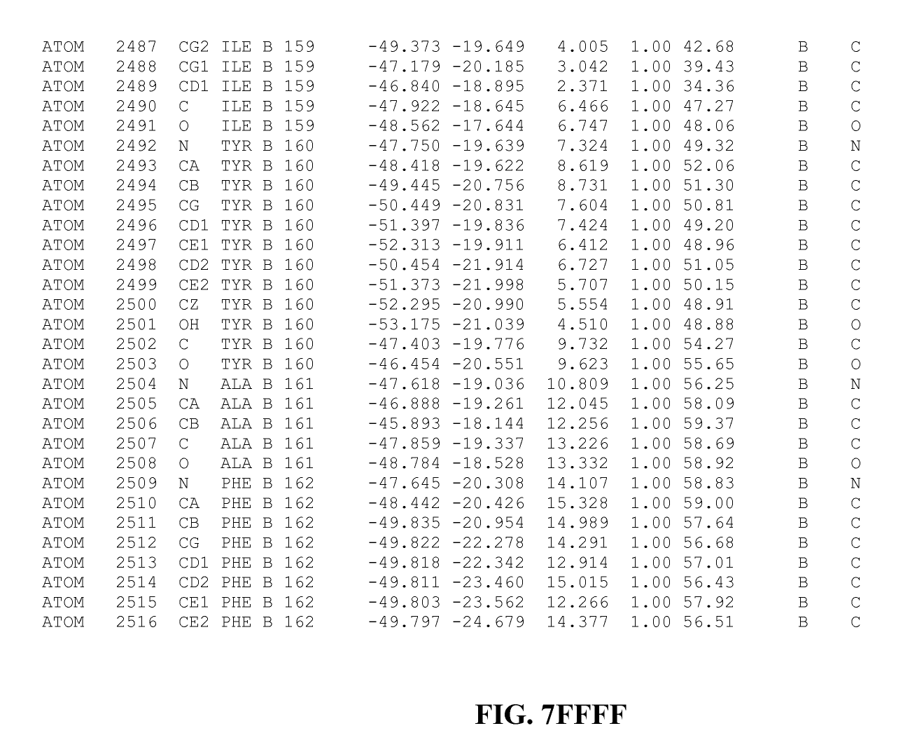

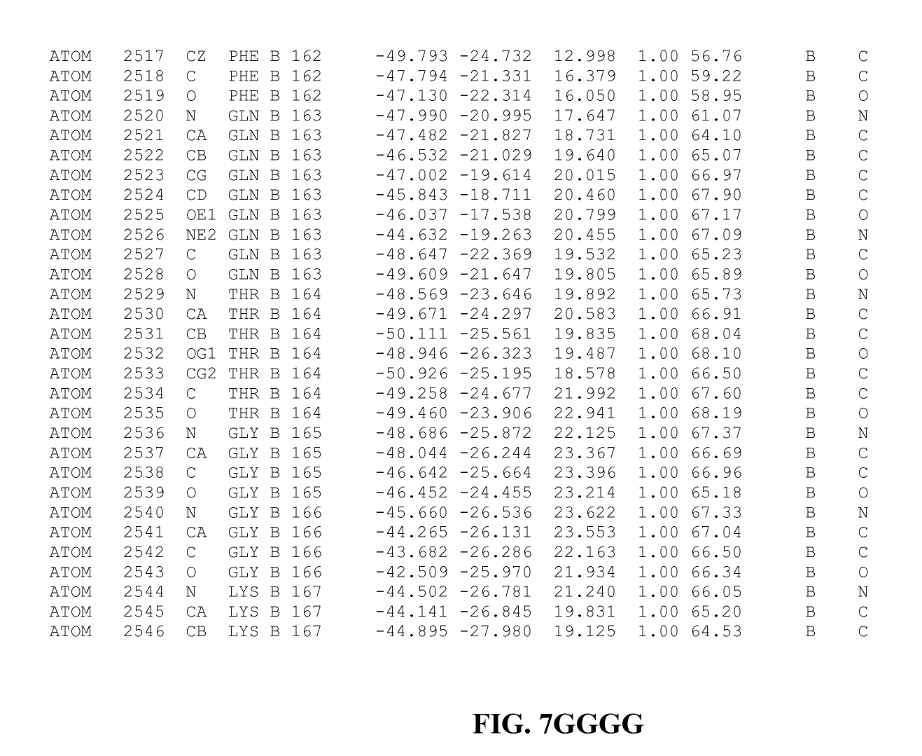

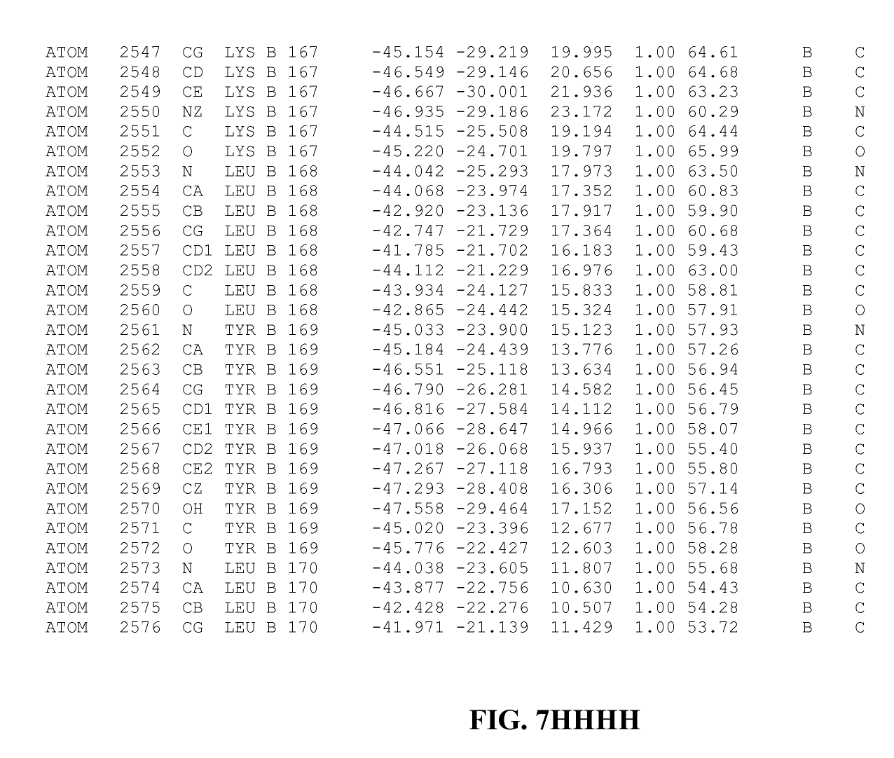

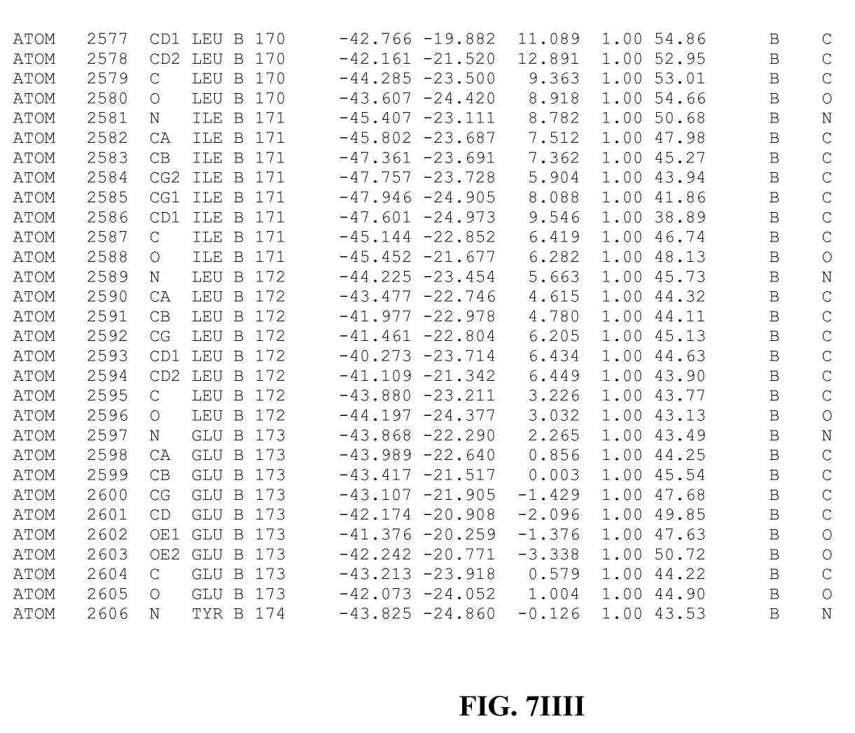

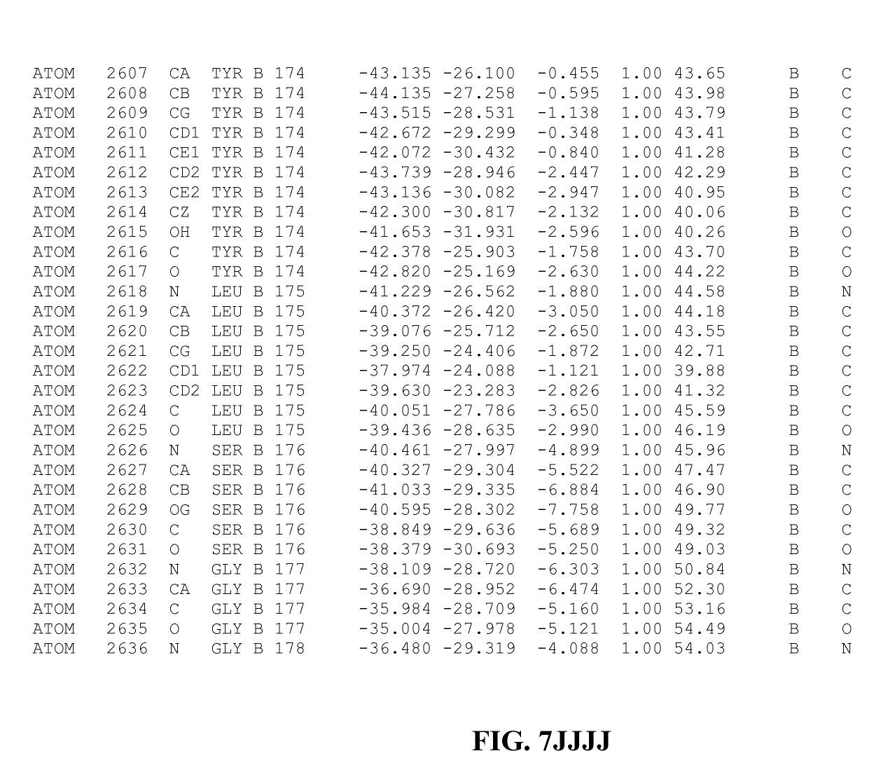

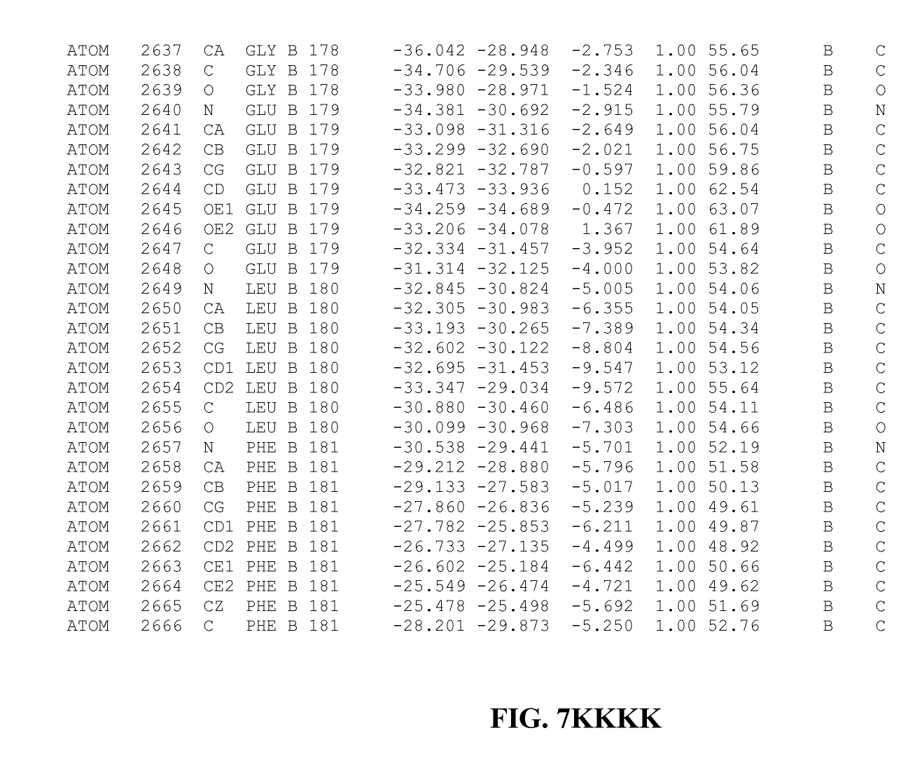

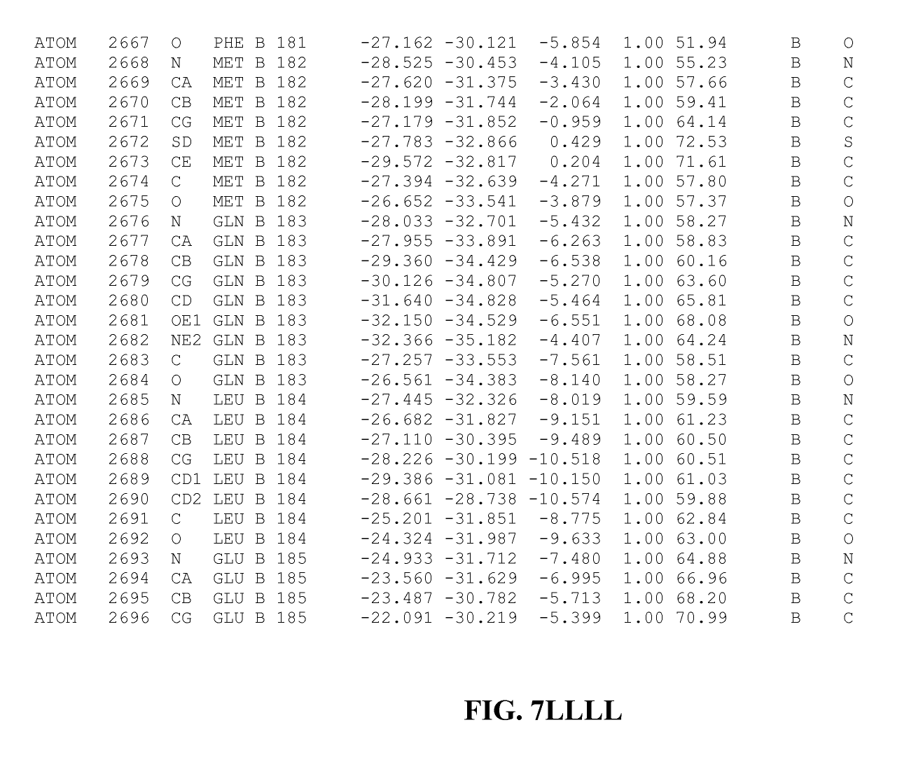

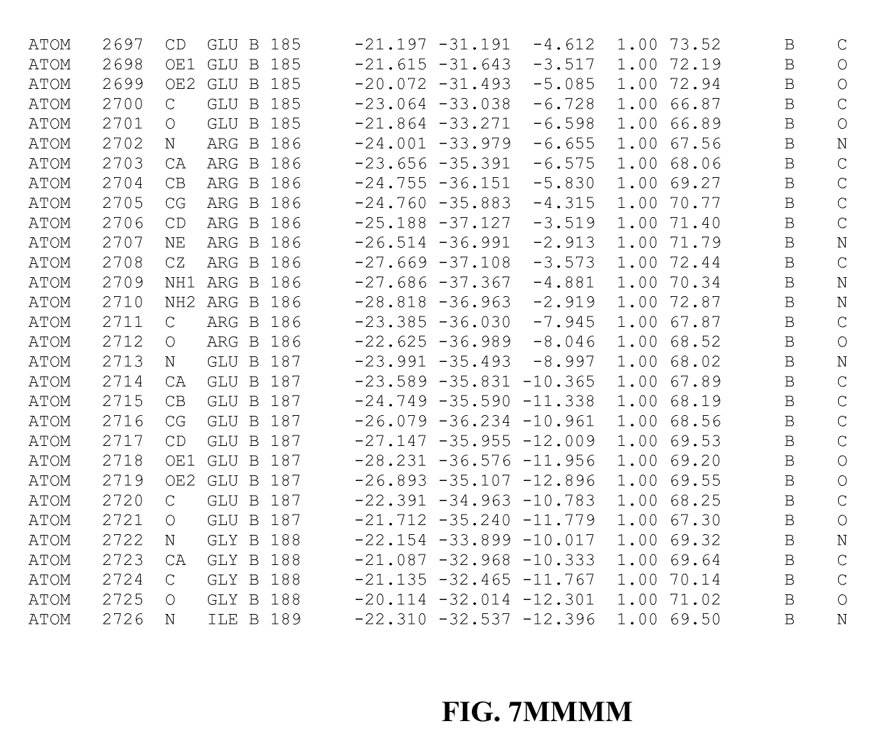

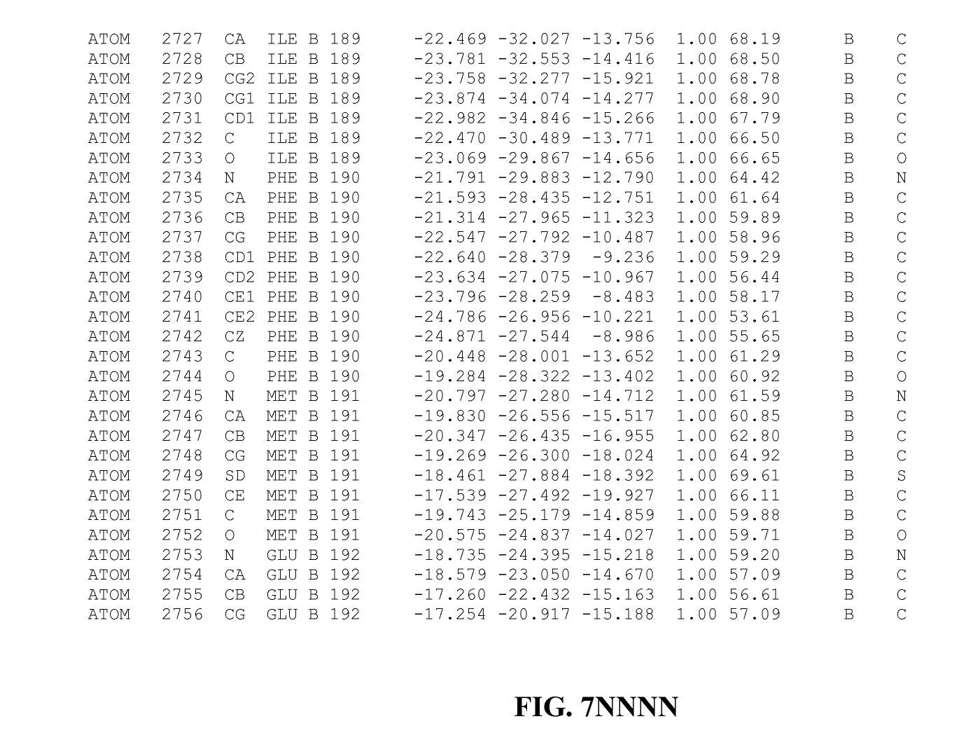

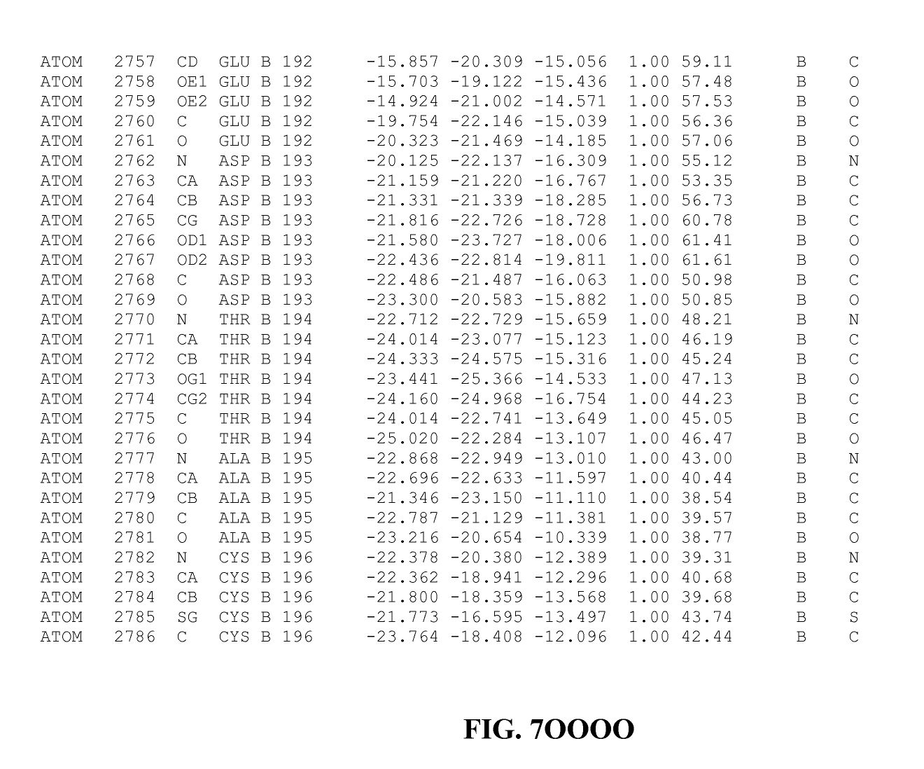

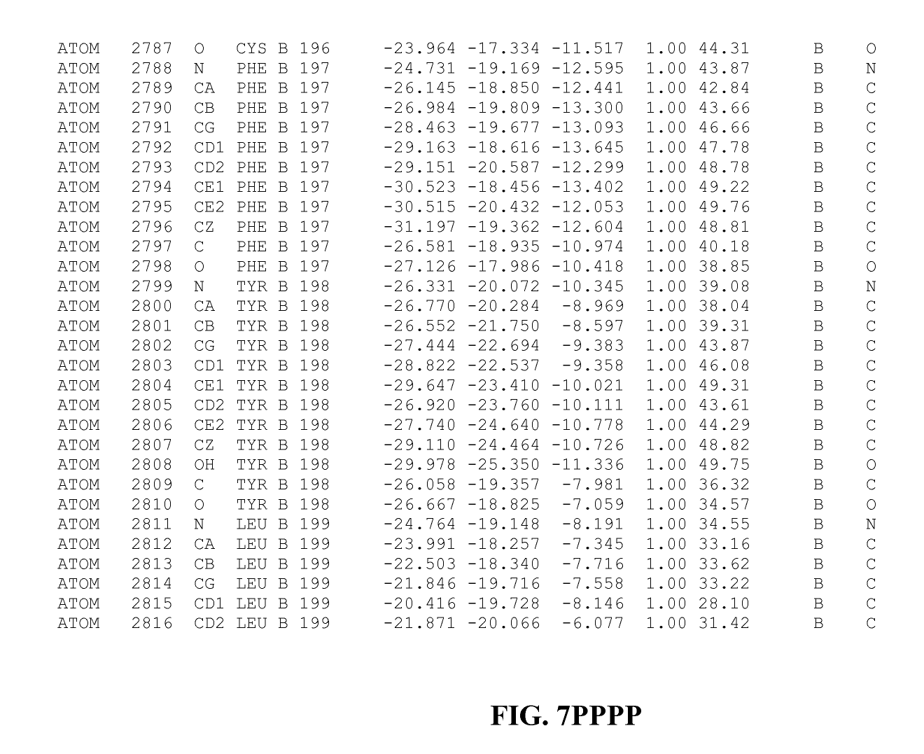

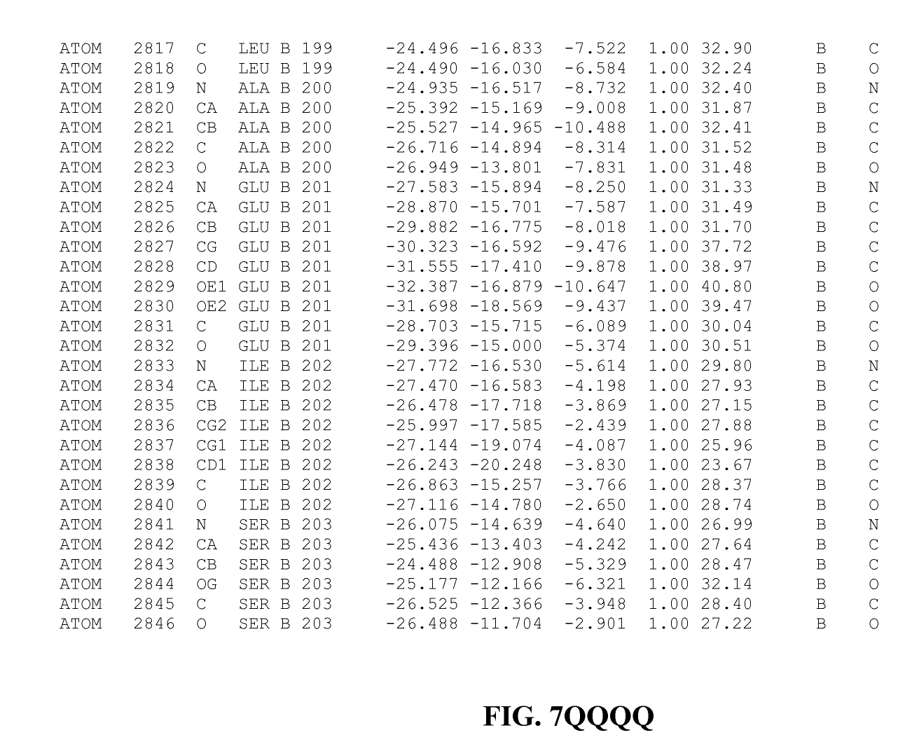

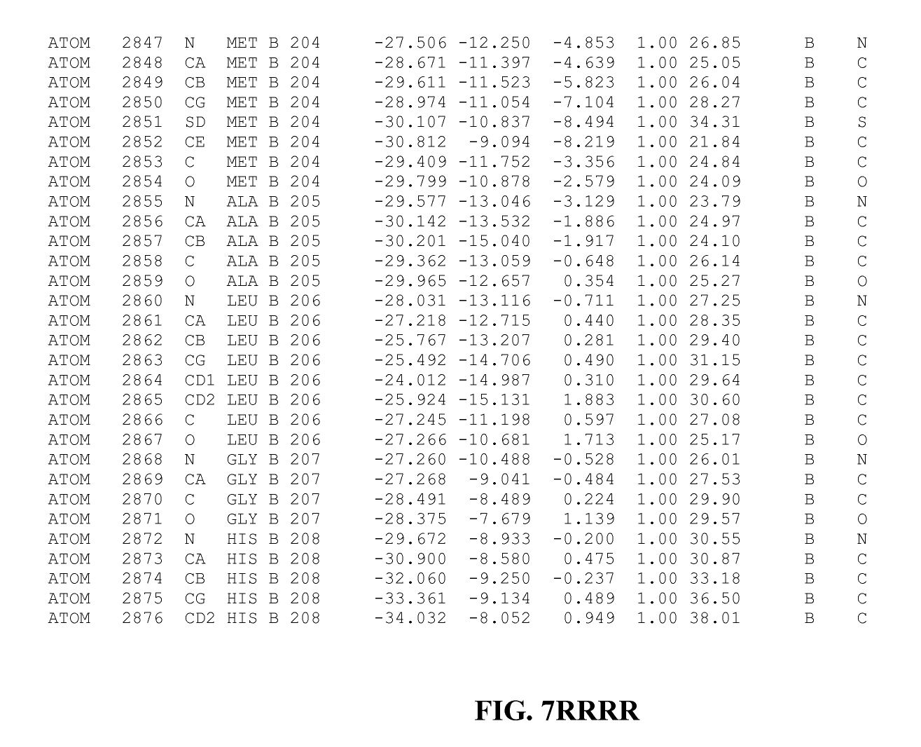

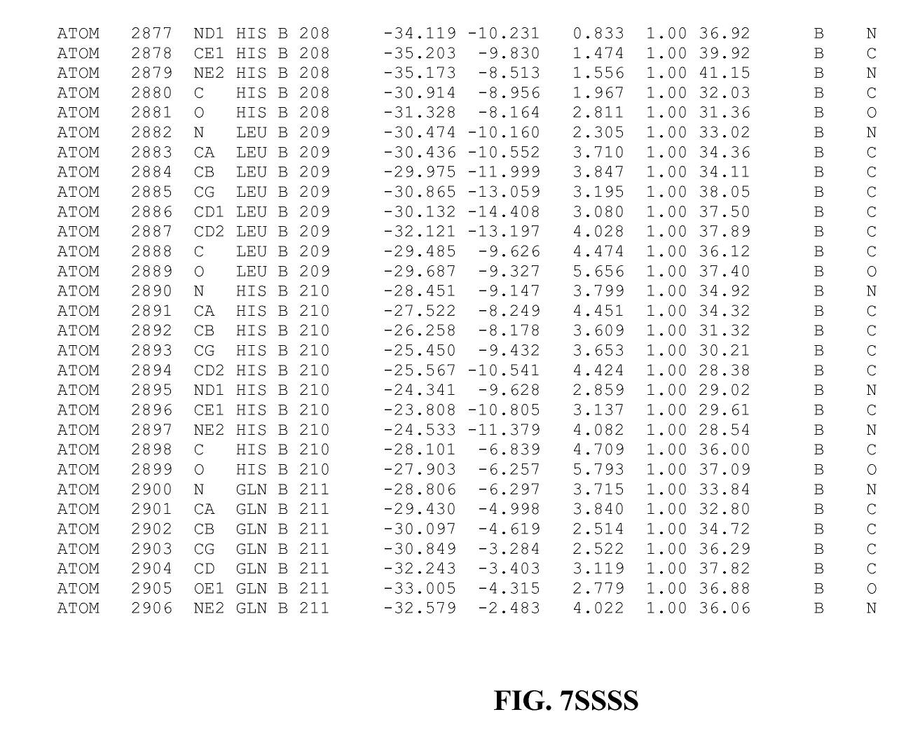

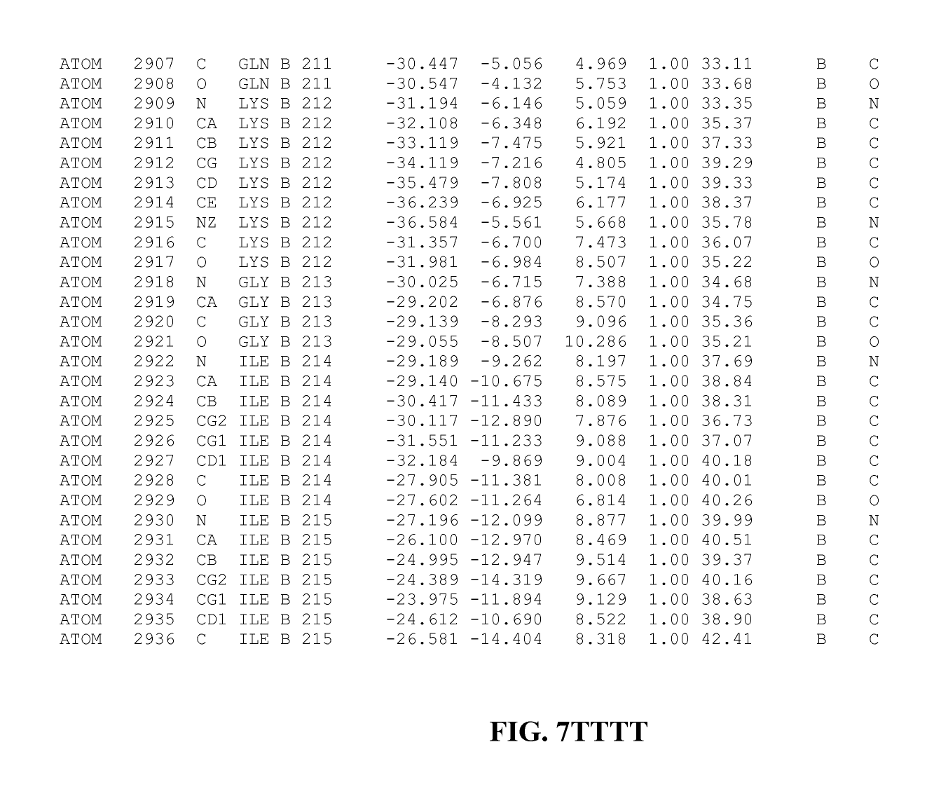

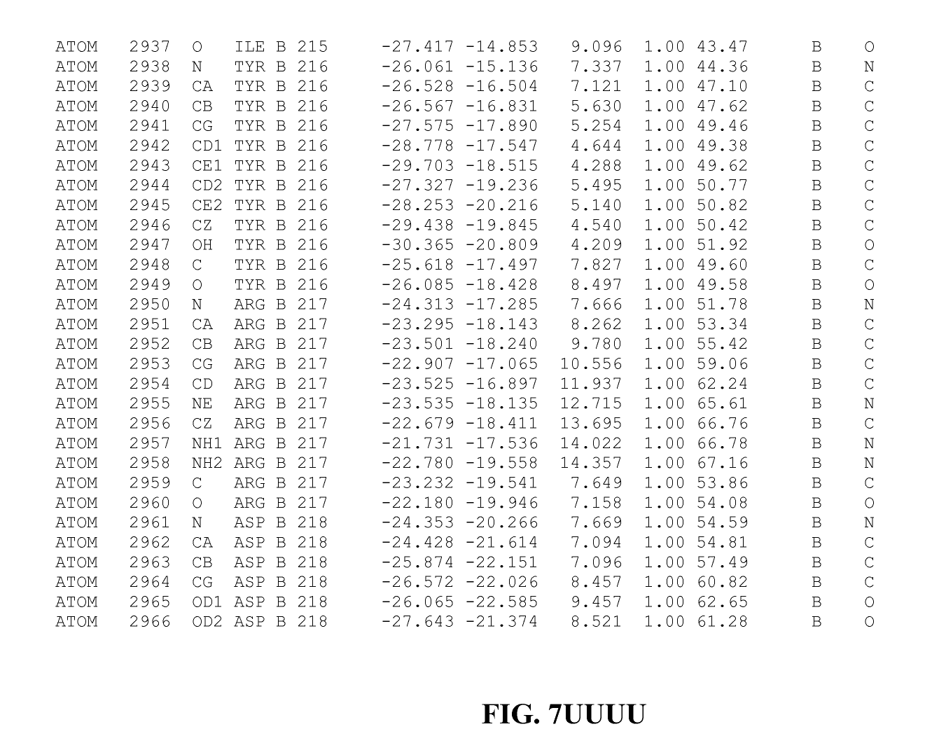

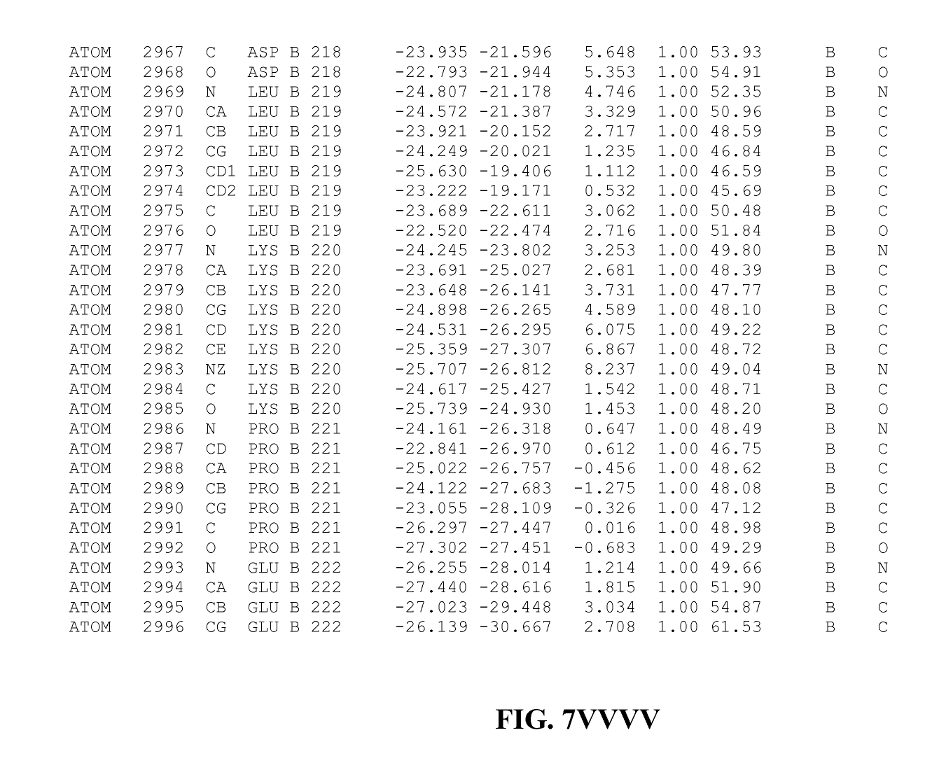

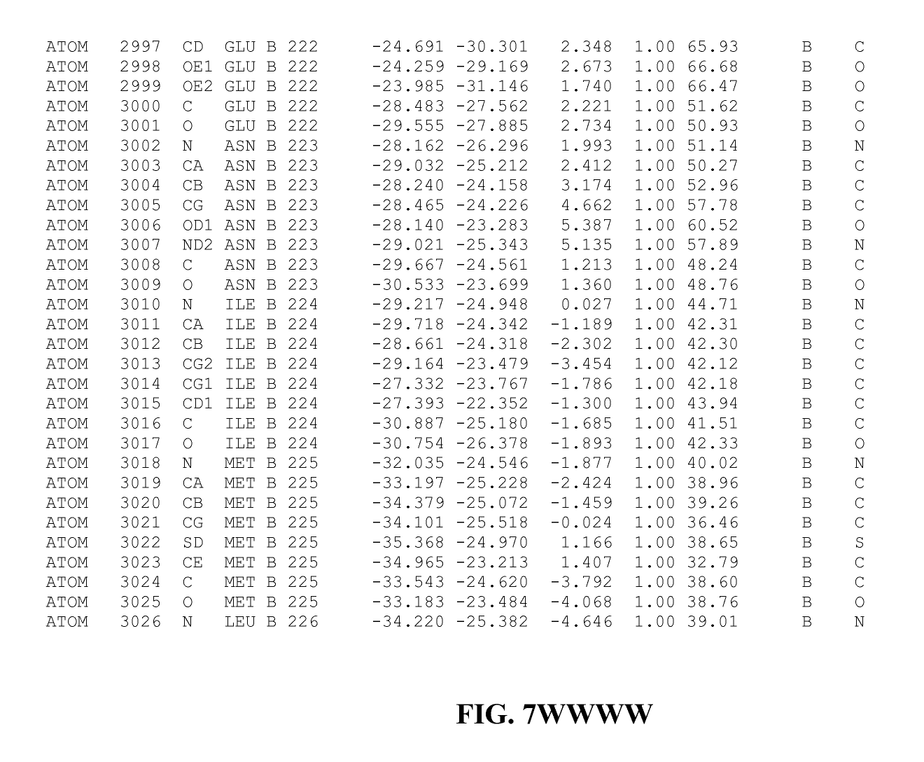

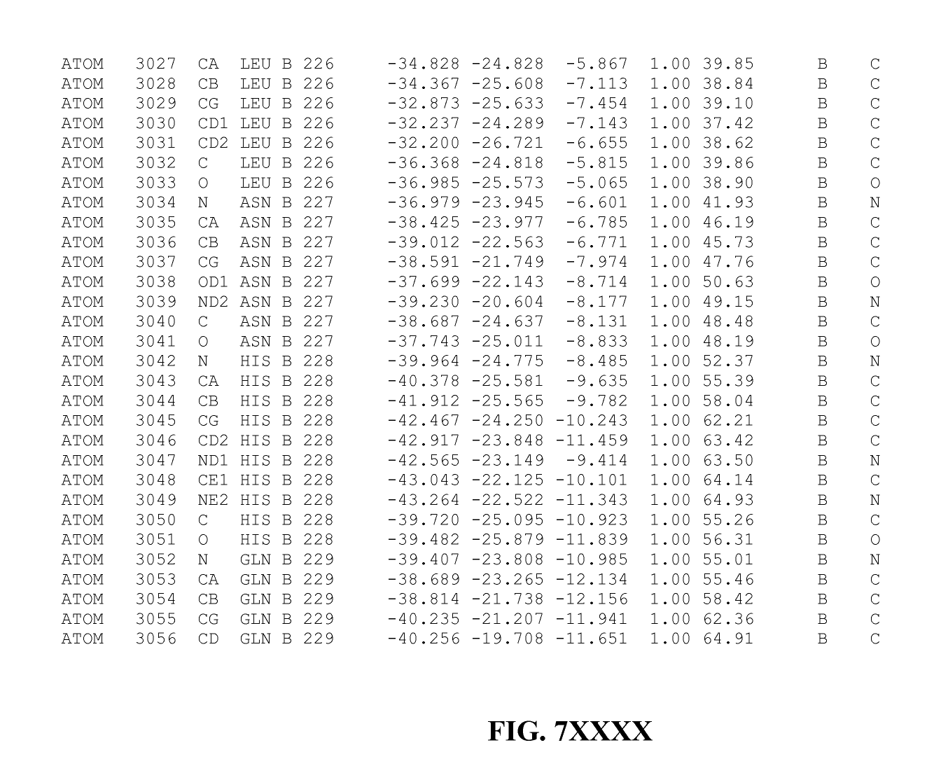

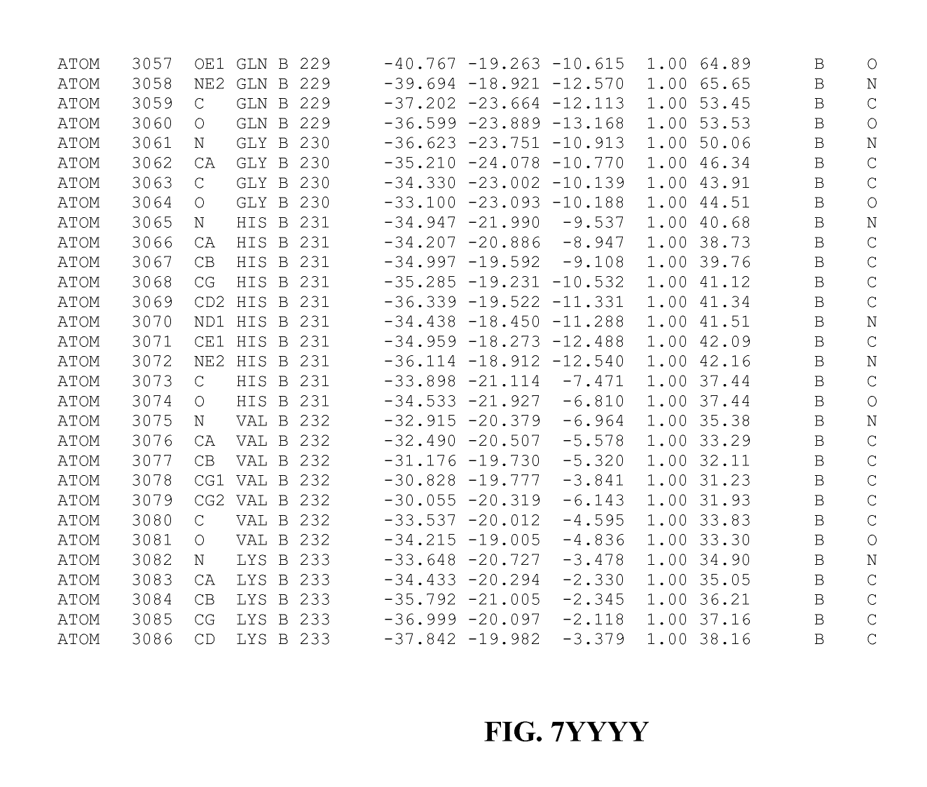

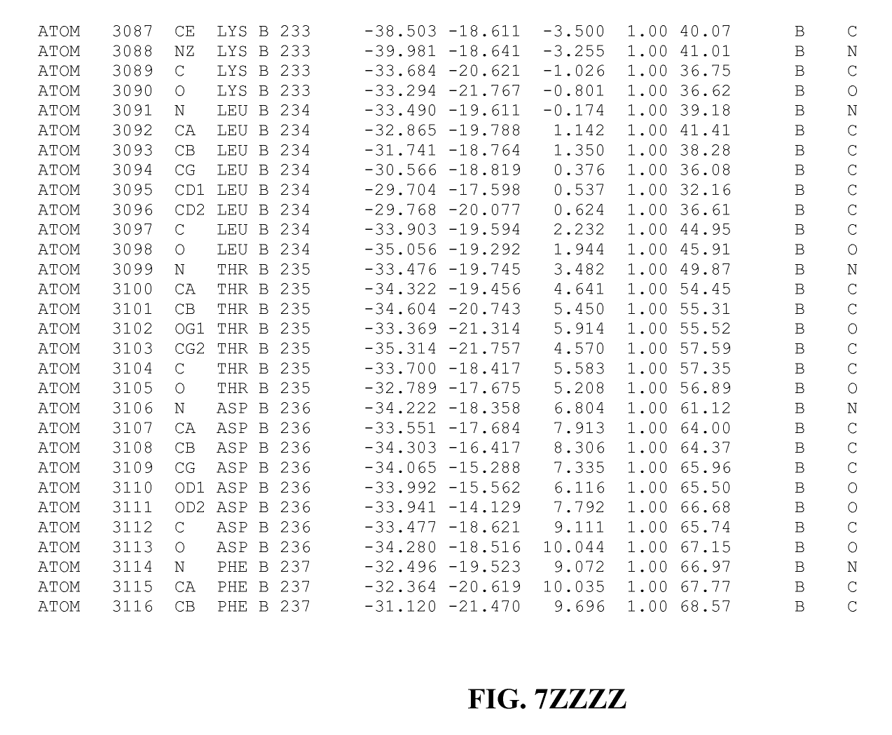

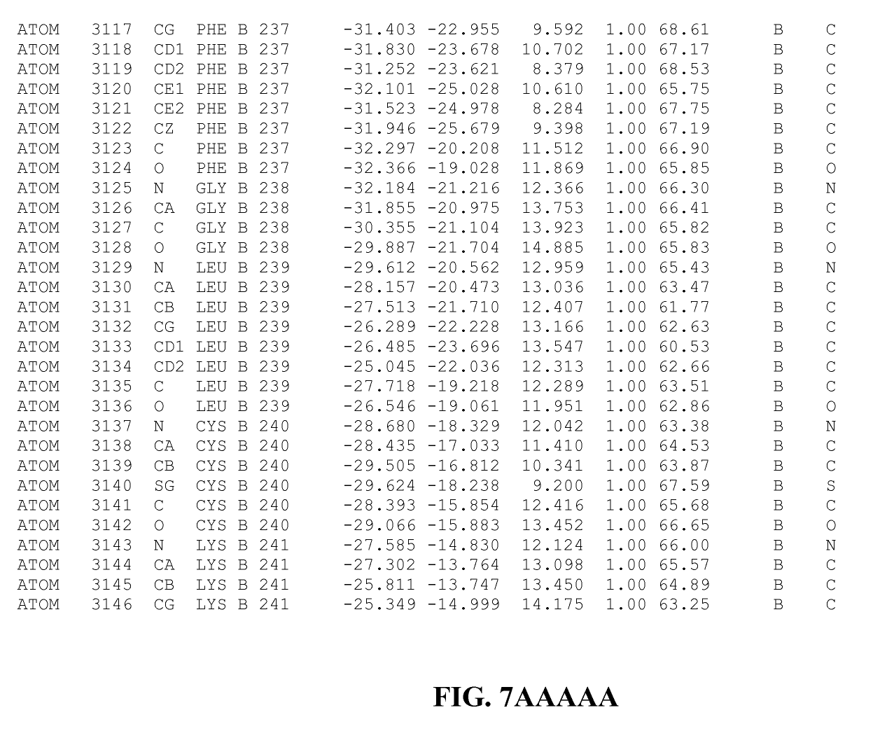

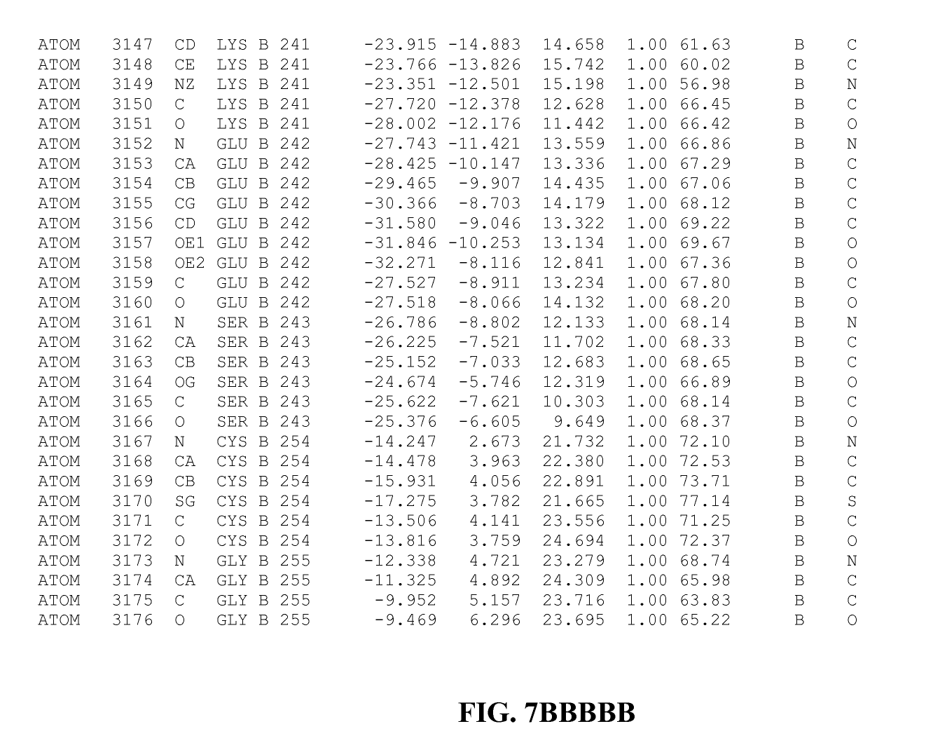

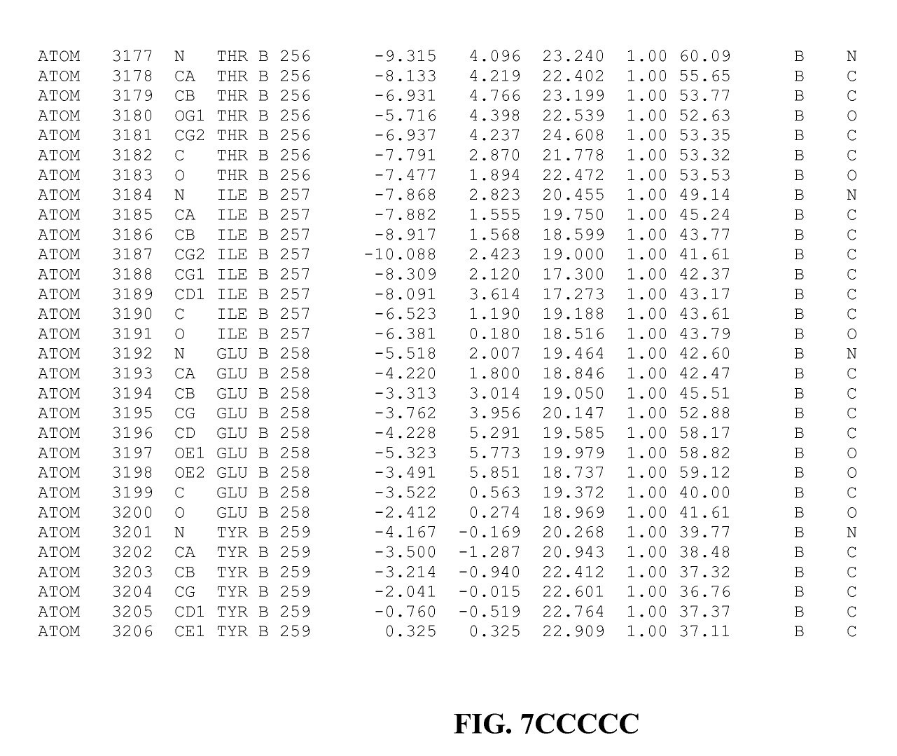

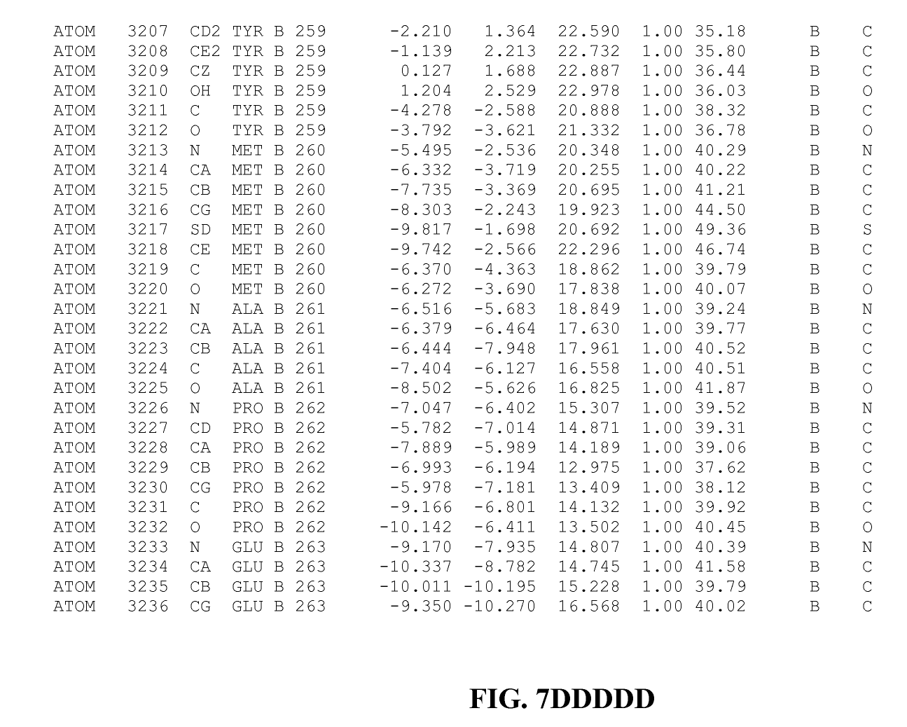

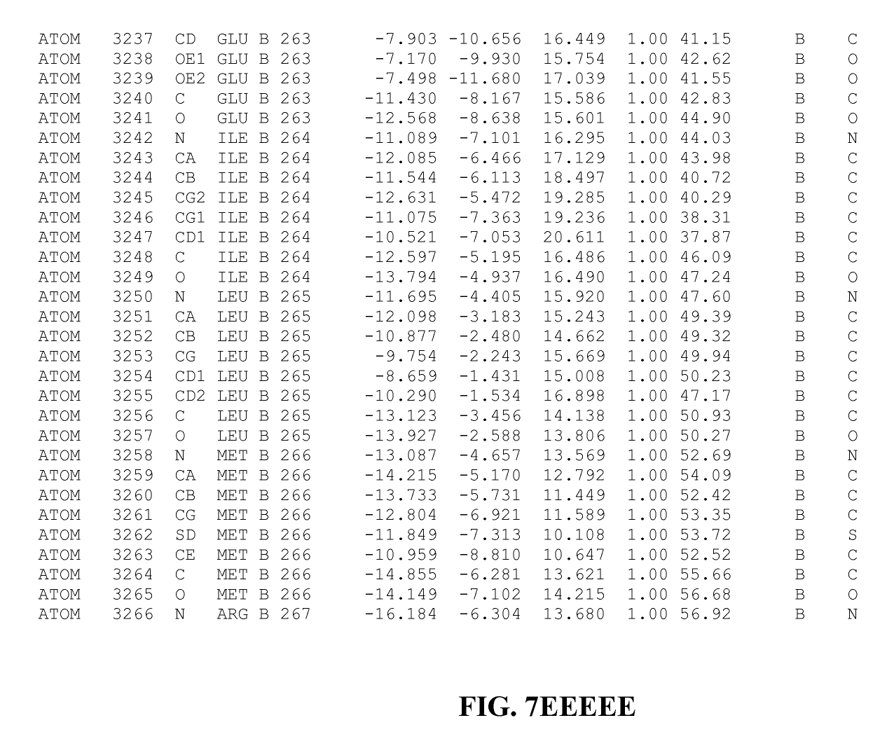

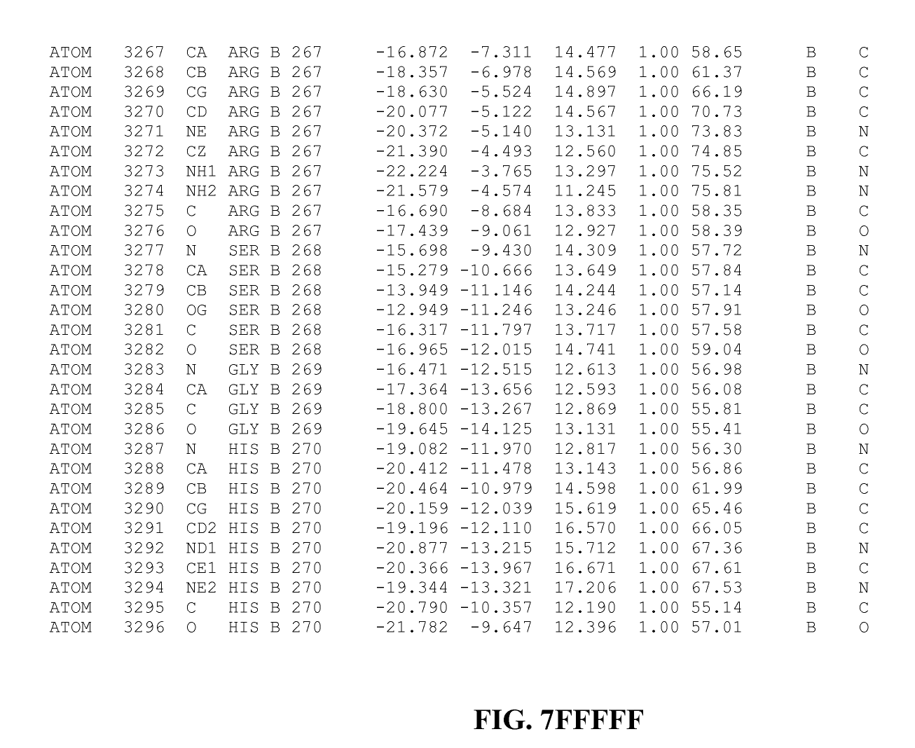

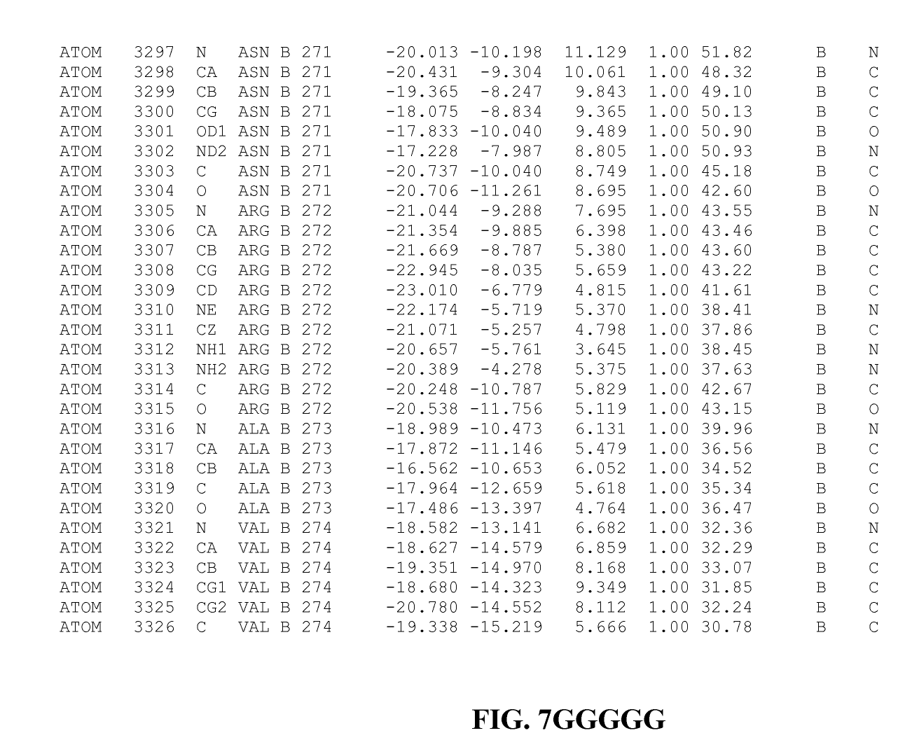

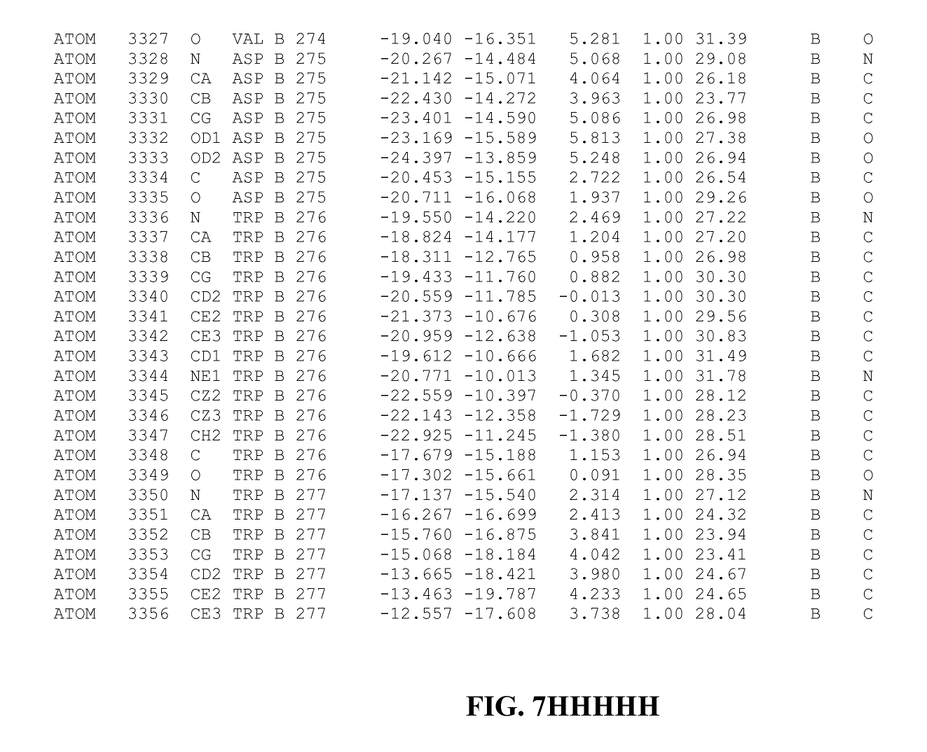

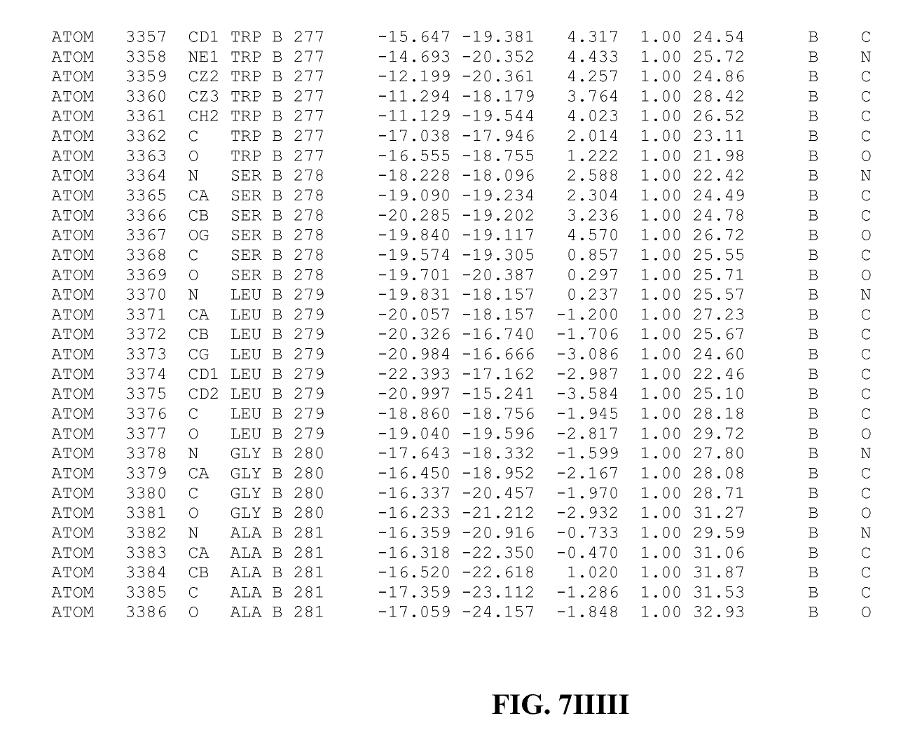

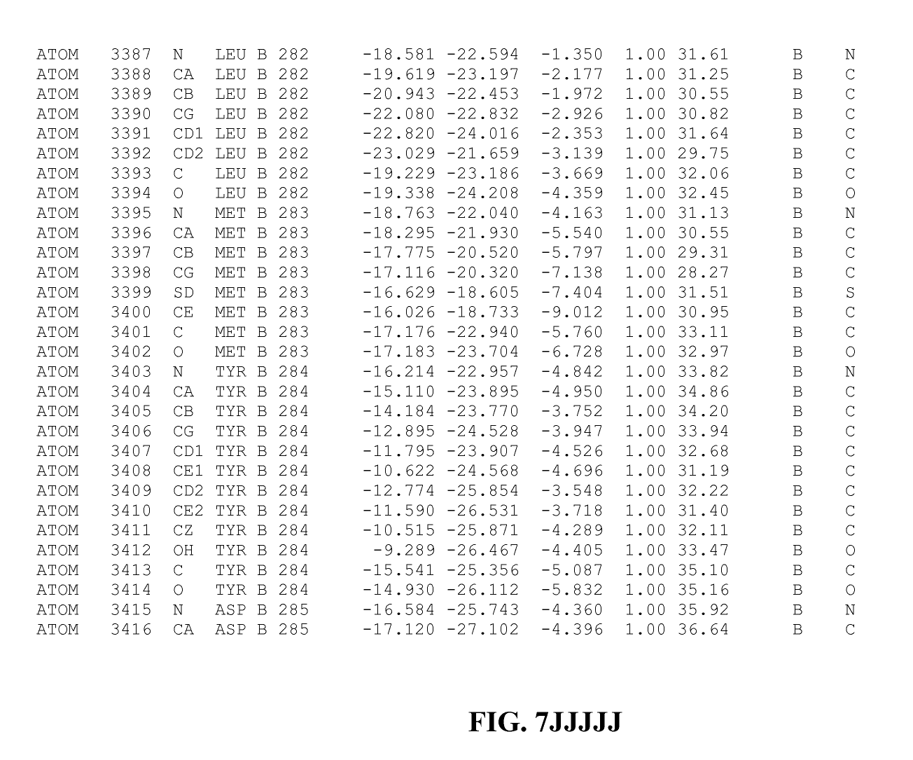

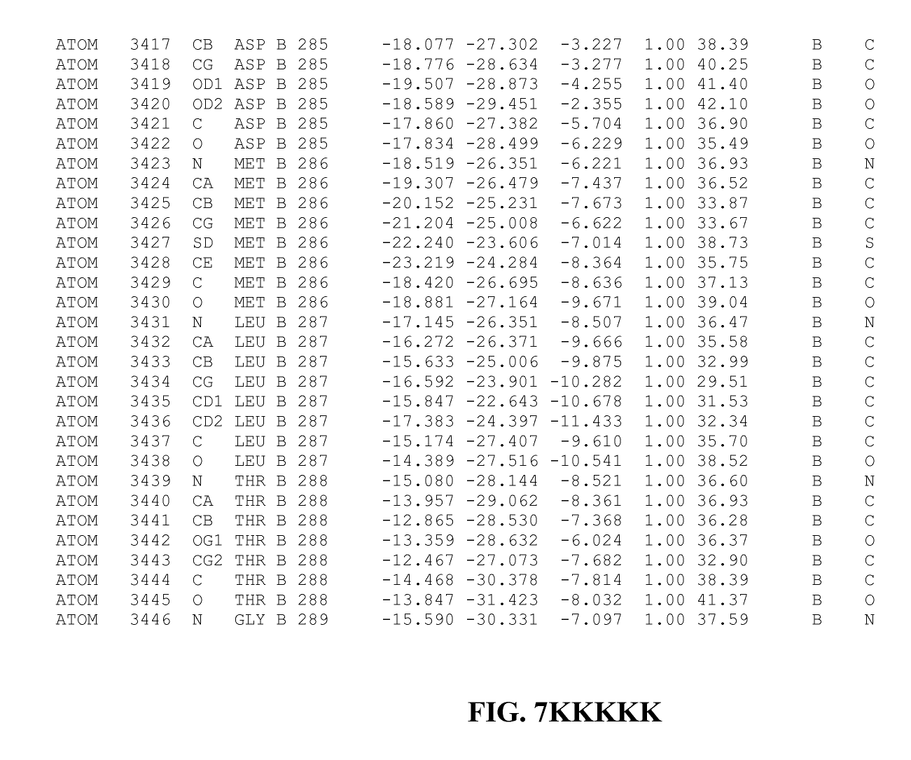

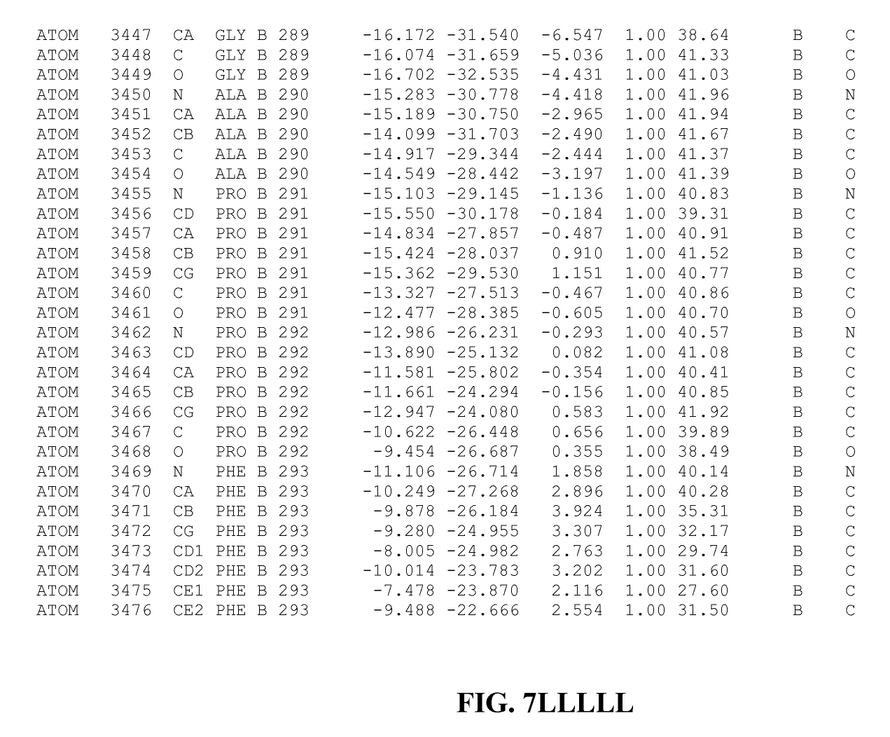

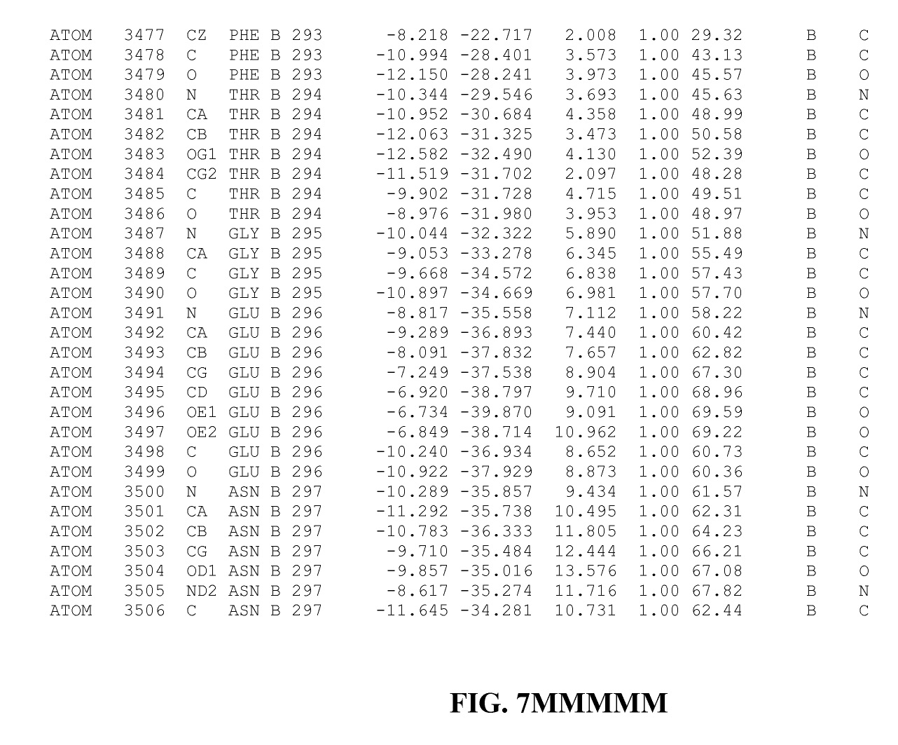

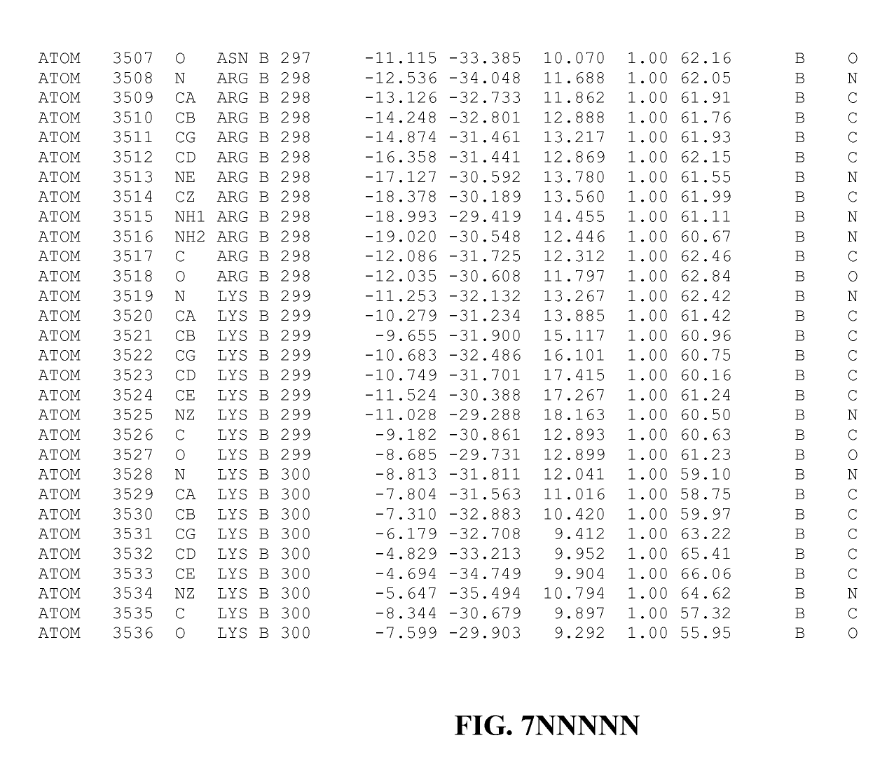

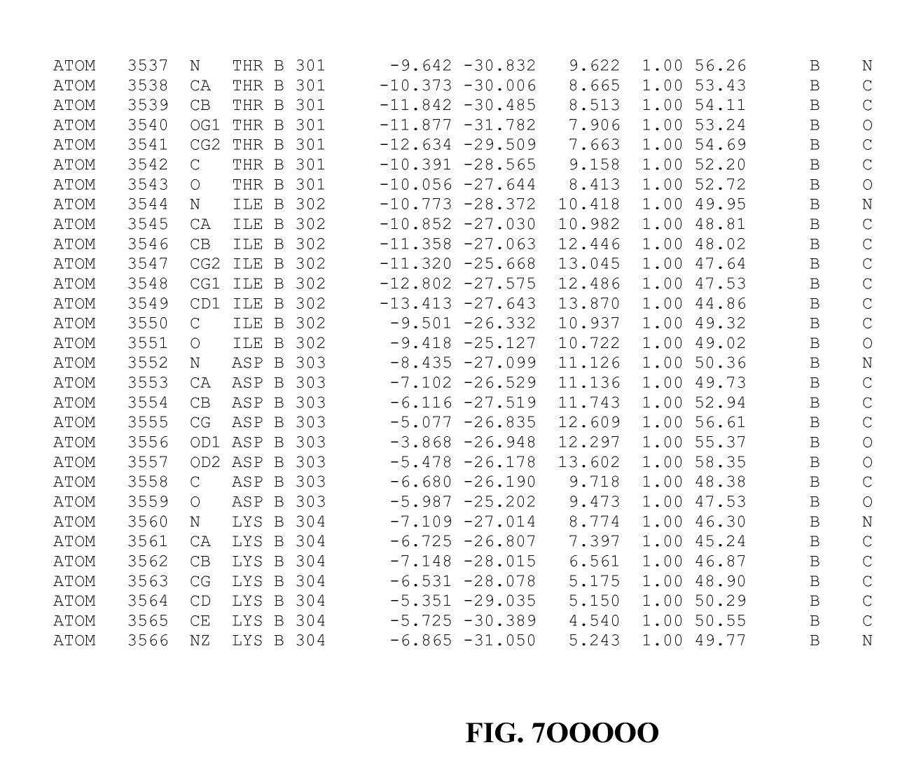

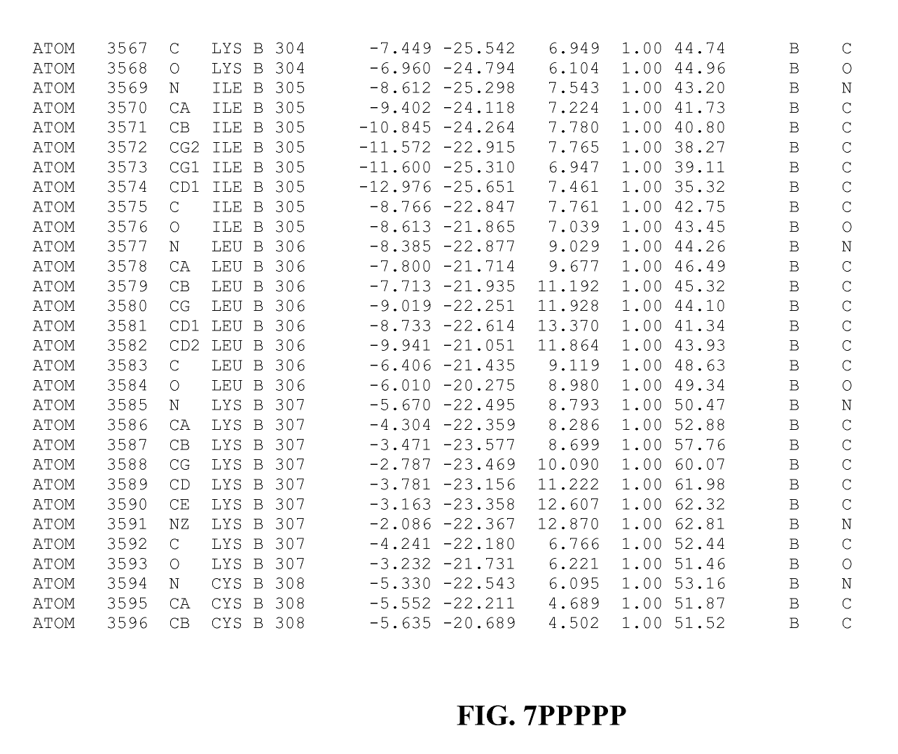

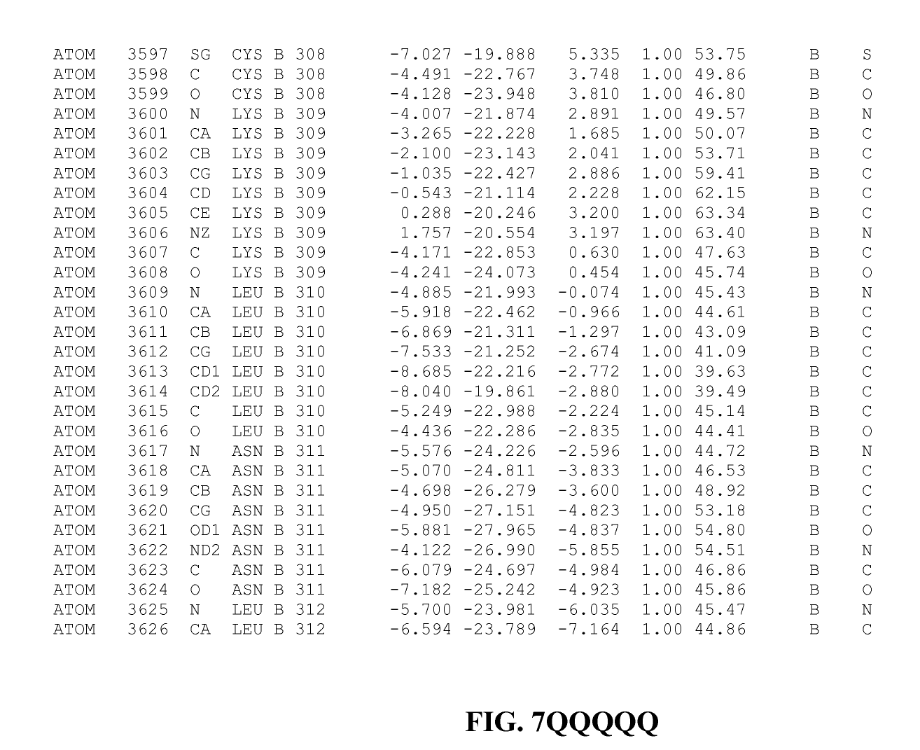

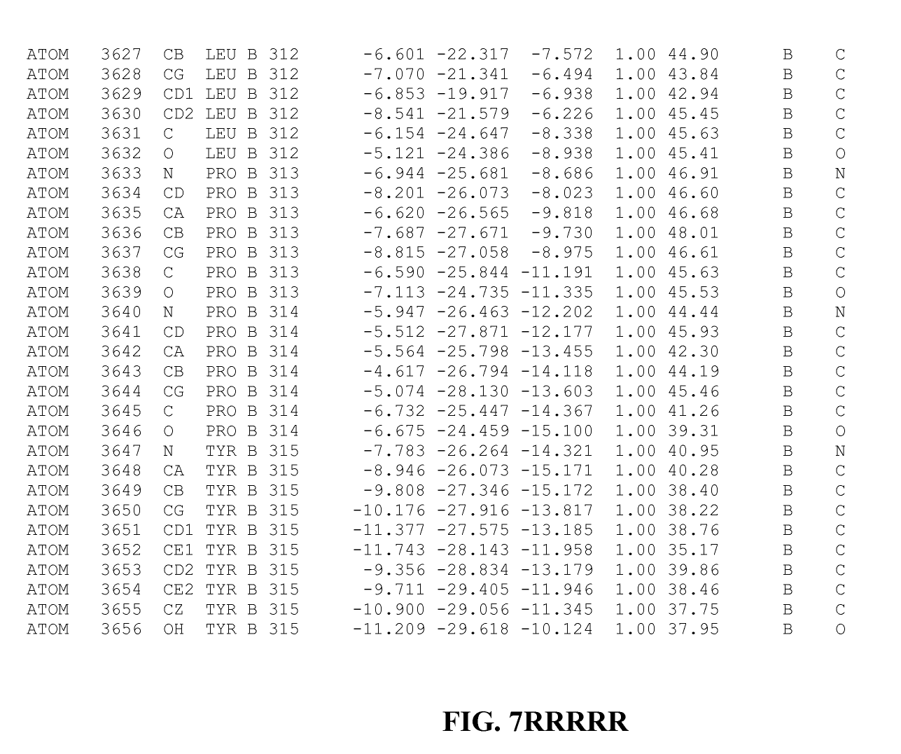

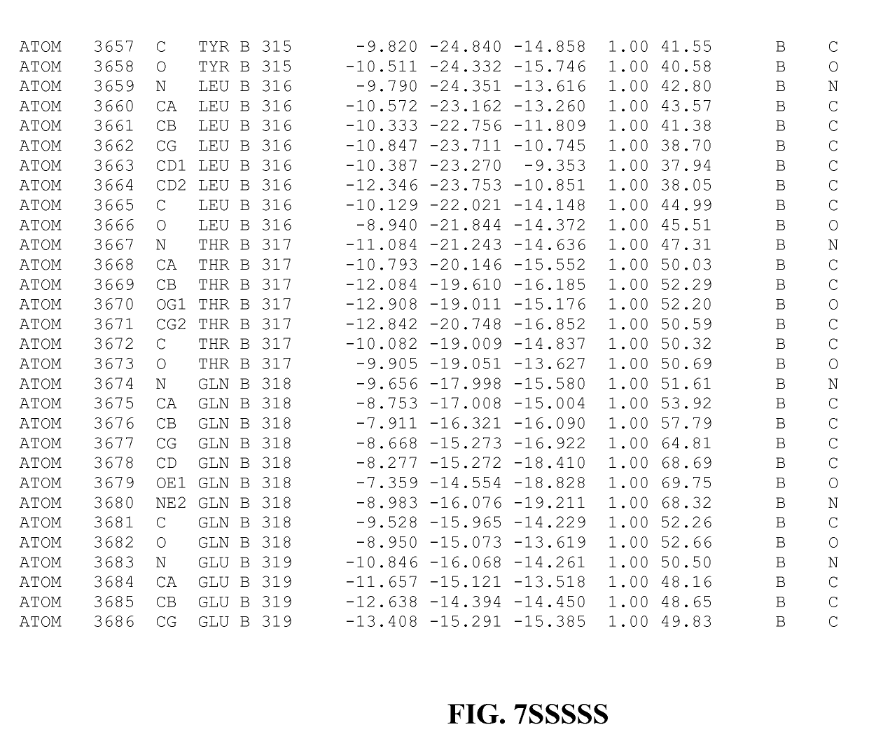

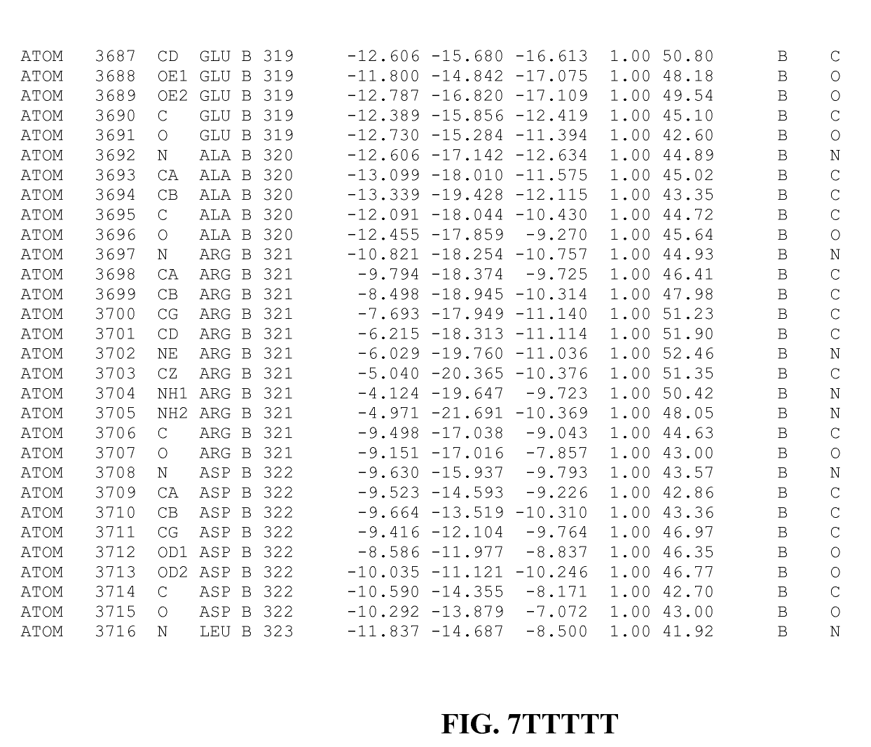

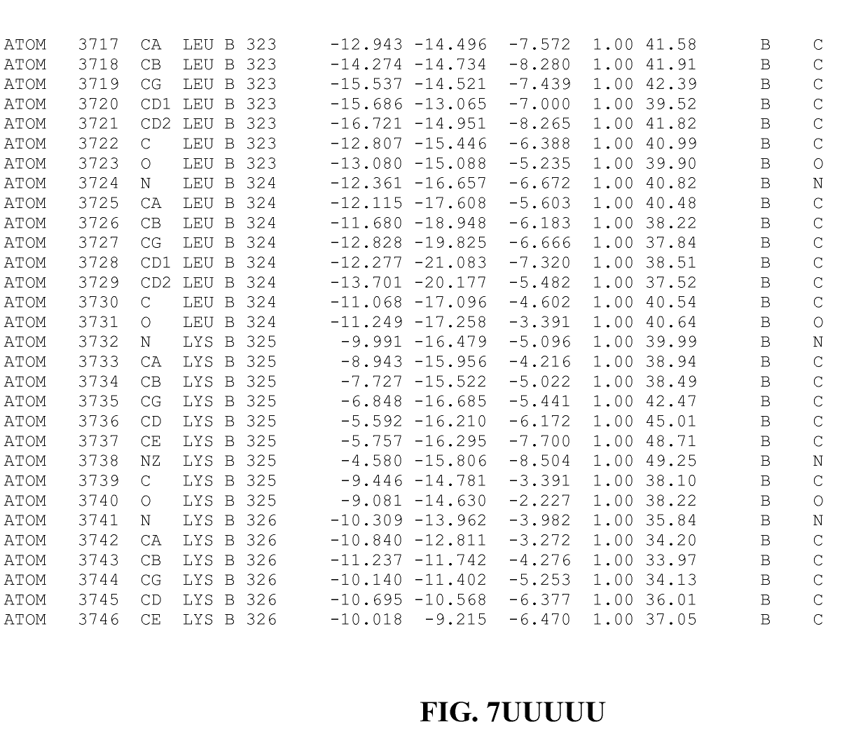

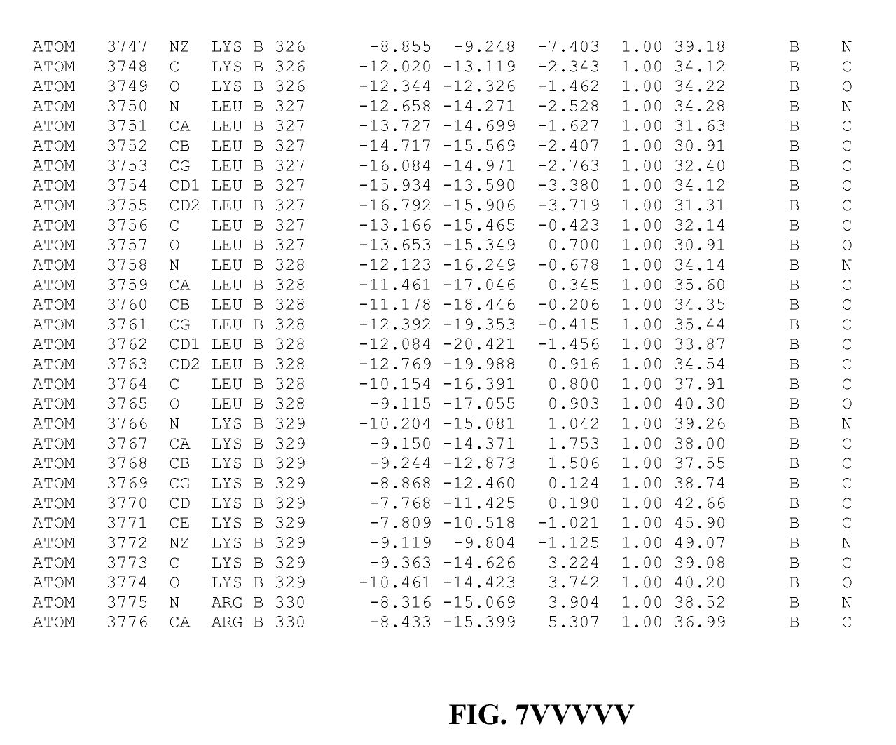

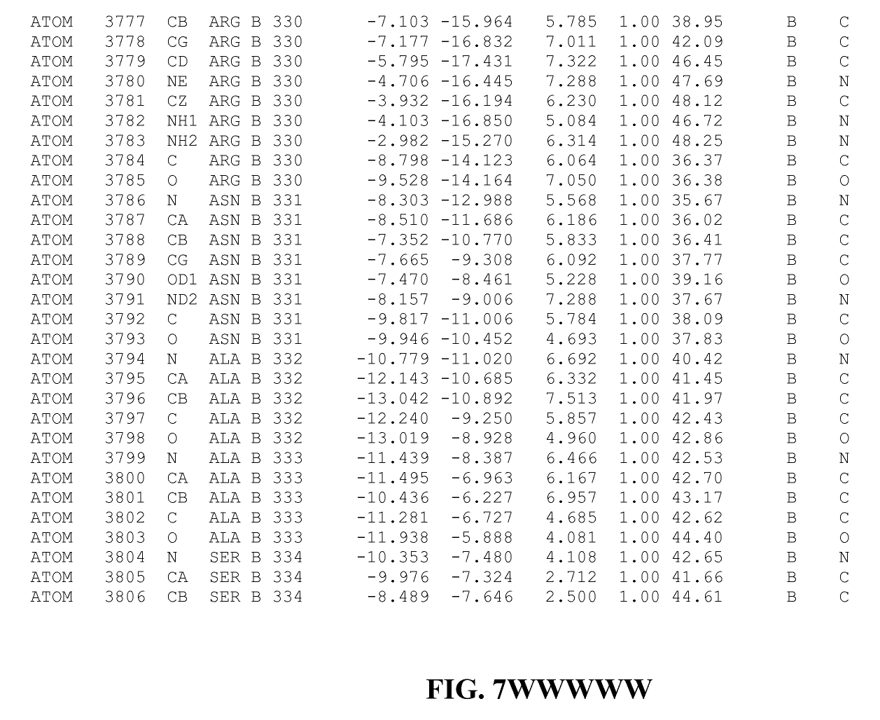

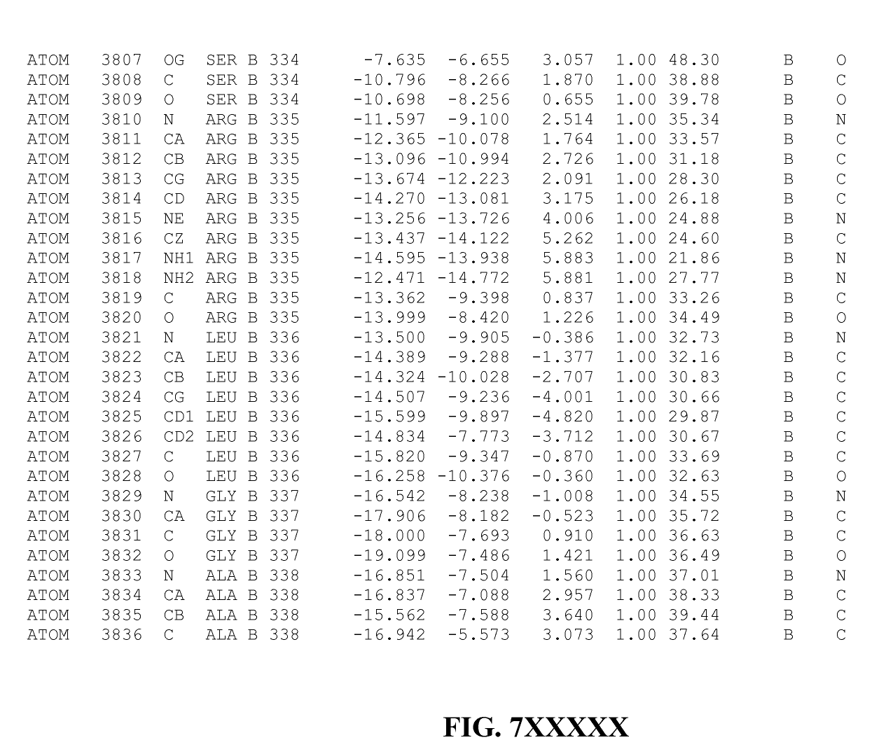

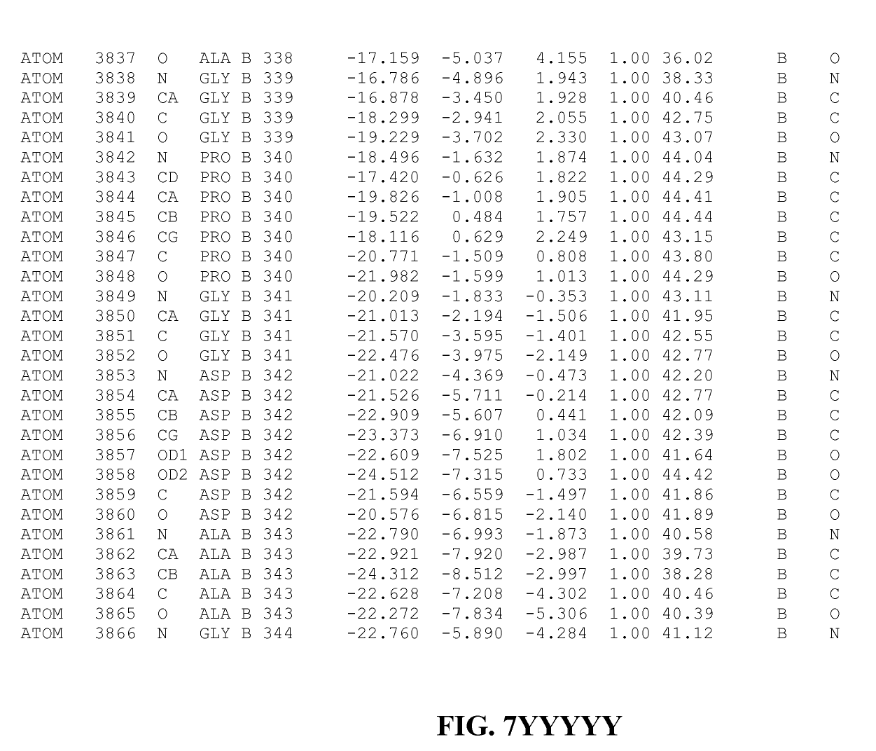

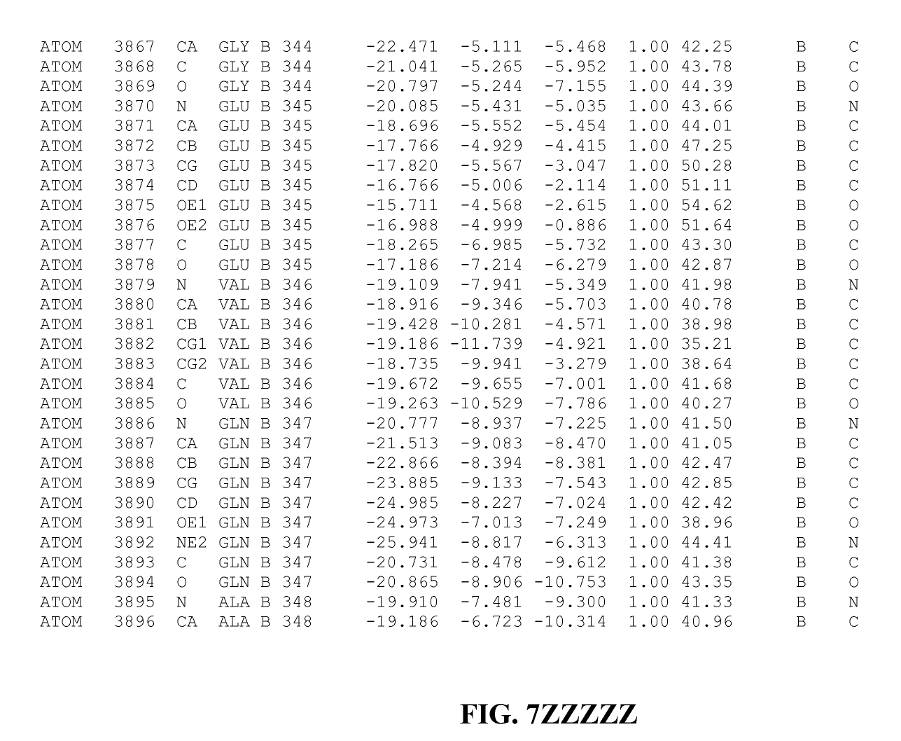

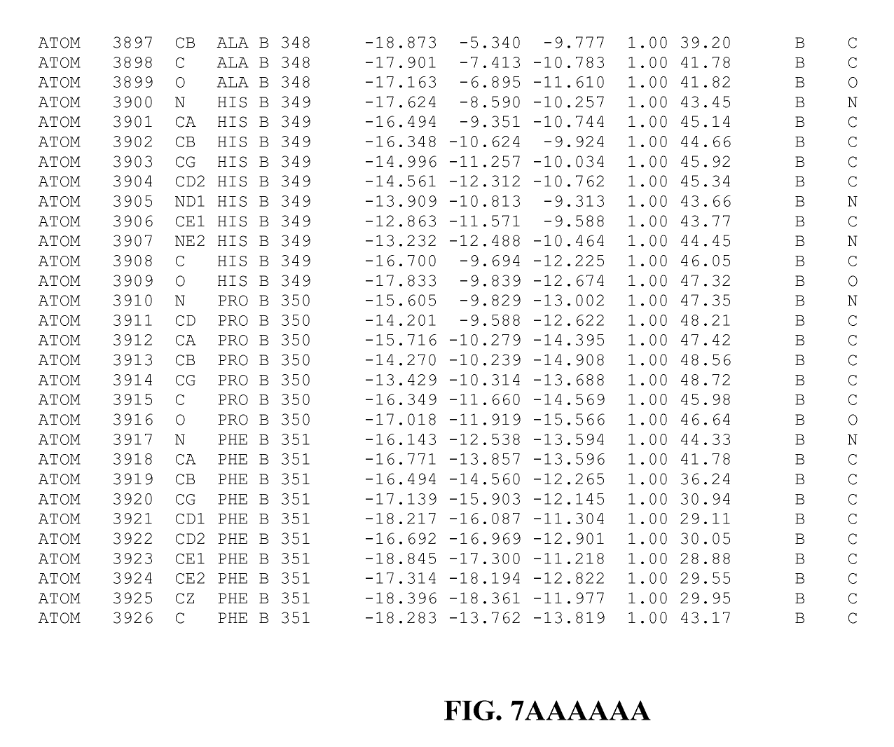

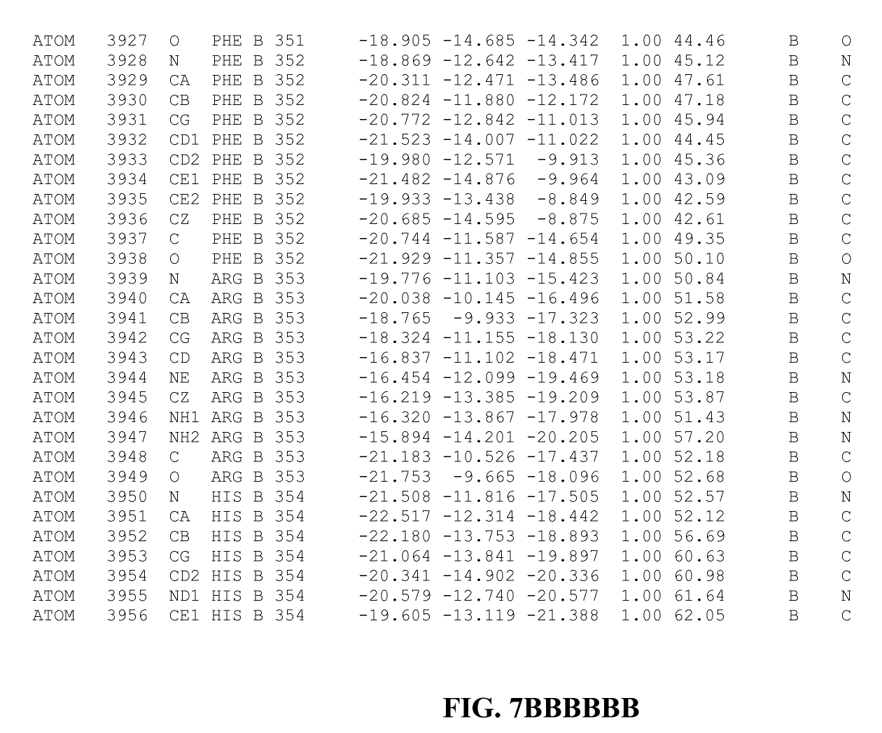

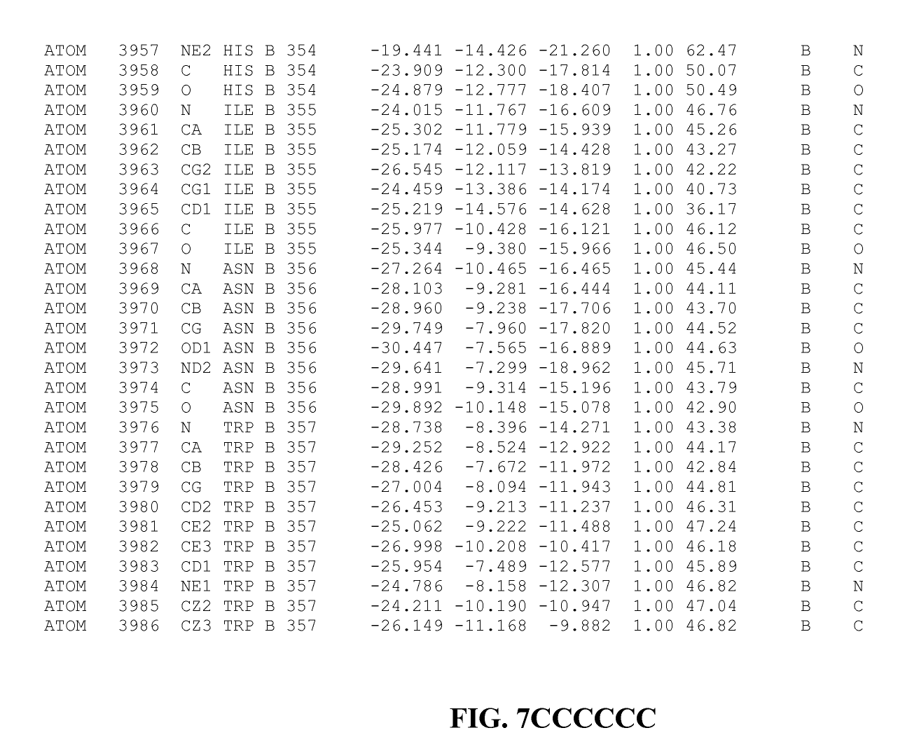

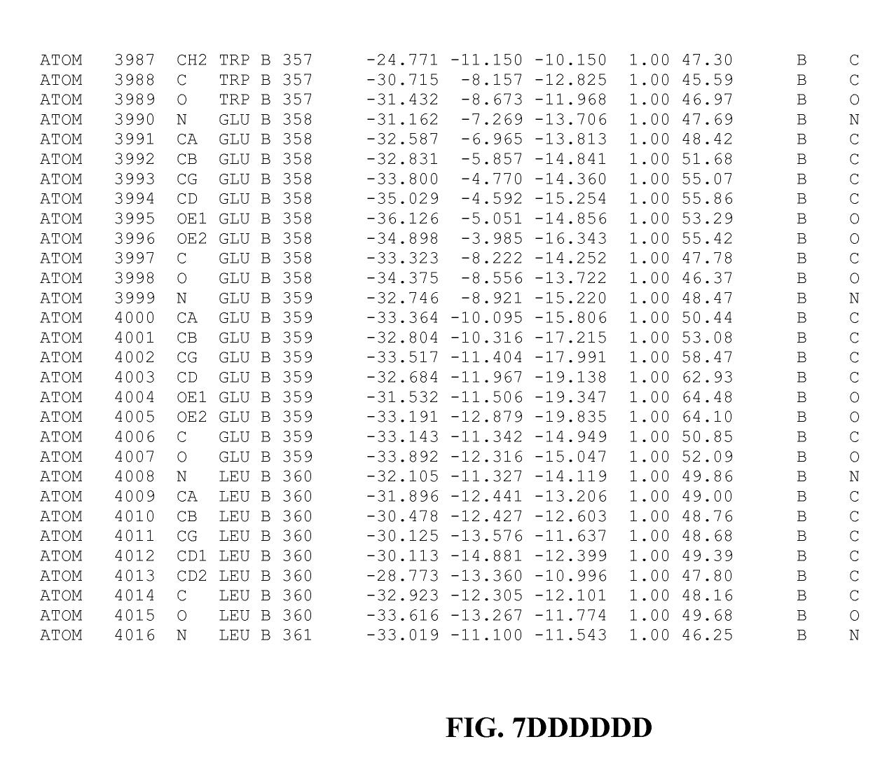

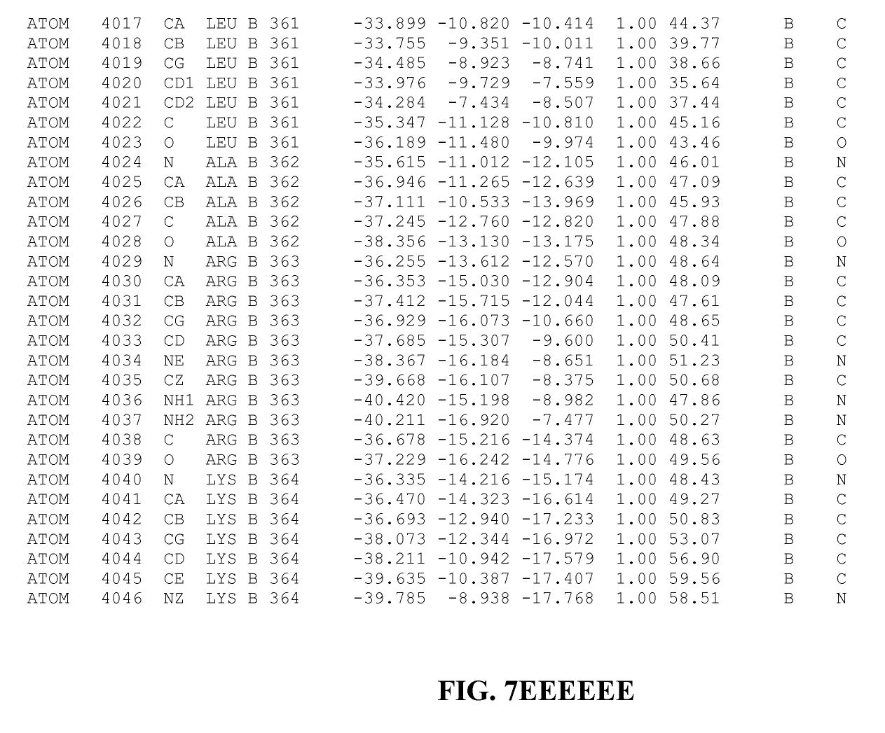

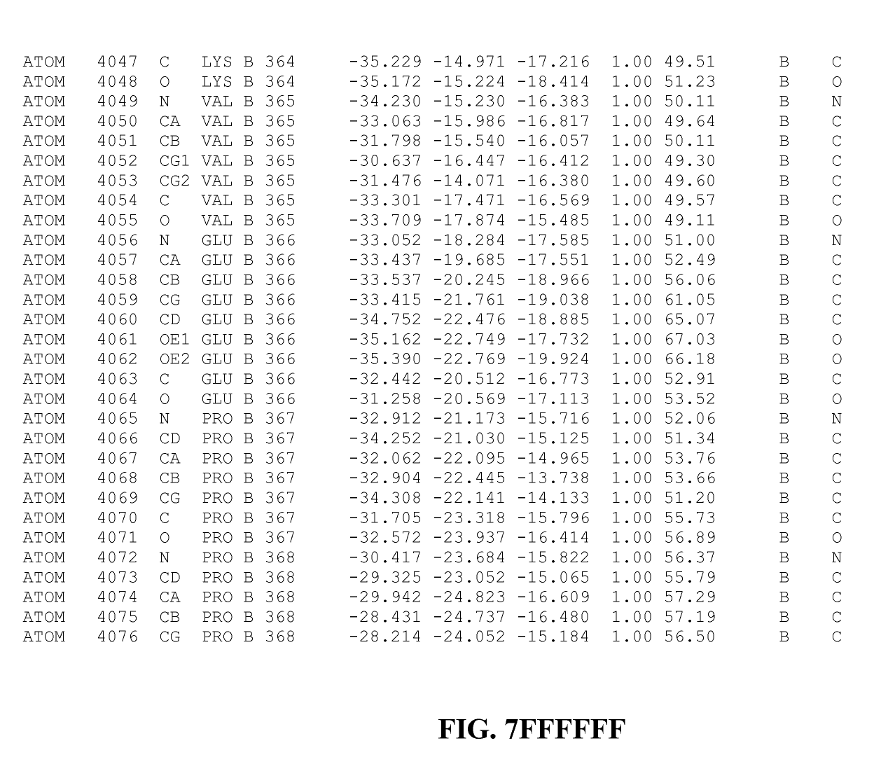

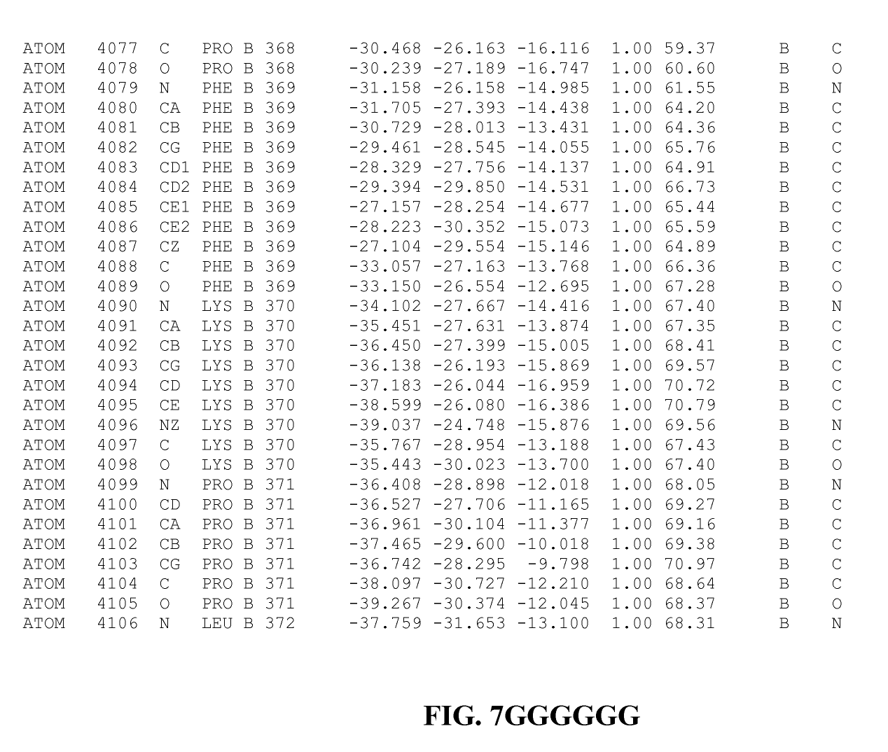

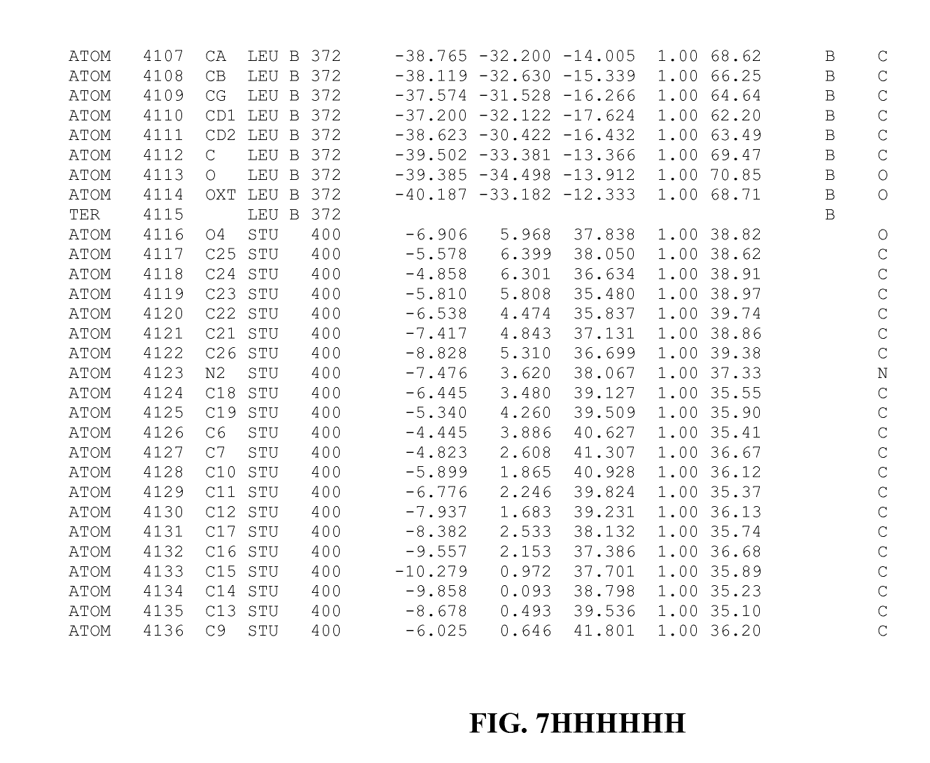

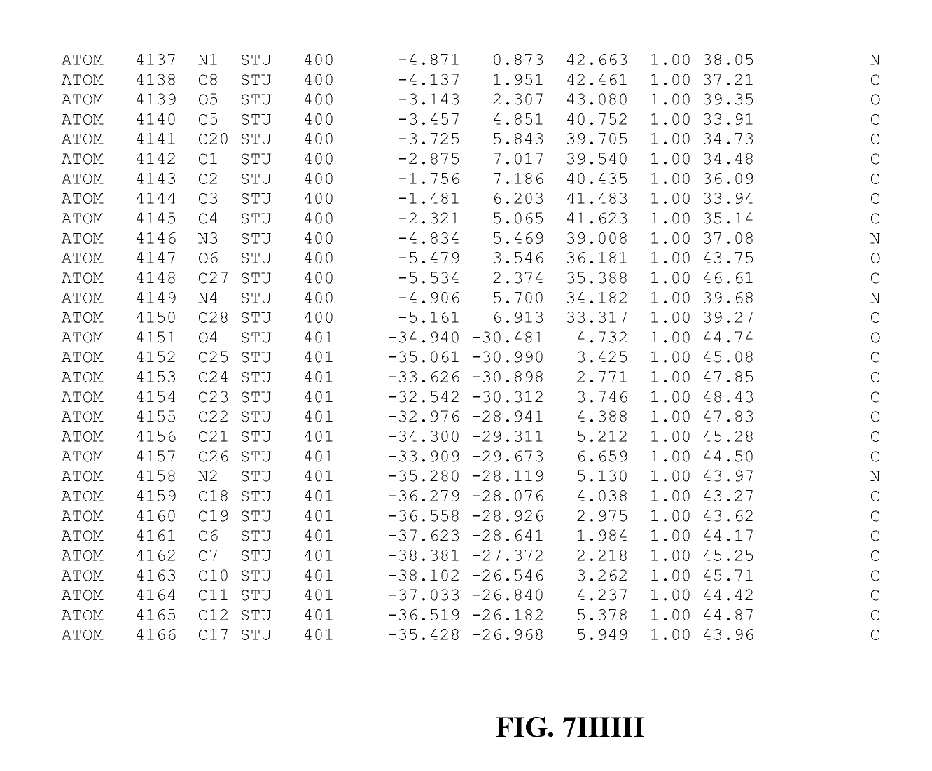

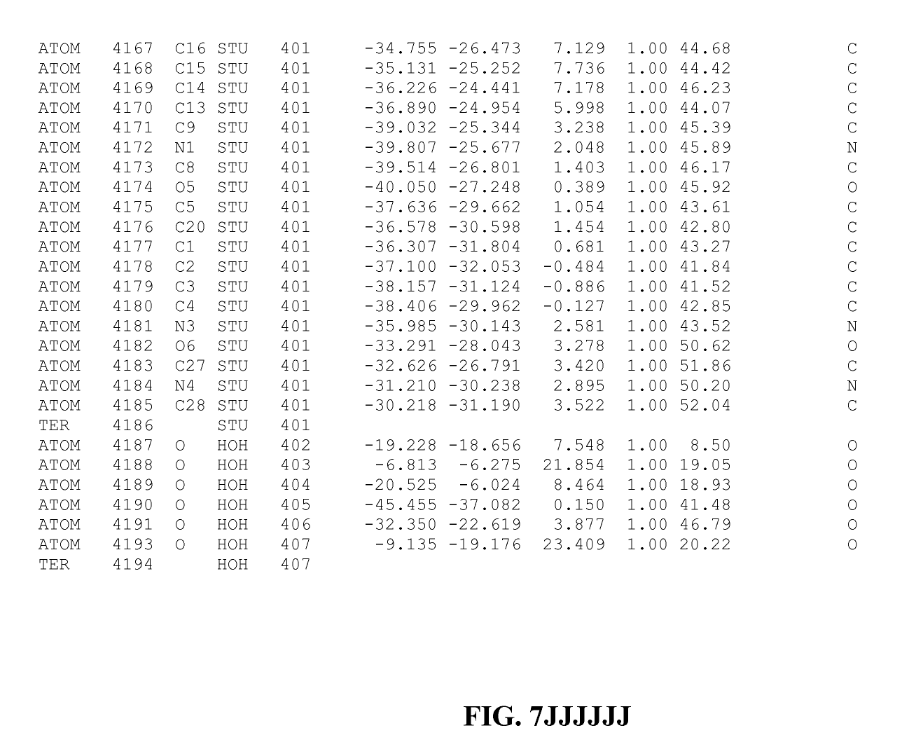

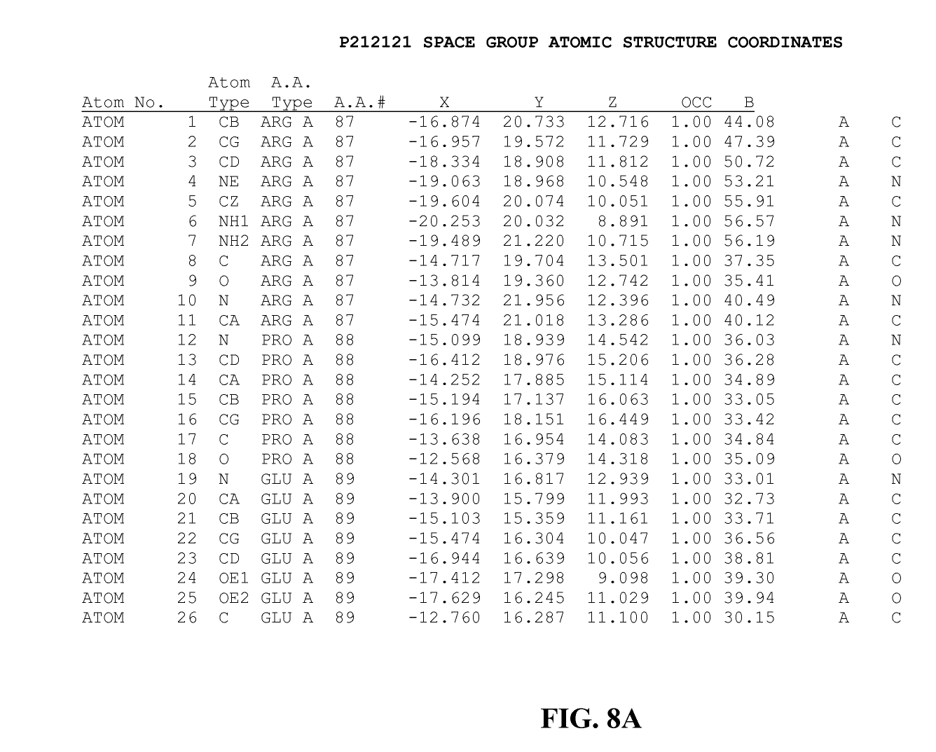

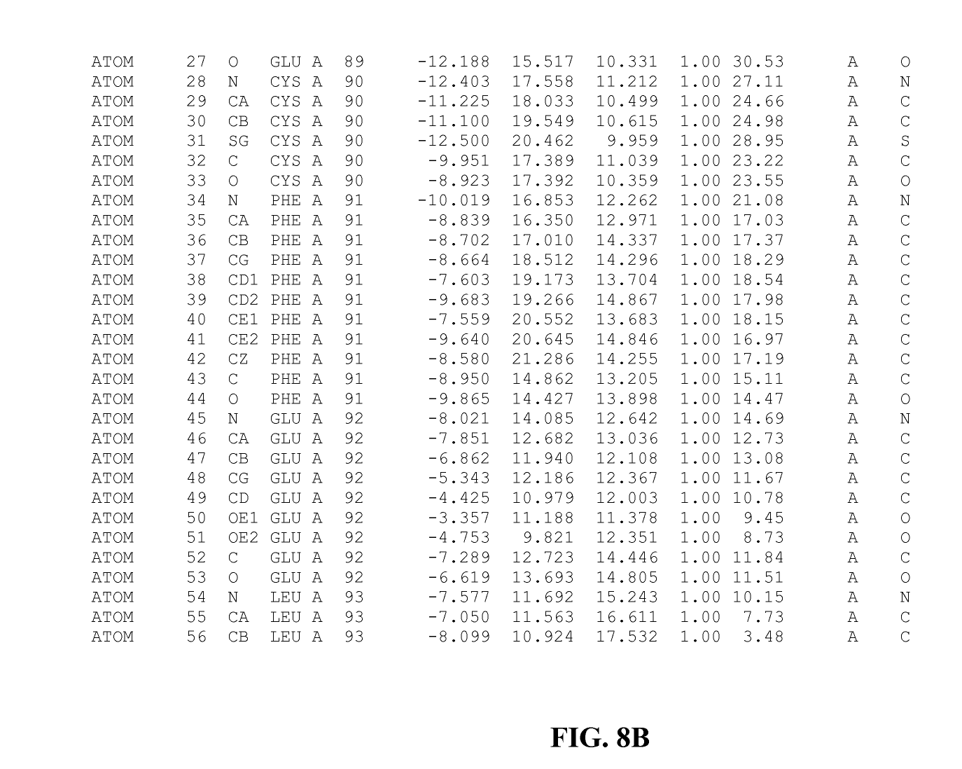

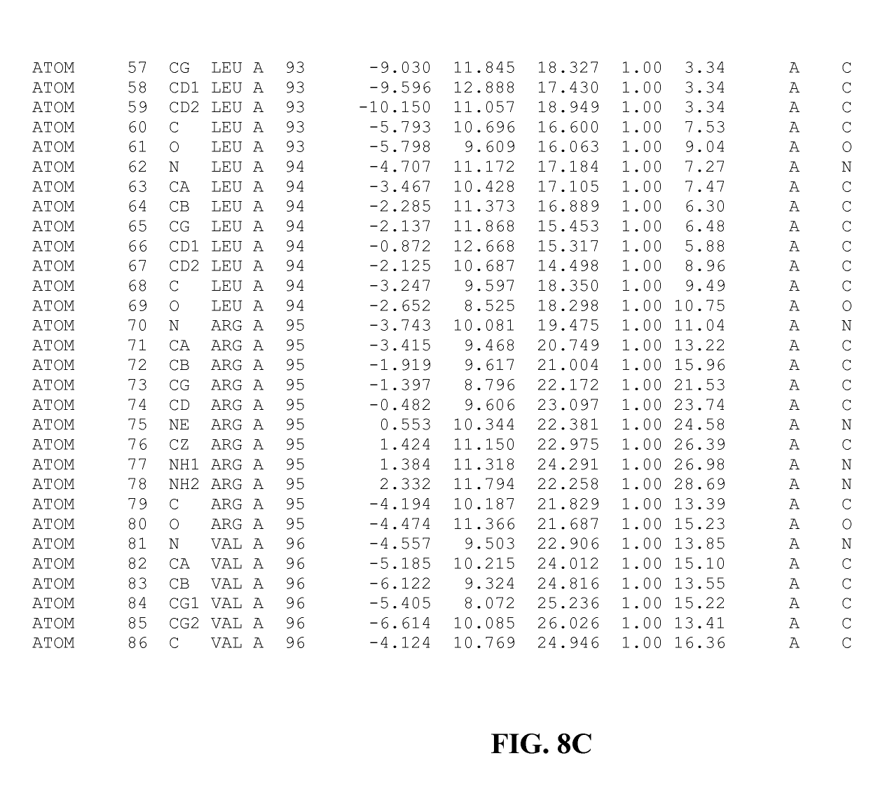

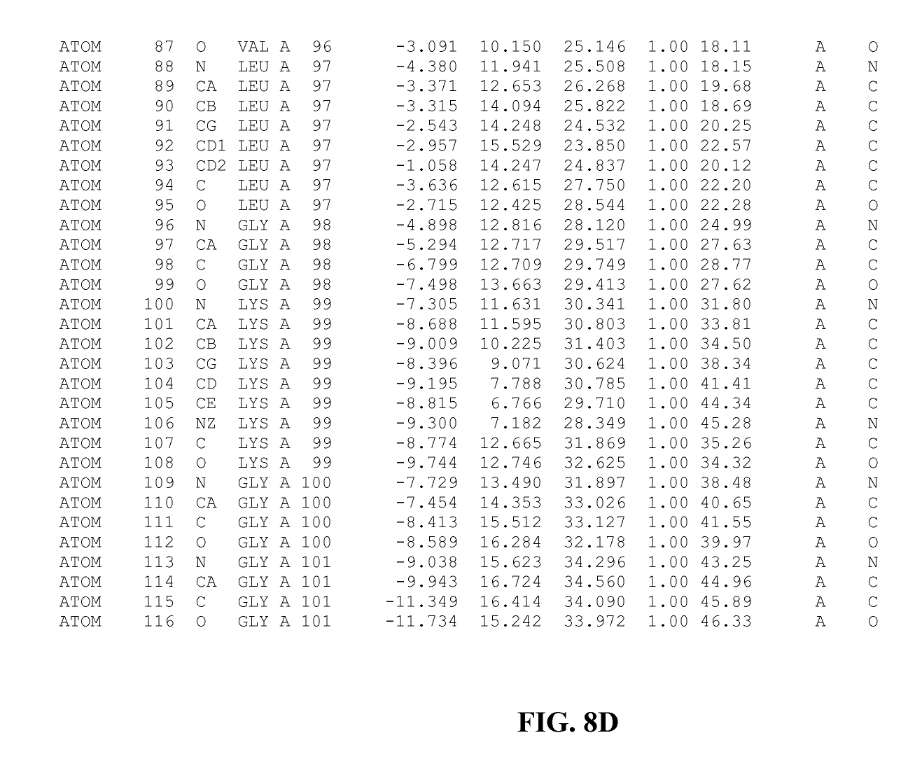

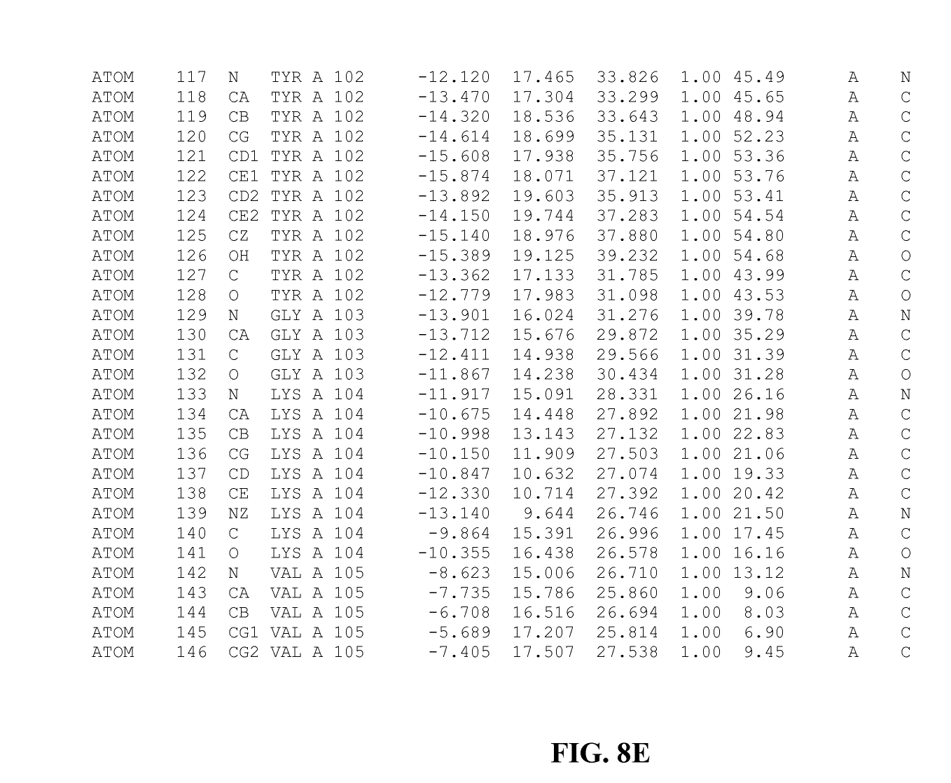

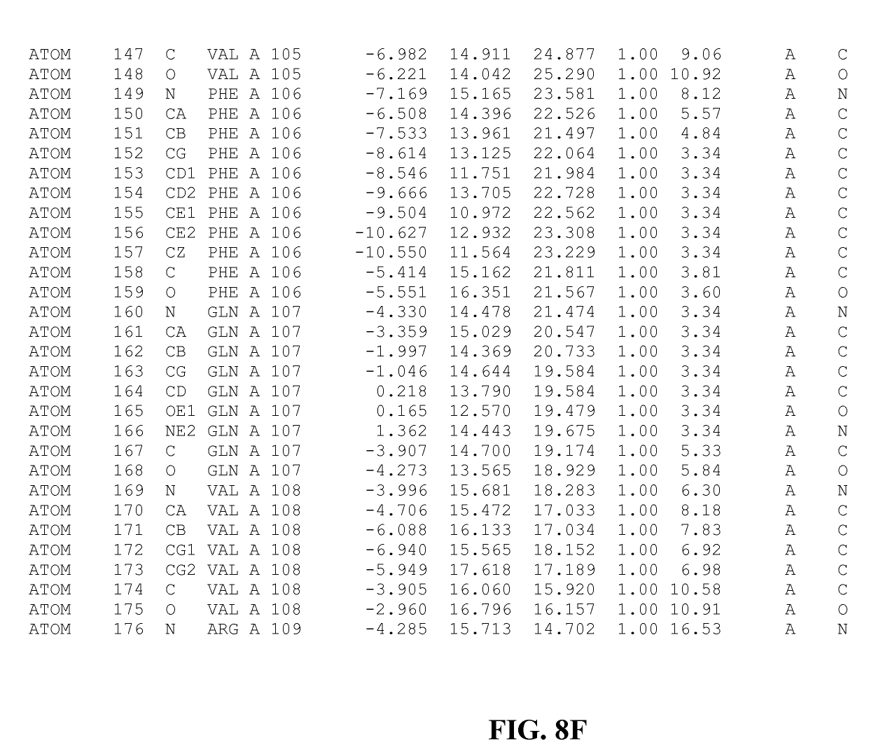

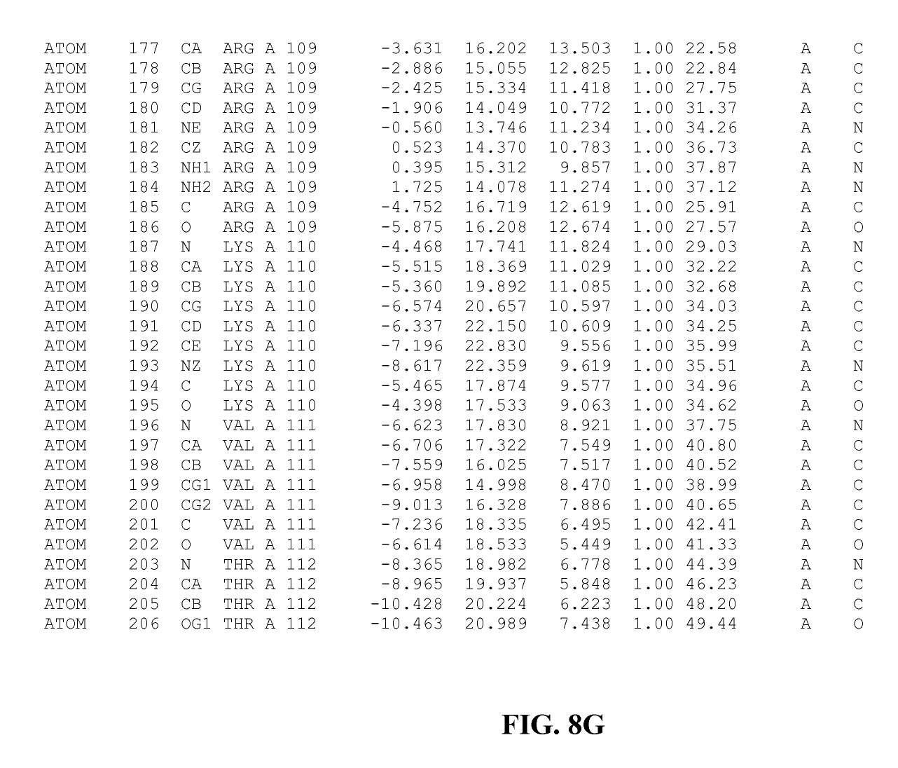

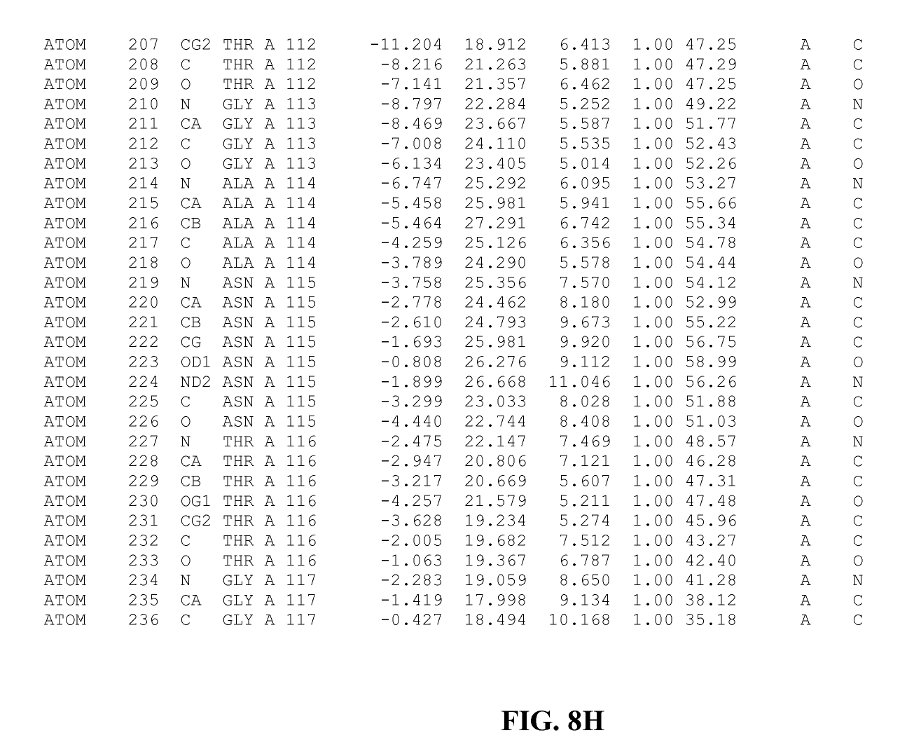

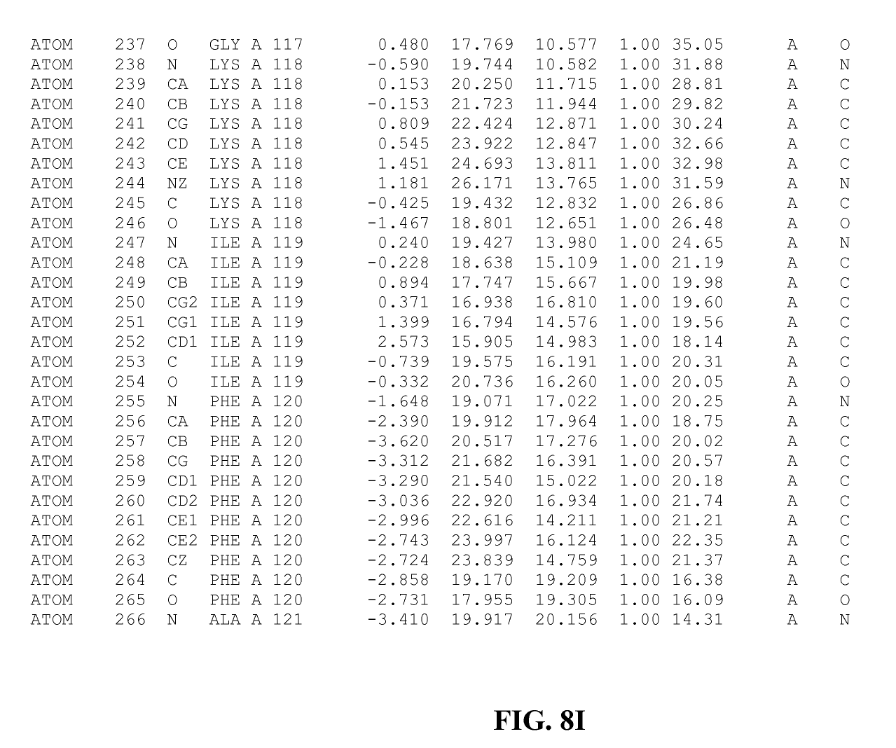

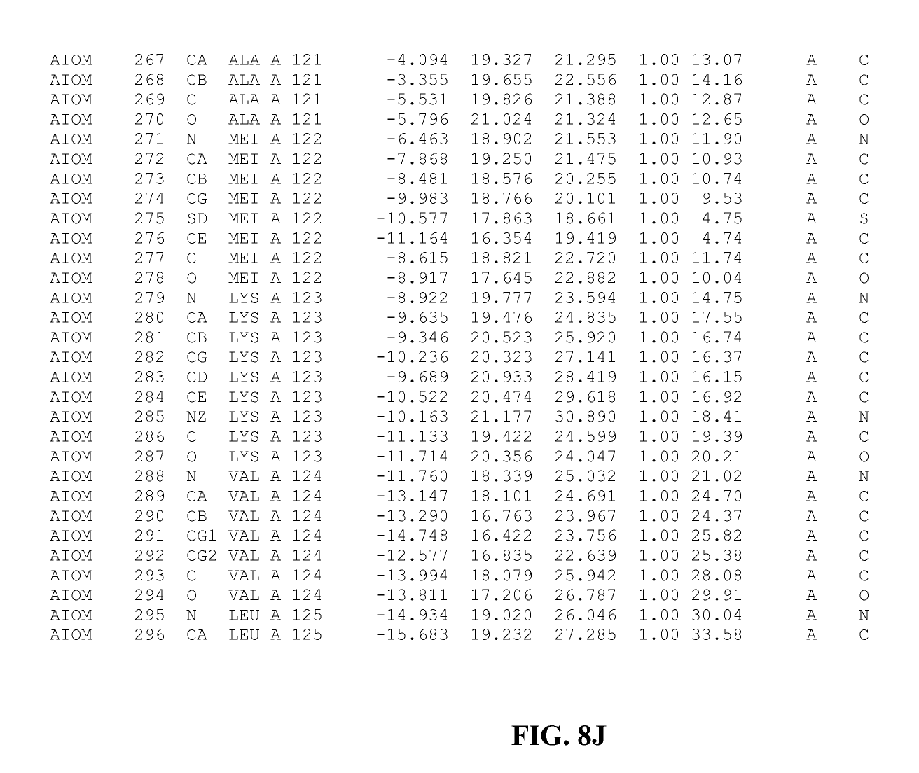

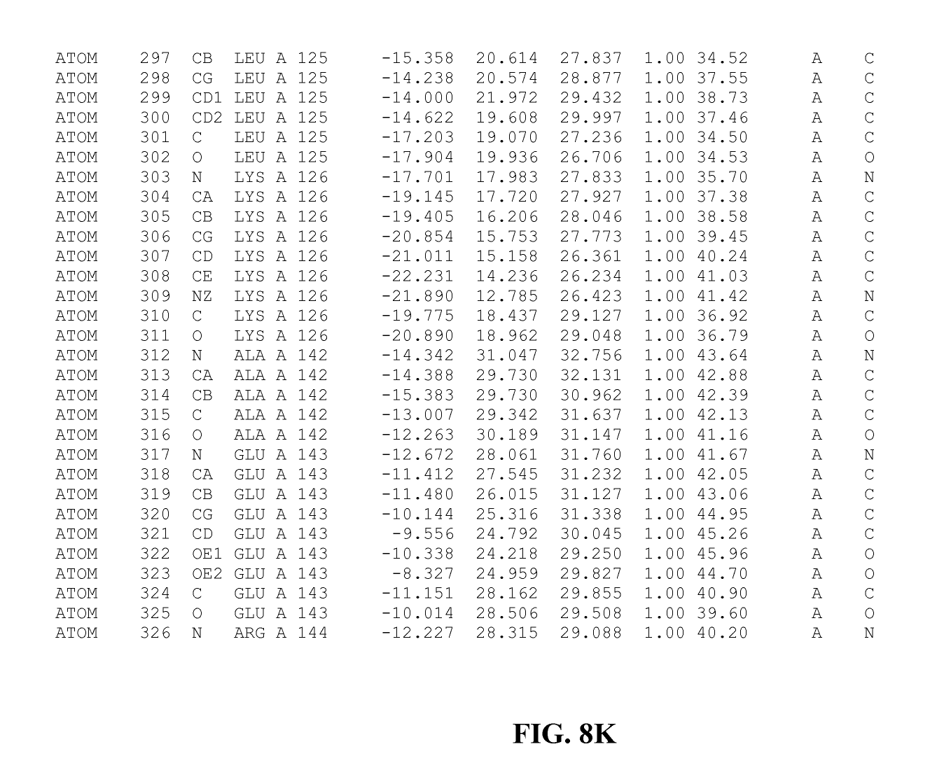

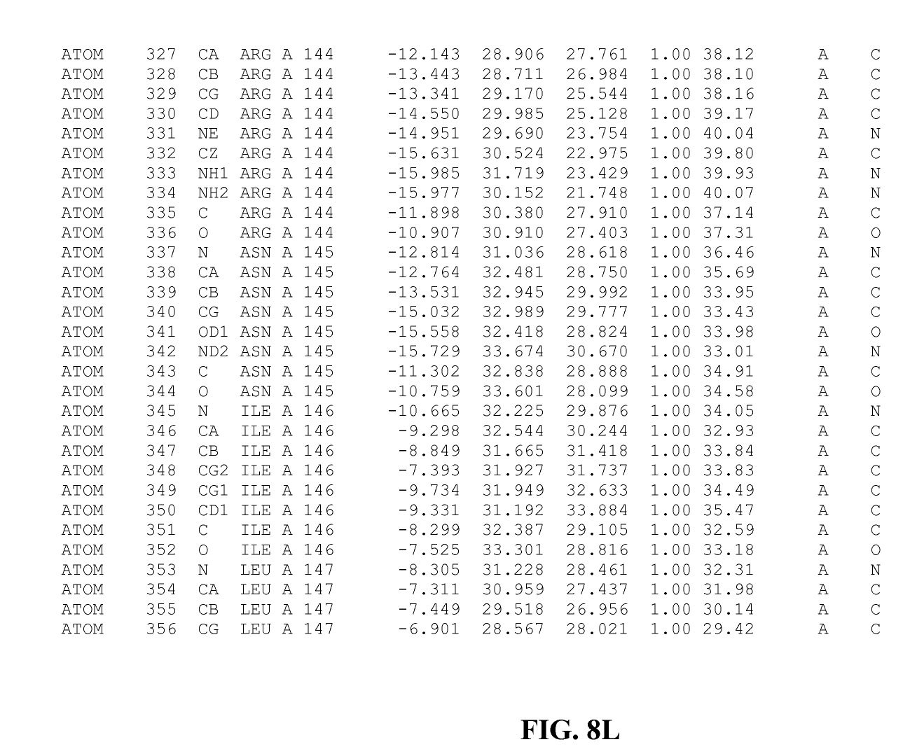

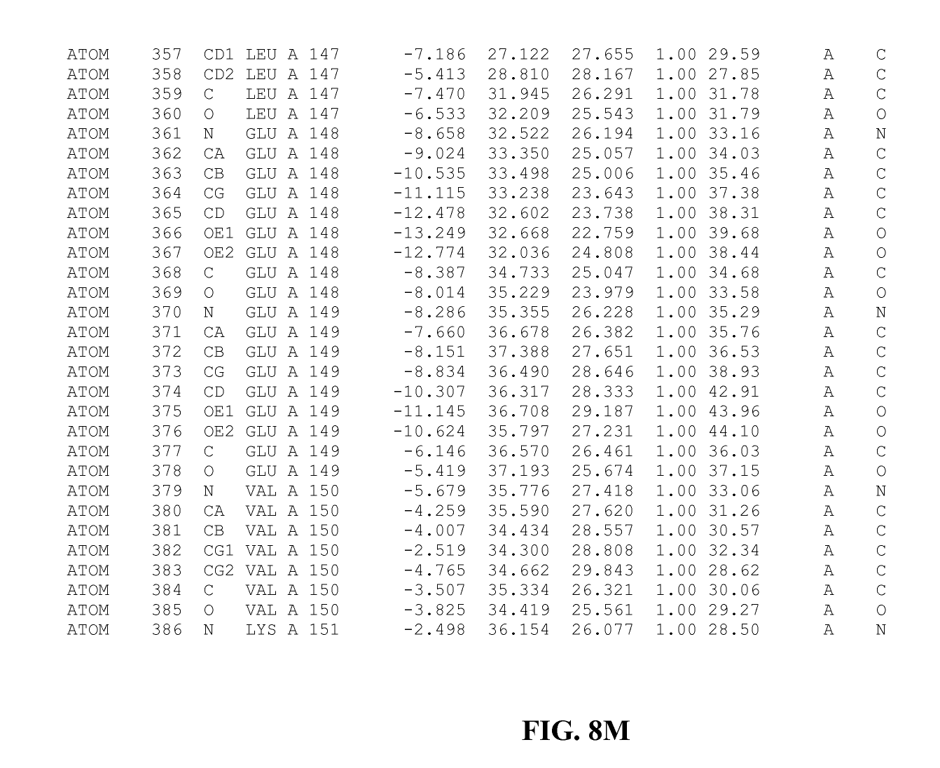

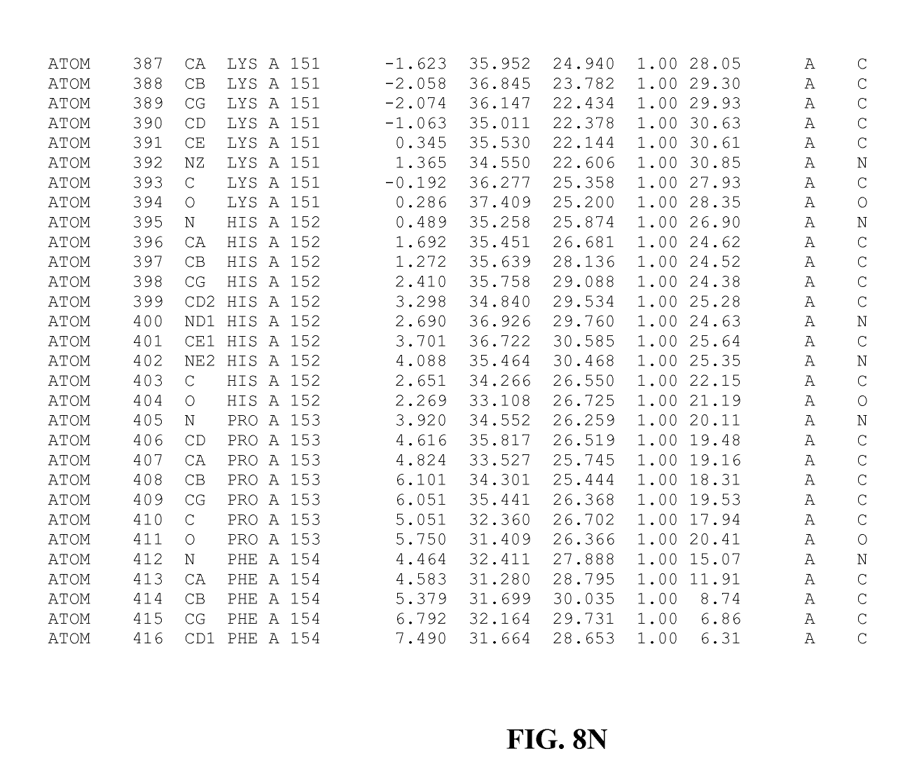

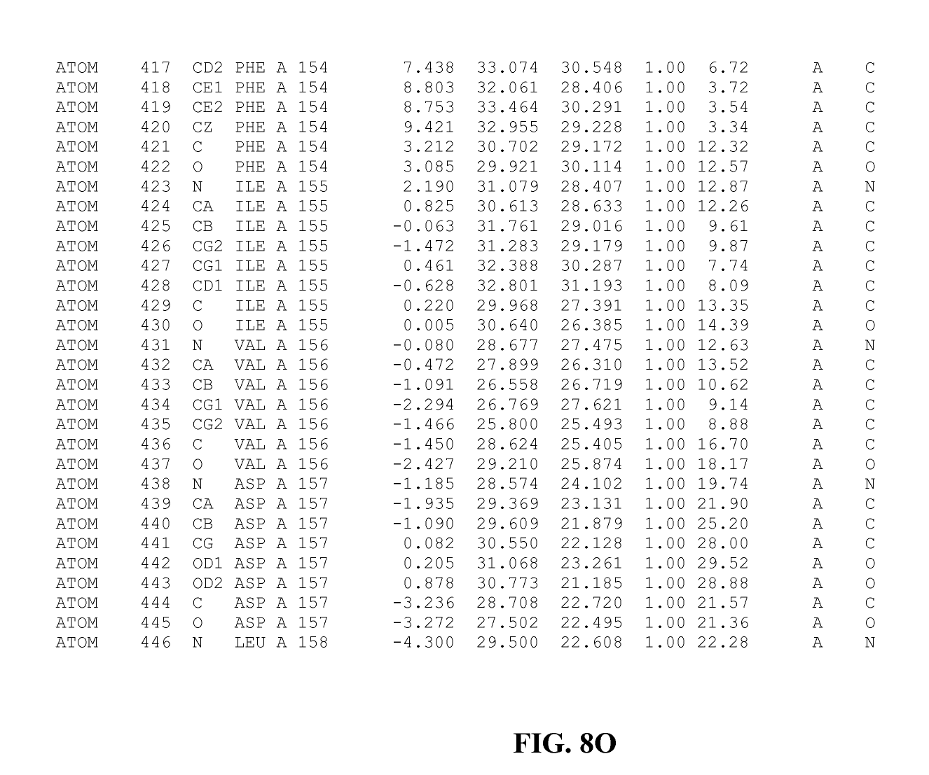

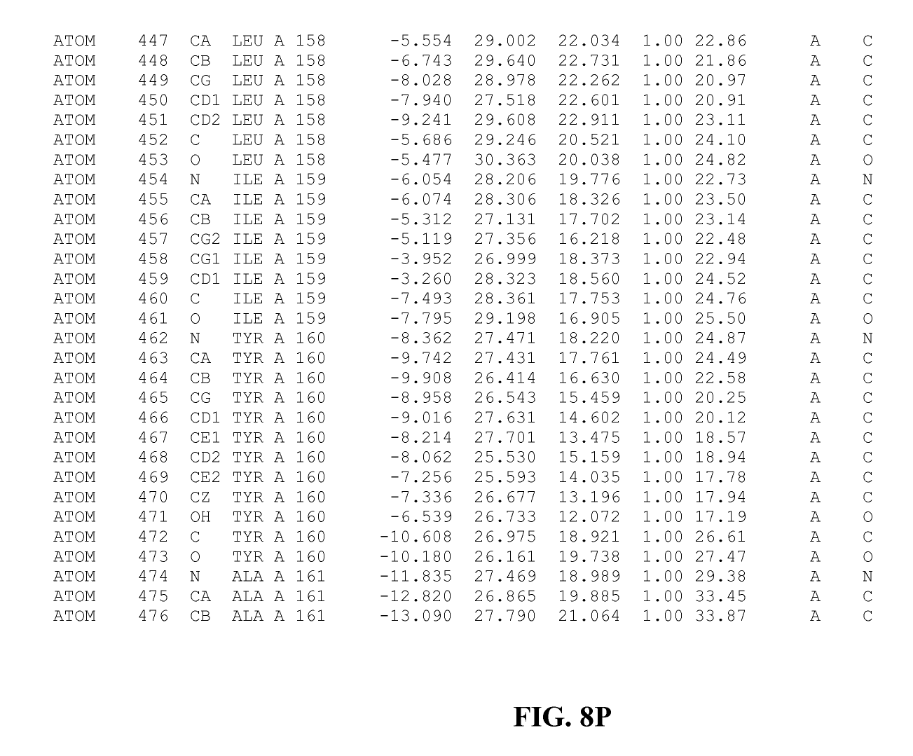

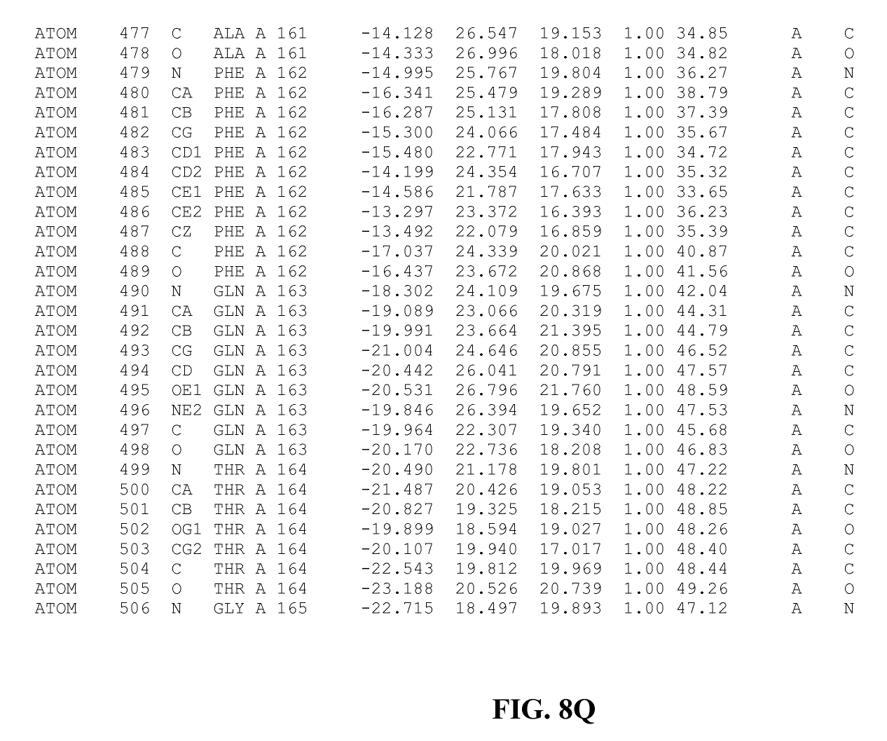

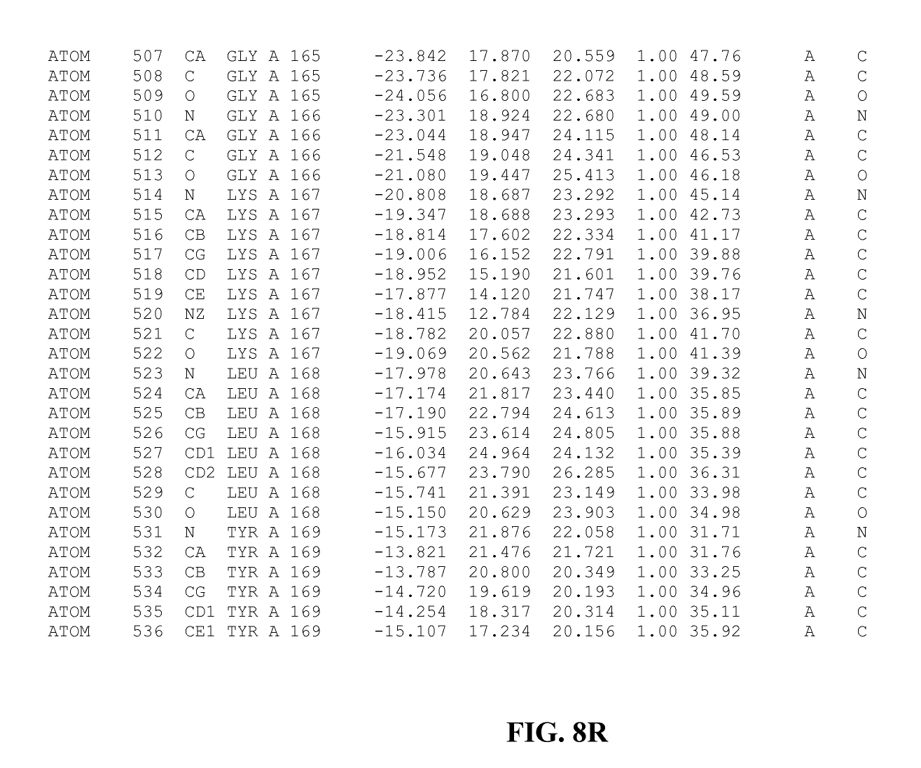

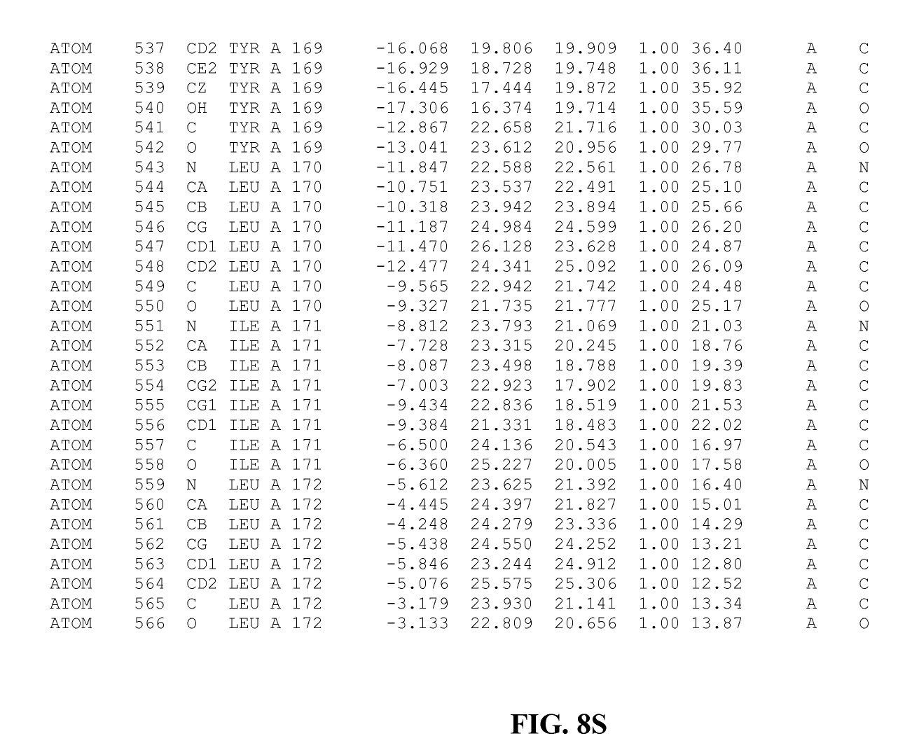

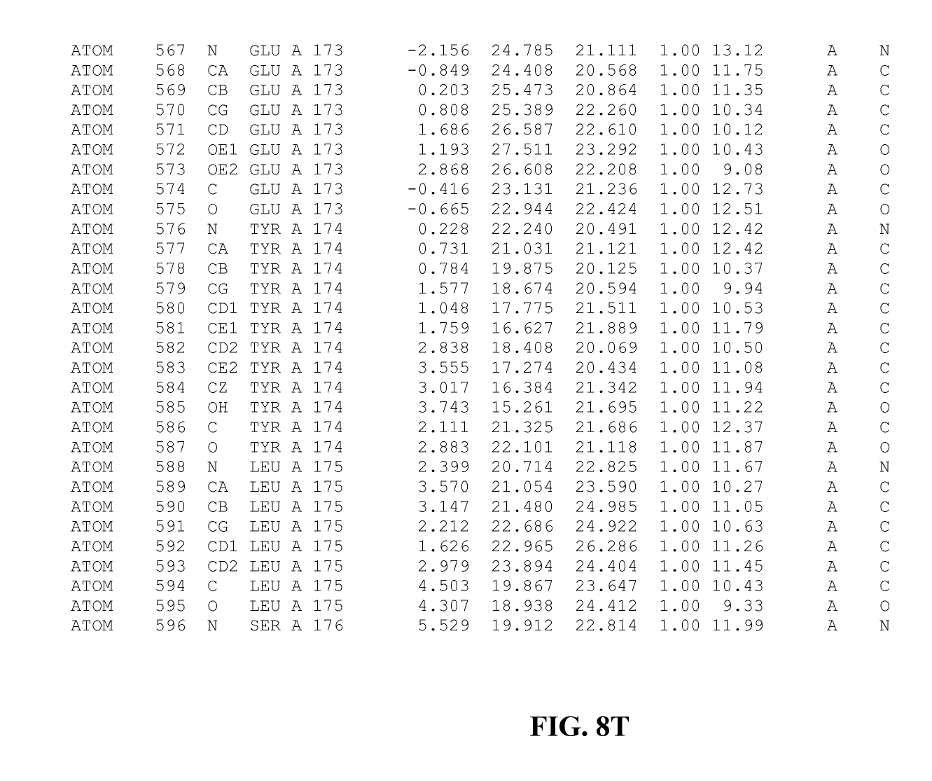

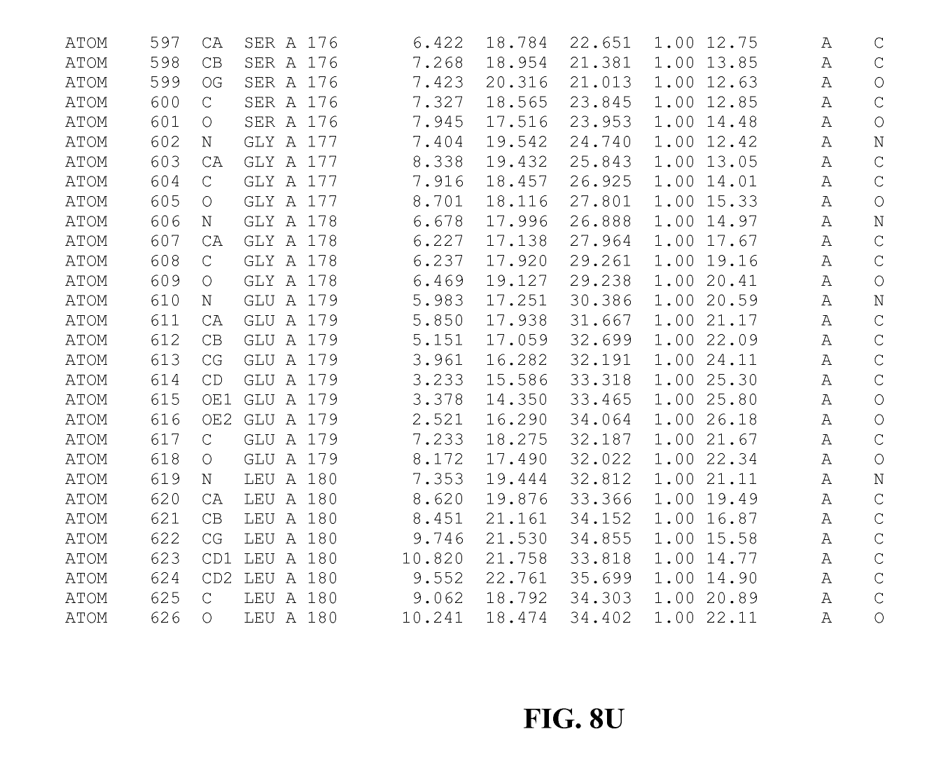

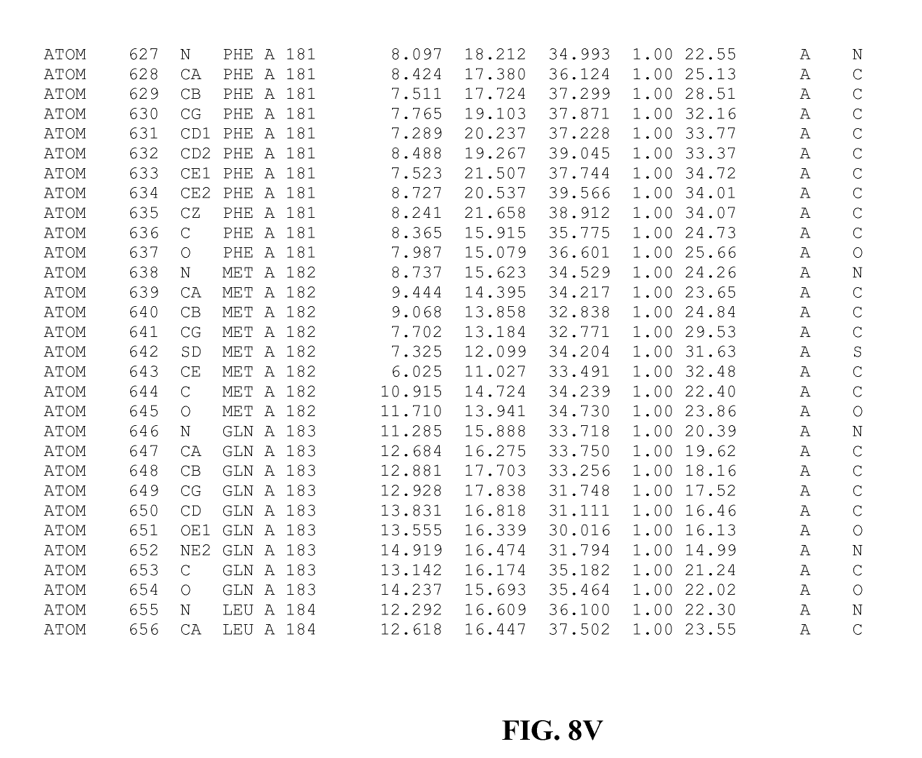

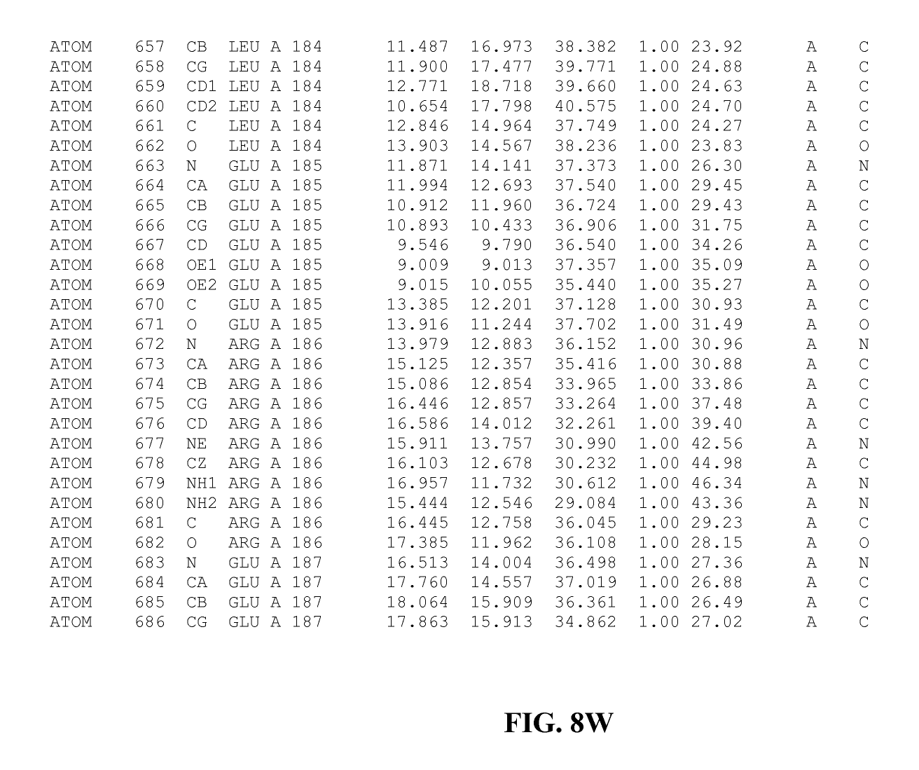

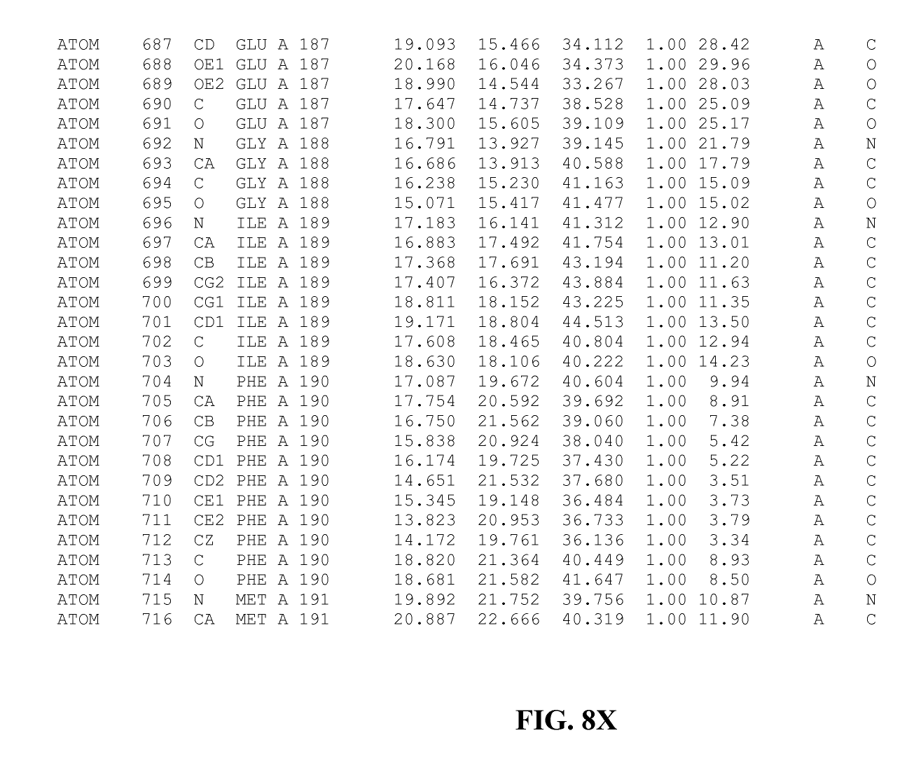

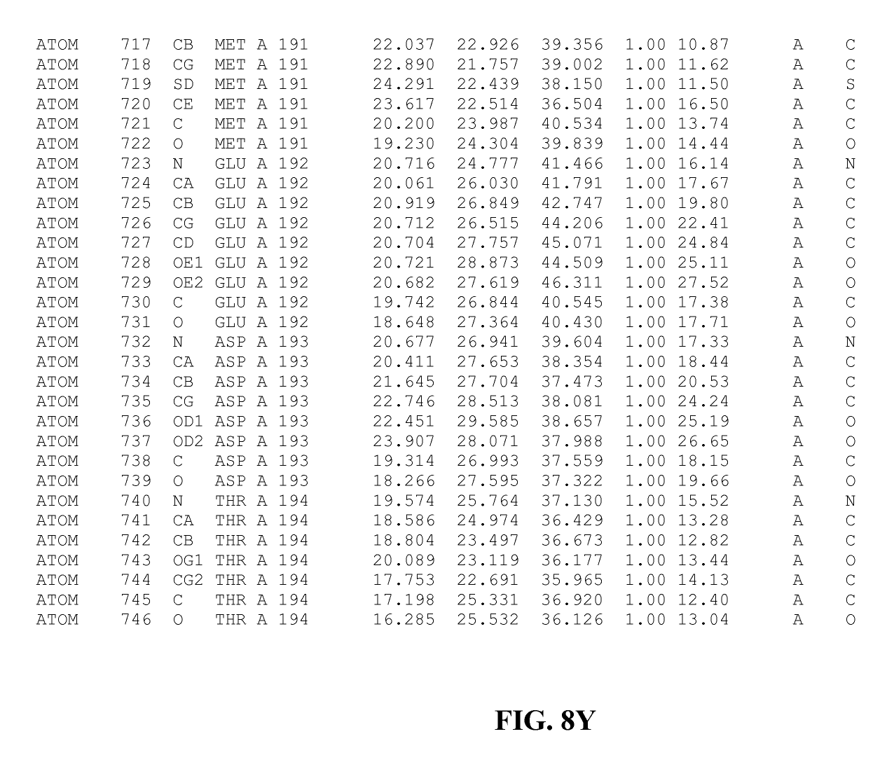

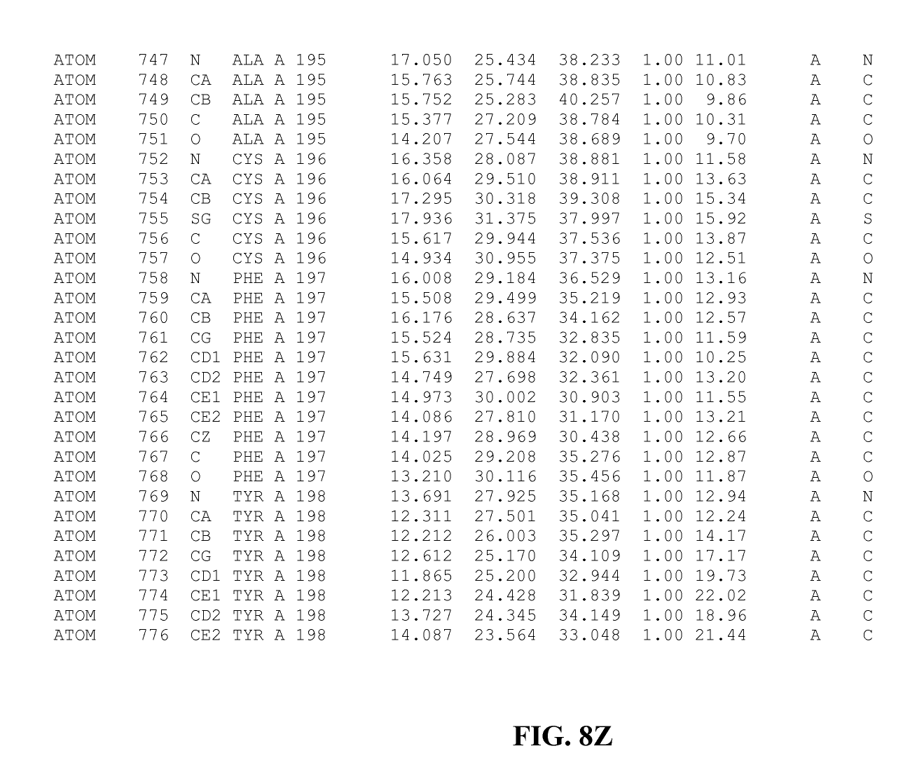

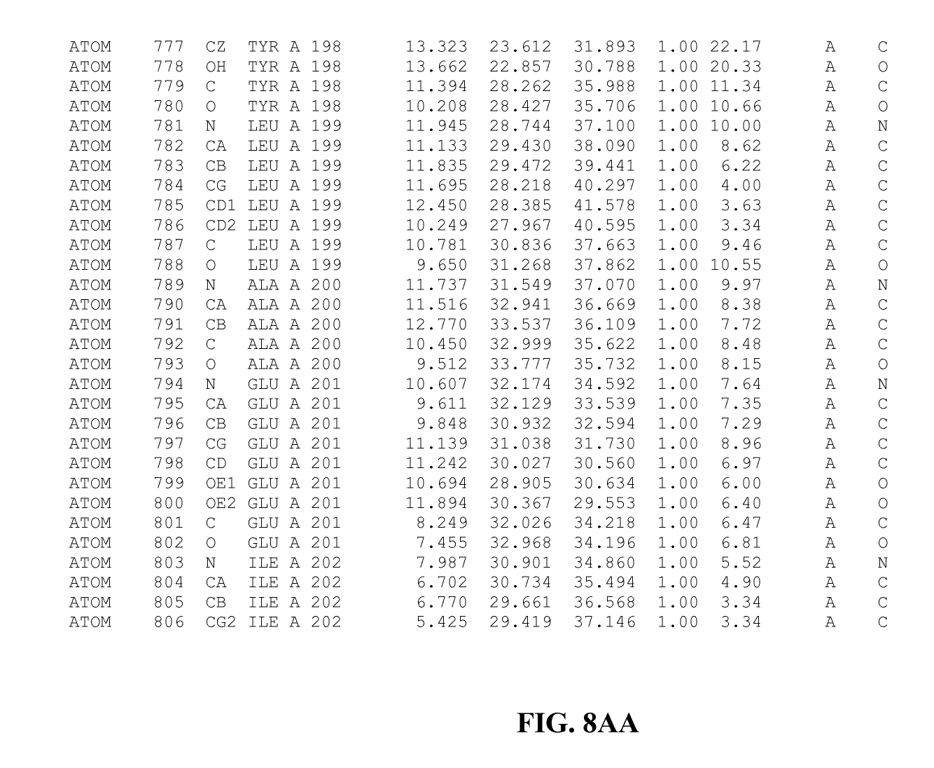

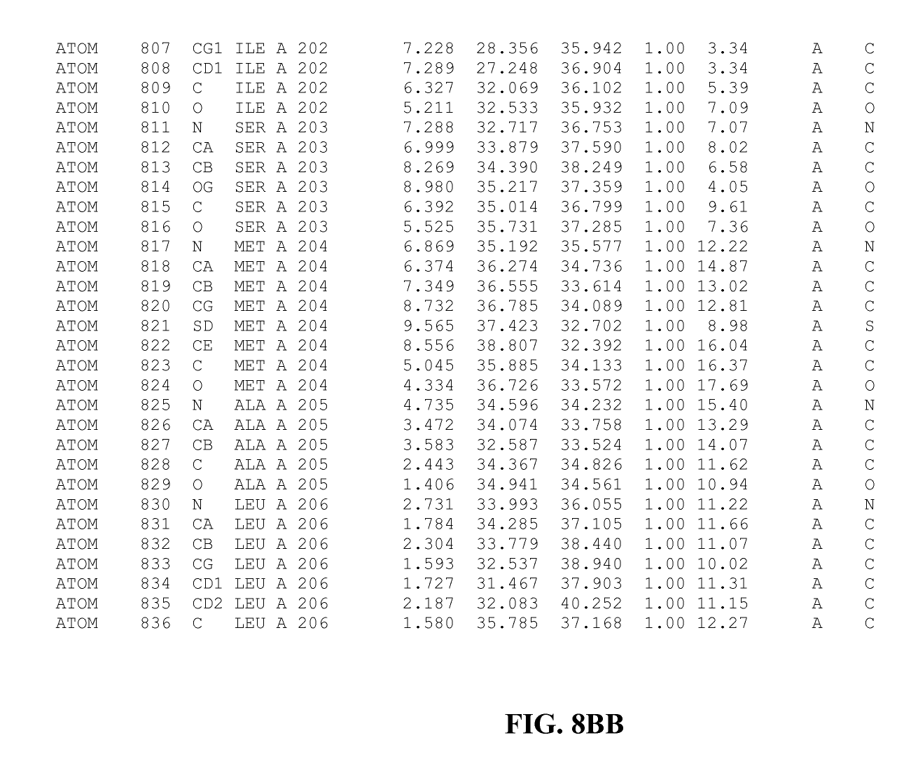

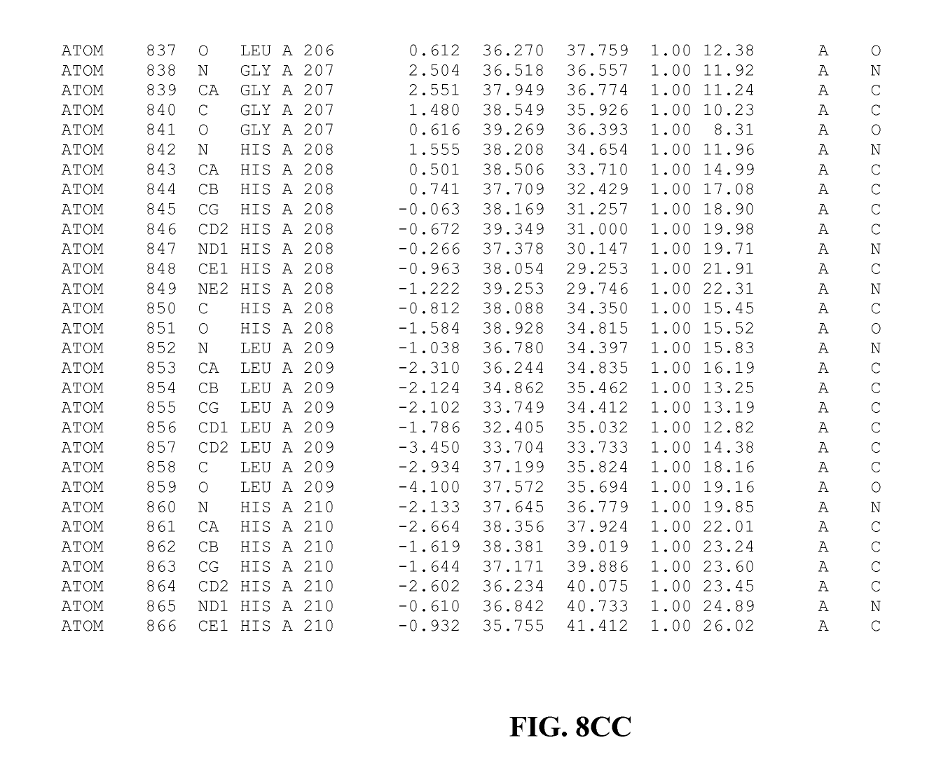

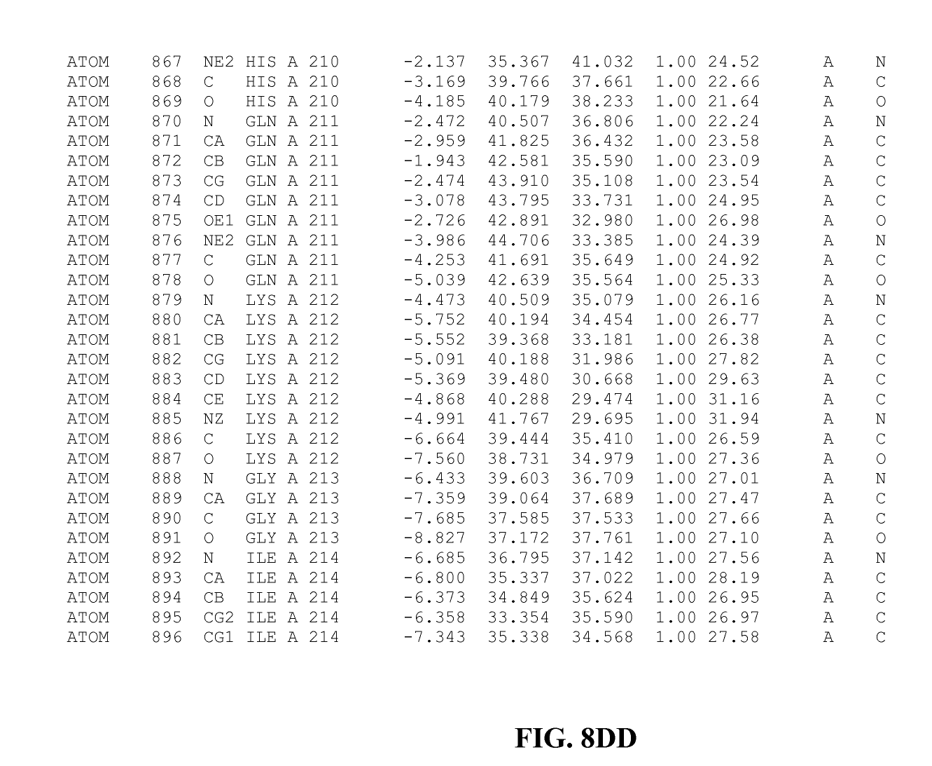

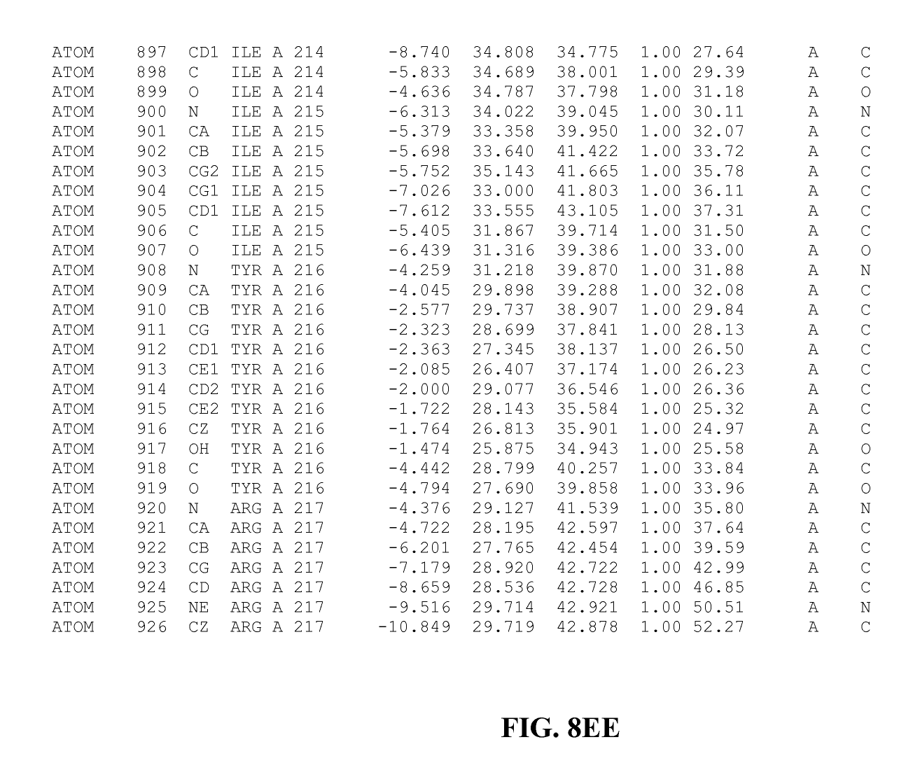

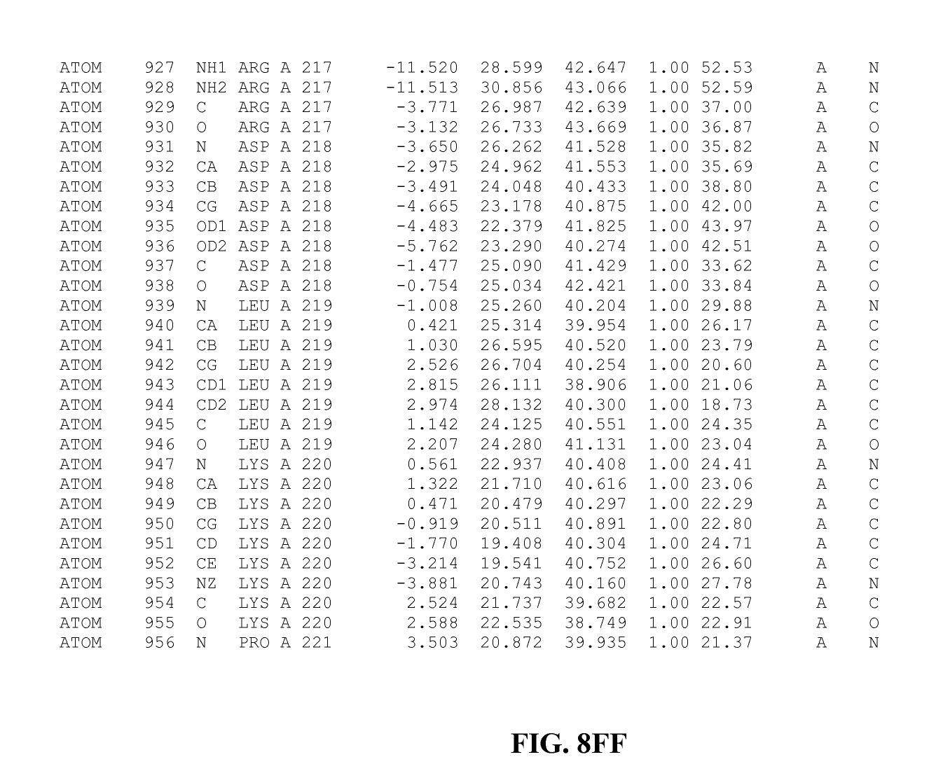

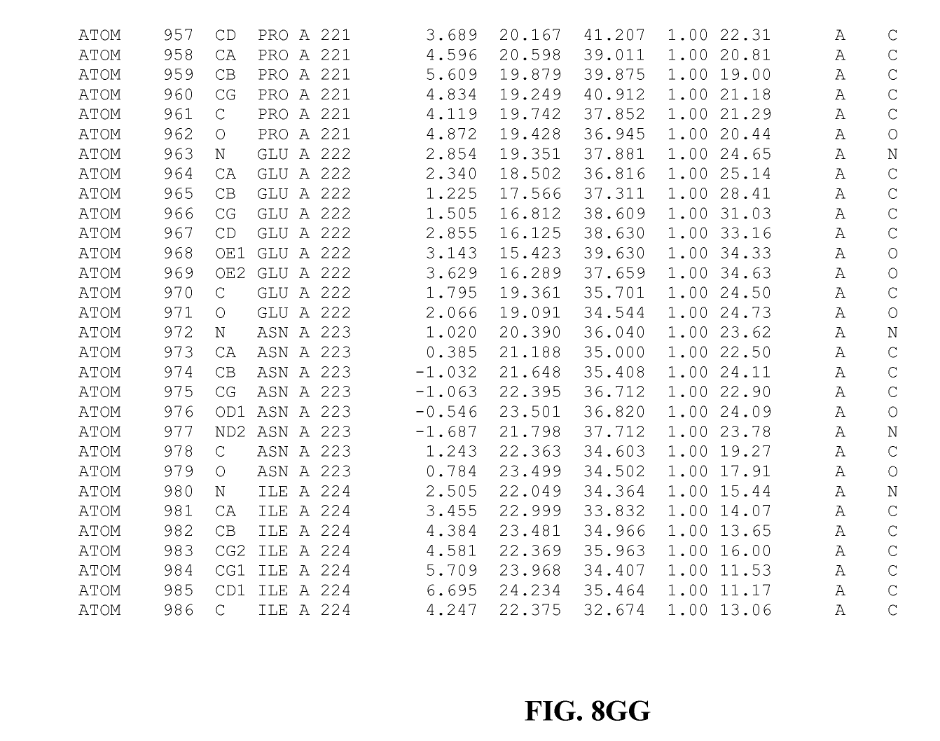

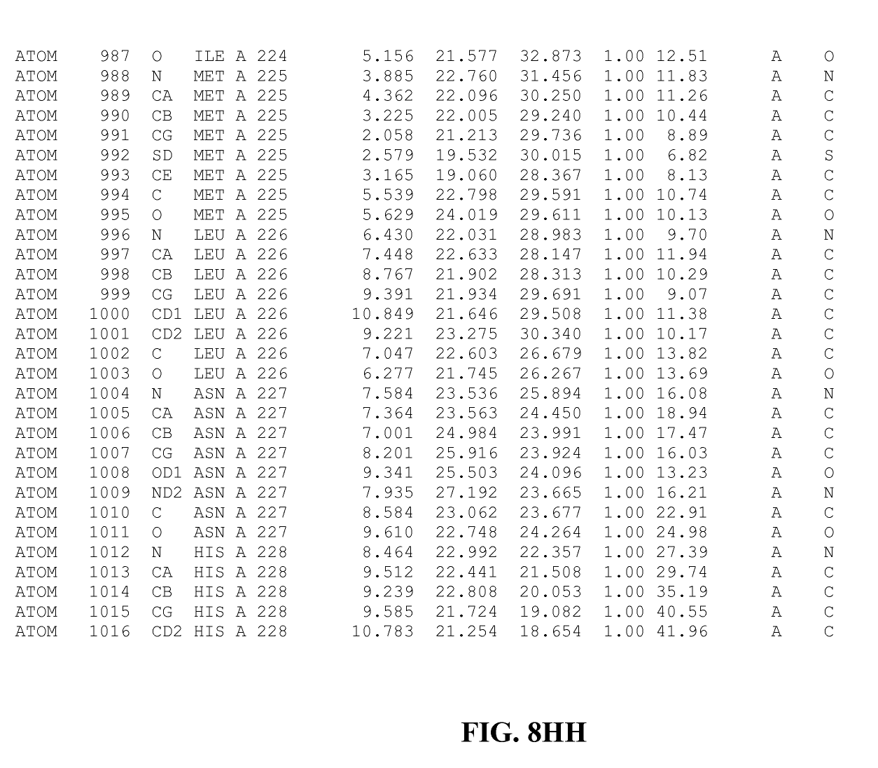

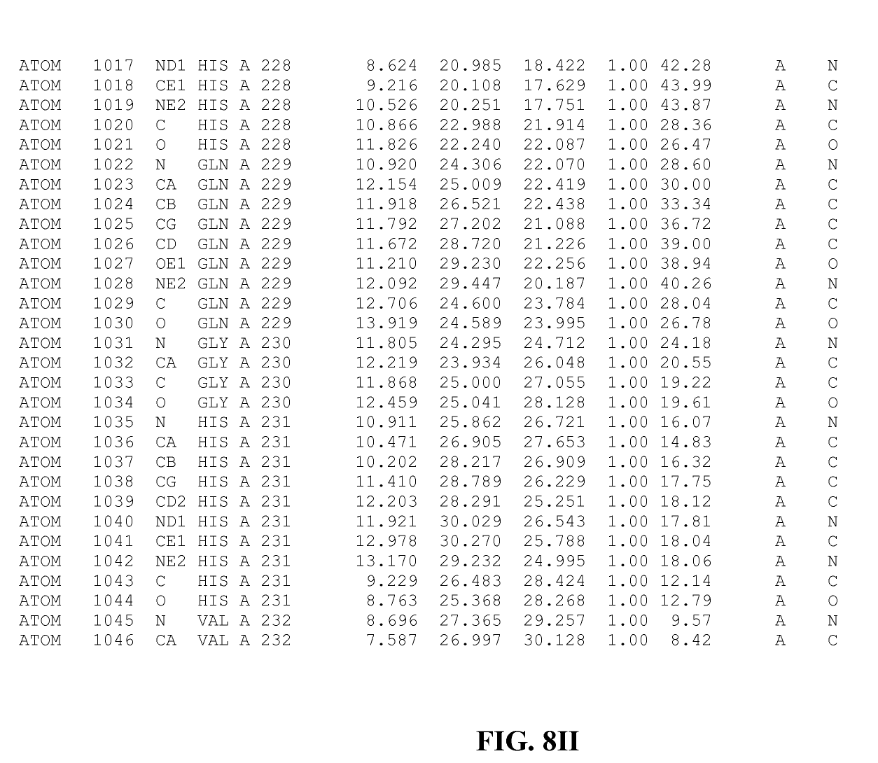

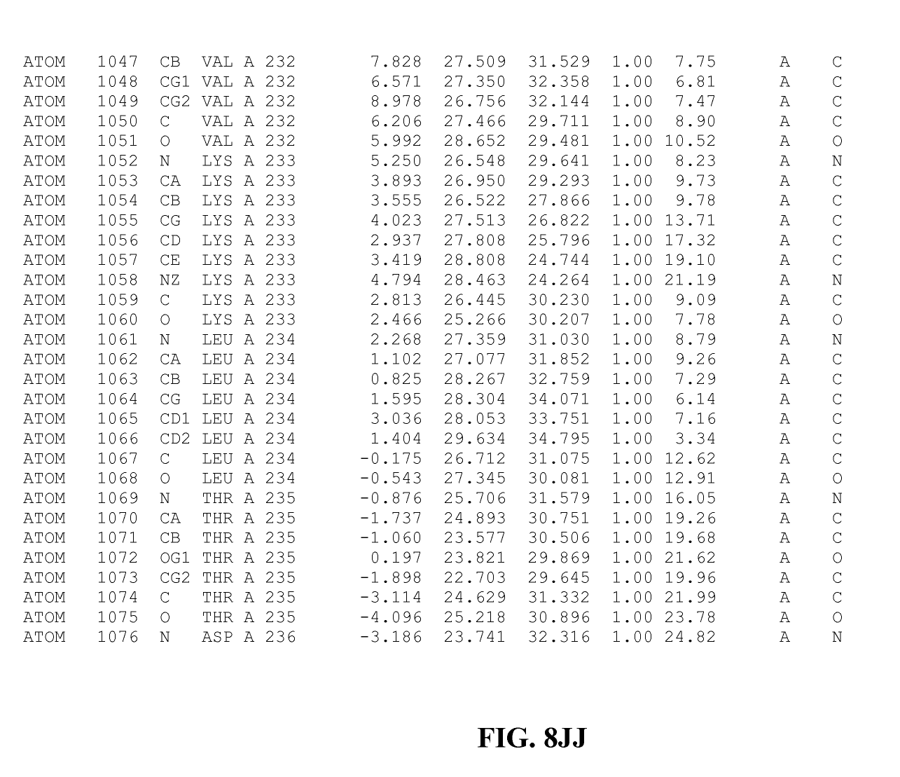

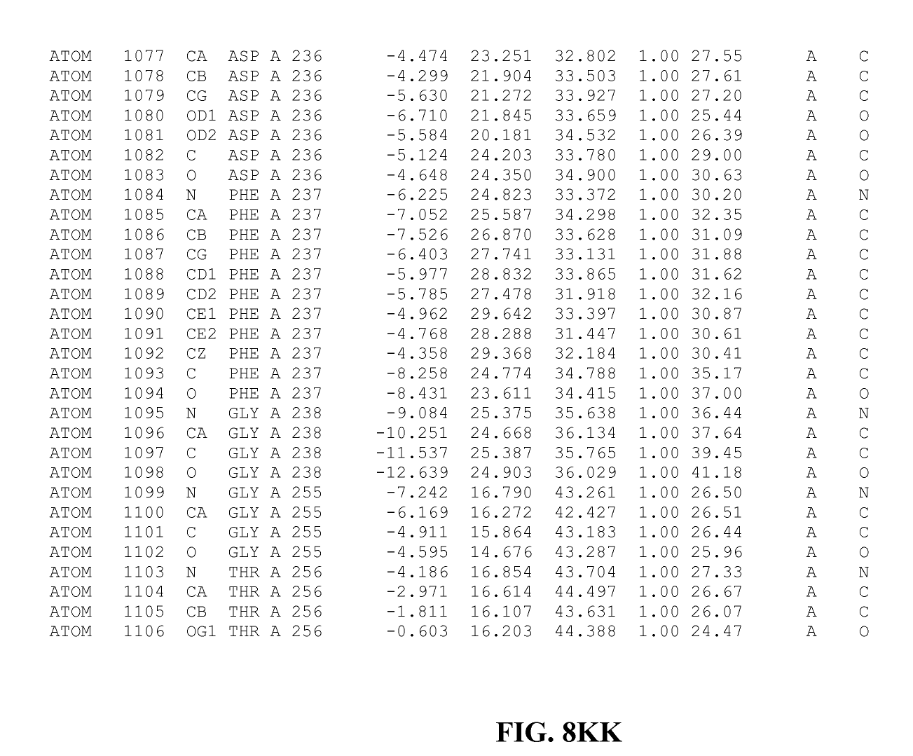

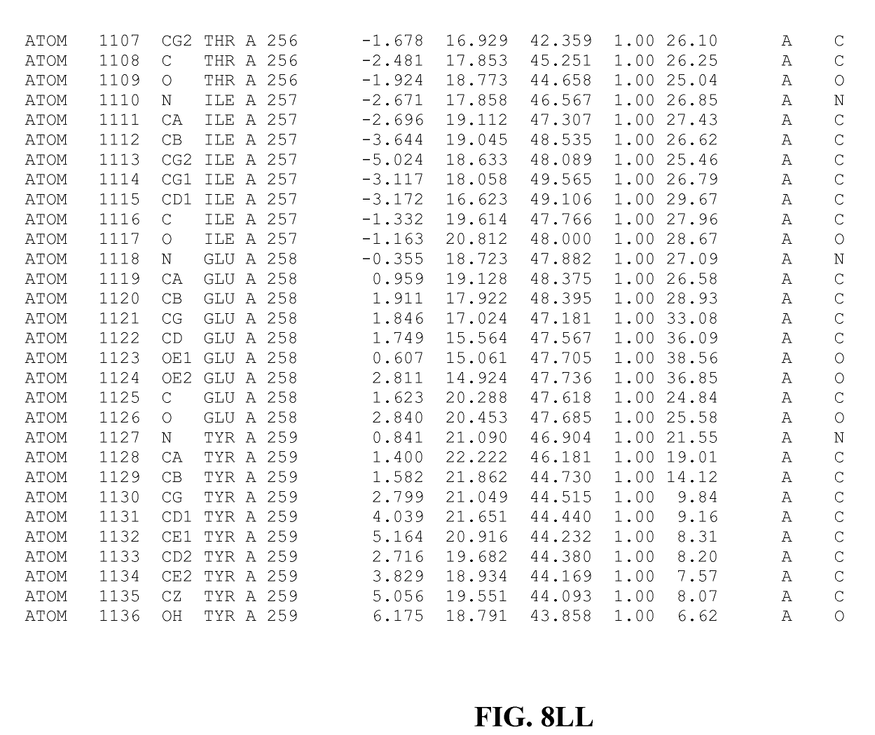

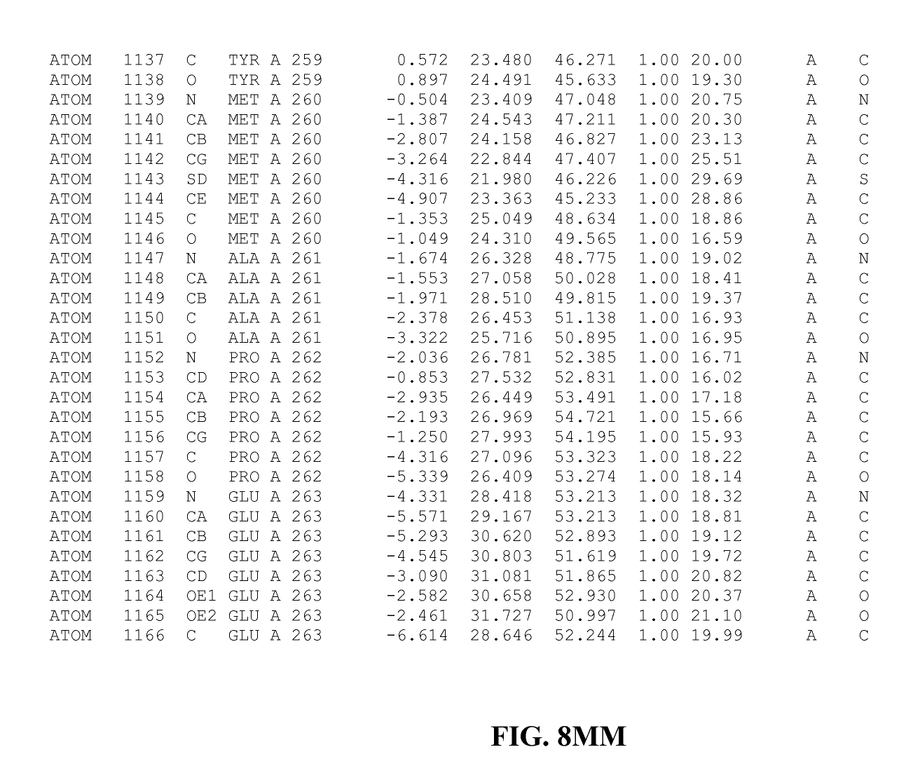

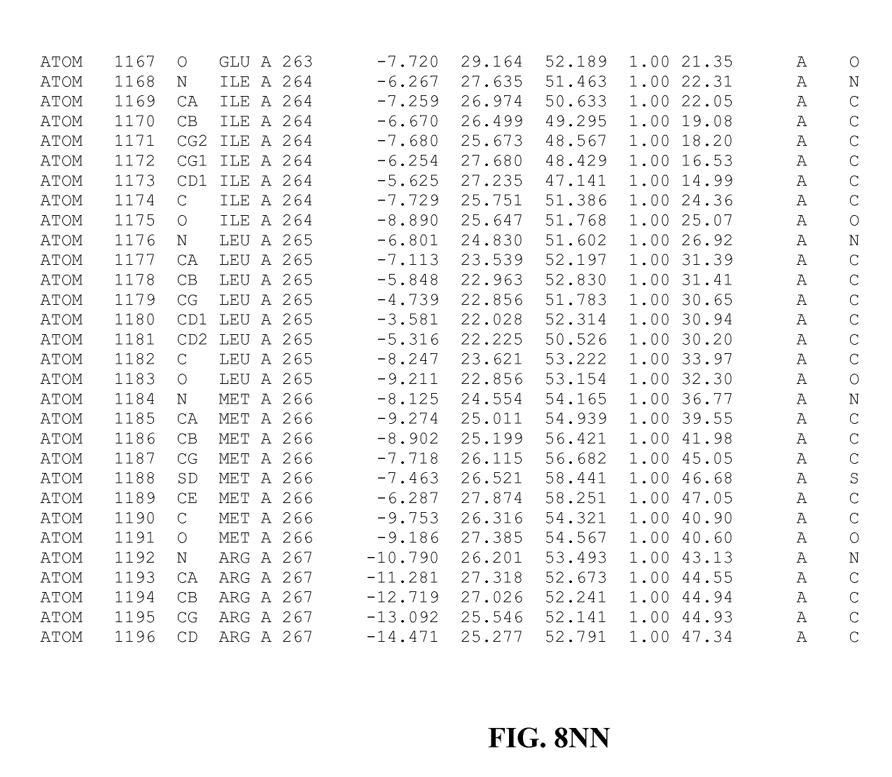

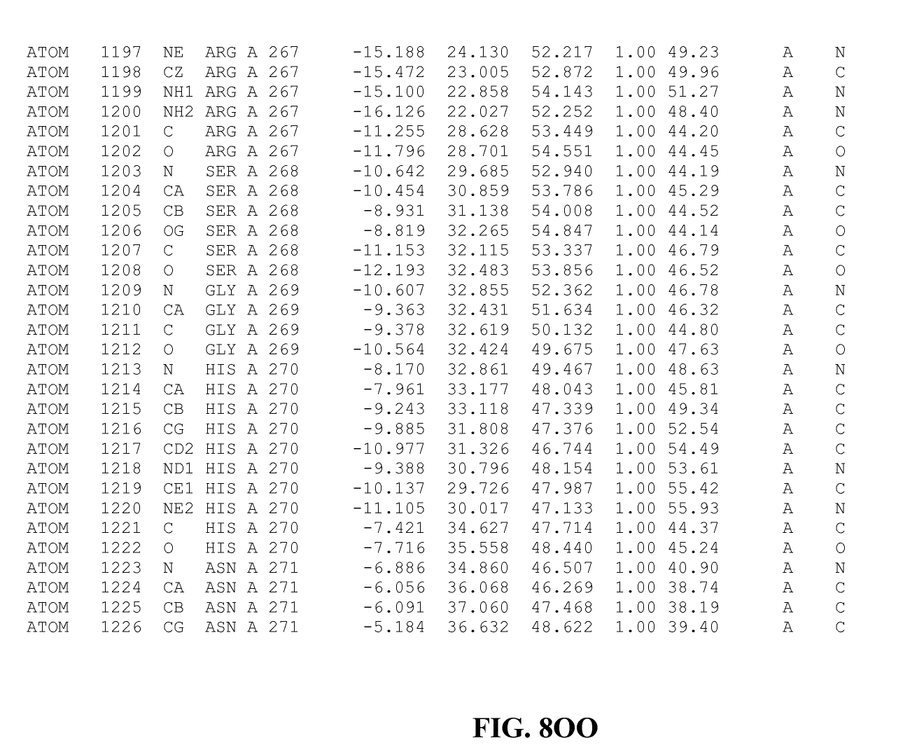

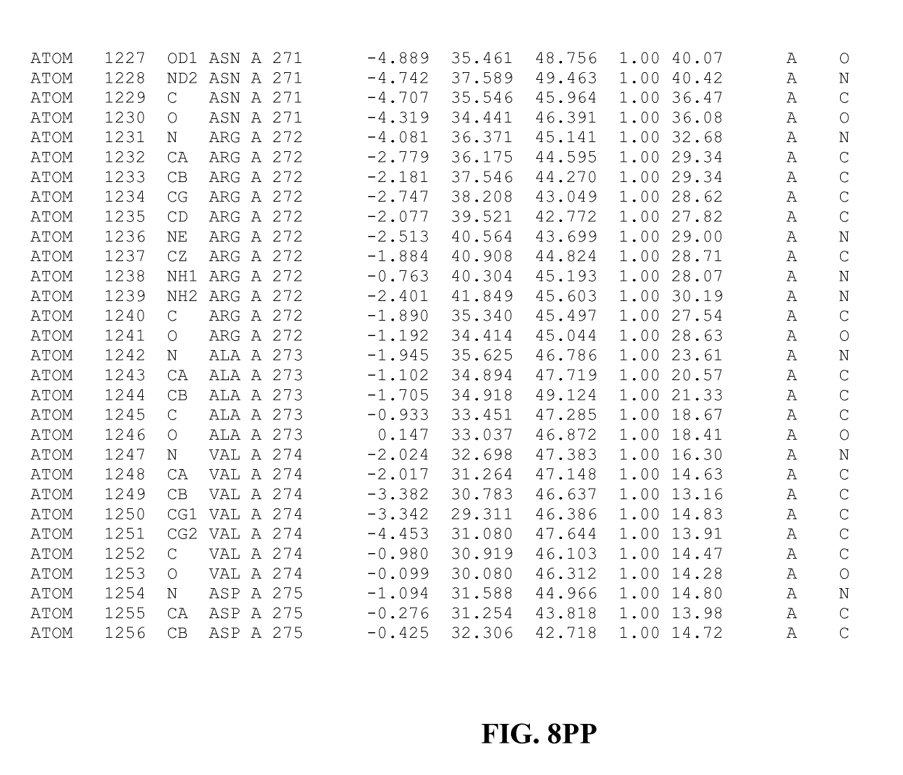

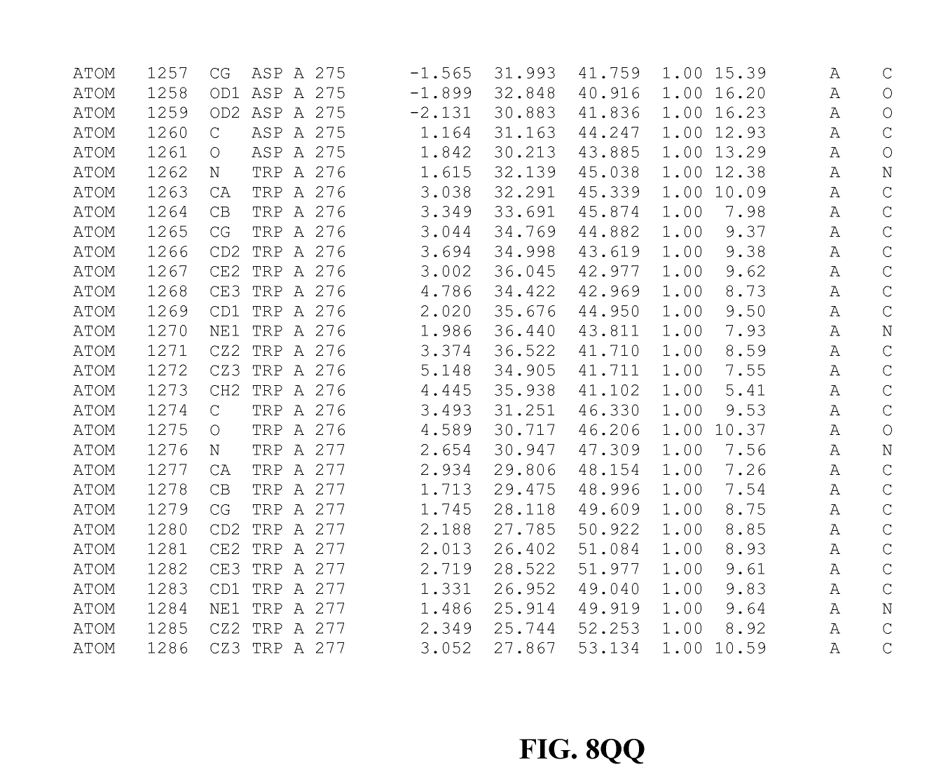

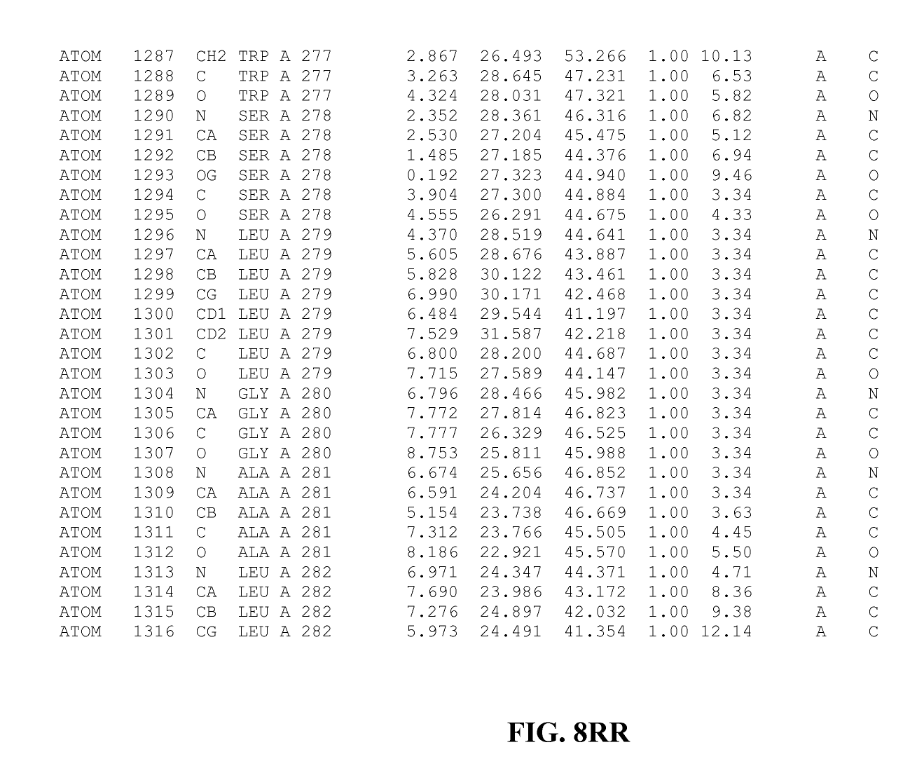

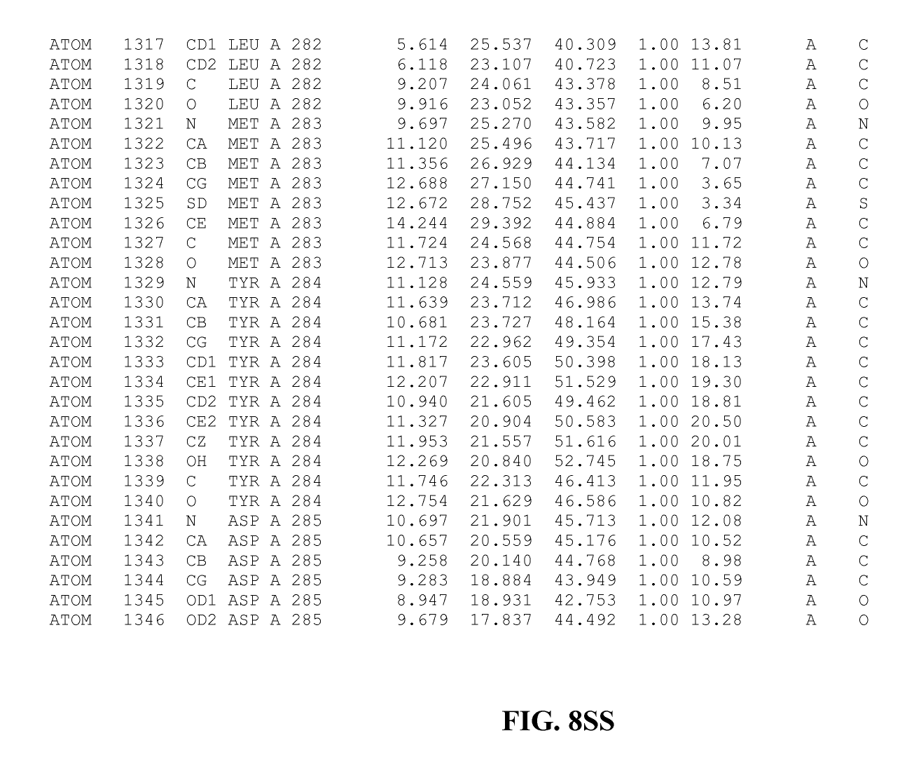

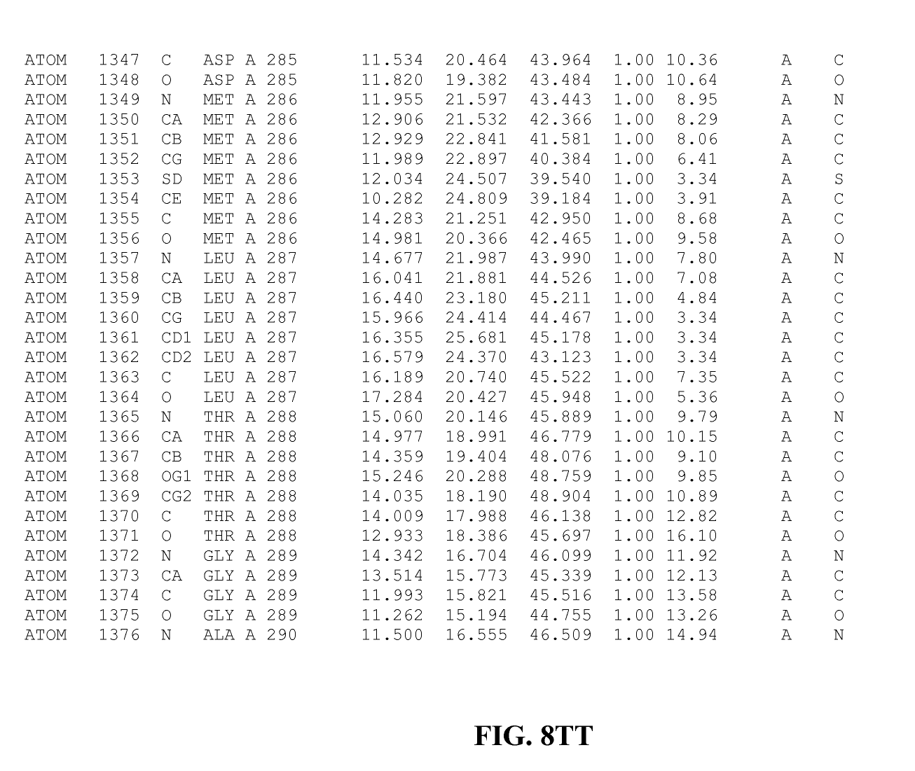

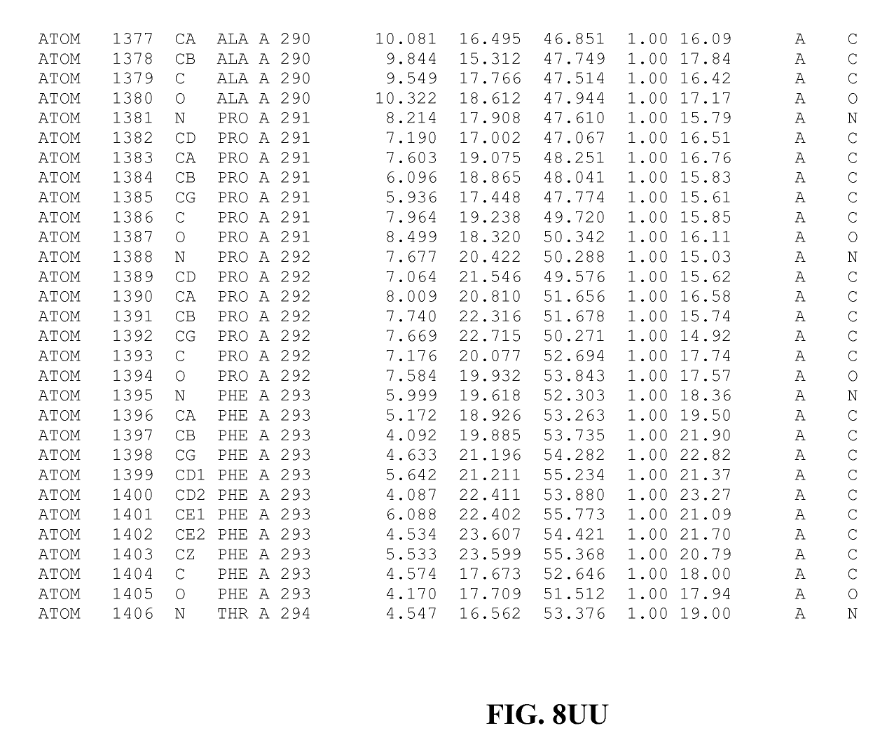

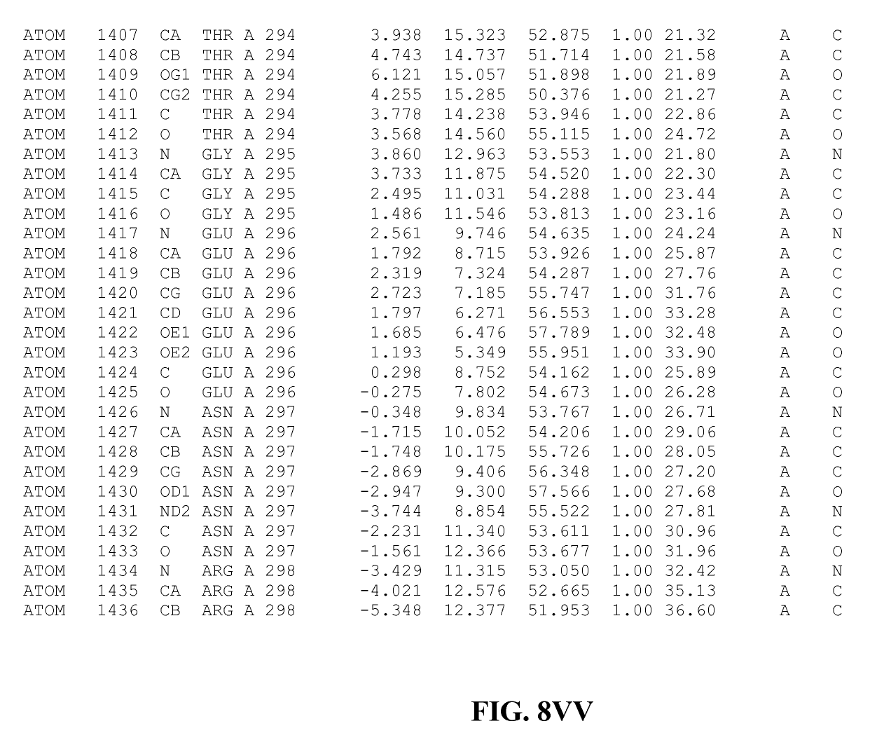

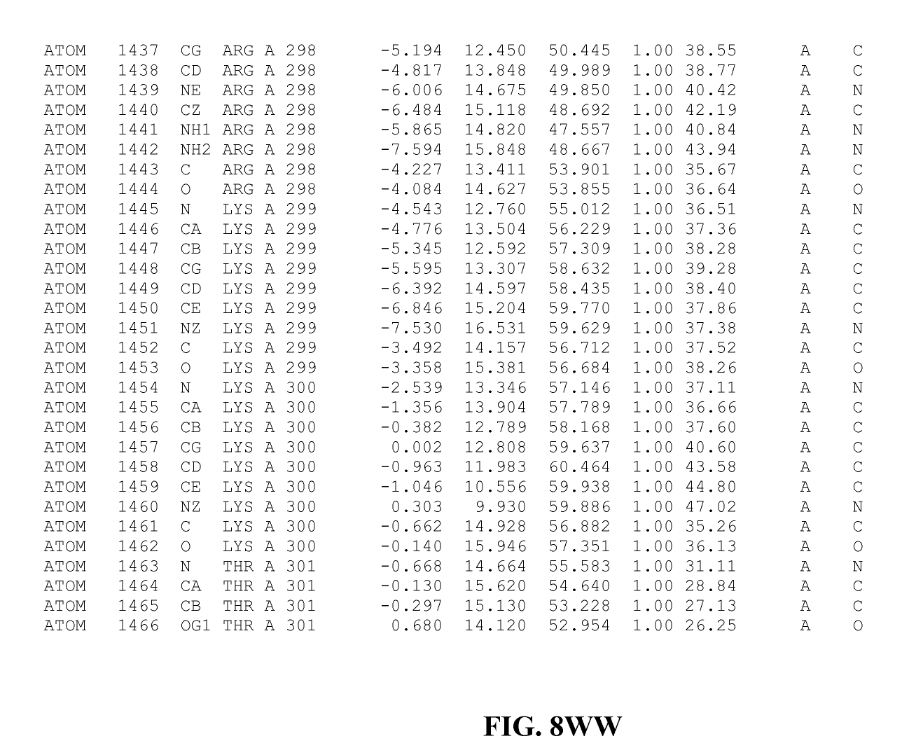

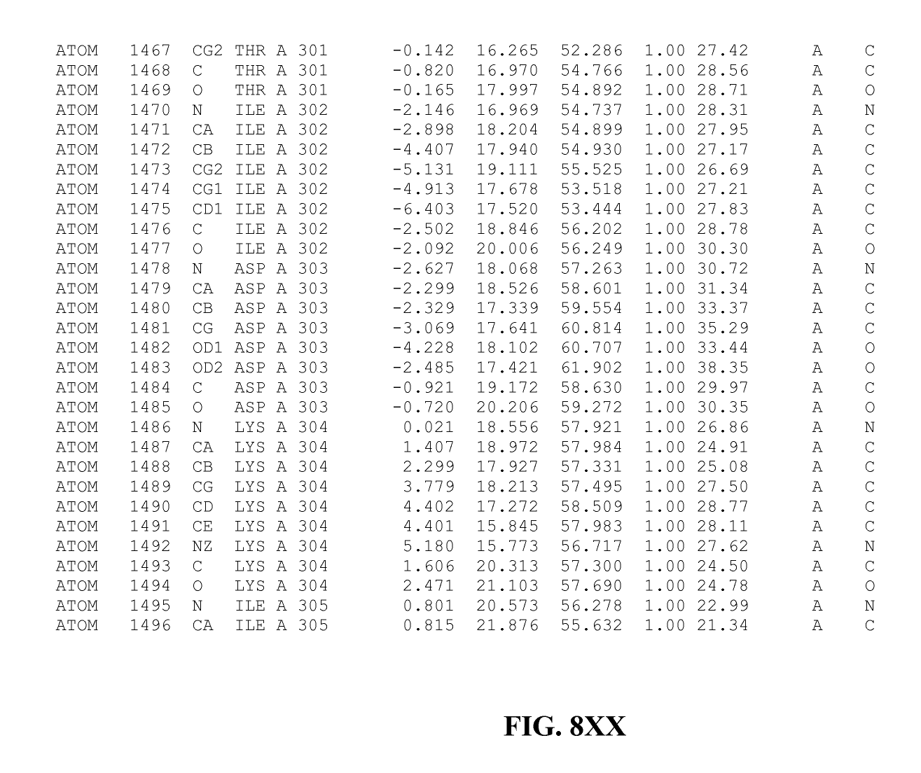

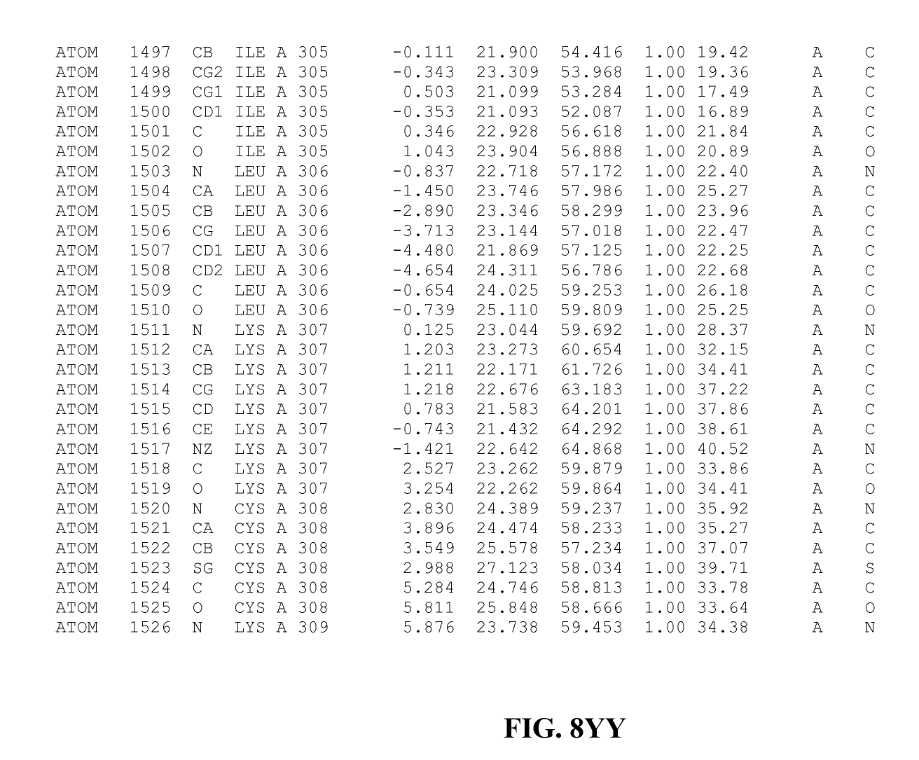

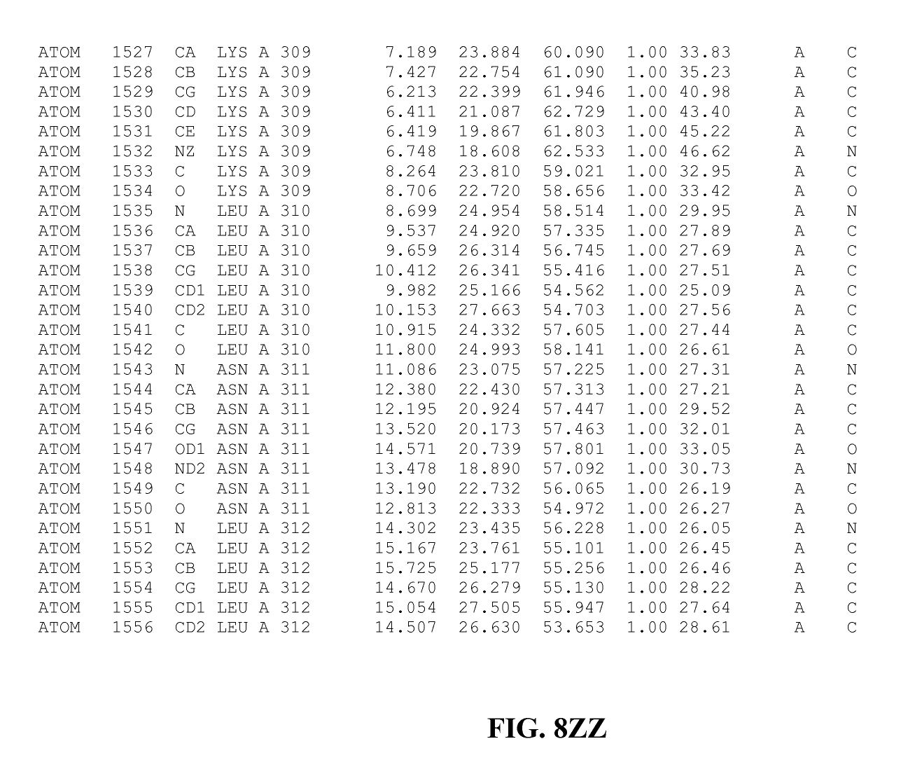

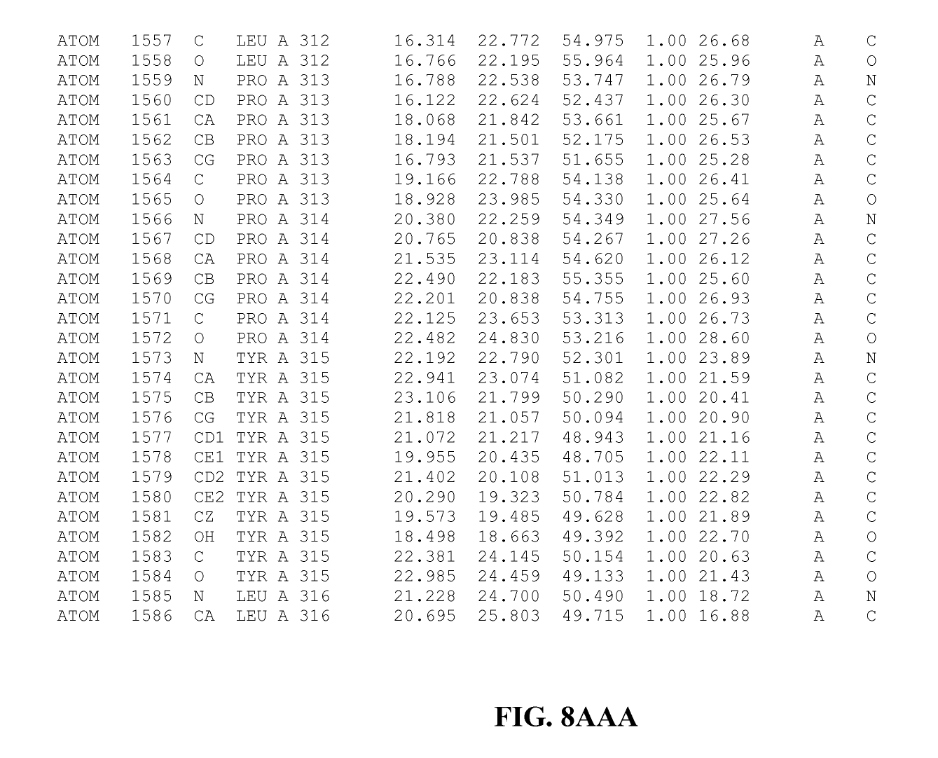

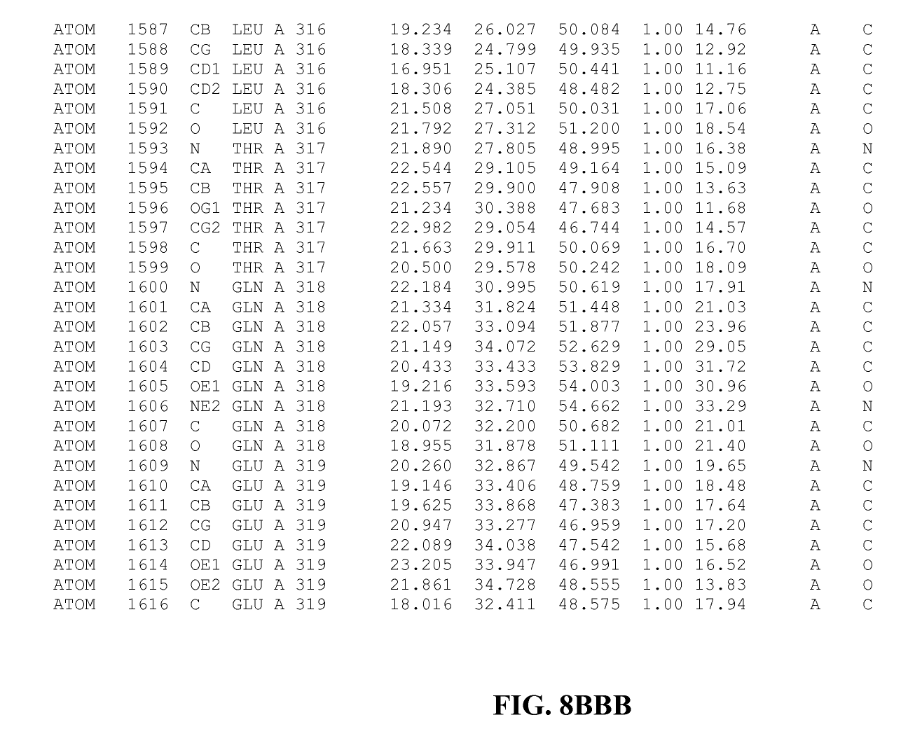

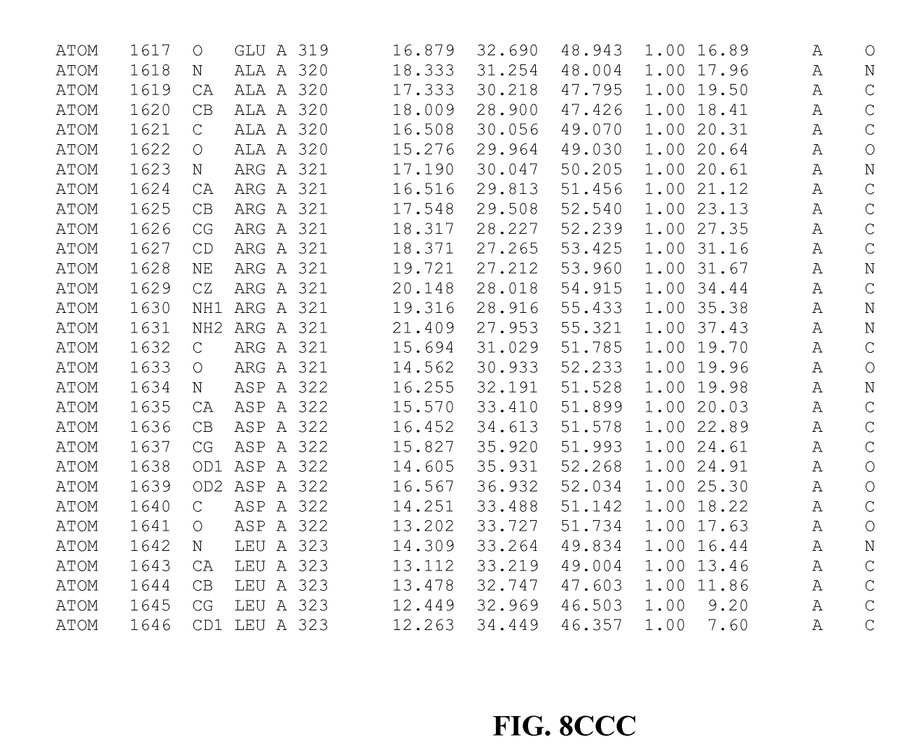

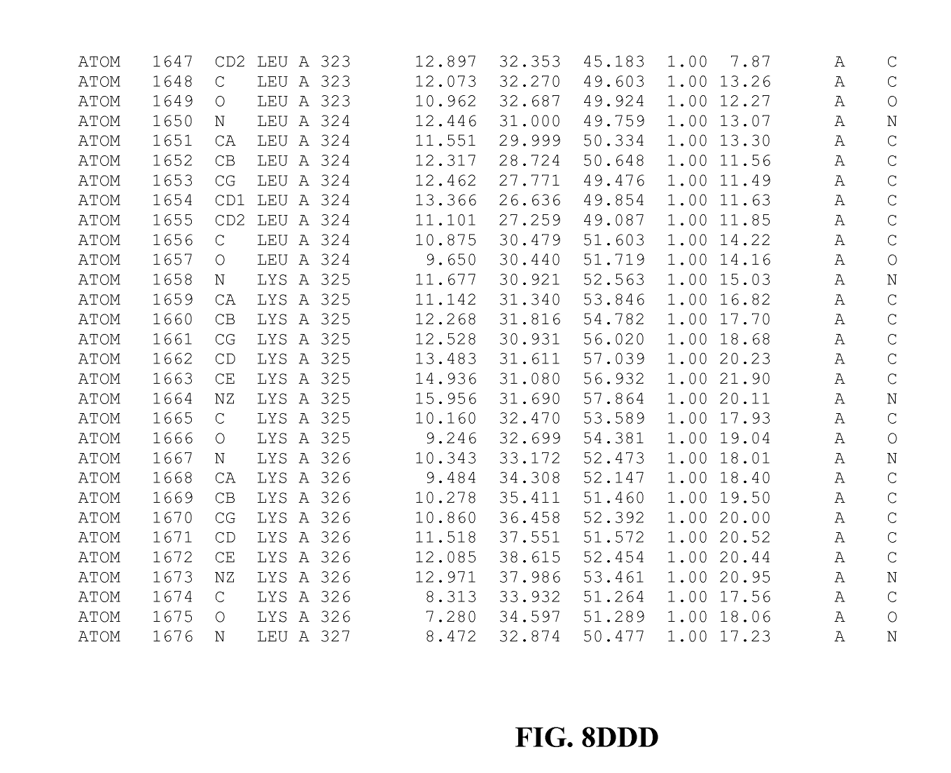

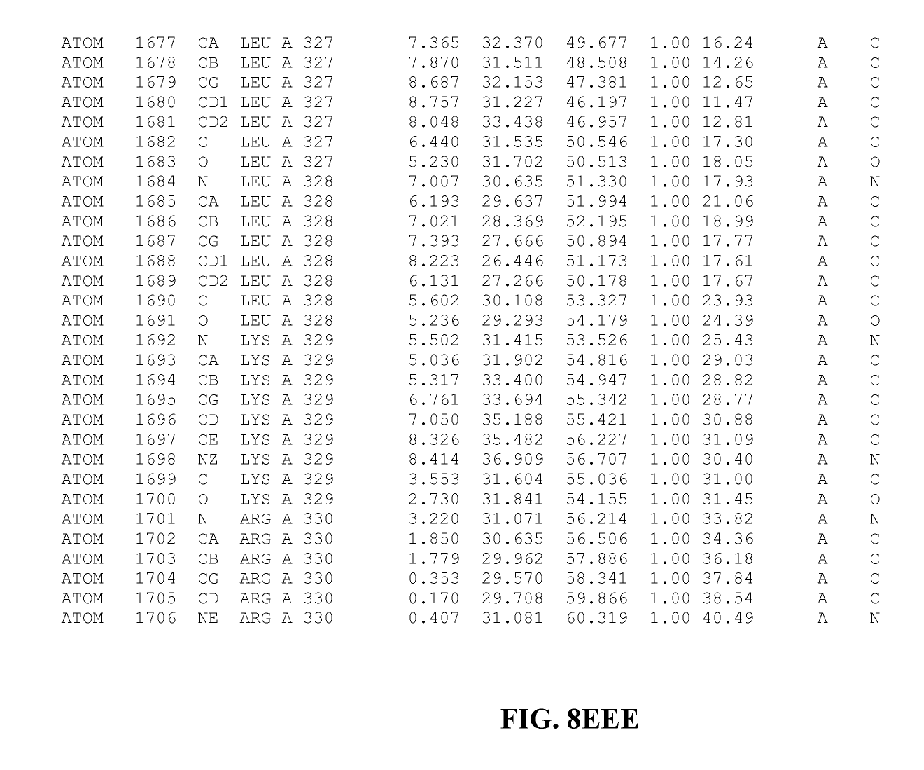

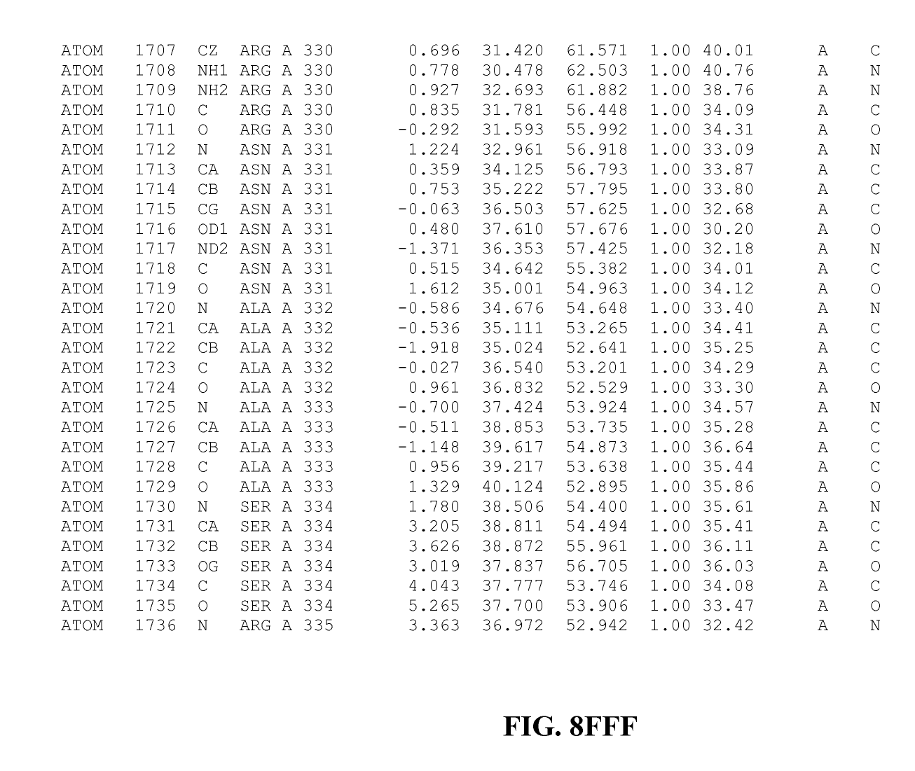

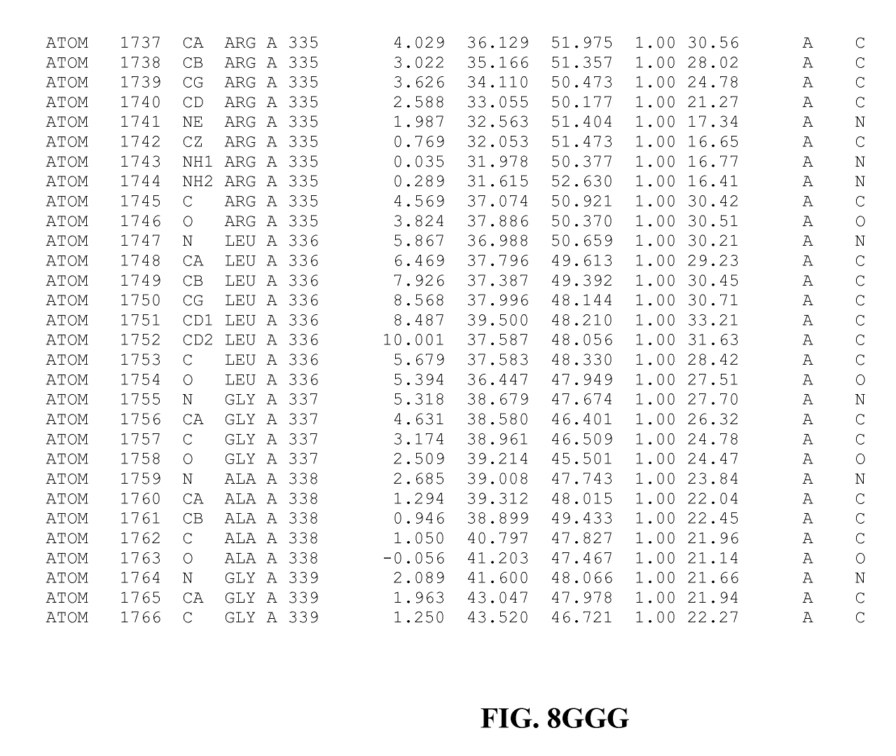

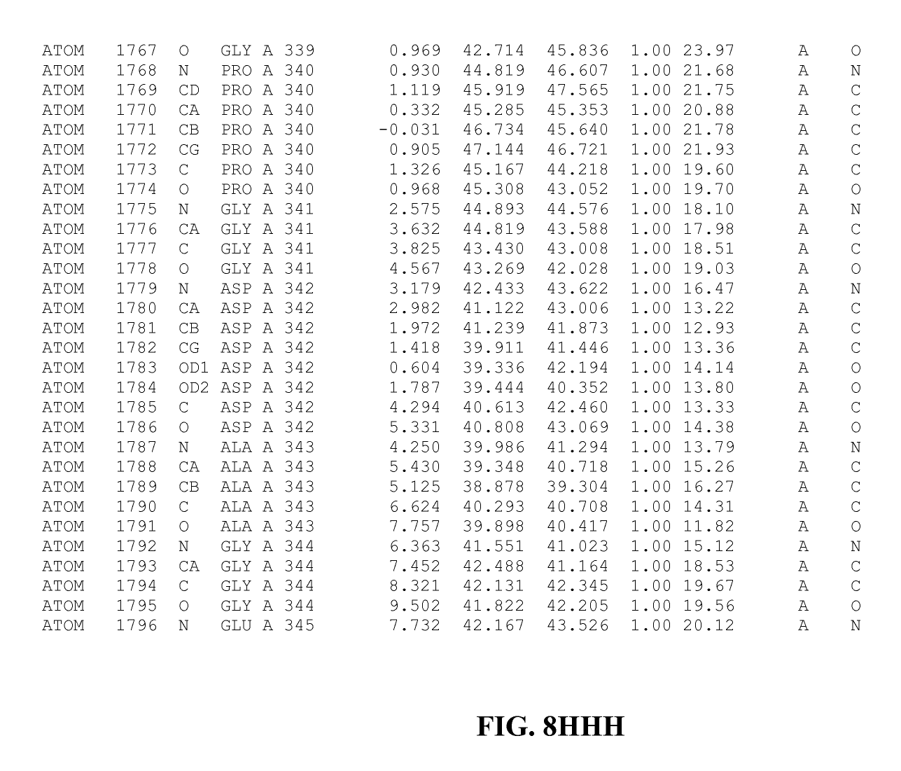

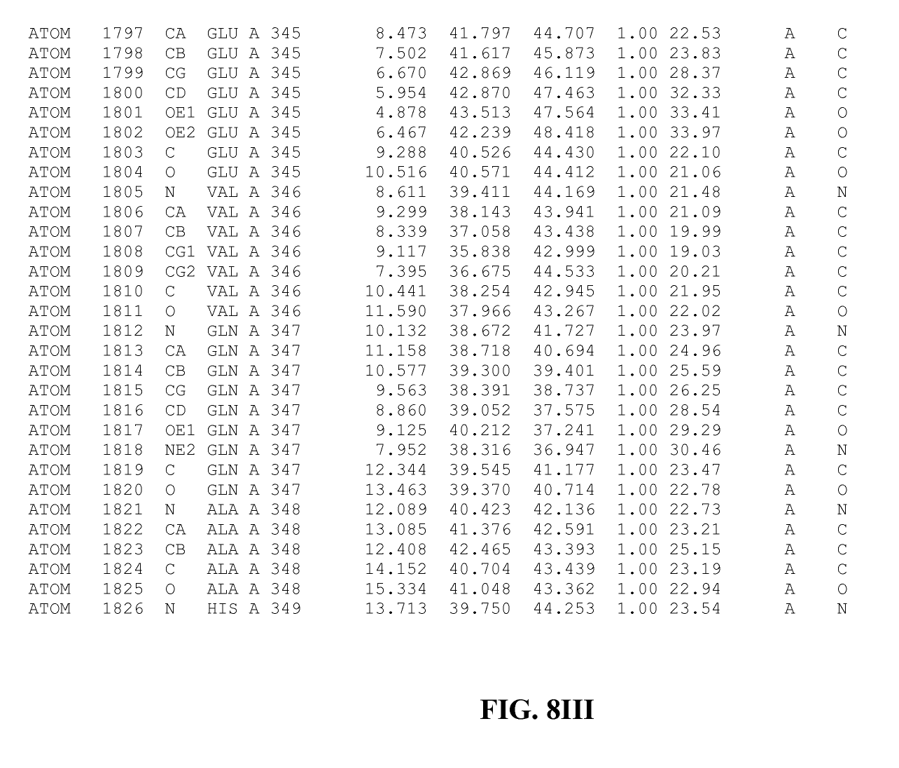

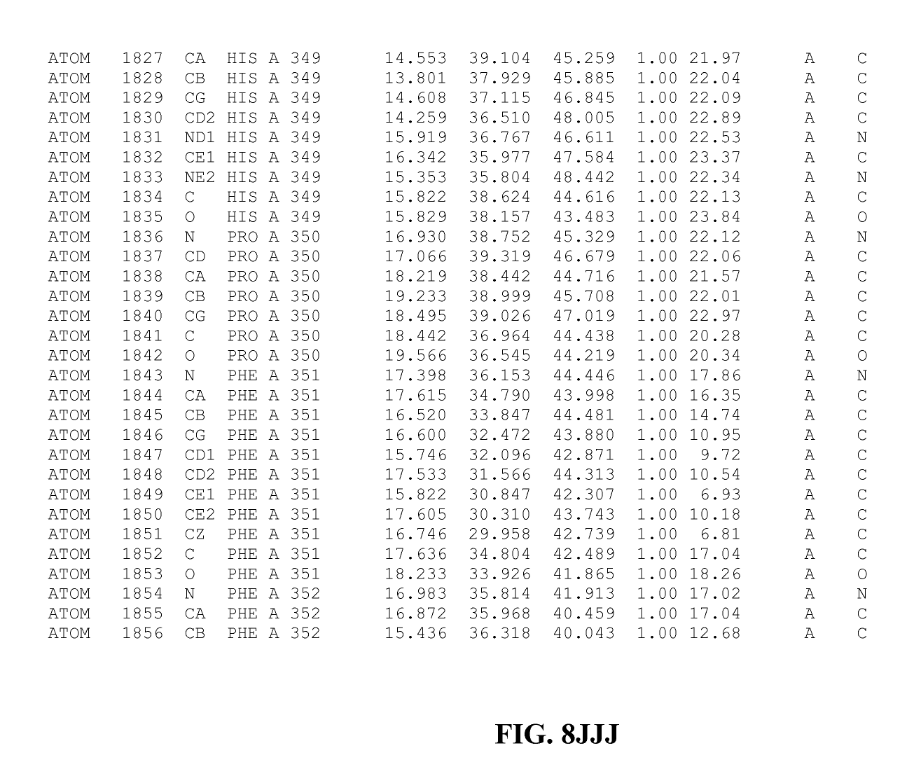

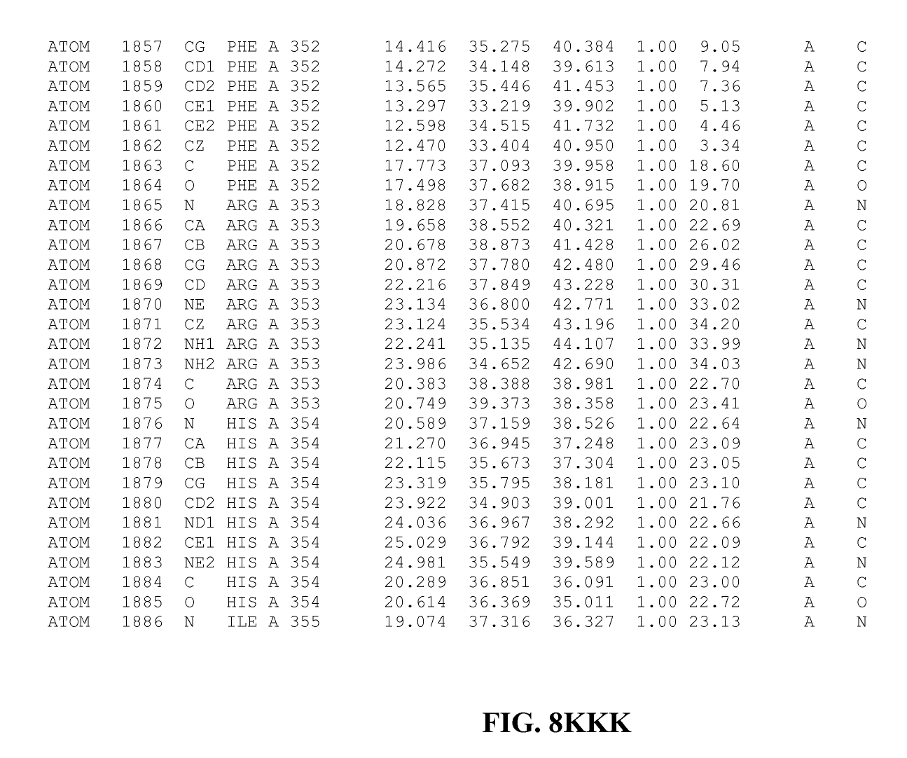

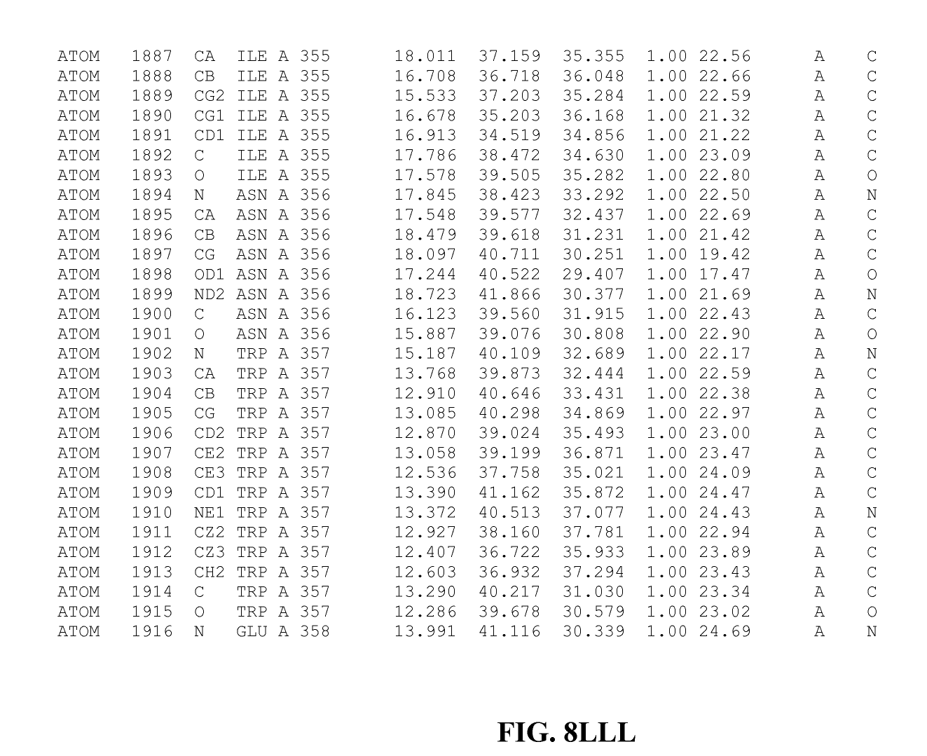

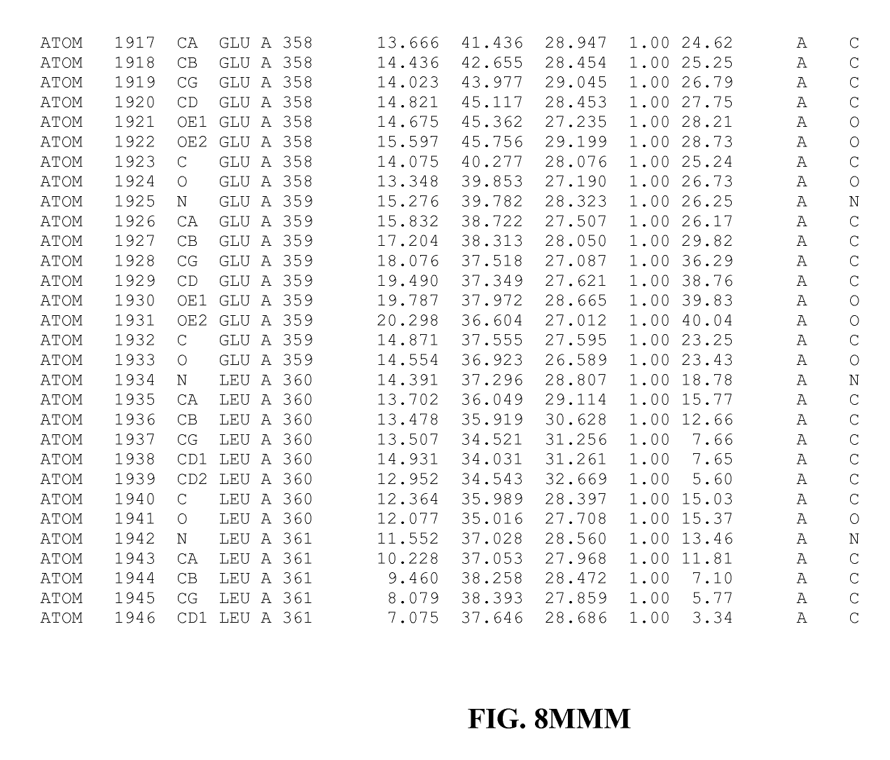

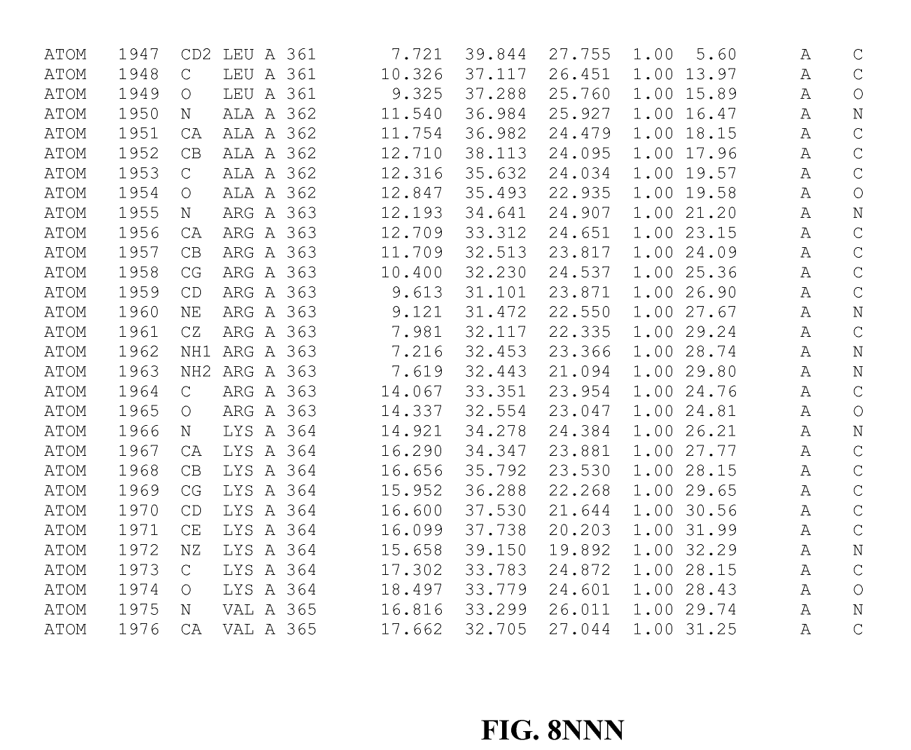

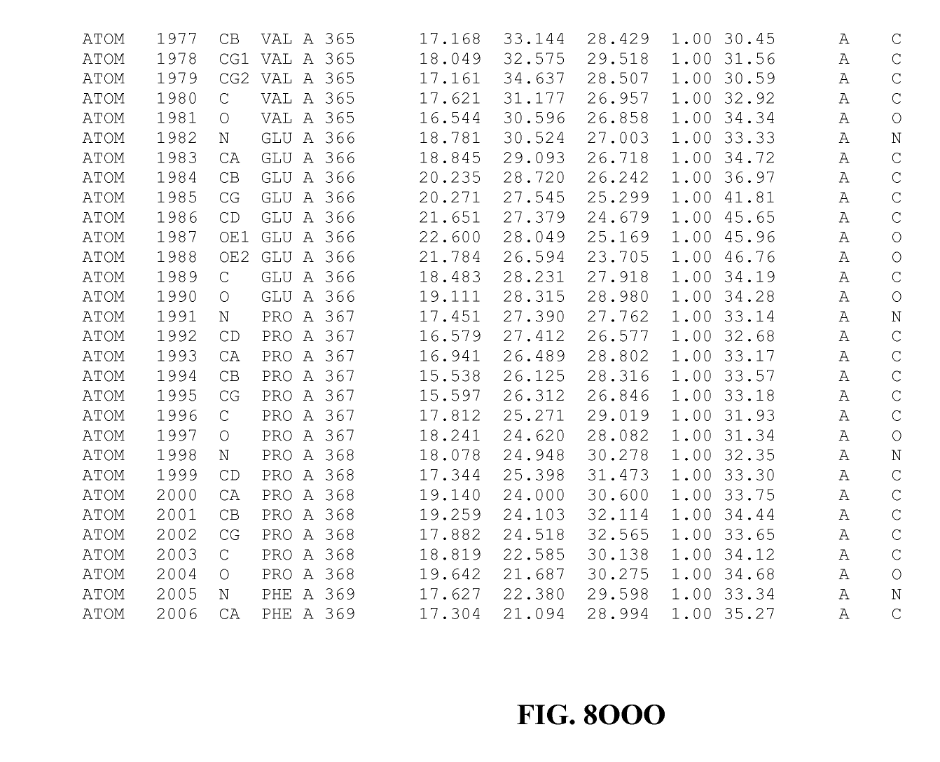

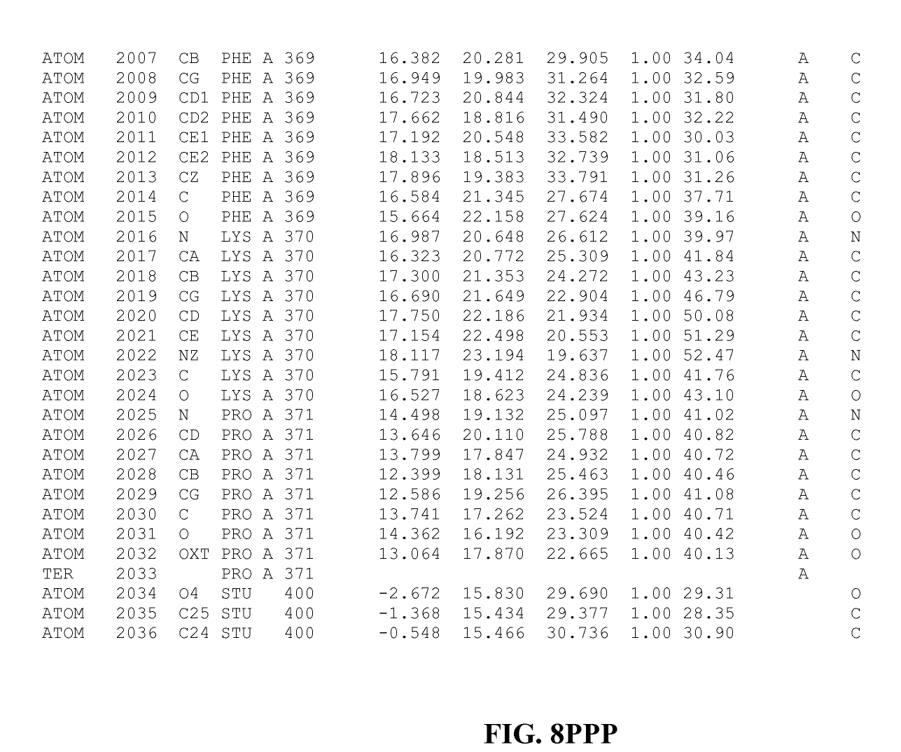

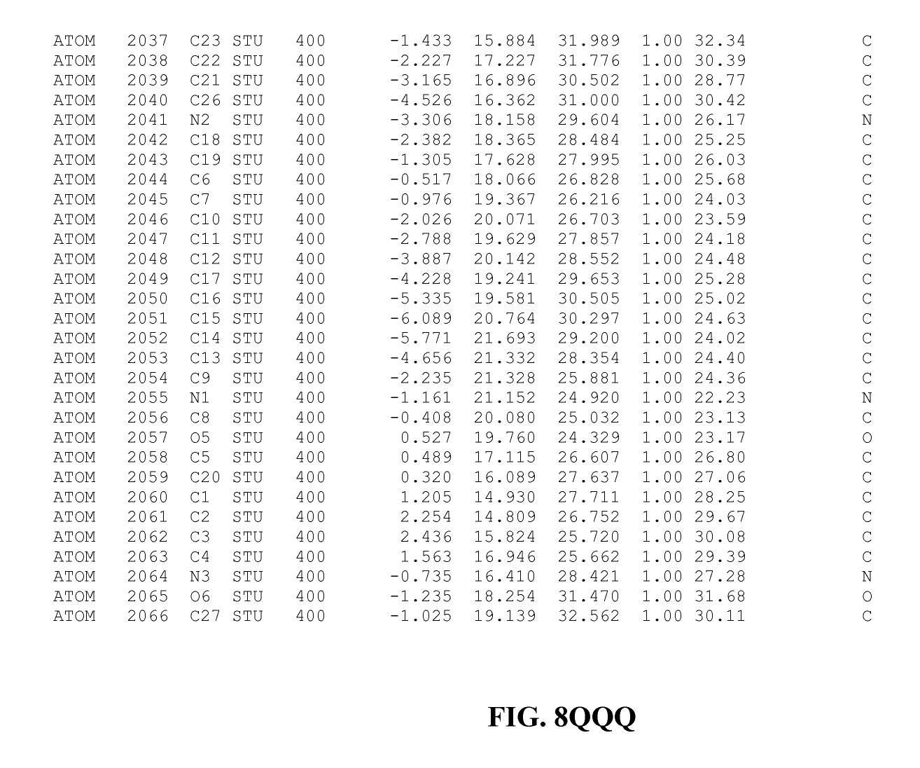

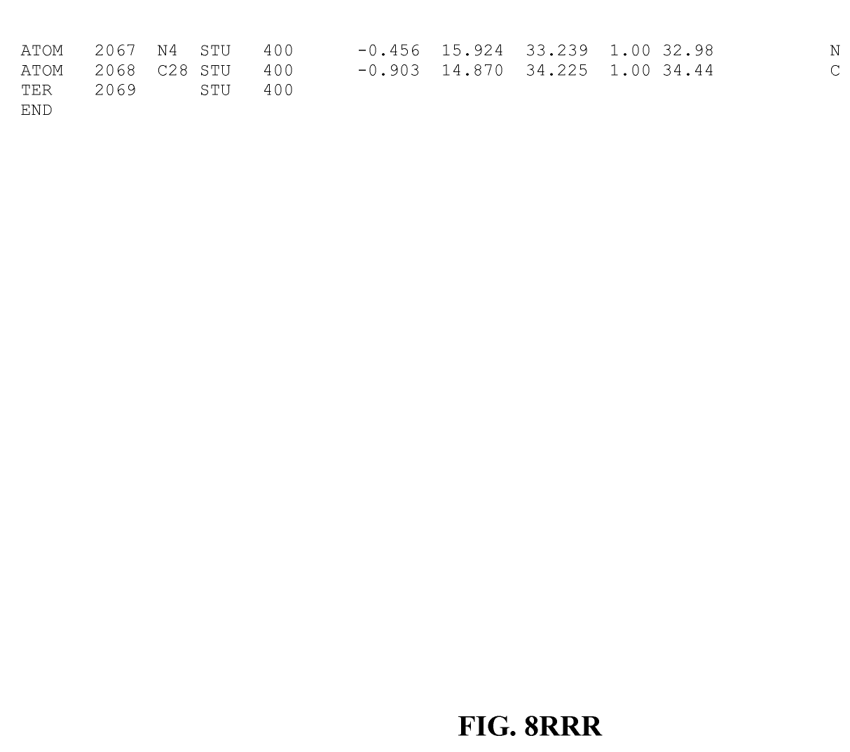

Following the Examples herein, the crystal structure of the p70S6K kinase domain polypeptide in complex with the ligand staurosporine was solved. Staurosporine (antibiotic AM-2282) is a natural product originally isolated in 1977 from Streptomyces staurosporeus. The Systematic (IUPAC) name of staurosporine is (9S,10R,11R,13R)-2,3,10,11,12,13-Hexahydro-10-methoxy-9-methyl-11-(methylamino)-9,13-epoxy-1H,9H-diindolo[1,2,3-gh:3′,2′,1′-lm]pyrrolo[3,4-j][1,7]benzodiazonin-1-one. The main activity of staurosporine is the inhibition of protein kinases through the prevention of ATP binding to the kinase. This is achieved through the stronger affinity of staurosporine than ATP to the ATP-binding site on the kinase. A summary of the crystal's attributes for p70S6K1 kinase domain bound to staurosporine are listed in Table 1 and the three dimensional structure coordinates for space groups P2₁ and P2₁2₁2₁ for p70S6K1 kinase domain bound to staurosporine are shown in FIGS. 7 and 8, respectively. Ribbon diagrams of the p70S6K kinase domain bound to staurosporine based upon the coordinates for space groups P2₁ and P2₁2₁2₁ are shown in FIGS. 5 and 6, respectively.

FIGS. 5 and 6 show that staurosporine binds to the hydrophobic cleft between the N- and C-lobes. One side of the hydrophobic core of the indolocarbazole makes many favorable van der Waals contacts with the backbone and side chains of residues from the N-terminal domain, which includes the backbone and side chains of Leucine at position 97, Valine at position 105, Alanine at position 121, Lysine at position 123, Leucine at position 172, and Tyrosine at position 174. The other side of the indolocarbazole makes van der Waals interactions with the side chains of Leucine at position 175, Methionine at position 225, Threonine at position 235, and Aspartic acid at position 236 from the C-terminal domain. The N1 atom of staurosporine on the lactam moiety makes a hydrogen bond (3.0 Å) to the backbone carbonyl oxygen of Glutamic acid at position 173, while O5 atom of the inhibitor accepts a hydrogen bond (2.6 Å) from the amide nitrogen of Leucine at position 175 in the linker region. The tetrahydropyran ring group sits in the ribose-binding pocket surrounded by Glycine at position 98, Glycine at position 100, Glutamic acid at position 179, Glutamic acid at position 222, Asparagine at position 223, Methionine at position 225, and Aspartic acid at position 236. The N4 atom of staurosporine hydrogen bonds to Glutamic acid at position 179 of the p70S6K1 kinase domain.

TABLE 1
Data collection and refinement statistics.

| | | |
|---|---|---|
| Data Collection | | |
| Space group | P2₁ | P2₁2₁2₁ |
| Unit-cell parameters | a = 78.6, b = 62.9, | a = 56.6, b = 63.0, |
| (Å, °) | c = 87.0, beta = 94.3 | c = 98.3 |
| Resolution (Å) | 2.8 (2.8-2.9) | 3.4 (3.52-3.40) |
| Observed reflections | 73002 | 34120 |
| Unique reflections | 20565 (1733) | 5002 (489) |
| Completeness (%) | 98 (84) | 100 (100) |
| R_{sym} (%)^{a} | 4.9 (28.2) | 9.4 (32.4) |
| <I/σ (I)> | 14.3 | 10.5 |
| Refinement | | |
| Resolution limits (Å) | 2.8 | 3.4 |
| Reflections used | 17912 | 4522 |
| R factor (%)^{b} | 21.3 | 26.5 |
| R_{free} (%) | 27.4 | 35.0 |
| No. of solvent | 6 | 0 |
| molecules | | |
| r.m.s.d. bond length (Å)^{c} | 0.009 | 0.011 |
| r.m.s.d. bond angle (°) | 1.02 | 1.38 |
| Ramachandran plot | | |
| statistics (%) | | |
| Residues in most | 85.3% | 75.7% |
| favored region | | |
| Residues in additional | 11.9% | 21.5% |
| allowed region | | |

^{a}R_{sym} (%) = Σ|Iᵢ − <I>|/Σ Iᵢ
^{b}R factor (%) = Σ||Fₒ| − |F_{c}||/Σ|Fₒ|, where Fo and Fc are observed and calculated structure factors; R_{free} was calculated from a randomly chosen 10% of reflections excluded from refinement, and R factor was calculated for the remaining 90% of reflections.
^{c}r.m.s.d. is the root-mean-square deviation from ideal geometry.

It should be recognized that the above-described crystallization conditions can be varied. Such variations may be used alone or in combination, and include polypeptide solutions containing polypeptide concentrations between 1 mg/mL and 60 mg/mL, any commercially available buffer systems which can maintain pH from about 5.0 to about 6.5, for example Bis-Tris or Na citrate concentrations between about 100 mM to about 200 mM, dithiothreitol concentrations between 0 mM and 20 mM, substitution of dithiothreitol with beta-mercaptoethanol or other art-recognized equivalents; Li₂SO₄ between 200 and 500 mM, and reservoir solutions containing polyethylene glycol concentrations between about 10% and about 30%, polyethylene glycol average molecular weights between about 1,000 and about 20,000 Daltons, any commercially available buffer systems which can maintain pH from about 5.0 to about 6.5, dithiothreitol concentrations between 0 mM and 20 mM, substitution of dithiothreitol with beta-mercaptoethanol or other art-recognized —SH group containing equivalents, or substitution of staurosporine with other protein kinase inhibitors, and temperature ranges between 4 and 20° C.

Derivative crystals of the p70S6K1 kinase domain can be obtained by soaking apo or co-crystals in mother liquor containing salts of heavy metal atoms, according to procedures known to those of skill in the art of X-ray crystallography.

Co-crystals of the invention can be obtained by soaking an apo crystal in mother liquor containing a ligand that binds to the kinase domain, or can be obtained by co-crystallizing the p70S6K1 kinase domain polypeptide in the presence of one or more ligands that bind to the kinase domain.

The present invention is also directed to machine-readable data storage media having data storage material encoded with machine-readable data that comprises structure coordinates for the p70S6K1 kinase domain. The present invention is also directed to a machine readable data storage media having data storage material encoded with machine readable data, which, when read by an appropriate machine, can display a three dimensional representation of a structure of the p70S6K1 kinase domain.

All or a portion of the p70S6K1 kinase domain coordinate data shown in FIGS. 7A-7JJJJJJ and 8A-8RRR, when used in conjunction with a computer programmed with software to translate those coordinates into the three-dimensional structure of the p70S6K1 kinase domain may be used for a variety of purposes, especially for purposes relating to drug discovery. Software for generating three-dimensional graphical representations are known and commercially available. The ready use of the coordinate data requires that it be stored in a computer-readable format. Thus, in accordance with the present invention, data capable of being displayed as the three-dimensional structure of the p70S6K1 kinase domain and/or portions thereof and/or their structurally similar variants may be stored in a machine-readable storage medium, which is capable of displaying a graphical three-dimensional representation of the structure.

Another embodiment of this invention provides a machine-readable data storage medium, comprising a data storage material encoded with machine readable data which, when used by a machine programmed with instructions for using said data, displays a graphical three-dimensional representation comprising the p70S6K1 kinase domain or variant thereof.

Optionally, a computer system is provided in combination with the machine-readable data storage medium provided herein. In one embodiment, the computer system comprises a working memory for storing instructions for processing the machine-readable data; a processing unit coupled to the working memory and to the machine-readable data storage medium, for processing the machine-readable data into the three-dimensional representation; and an output hardware coupled to the processing unit, for receiving the three-dimensional representation.

Uses of the Three Dimensional Structure of p70S6K1 Kinase Domain

The three-dimensional crystal structure of the present invention may be used to identify p70S6K1 kinase domain binding sites, be used as a molecular replacement model to solve the structure of unknown crystallized proteins, to design mutants having desirable binding properties, and ultimately, to design, characterize, identify entities capable of interacting with the p70S6K1 kinase domain and other structurally similar proteins as well as other uses that would be recognized by one of ordinary skill in the art. Such entities may be chemical entities or proteins. The term “chemical entity”, as used herein, refers to chemical compounds, complexes of at least two chemical compounds, and fragments of such compounds.

The p70S6K1 kinase domain structure coordinates provided herein are useful for screening and identifying drugs that inhibit p70S6K1 and other structurally similar proteins. For example, the structure encoded by the data may be computationally evaluated for its ability to associate with putative substrates or ligands. Such compounds that associate with p70S6K1 kinase domain may inhibit p70S6K1 kinase activity, and are potential drug candidates. Additionally or alternatively, the structure encoded by the data may be displayed in a graphical three-dimensional representation on a computer screen. This allows visual inspection of the structure, as well as visual inspection of the structure's association with the compounds.

Thus, according to another embodiment of the present invention, a method is provided for evaluating the potential of an entity to associate with the p70S6K1 kinase domain or variant thereof by using all or a portion of the structure coordinates provided in FIGS. 7A-7JJJJJJ or 8A-8RRR or functional equivalents thereof. A method is also provided for evaluating the potential of an entity to associate with the p70S6K1 kinase domain or variant thereof by using structure coordinates similar to all or a portion of the structure coordinates provided in FIG. 7A-7JJJJJJ or 8A-8RRR or functional equivalents thereof.

The method may optionally comprise the steps of: creating a computer model of all or a portion of a protein structure (e.g., a binding pocket) using structure coordinates according to the present invention; performing a fitting operation between the entity and the computer model; and analyzing the results of the fitting operation to quantify the association between the entity and the model. The portion of the protein structure used optionally comprises all of the amino acids listed in FIG. 7A-7JJJJJJ or 8A-8RRR that are present in the structure coordinates being used.

With the structure provided herein, the present invention for the first time permits the use of molecular design techniques to identify, select or design potential inhibitors of p70S6K1, based on the structure of an p70S6K1 kinase domain. Such a predictive model is valuable in light of the high costs associated with the preparation and testing of the many diverse compounds that may possibly bind to the p70S6K1.

According to this invention, a potential p70S6K1 inhibitor may now be evaluated for its ability to bind an p70S6K1 kinase domain prior to its actual synthesis and testing. If a proposed entity is predicted to have insufficient interaction or association with the binding pocket, preparation and testing of the entity can be obviated. However, if the computer modeling indicates a strong interaction, the entity may then be obtained and tested for its ability to bind.

A potential inhibitor of a p70S6K1 may be computationally evaluated using a series of steps in which chemical entities or fragments are screened and selected for their ability to associate with the p70S6K1 kinase domain.

One skilled in the art may use one of several methods to screen entities (whether chemical or protein) for their ability to associate with a p70S6K1 kinase domain. This process may begin by visual inspection of, for example, a p70S6K1 kinase domain on a computer screen based on the p70S6K1 kinase domain structure coordinates in FIG. 7A-7JJJJJJ or 8A-8RRR or other coordinates which define a similar shape generated from the machine-readable storage medium. Selected fragments or chemical entities may then be positioned in a variety of orientations, or docked, within that binding pocket as defined above. Docking may be accomplished using software such as Quanta and Sybyl, followed by energy minimization and molecular dynamics with standard molecular mechanics force fields, such as CHARMM and AMBER.

Specialized computer programs may also assist in the process of selecting entities. These include: GRID (Goodford, “A Computational Procedure for Determining Energetically Favorable Binding Sites on Biologically Important Macromolecules”, J. Med. Chem., 28, pp. 849-857 (1985)). GRID is available from Oxford University, Oxford, UK; MCSS (Miranker et al., “Functionality. Maps of Binding Sites: A Multiple Copy Simultaneous Search Method.” Proteins: Structure, Function and Genetics, 11, pp. 29-34 (1991)). MCSS is available from Molecular Simulations, San Diego, Calif.; AUTODOCK (Goodsell et al., “Automated Docking of Substrates to Proteins by Simulated Annealing”, Proteins: Structure, Function, and Genetics, 8, pp. 195-202 (1990)). AUTODOCK is available from Scripps Research Institute, La Jolla, Calif.; & DOCK (Kuntz et al., “A Geometric Approach to Macromolecule-Ligand Interactions”, J. Mol. Biol., 161, pp. 269-288 (1982)). DOCK is available from University of California, San Francisco, Calif.

Once suitable entities have been selected, they can be designed or assembled. Assembly may be preceded by visual inspection of the relationship of the fragments to each other on the three-dimensional image displayed on a computer screen in relation to the structure coordinates of the p70S6K1 kinase domain. This may then be followed by manual model building using software such as MOE, QUANTA or Sybyl (Tripos Associates, St. Louis, Mo.).

Useful programs to aid one of skill in the art in connecting the individual chemical entities or fragments include: CAVEAT (Bartlett et al., “CAVEAT: A Program to Facilitate the Structure-Derived Design of Biologically Active Molecules”, in “Molecular Recognition in Chemical and Biological Problems”, Special Pub., Royal Chem. Soc., 78, pp. 182-196 (1989); Lauri and Bartlett, “CAVEAT: a Program to Facilitate the Design of Organic Molecules”, J. Comput. Aided Mol. Des., 8, pp. 51-66 (1994)). CAVEAT is available from the University of California, Berkeley, Calif.; 3D Database systems such as ISIS (MDL Information Systems, San Leandro, Calif.). This area is reviewed in Martin, “3D. Database Searching in Drug Design”, J. Med. Chem., 35, pp. 2.145-2154 (1992); HOOK (Eisen et al., “HOOK: A Program for Finding Novel Molecular Architectures that Satisfy the Chemical and Steric Requirements of a Macromolecule Binding Site”, Proteins: Struct., Funct., Genet., 19, pp. 199-221 (1994). HOOK is available from Molecular Simulations, San Diego, Calif.

Instead of proceeding to build an inhibitor of a p70S6K1 kinase domain in a step-wise fashion one fragment or entity at a time as described above, inhibitory or other p70S6K1 kinase domain binding compounds may be designed as a whole or “de novo” using either an empty binding site or optionally including some portion(s) of a known inhibitor(s). There are many de novo ligand design methods including: LUDI (Bohm, “The Computer Program LUDI: A New Method for the De Novo Design of Enzyme Inhibitors”, J. Comp. Aid. Molec. Design, 6, pp. 61-78 (1992)). LUDI is available from Molecular Simulations Incorporated, San Diego, Calif.; LEGEND (Nishibata et al., Tetrahedron, 47, p. 8985 (1991)). LEGEND is available from Molecular Simulations Incorporated, San Diego, Calif.; LEAPFROG (available from Tripos Associates, St. Louis, Mo.); & SPROUT (Gillet et al., “SPROUT: A Program for Structure Generation)”, J. Comput. Aided Mol. Design, 7, pp. 127-153 (1993)). SPROUT is available from the University of Leeds, UK.

Other molecular modeling techniques may also be employed in accordance with this invention (See, for example, Cohen et al., “Molecular Modeling Software and Methods for Medicinal Chemistry, J. Med. Chem., 33, pp. 883-894 (1990); See also, Navia and Murcko, “The Use of Structural Information in Drug Design”, Current Opinions in Structural Biology, 2, pp. 202-210 (1992); Balbes et al., “A Perspective of Modern Methods in Computer-Aided Drug Design”, in Reviews in Computational Chemistry, Vol. 5, Lipkowitz and Boyd, Eds., VCH, New York, pp. 337-380 (1994); See also, Guida, “Software For Structure-Based Drug Design”, Curr. Opin. Struct. Biology, 4, pp. 777-781 (1994)).

Once an entity has been designed or selected, for example, by the above methods, the efficiency with which that entity may bind to a p70S6K1 kinase domain may be tested and optimized by computational evaluation. For example, an effective p70S6K1 kinase domain inhibitor preferably demonstrates a relatively small difference in energy between its bound and free states (i.e., a small deformation energy of binding). Thus, the most efficient p70S6K1 kinase domain inhibitors should preferably be designed with deformation energy of binding of not greater than about 10 kcal/mole, more preferably, not greater than 7 kcal/mole. p70S6K1 kinase domain inhibitors may interact with the domain in more than one of multiple conformations that are similar in overall binding energy. In those cases, the deformation energy of binding is taken to be the difference between the energy of the free entity and the average energy of the conformations observed when the inhibitor binds to the protein.

An entity designed or selected as binding to a p70S6K1 kinase domain may be further computationally optimized so that in its bound state it would preferably lack repulsive electrostatic interaction with the target enzyme and with the surrounding water molecules. Such non-complementary electrostatic interactions include repulsive charge-charge, dipole-dipole and charge-dipole interactions.

Specific computer software is available in the art to evaluate compound deformation energy and electrostatic interactions. Examples of programs designed for such uses include: Gaussian 94, revision C (Frisch, Gaussian, Inc., Pittsburgh, Pa. 1995); AMBER, version 4.1 (Kollman, University of California at San Francisco, 1995); QUANTA/CHARMM (Molecular Simulations, Inc., San Diego, Calif. 1995); Insight II/Discover (Molecular Simulations, Inc., San Diego, Calif. 1995); DelPhi (Molecular Simulations, Inc., San Diego, Calif. 1995); and AMSOL (Quantum Chemistry Program Exchange, Indiana University). These programs may be implemented, for instance, using a Silicon Graphics workstation such as an Indigo.sup.2 with “IMPACT” graphics. Other hardware systems and software packages will be known to those skilled in the art.

Another approach provided by this invention, is the computational screening of small molecule databases for chemical entities or compounds that can bind in whole, or in part, to a p70S6K1 kinase domain. In this screening, the quality of fit of such entities to the binding site may be judged either by shape complementarities or by estimated interaction energy (Meng et al., J. Comp. Chem., 13, 505-524 (1992)).

According to another embodiment, the invention provides compounds that associate: with a p70S6K1 kinase domain produced or identified by various methods set forth above.

The structure coordinates set forth in FIG. 7A-7JJJJJJ or 8A-8RRR can also be used to aid in obtaining structural information about another crystallized molecule or molecular complex. This may be achieved by any of a number of well-known techniques, including molecular replacement.

For example, a method is also provided for utilizing molecular replacement to obtain structural information about a protein whose structure is unknown comprising the steps of: generating an X-ray diffraction pattern of a crystal of the protein whose structure is unknown; generating a three-dimensional electron density map of the protein whose structure is unknown from the X-ray diffraction pattern by using at least a portion of the structure coordinates set forth in FIG. 7A-7JJJJJJ or 8A-8RRR as a molecular replacement model.

By using molecular replacement, all or part of the structure coordinates of the p70S6K1 kinase domain provided by this invention (and set forth in FIG. 7A-7JJJJJJ or 8A-8RRR) can be used to determine the structure of another crystallized molecule or molecular complex more quickly and efficiently than attempting an ab initio structure determination. One particular use includes use with other structurally similar proteins. Molecular replacement provides an accurate estimation of the phases for an unknown structure. Phases are a factor in equations used to solve crystal structures that cannot be determined directly. Obtaining accurate values for the phases, by methods other than molecular replacement, is a time-consuming process that involves iterative cycles of approximations and refinements and greatly hinders the solution of crystal structures. However, when the crystal structure of a protein containing at least a homologous portion has been solved, the phases from the known structure provide a satisfactory estimate of the phases for the unknown structure.

Thus, this method involves generating a preliminary model of a molecule or molecular complex whose structure coordinates are unknown, by orienting and positioning the relevant portion of the p70S6K1 kinase domain according to FIG. 7A-7JJJJJJ or 8A-8RRR within the unit cell of the crystal of the unknown molecule or molecular complex so as best to account for the observed X-ray diffraction pattern of the crystal of the molecule or molecular complex whose structure is unknown. Phases can then be calculated from this model and combined with the observed X-ray diffraction pattern amplitudes to generate an electron density map of the structure whose coordinates are unknown. This, in turn, can be subjected to any well-known model building and structure refinement techniques to provide a final, accurate structure of the unknown crystallized molecule or molecular complex (Lattman, “Use of the Rotation and Translation Functions”, in Meth. Enzymol., 115, pp. 55-77 (1985); Rossmann, ed., “The Molecular Replacement Method”, Int. Sci. Rev. Ser., No. 13, Gordon & Breach, New York (1972)).

The structure of any portion of any crystallized molecule or molecular complex that is sufficiently homologous to any portion p70S6K1 kinase domain can be resolved by this method.

In one embodiment, the method of molecular replacement is utilized to obtain structural information about the present invention and any other p70S6K1-like molecule. The structure coordinates of p70S6K1 kinase domain, as provided by this invention, are particularly useful in solving the structure of other isoforms of p70S6K1 or p70S6K1 complexes.

The structure coordinates of p70S6K1 kinase domain as provided by this invention are useful in solving the structure of p70S6K1 kinase domain variants that have amino acid substitutions, additions and/or deletions (referred to collectively as “p70S6K1 kinase domain mutants”, as compared to naturally occurring p70S6K1 kinase domain). These p70S6K1 kinase domain mutants may optionally be crystallized in co-complex with a ligand, such as an inhibitor, substrate analogue or a suicide substrate. The crystal structures of a series of such complexes may then be solved by molecular replacement and compared with that of p70S6K1 kinase domain. Potential sites for modification within the various binding sites of the enzyme may thus be identified. This information provides an additional tool for determining the most efficient binding interactions such as, for example, increased hydrophobic interactions, between p70S6K1 kinase domain and a ligand. It is noted that the ligand may be the protein's natural ligand or may be a potential agonist or antagonist of a protein.

All of the complexes referred to above may be studied using well-known X-ray diffraction techniques and may be refined versus 1.5-3.5 Å resolution X-ray data to an R value of about 0.22 or less using computer software, such as X-PLOR (Brunger et al., X-PLOR, Version 3.1, A system for X-ray crystallography and NMR, Yale University, (1992)), CNS (Brunger et al., Crystallography & NMR System (CNS), A new software suite for macromolecular structure determination, Acta Cryst. D54: 905-921(1998)), TNT (Tronrud et al., An efficient general-Purpose least-squares refinement program for macromolecular structures, Acta Cryst. A43, 489-501 (1987)), Buster (Bricogne, The Bayesian Statistical Viewpoint on Structure Determination: Basic Concepts and Examples”, in Methods in Enzymology, 276A, 361-423. Carter & Sweet, eds. (1997)) and Refmac (Murshudov et al., Refinement of macromolecular structures by the maximum-likelihood method, Acta Cryst D53 :240-255 (1997)) (See, e.g., Blundell & Johnson, supra; Meth. Enzymol., Vol. 114 & 115, Wyckoff et al., eds., Academic Press (1985) ). This information may thus be used to optimize known p70S6K1 inhibitors, and more importantly, to design new p70S6K1 inhibitors.

The structure coordinates described above may also be used to derive the dihedral angles, phi and psi, that define the conformation of the amino acids in the protein backbone. As will be understood by those skilled in the art, the phiₙ angle refers to the rotation around the bond between the alpha-carbon and the nitrogen, and the psiₙ angle refers to the rotation around the bond between the carbonyl carbon and the alpha-carbon. The subscript “n” identifies the amino acid whose conformation is being described([for a general reference, See Blundell and Johnson, Protein Crystallography, Academic Press, London, 1976).

Uses of the Crystal and Diffraction Pattern of p70S6K1 Kinase Domain

Crystals, crystallization conditions, and the diffraction pattern of p70S6K1 kinase domain that can be generated from the crystals also have a range of uses. One particular use relates to screening entities that are not known ligands of the p70S6K1 kinase domain for their ability to bind to the p70S6K1 kinase domain. For example, with the availability of crystallization conditions, crystals and diffraction patterns of the p70S6K1 kinase domain provided according to the present invention, it is possible to take a crystal of the p70S6K1 kinase domain; expose the crystal to one or more entities that may be a ligand of the p70S6K1 kinase domain; and determine whether a ligand/p70S6K1 kinase domain complex is formed. The crystals of the p70S6K1 kinase domain may be exposed to potential ligands by various methods, including but not limited to, soaking a crystal in a solution of one or more potential ligands or co-crystallizing the p70S6K1 kinase domain in the presence of one or more potential ligands. Given the structure coordinates provided herein, once a ligand complex is formed, the structure coordinates can be used as a model in molecular replacement in order to determine the structure of the ligand complex.

Once one or more ligands are identified, structural information from the ligand/p70S6K1 kinase domain complex(es) may be used to design new ligands that bind tighter, bind more specifically, have better biological activity or have better safety profile than known ligands.

In one embodiment, a method is provided for identifying a ligand that binds to the p70S6K1 kinase domain comprising: (a) attempting to crystallize a protein that comprises an amino acid sequence wherein at least a portion of the sequence has 55%, 65%, 75%, 85%, 90%, 95%, 97%, 99% or greater identity with an amino acid sequence between amino acid 90 and 360 of SEQ ID NO:7: in the presence of one or more entities; (b) if crystals of the protein are obtained in step (a), obtaining an X-ray diffraction pattern of the protein crystal; and (c) determining whether a ligand/protein complex was formed by comparing an X-ray diffraction pattern of a crystal of the protein formed in the absence of the one or more entities to the crystal formed in the presence of the one or more entities.

In one embodiment, a method is provided for identifying a ligand that binds to the p70S6K1 kinase domain comprising: (a) attempting to crystallize a protein that comprises an amino acid sequence wherein at least a portion of the sequence has 55%, 65%, 75%, 85%, 90%, 95%, 97%, 99% or greater identity with an amino acid sequence between amino acid 75 and 399 of SEQ ID NO:7: in the presence of one or more entities; (b) if crystals of the protein are obtained in step (a), obtaining an X-ray diffraction pattern of the protein crystal; and (c) determining whether a ligand/protein complex was formed by comparing an X-ray diffraction pattern of a crystal of the protein formed in the absence of the one or more entities to the crystal formed in the presence of the one or more entities.

In another embodiment, a method is provided for identifying a ligand that binds to the p70S6K1 kinase domain comprising: soaking a crystal of a protein wherein at least a portion of the protein has 55%, 65%, 75%, 85%, 90%, 95%, 97%, 99% or greater amino acid sequence identity with an amino acid sequence between amino acid 90 and 360 of SEQ ID NO:7 with one or more entities; determining whether a ligand/protein complex was formed by comparing an X-ray diffraction pattern of a crystal of the protein that has not been soaked with the one or more entities to the crystal that has been soaked with the one or more entities.

In another embodiment, a method is provided for identifying a ligand that binds to the p70S6K1 kinase domain comprising: soaking a crystal of a protein wherein at least a portion of the protein has 55%, 65%, 75%, 85%, 90%, 95%, 97%, 99% or greater amino acid sequence identity with an amino acid sequence between amino acid 75 and 399 of SEQ ID NO:7 with one or more entities; determining whether a ligand/protein complex was formed by comparing an X-ray diffraction pattern of a crystal of the protein that has not been soaked with the one or more entities to the crystal that has been soaked with the one or more entities.

Optionally, the method may further comprise converting the diffraction patterns into electron density maps using phases of the protein crystal and comparing the electron density maps.

Libraries of “shape-diverse” compounds may optionally be used to allow direct identification of the ligand-receptor complex even when the ligand is exposed as part of a mixture. According to this variation the need for time-consuming de-convolution of a hit from the mixture is avoided. More specifically, the calculated electron density function reveals the binding event, identifies the bound compound and provides a detailed 3-D structure of the ligand-receptor complex. Once a hit is found, one may optionally also screen a number of analogs or derivatives of the hit for tighter binding or better biological activity by traditional screening methods. The hit and information about the structure of the target may also be used to develop analogs or derivatives with tighter binding or better biological activity. It is noted that the ligand-p70S6K1 kinase domain complex may optionally be exposed to additional iterations of potential ligands so that two or more hits can be linked together to make a more potent ligand. Screening for potential ligands by co-crystallization and/or soaking is further described in U.S. Pat. No. 6,297,021.

The following examples are intended to promote a further understanding of the present invention.

### EXAMPLE 1

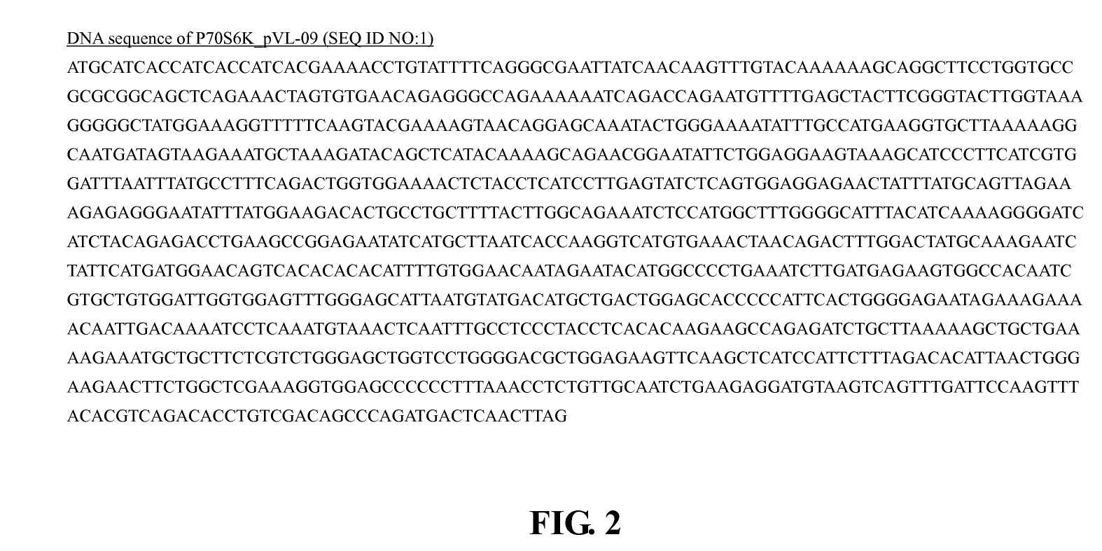

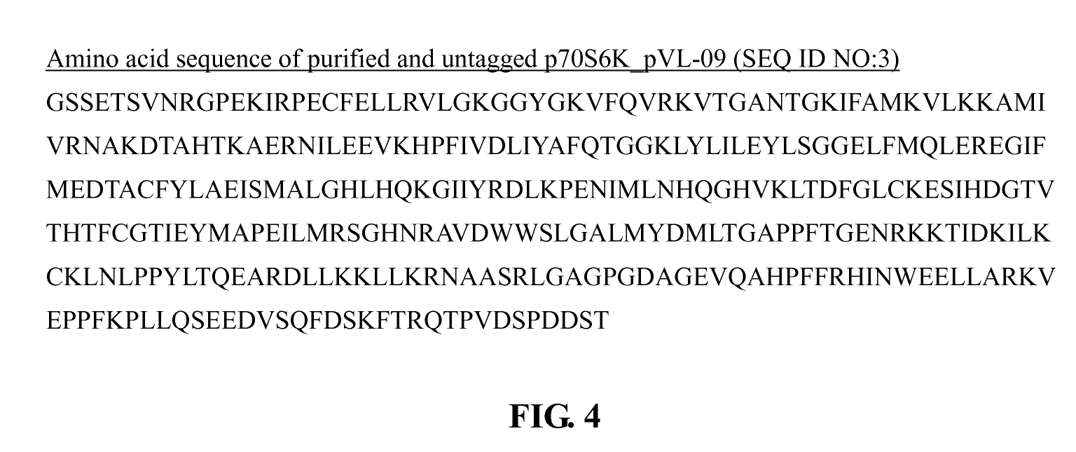

This example illustrates a method for Crystallization of p70S6K1 kinase domain. The p70S6K1 kinase domain was cloned as the variant shown in FIG. 2 The encoded kinase domain is shown in FIGS. 4.

The template used for PCR amplification of the kinase domain was a full length p70S6k cDNA clone (purchased from Open Biosystems; accession # NM_{—}003161; SEQ ID NO:7). The template was used to PCR amplify the kinase domain spanning amino acid residues 75-399. The 5′ primers that encode the Gateway recombination sites and thrombin cleavage site just before the start of the open reading frame (ORF) and the 3′ primers encoding the end of the ORF followed by STOP codon were used. The nucleotide sequences of the primers used are v9 5′ primer: 5′-GGGGACAAGT TTGTACAAAA AAGCAGGCTT CCTGGTGCCG CGCGGCAGCT CAGAAACTAG TGTGAACAGA G-3′ (SEQ ID NO:4); and v9 3′Primer: 5′-GGGGACCACT TTGTACAAGA AAGCTGGGTC CTAAGTTGAG TCATCTGGGC TGTCG-3′ (SEQ ID NO:5)

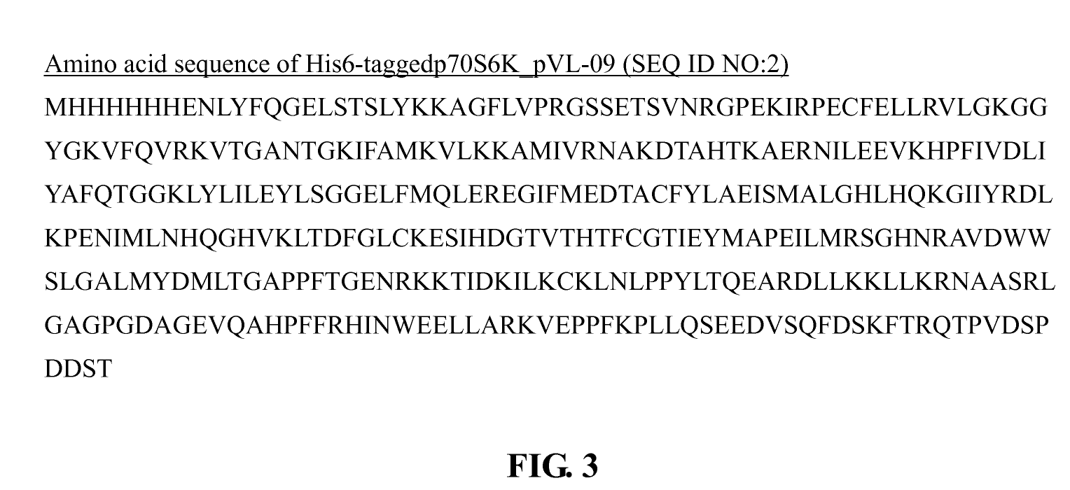

The p70S6K1 kinase domain was PCR amplified using Invitrogen's Accuprime High Fidelity Taq polymerase (#12346-086). PCR products were cloned into pDONR-ZEO entry vector using BP Clonase (Invitrogen) creating an entry clone. The entry clone was shuttled into baculovirus expression transfer vector pVL1392-N-6His-DEST (modified pVL1392 vector purchased from Pharmingen) using LR Clonase (Invitrogen). The expression vector was sequenced to confirm that no mutations had been introduced either via PCR or cloning. The amino acid sequence of variant 9 with the N-terminal His-6 tag provided by the vector is shown in FIG. 3.

Expression of p70S6K

Insect cells were used for expression of the p70S6K1 kinase domain. Recombinant baculovirus vector encoding variant 9 of the p70S6K1 kinase domain was made by cotransfecting the above expression vectors with BacMagic viral DNA (EMD biosciences) into Sf 21 cells. Recombinant viruses encoding the p70S6K1 kinase domain were amplified to P2 and titered using an antibody assay. The p70S6K1 kinase domain was expressed in the Sf21 insect cell at MOI's 0.07 and the infection was done at cell density of 1×10⁶. The cells were harvested at 72 hours post infection and the cell paste was stored at −70° C. until use.

Purification of p70S6K1

The p70S6K1 kinase domain was purified using the following protocol.

Cell Pellets were resuspended (4.5 ml/g of cell weight) in Lysis buffer (25 mM HEPES, pH 7.0, 350 mM Li₂SO₄, 25 mM imidazole 10% glycerol, 0.5% Thesit, 2 mM TCEP, protease inhibitor cocktails, 1 mM AEBSF, 50U/g benzonase, 2 mM MgCl₂) and disrupted by microfluidizer. The insoluble material was removed by centrifugation. The supernatant was loaded on to a 15 mL HisTrap FF crude column (GE healthcare) and the detergent removed by 40 column volumes (CV) wash with buffer A (25 mM Hepes, pH7, 350 mM Li₂SO₄, 10% glycerol, 2 mM TCEP, 50 mM imidazole). The protein was eluted using a linear imidazole gradient from 50 to 500 mM imidazole and the fractions containing the P70S6K1 were pooled. The protein was exchanged into dialysis buffer (25 mM Hepes, pH7, 350 mM Li₂SO₄, 10% glycerol, 1 mM TCEP, 2 mM MnCl₂, 800 u/mg lambda phosphatase (NEB)). After buffer exchange, the His-tag was cleaved by thrombin cleavage reaction (20 U/mg). The cleaved protein was loaded on to a 10 mL HisTrap FF crude column and eluted using a linear imidazole gradient. Purified P70S6K1 was pooled, concentrated and loaded on to a Hiload 26/60 Superdex 75 pg column (GE healthcare) that had been equilibrated in buffer (25 mM Hepes pH7.0, 250 mM Li₂SO₄, 5% glycerol, 2 mM TCEP). The amino acid sequence of final purified and untagged p70S6K is shown in FIG. 4.

Crystallization of p70S6K1 Variant 9

Crystallization was carried out as described as follows. The purified p70S6K1 kinase domain samples were concentrated to 15 mg/mL and the ligand staurosporine (Calbiochem) was added to a final concentration of 1.0 mM. Orthorhombic and monoclinic crystal forms were grown by vapor diffusion at 20° C. by mixing equal volume of protein solution with a crystallization solution consisting of 20-35% (w/v) PEG3350, 100-200 mM Bis-Tris, pH 5-6, and 200-300 mM Li₂SO₄.

### EXAMPLE 2

Structure Determination of p70S6K1 Variant 9

The crystals were transferred to a cryoprotection buffer comprising 15%-20% (w/v) glycerol with 20-35% (w/v) PEG3350 (corresponding to the crystallization condition), 200-300 mM Li₂SO₄, 100-200 mM Bis-Tris pH 5-6. Crystals were frozen by plunging into liquid nitrogen at 100° K. The crystals attributes are listed in Table 1.

X-ray diffraction data were collected at Industrial Macromolecular Crystallographer Association's beamline 17-ID at the Advanced Photon Source for monoclinc and orthorhombic forms. Data were processed using HKL2000. The structure was determined by molecular replacement using the program AMoRe (CCP4 package) using P70S6K1 homology model as a search model. The P70S6K1 homology model was based on RSK1 structure. The structure was refined using CNX. Model building was performed with coot (CCP4 package ).

The crystals attributes are listed in Table 1 and the three dimensional structure coordinates for the P2₁ space group and P2₁2₁2₁ space group are shown in FIGS. 7 and 8. As shown in Table 1, the protein crystal has a crystal lattice in a P2₁2₁2₁ space group and has unit cell dimensions of ±5% of a=56.6 Å, b=63.0 Å, and c=98.3 Å and has a crystal lattice in a P2₁ space group and has unit cell dimensions of ±5% of a=78.6 Å, b=62.9 Å, c=87.0 Å, and β=94.3 Å. FIG. 5 shows a ribbon diagram of the p70S6K1 kinase domain having a crystal lattice in a P2₁ space group. FIG. 6 shows a ribbon diagram of the p70S6K1 kinase domain having a crystal lattice in a P2₁2₁2₁ space group.

FIGS. 5 and 6 also show staurosporine bound to the hydrophobic cleft between the N- and C-lobes. One side of the hydrophobic core of the indolocarbazole makes many favorable van der Waals contacts with the backbone and side chains of residues from the N-terminal domain, which includes the backbone and side chains of Leu97, Val105, Ala121, Lys123, Leu172 and Tyr174. The other side of the indolocarbazole makes van der Waals interactions with the side chains of Leu175, Met225, Thr235 and Asp236 from both the C-terminal domains. The N₁ atom of staurosporine on the lactam moiety makes a hydrogen bond (3.0 Å) to the backbone carbonyl oxygen of Glu173, while O5 atom of the inhibitor accepts a hydrogen bond (2.6 Å) from the amide nitrogen of Leu175 in the linker region. The tetrahydropyran ring group sits in the ribose-binding pocket surrounded by Gly98, Gly100, Glu179, Glu222, Asn223, Met225 and Asp236. The N4 atom of staurosporine hydrogen bonds to Glu179 of the p70S6K1 kinase domain.

While the present invention is described herein with reference to illustrated embodiments, it should be understood that the invention is not limited hereto. Those having ordinary skill in the art and access to the teachings herein will recognize additional modifications and embodiments within the scope thereof. Therefore, the present invention is limited only by the claims attached herein.

| | | |
|---|---|---|
| SEQ ID NO. | DESCRIPTION | SEQUENCE |

| | | |
|---|---|---|
| 1 | DNA sequence of | ATGCATCACCATCACCATCACGAAAACCTGTATTTTC |
| | P70S6K_pVL-09 | AGGGCGAATTATCAACAAGTTTGTACAAAAAAGCAGG |
| | | CTTCCTGGTGCCGCGCGGCAGCTCAGAAACTAGTGTG |
| | | AACAGAGGGCCAGAAAAAATCAGACCAGAATGTTTTG |
| | | AGCTACTTCGGGTACTTGGTAAAGGGGGCTATGGAAA |
| | | GGTTTTTCAAGTACGAAAAGTAACAGGAGCAAATACT |
| | | GGGAAAATATTTGCCATGAAGGTGCTTAAAAAGGCAA |
| | | TGATAGTAAGAAATGCTAAAGATACAGCTCATACAAA |
| | | AGCAGAACGGAATATTCTGGAGGAAGTAAAGCATCCC |
| | | TTCATCGTGGATTTAATTTATGCCTTTCAGACTGGTG |
| | | GAAAACTCTACCTCATCCTTGAGTATCTCAGTGGAGG |
| | | AGAACTATTTATGCAGTTAGAAAGAGAGGGAATATTT |
| | | ATGGAAGACACTGCCTGCTTTTACTTGGCAGAAATCT |
| | | CCATGGCTTTGGGGCATTTACATCAAAAGGGGATCAT |
| | | CTACAGAGACCTGAAGCCGGAGAATATCATGCTTAAT |
| | | CACCAAGGTCATGTGAAACTAACAGACTTTGGACTAT |
| | | GCAAAGAATCTATTCATGATGGAACAGTCACACACAC |
| | | ATTTTGTGGAACAATAGAATACATGGCCCCTGAAATC |
| | | TTGATGAGAAGTGGCCACAATCGTGCTGTGGATTGGT |
| | | GGAGTTTGGGAGCATTAATGTATGACATGCTGACTGG |
| | | AGCACCCCCATTCACTGGGGAGAATAGAAAGAAAACA |
| | | ATTGACAAAATCCTCAAATGTAAACTCAATTTGCCTC |
| | | CCTACCTCACACAAGAAGCCAGAGATCTGCTTAAAAA |
| | | GCTGCTGAAAAGAAATGCTGCTTCTCGTCTGGGAGCT |
| | | GGTCCTGGGGACGCTGGAGAAGTTCAAGCTCATCCAT |
| | | TCTTTAGACACATTAACTGGGAAGAACTTCTGGCTCG |
| | | AAAGGTGGAGCCCCCCTTTAAACCTCTGTTGCAATCT |
| | | GAAGAGGATGTAAGTCAGTTTGATTCCAAGTTTACAC |
| | | GTCAGACACCTGTCGACAGCCCAGATGACTCAACTTA |
| | | G |
| 2 | Amino acid | MHHHHHHENLYFQGELSTSLYKKAGFLVPRGSSETSV |
| | sequence of His6- | NRGPEKIRPECFELLRVLGKGGYGKVFQVRKVTGANT |
| | taggedp70S6K_p | GKIFAMKVLKKAMIVRNAKDTAHTKAERNILEEVKHP |
| | VL-09 | FIVDLIYAFQTGGKLYLILEYLSGGELFMQLEREGIF |
| | | MEDTACFYLAEISMALGHLHQKGIIYRDLKPENIMLN |
| | | HQGHVKLTDFGLCKESIHDGTVTHTFCGTIEYMAPEI |
| | | LMRSGHNRAVDWWSLGALMYDMLTGAPPFTGENRKKT |
| | | IDKILKCKLNLPPYLTQEARDLLKKLLKRNAASRLGA |
| | | GPGDAGEVQAHPFFRHINWEELLARKVEPPFKPLLQS |
| | | EEDVSQFDSKFTRQTPVDSPDDST |
| 3 | Amino acid | GSSETSVNRGPEKIRPECFELLRVLGKGGYGKVFQVR |
| | sequence of | KVTGANTGKIFAMKVLKKAMIVRNAKDTAHTKAERNI |
| | purified and | LEEVKHPFIVDLIYAFQTGGKLYLILEYLSGGELFMQ |
| | untagged | LEREGIFMEDTACFYLAEISMALGHLHQKGIIYRDLK |
| | p70S6K_pVL-09 | PENIMLNHQGHVKLTDFGLCKESIHDGTVTHTFCGTI |
| | | EYMAPEILMRSGHNRAVDWWSLGALMYDMLTGAPPFT |
| | | GENRKKTIDKILKCKLNLPPYLTQEARDLLKKLLKRN |
| | | AASRLGQGPGDAGEVQAHPFFRHINWEELLARKVEPP |
| | | FKPLLQSEEDVSQFDSKFTRQTPVDSPDDST |
| 4 | v9 5′ primer | GGGGACAAGT TTGTACAAAA AAGCAGGCTT |
| | | CCTGGTGCCG CGCGGCAGCT CAGAAACTAG |
| | | TGTGAACAGA G |
| 5 | v9 3′Primer | GGGGACCACT TTGTACAAGA AAGCTGGGTC |
| | | CTAAGTTGAG TCATCTGGGC TGTCG |
| 6 | Kinase substrate | KKRNRTLSVA |
| 7 | P70S6K1 | MRRRRRRDGFYPAPDFRDREAEDMAGVFDIDLDQPED |
| | | AGSEDELEEGGQLNESMDHGGVGPYELGMEHCEKFEI |
| | | SETSVNRGPEKIRPECFELLRVLGKGGYGKVFQVRKV |
| | | TGANTGKIFAMKVLKKAMIVRNAKDTAHTKAERNILE |
| | | EVKHPFIVDLIYAFQTGGKLYLILEYLSGGELFMQLE |
| | | REGIFMEDTACFYLAEISMALGHLHQKGIIYRDLKPE |
| | | NIMLNHQGHVKLTDFGLCKESIHDGTVTHTFCGTIEY |
| | | MAPEILMRSGHNRAVDWWSLGALMYDMLTGAPPFTGE |
| | | NRKKTIDKILKCKLNLPPYLTQEARDLLKKLLKRNAA |
| | | SRLGAGPGDAGEVQAHPFFRHINWEELLARKVEPPFK |
| | | PLLQSEEDVSQFDSKFTRQTPVDSPDDSTLSESANQV |
| | | FLGFTYVAPSVLESVKEKFSFEPKIRSPRRFIGSPRT |
| | | PVSPVKFSPGDFWGRGASASTANPQTPVEYPMETSGI |
| | | EQMDVTMSGEASAPLPIRQPNSGPYKKQAFPMISKRP |
| | | EHLRMNL |

What is claimed:

1. A composition comprising a 70kDa ribosomal protein S6 kinase polypeptide 1 (p70S6K1) kinase domain-ligand complex in crystalline form wherein the crystal has a crystal lattice in a P2₁2₁2₁ space group and has unit cell dimensions of ±5% of a=56.6 Å, b=63.0 Å, and c=98.3 Å or P2₁ space group and has unit cell dimensions of ±5% of a=78.6 Å, b=62.9 Å, c=87.0 Å, and β=94.3 Å.

2. The composition of claim 1 wherein the ligand is an inhibitor of the p70S6K1 kinase activity.

3. The composition of claim 1 wherein the inhibitor inhibits binding of ATP to the p70S6K1 kinase domain.

4. The composition of claim 1 wherein the ligand is staurosporine.

5. The composition of claim 1 wherein the p70SK1 kinase domain comprises an amino acid sequence between amino acid 90 to amino acid 360 of SEQ ID NO:7.

6. The composition of claim 1 wherein the p70SK1 kinase domain comprises one or more amino acid substitutions, insertions, or deletions.

7. A method for identifying a compound that associates with at least a portion of 70kDa ribosomal protein S6 kinase polypeptide 1 (p70S6K1) kinase domain and inhibits kinase activity of the p70S6K1, the method comprising the steps of:
(a) obtaining a crystallized complex of p70S6K1 kinase domain complexed with a known inhibitor of the p70S6K1 kinase activity, the crystal belonging to a space group P2₁2₁2₁ and has unit cell dimensions of ±5% of a=56.6 Å, b=63.0 Å, and c=98.3 Å or a P2₁ space group and has unit cell dimensions of ±5% of a=78.6 Å, b=62.9 Å, c=87.0 Å, and β=94.3 Å;
(b) obtaining the structural coordinates of the crystallized complex of step (a);
(c) generating a three dimensional model of the p70S6K1 kinase domain using the structural coordinates of the amino acids generated in step (b), ±. a root mean square deviation from the backbone atoms of said amino acids of not more than 1.5 angstrom;
(d) determining an active site of the p70S6K1 kinase domain from the three dimensional model;
(e) performing computer modeling analysis to identify said compound that associates with the p70S6K1 kinase domain; and
(f) synthesizing the compound and contacting the compound with the p70S6K1 kinase to determine the ability of the compound to inhibit the kinase activity of the p70S6K1.

8. The method of claim 7 wherein the step of performing computer modeling analysis to identify said compound that associates with p70S6K1 kinase domain comprises identifying said compound from a library of compounds.

9. The method of claim 7 wherein the step of performing computer modeling analysis to identify said compound that associates with the p70S6K1 kinase domain comprises identifying said compound in a database.

10. The method of claim 7 wherein the step of performing computer modeling analysis to identify said compound that associates with the p70S6K1 kinase domain comprises designing the compound from a known p70S6K1 antagonist or partial antagonist.

11. The method of claim 7 wherein the known inhibitor is staurosporine.

12. The method of claim 7 wherein the p70SK1 kinase domain comprises an amino acid sequence between amino acid 90 to amino acid 360 of SEQ ID NO:7.

13. The method of claim 7 wherein the p70SK1 kinase domain comprises one or more amino acid substitutions, insertions, or deletions.

14. A method for making a crystal of the 70kDa ribosomal protein S6 kinase polypeptide 1 (p70S6K1) kinase domain, comprising:
(a) providing a solution of the p70S6K1 kinase domain polypeptide and a known inhibitor of the kinase activity of the p70S6K1 kinase domain;
(b) mixing the solution with a crystallization solution comprising polyethylene glycol, a buffer, and Li₂SO₄; and
(c) incubating the mixture under conditions for promoter vapor diffusion for a time sufficient to produce the crystal of the p70S6K1 kinase domain wherein the crystal belongs to a space group P2₁2₁2₁ and has unit cell dimensions of ±5% of a=56.6 Å, b=63.0 Å, and c=98.3 Å or a P2₁ space group and has unit cell dimensions of ±5% of a=78.6 Å, b=62.9 Å, c=87.0 Å, and β=94.3 Å.

15. The method of claim 14 wherein the pH of the crystallization solution is between 4 and 7 pH units.

16. The method claim 14 wherein crystallization solution comprises about 20-35% (w/v) PEG3350, about 100-200 mM Bis-Tris, pH 5-6, about 200-300 mM Li₂SO₄.

17. The method claim 14 wherein crystallization solution consists essentially of 20-35% (w/v) PEG3350, 100-200 mM Bis-Tris, pH 5-6, 200-300 mM Li₂SO₄.

18. The method of claim 14 wherein the p70SK1 kinase domain comprises an amino acid sequence between amino acid 90 to amino acid 360 of SEQ ID NO:7.

19. The method of claim 14 wherein the p70SK1 kinase domain comprises one or more amino acid substitutions, insertions, or deletions.


SEQUENCE LISTING (7 sequences)

SEQ ID NO: 1 - DNA, 1074 bp, Artificial Sequence
  Encodes p70S6K1 kinase domain polypeptide Variant 9 (v9) comprising amino acid residues 75-399 of p70S6K1 with N-terminal His6 and thrombin cleavage site
atgcatcaccatcaccatcacgaaaacctgtattttcagggcgaattatcaacaagtttgtacaaaaaagcaggcttcctggtgccgcgcggcagctcagaaactagtgtgaacagagggccagaaaaaatcagaccagaatgttttgagctacttcgggtacttggtaaagggggctatggaaaggtttttcaagtacgaaaagtaacaggagcaaatactgggaaaatatttgccatgaaggtgcttaaaaaggcaatgatagtaagaaatgctaaagatacagctcatacaaaagcagaacggaatattctggaggaagtaaagcatcccttcatcgtggatttaatttatgcctttcagactggtggaaaactctacctcatccttgagtatctcagtggaggagaactatttatgcagttagaaagagagggaatatttatggaagacactgcctgcttttacttggcagaaatctccatggctttggggcatttacatcaaaaggggatcatctacagagacctgaagccggagaatatcatgcttaatcaccaaggtcatgtgaaactaacagactttggactatgcaaagaatctattcatgatggaacagtcacacacacattttgtggaacaatagaatacatggcccctgaaatcttgatgagaagtggccacaatcgtgctgtggattggtggagtttgggagcattaatgtatgacatgctgactggagcacccccattcactggggagaatagaaagaaaacaattgacaaaatcctcaaatgtaaactcaatttgcctccctacctcacacaagaagccagagatctgcttaaaaagctgctgaaaagaaatgctgcttctcgtctgggagctggtcctggggacgctggagaagttcaagctcatccattctttagacacattaactgggaagaacttctggctcgaaaggtggagcccccctttaaacctctgttgcaatctgaagaggatgtaagtcagtttgattccaagtttacacgtcagacacctgtcgacagcccagatgactcaacttag

SEQ ID NO: 2 - protein, 357 aa, Artificial Sequence
  Kinase domain with N-terminal His6 and thrombin cleavage site
MHHHHHHENLYFQGELSTSLYKKAGFLVPRGSSETSVNRGPEKIRPECFELLRVLGKGGYGKVFQVRKVTGANTGKIFAMKVLKKAMIVRNAKDTAHTKAERNILEEVKHPFIVDLIYAFQTGGKLYLILEYLSGGELFMQLEREGIFMEDTACFYLAEISMALGHLHQKGIIYRDLKPENIMLNHQGHVKLTDFGLCKESIHDGTVTHTFCGTIEYMAPEILMRSGHNRAVDWWSLGALMYDMLTGAPPFTGENRKKTIDKILKCKLNLPPYLTQEARDLLKKLLKRNAASRLGAGPGDAGEVQAHPFFRHINWEELLARKVEPPFKPLLQSEEDVSQFDSKFTRQTPVDSPDDST

SEQ ID NO: 3 - protein, 327 aa, Artificial Sequence
  Kinase domain after cleavage with thrombin
GSSETSVNRGPEKIRPECFELLRVLGKGGYGKVFQVRKVTGANTGKIFAMKVLKKAMIVRNAKDTAHTKAERNILEEVKHPFIVDLIYAFQTGGKLYLILEYLSGGELFMQLEREGIFMEDTACFYLAEISMALGHLHQKGIIYRDLKPENIMLNHQGHVKLTDFGLCKESIHDGTVTHTFCGTIEYMAPEILMRSGHNRAVDWWSLGALMYDMLTGAPPFTGENRKKTIDKILKCKLNLPPYLTQEARDLLKKLLKRNAASRLGAGPGDAGEVQAHPFFRHINWEELLARKVEPPFKPLLQSEEDVSQFDSKFTRQTPVDSPDDST

SEQ ID NO: 4 - DNA, 71 bp, Artificial Sequence
  v9 5′ PCR primer
ggggacaagtttgtacaaaaaagcaggcttcctggtgccgcgcggcagctcagaaactagtgtgaacagag

SEQ ID NO: 5 - DNA, 55 bp, Artificial Sequence
  v9 3′ PCR Primer
ggggaccactttgtacaagaaagctgggtcctaagttgagtcatctgggctgtcg

SEQ ID NO: 6 - protein, 10 aa, Artificial Sequence
  Kinase substrate
KKRNRTLSVA

SEQ ID NO: 7 - protein, 525 aa, Homo sapiens
  DOMAIN (75)...(399): Kinase domain
MRRRRRRDGFYPAPDFRDREAEDMAGVFDIDLDQPEDAGSEDELEEGGQLNESMDHGGVGPYELGMEHCEKFEISETSVNRGPEKIRPECFELLRVLGKGGYGKVFQVRKVTGANTGKIFAMKVLKKAMIVRNAKDTAHTKAERNILEEVKHPFIVDLIYAFQTGGKLYLILEYLSGGELFMQLEREGIFMEDTACFYLAEISMALGHLHQKGIIYRDLKPENIMLNHQGHVKLTDFGLCKESIHDGTVTHTFCGTIEYMAPEILMRSGHNRAVDWWSLGALMYDMLTGAPPFTGENRKKTIDKILKCKLNLPPYLTQEARDLLKKLLKRNAASRLGAGPGDAGEVQAHPFFRHINWEELLARKVEPPFKPLLQSEEDVSQFDSKFTRQTPVDSPDDSTLSESANQVFLGFTYVAPSVLESVKEKFSFEPKIRSPRRFIGSPRTPVSPVKFSPGDFWGRGASASTANPQTPVEYPMETSGIEQMDVTMSGEASAPLPIRQPNSGPYKKQAFPMISKRPEHLRMNL